(19) United States
(12) Patent Application Publication
(10) Pub. No.: US 2025/0129031 A1
(43) Pub. Date: Apr. 24, 2025

(54) 1,2,3-TRIAZOLE CARBONYL SULFONYLAMIDE COMPOUNDS AND USE THEREOF

(71) Applicant: PI INDUSTRIES LTD., Udaipur -Rajasthan (IN)

(72) Inventors: Robindro Singh SARANGTHEM, Udaipur, Rajasthan (IN); Vishal A. MAHAJAN, Udaipur, Rajasthan (IN); Amresh BAITHA, Udaipur, Rajasthan (IN); Amol Dnyaneshwar KALWAGHE, Udaipur, Rajasthan (IN); Kiran MORYA, Udaipur, Rajasthan (IN); Jagadish PABBA, Udaipur, Rajasthan (IN); Alexander G.M. KLAUSENER, Pulheim (DE)

(21) Appl. No.: 18/833,125

(22) PCT Filed: Jan. 25, 2023
(86) PCT No.: PCT/IB2023/050618

§ 371 Date: Jul. 25, 2024

(30) Foreign Application Priority Data
Jan. 27, 2022 (IN) 202211004549

(51) Int. Cl. C07D 249/04; A01N 43/647; A01P 5/00

(52) U.S. Cl. CPC C07D 249/04; A01N 43/647; A01P 5/00

(57) ABSTRACT

The present invention provides novel 1,2,3-triazole carbonyl sulfonylamide compounds of formula (I),

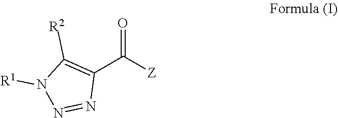

wherein,
R¹, R² and Z are as defined in the detailed description. The present invention further relates to their preparation and their use to protect crops against undesired pests such as nematodes.

### FIELD OF THE INVENTION

The present invention relates to novel nematicidal compounds of formula (I). More particularly, the present invention relates to novel 1,2,3-triazole carbonyl sulfonylamide compounds of formula (I) and a process for the preparation thereof. The present invention further relates to a composition and a combination comprising such novel 1,2,3-triazole carbonyl sulfonylamide compounds, and to their use as crop protection agents for the control of pests such as plant parasitic nematodes.

### BACKGROUND OF THE INVENTION

The control of damages to plants caused by phytopathogenic microorganisms and animal pests, in particular plant-parasitic nematodes, is extremely important to achieve high crop efficiency. Nematodes cause a substantial loss in agricultural food and industrial crops, and are therefore combated with chemical compounds having nematicidal activity. Even though many products are commercially available to control such damage, the need continues for new compounds which are more effective, less toxic, less costly, environmentally safer and/or have a different mode of action.

In the literature, sulfonylamides and their suitability as agrochemicals are described. For example, patent application EP0244166 discloses fused triazole acyl sulfonamides as herbicides and/or plant growth regulators.

Further prior art documents WO2010/129500, WO2012/054233 and WO2015/169776 describe fused imidazolopyridine acyl sulfonamide compounds that allow the controlling of harmful nematodes.

In addition to the above prior art documents, WO2017157735 and WO2018/083288 discloses disubstituted pyrazole/imidazole acyl sulfonylamides that can be used as nematicides.

Under agricultural conditions, the effectiveness of the substituted sulfonylamides, described in the prior art, is not fully satisfactory in various aspects such as nematicidal activity, application rates, treatment cost and toxicity. Therefore, it is always of high interest to find novel pesticidal compounds for the control of pests such as nematodes with better economical and environmental properties. The aim is to provide novel compounds being more active at lower application rates and environmentally safer whilst at the same time maintaining an effectiveness, long lasting activity and environmental and human health related compatibility, at least in an equivalent or if possible improved way compared to the already known compounds.

The present invention describes compounds of formula (I) which display the above-mentioned effects or advantages. Compounds of formula (I) are fulfilling this purpose, namely novel 1,2,3-triazole carbonyl sulfonylamide compounds with a substituted heterocyclic ring, by exhibiting surprisingly significant higher activity levels against undesired pests such as phytoparasitic nematodes.

### SUMMARY OF THE INVENTION

The present invention provides novel 1,2,3-triazole carbonyl sulfonylamide compounds of formula (I),

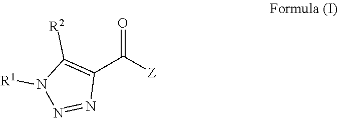

wherein, R¹, R² and Z are as defined in the detailed description.

In one embodiment, the present invention provides a process for preparing the compounds of formula (I).

In another embodiment, the present invention provides a composition for controlling or preventing phytoparasitic organisms such as nematodes comprising a biologically effective amount of a compound of formula (I) or salts, stereoisomers, polymorphs or N-oxides thereof and at least one additional component selected from the group consisting of surfactants and auxiliaries.

In yet another embodiment, the composition further comprises at least one additional biologically active and compatible compound selected from fungicides, insecticides, nematicides, acaricides, biopesticides, herbicides, plant growth regulators, antibiotics, fertilizers or nutrients.

In still another embodiment, the present invention provides the use of the compounds of formula (I) or salts, stereoisomers, polymorphs or N-oxides, compositions or combinations thereof, for the control of phytoparasitic organisms such as nematodes.

In yet still another embodiment, the present invention provides a method of combating phytoparasitic organisms such as nematodes with a biologically effective amount of the compounds of formula (I) or salts, stereoisomers, polymorphs or N-oxides thereof as well as compositions or combinations thereof.

### DETAILED DESCRIPTION OF THE INVENTION

### Definitions

The terminologies used in the present disclosure are for illustrative purposes only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “pesticide” in each case also always comprises the term “crop protection agent”.

The term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora.

The term “nematode” refers to a living organism of the Phylum Nematoda. The term “helminths” includes roundworms, heartworms, phytophagous nematodes (Nematoda), flukes (Trematoda), acanthocephala and tapeworms (Cestoda).

In the context ofthis disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from an invertebrate parasitic pest by administering a parasiticidally effective (i.e. biologically effective) amount of a compound of the present invention, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility, or lessened ability to remain on or in the host animal, reduced feeding and inhibition of reproduction. These effects on invertebrate parasite pests provide control (including prevention, reduction, or elimination) of a parasitic infestation or infection of the animal.

The term “stereoisomer” refers to isomers of the same configuration that differ in the arrangement of atoms in space. Enantiomers and diastereomers are examples of stereoisomers.

The term “enantiomer” refers to a pair of molecular species that are mirror images of each other and are not superimposed. The term “diastereomer” refers to a stereoisomer that is not a mirror image. The term “racemate” or “racemic mixture” refers to a composition consisting of an equimolar amount of two enantiomeric species, wherein the composition is not optically active.

The compounds of the present disclosure may be present either in pure form or as mixtures of different possible isomeric forms such as stereoisomers or constitutional isomers. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technology to separate, enrich, and/or to selectively prepare said isomers.

The meaning of various terms used in the description shall now be illustrated:

In the above description, the term “alkyl”, used either alone or in compound words such as “alkylthio” or “haloalkyl” or —N(alkyl) or alkylcarbonylalkyl or alkylsuphonylamino includes straight-chain or branched C₁ to C₁₂ alkyl, preferably C₁ to C₈ alkyl, more preferably C₁ to C₆ alkyl. Non limiting examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxyl, halogen, carbonyl, carbonyloxy and the like, are at the end.

The term “alkenyl”, used either alone or in compound words includes straight-chain or branched C₂ to C₁₂ alkenes, preferably C₂ to C₁₈ alkenes, more preferably C₂ to C₆ alkenes. Non limiting examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. The term “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

The term “alkynyl”, used either alone or in compound words includes branched or straight-chain C₂ to C₁₂ alkynes, preferably C₂ to C₁₈ alkynes, more preferably C₂ to C₆ alkynes. Non limiting examples of alkynes include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. The term “Alkynyl” can also include moieties comprised of multiple triple bonds such as 2,5-hexadiynyl.

The term “cyclic alkyl” or “cycloalkyl” means alkyl closed to form a ring. Non limiting examples include but are not limited to cyclopropyl, cyclobutyl, cyclopentyl and cyclohexyl.

This definition also applies to cycloalkyl as a part of a composite substituent, for example cycloalkylalkyl etc., unless specifically defined elsewhere.

The term “cycloalkylcycloalkyl” denotes cycloalkyl substitution on another cycloalkyl ring, wherein each cycloalkyl ring independently has from 3 to 7 carbon atom ring members.

Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

The term “alkoxy” used either alone or in compound words included C₁ to C₁₂ alkoxy, more preferably C₁ to C₁₈ alkoxy, most preferably C₁ to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkynylalkoxy, etc., unless specifically defined elsewhere.

The term “cycloalkoxy”, is also defined analogously. Non limiting examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

Non limiting examples of “alkylsulfinyl” include but are not limited to methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, hexylsulfinyl, 1,1-dimethylpropylsulfinyl, 1,2-dimethylpropylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl and the different isomers. The term “arylsulfinyl” includes Ar—S(O), wherein Ar can be any carbocycle or heterocycle. This definition also applies to alkylsulfinyl as a part of a composite substituent, for example haloalkylsulfinyl etc., unless specifically defined elsewhere.

Non limiting examples of “alkylsulfonyl” include but are not limited to methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 2,2-dimethylpropylsulfonyl, 1-ethylpropylsulfonyl, hexylsulfonyl, 1,1-dimethylpropylsulfonyl, 1,2-dimethylpropylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl and the different isomers.

The term “arylsulfonyl” includes Ar—S(O)₂, wherein Ar can be any carbocycle or heterocycle.

This definition also applies to alkylsulfonyl as a part of a composite substituent, for example alkylsulfonylalkyl etc., unless defined elsewhere.

The term “halogen”, either alone or in compound words such as “haloalkyl”, includes fluorine, chlorine, bromine, or iodine. Further, when used in compound words such as “haloalkyl”, said alkyl may be partially or fully substituted with halogen atoms which may be the same or different.

Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl” and “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The terms “haloalkylthio” or “haloalkylsulfanyl” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Non limiting examples of “haloalkylsulfinyl” include CF₃S(O), CCl₃S(O), CF₃CH₂S(O) and CF₃CF₂S(O). Non limiting examples of “haloalkylsulfonyl” include CF₃S(O)₂, CCl₃S(O)₂, CF₃CH₂S(O)₂ and CF₃CF₂S(O)₂.

The term “ring” or “ring system” as a component of formula (I) is carbocyclyl or heterocyclyl.

The term “ring system” denotes one or more rings.

The term “bicyclic ring or ring system” denotes a ring system consisting of two or more common atom.

The term “aromatic” indicates that the Hueckel rule is satisfied, and the term “non-aromatic” indicates that the Hueckel rule is not satisfied.

The terms “carbocycle” or “carbocyclic” or “carbocyclyl” include an “aromatic carbocyclic ring system” and a “nonaromatic carbocylic ring system” or polycyclic or bicyclic (spiro, fused, bridged, nonfused) ring compounds in which the ring may be aromatic or non-aromatic (where aromatic indicates that the Huckel rule is satisfied, and non-aromatic indicates that the Huckel rule is not satisfied).

Non limiting examples of a non-aromatic carbocyclic ring system are cyclopropyl, cyclobutyl, cyclopentyl, norbornyl and the like.

Non limiting examples of an aromatic carbocyclic ring system are phenyl, naphthyl and the like.

The term “aryl” as used herein is a group that contains any carbon-based aromatic group including, but not limited to phenyl, naphthalene, biphenyl and the like; preferably phenyl. The aryl group can be substituted or unsubstituted. In addition, the aryl group can be a single ring structure or comprise multiple ring structures that are either fused ring structures or attached via one or more bridging groups such as a carbon-carbon bond.

The term “aralkyl” refers to aryl hydrocarbon radicals including an alkyl portion as defined above. Examples include benzyl, phenylethyl, and 6-naphthylhexyl. As used herein, the term “aralkenyl” refers to aryl hydrocarbon radicals including an alkenyl portion, as defined above, and an aryl portion, as defined above. Examples include styryl, 3-(benzyl) prop-2-enyl, and 6-naphthylhex-2-enyl.

The term “hetero” in connection with rings refers to a ring in which at least one ring atom is not carbon and which can contain 1 to 4 heteroatoms independently selected from the group consisting of nitrogen, oxygen and sulfur, provided that each ring contains no more than 4 nitrogens, no more than 2 oxygens and no more than 2 sulfurs.

The terms “heterocycle” or “heterocyclic” includes an “aromatic heterocycle” or a “heteroaryl ring system” and a “nonaromatic heterocycle ring system” or polycyclic or bicyclic (spiro, fused, bridged, non-fused) ring compounds in which the ring may be aromatic or non-aromatic, wherein the heterocycle ring contains at least one heteroatom selected from N, O, S(O)₀₋₂, and or a C ring member of the heterocycle may be replaced by C(═O), C(═S), C(═CR*R*) and C═NR*, * indicates integers.

The terms “non-aromatic heterocycle” or “non-aromatic heterocyclic” means three- to fifteen-membered, preferably three- to twelve-membered, saturated or partially unsaturated heterocycles containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; for example (but not limited to) oxiranyl, aziridinyl, oxetanyl, azetidinyl, thietanyl, 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-1-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, pyrrolinyl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, piperidinyl, 2-piperidinyl, 3-piperidinyl, 4-piperidinyl, pyrazynyl, morpholinyl, thiomorphlinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, cycloserines. This definition also applies to heterocyclyl as a part of a composite substituent, for example heterocyclylalkyl etc., unless specifically defined elsewhere.

The term “heteroaryl” means 5 or 6-membered, fully unsaturated monocyclic ring systems containing one to four heteroatoms from the group of oxygen, nitrogen and sulphur; if the ring contains more than one oxygen atom, they are not directly adjacent; 5-membered heteroaryl containing one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example (but not limited thereto) furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,2,3-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited to) 1-pyrrolyl, 1-pyrazolyl, 1,2,4-triazolyl, 1-imidazolyl, 1,2,3-triazolyl and 1,3,4-triazolyl. 6-membered heteroaryl which contains one to four nitrogen atoms: 6-membered heteroaryl groups which, in addition to carbon atoms, may contain, respectively, one to three and one to four nitrogen atoms as ring members, for example (but not limited thereto) 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl; This definition also applies to heteroaryl as a part of a composite substituent, for example heteroarylalkyl etc., unless specifically defined elsewhere.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: TL Gilchrist in Comprehensive Organic Synthesis, vol. 7, pp 748-750.

The term “amide” means A-R′C═ONR″—B, wherein R′ and R″ indicates substituents and A and B indicate any group.

The term “thioamide” means A-R′C═SNR″—B, wherein R′ and R″ indicates substituents and A and B indicate any group.

The term “leaving group (LG)” means, all substituents which have sufficient nucleofilicity under the prevailing reaction conditions or a nucleophilically replaceable group; by way of example, halogens (chloro, iodo, bromo), triflate, nosylate, mesylate, tosylate or SCh-Me may be mentioned as suitable leaving groups

Examples for illustrating “Cᵢ-Cⱼ-cycloalkyl-Cᵢ-Cⱼ-alkyl” includes but are not limited to cyclopropyl-methyl, cyclopropyl-ethyl, cyclobutyl-methyl, cyclobutyl-ethyl, cyclopentyl-methyl and cyclopentyl-ethyl.

The total number of carbon atoms in a substituent group is indicated by the “Cᵢ-Cⱼ” prefix where i and j are numbers from 1 to 21. For example, C₁-C₄ alkoxy designates methoxy through butyloxy. In the above recitations, when a compound of formula (I) is comprised of one or more heterocyclic rings, all substituents are attached to these rings through any available carbon or nitrogen by replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript indicates a range, e. g. (R)ᵢ₋ⱼ, then the number of substituents may be selected from the integers between i and j inclusive.

When a group contains a substituent which can be hydrogen, for example R¹ or R², then, when this substituent is taken as hydrogen, it is recognized that this is equivalent to said group being unsubstituted.

Accordingly, the present invention provides a compound of formula (I),

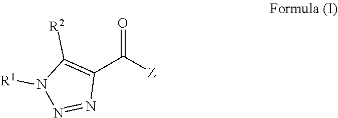

wherein,
R is

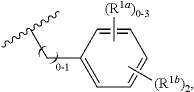

wherein [symbol] represents the point of attachment to the triazole ring;
   or
R¹ is selected from the group consisting of C₁-C₆-haloalkyl, C₂-C₆-alkenyl, C₂-C₆-alkynyl, C₃-C₈-cycloalkyl, C₃-C₈-halocycloalkyl and C₃-C₈-cycloalkyl-C₁-C₆-alkyl; wherein said C₁-C₆-haloalkyl, C₂-C₆-alkenyl, C₂-C₆-alkynyl, C₃-C₈-cycloalkyl, C₃-C₈-halocycloalkyl and C₃-C₈-cycloalkyl-C₁-C₆-alkyl groups of R¹ may optionally be substituted by one or more groups selected from the group consisting of halogen, CN, R^{6a}, OR^{6a}, S(O)ₙR^{6a}, N(R^{6a})₂ and COOR^{6a};
R^{1a} is selected from the group consisting of hydrogen, halogen, CN, OR^{6a}, C₁-C₆-alkyl and C₁-C₆-haloalkyl; wherein each group of R^{1a} may optionally be substituted by one or more groups selected from the group consisting of halogen, CN, R^{6a}, OR^{6a}, S(O)ₙR^{6a}, N(R^{6a})₂, COOR^{6a} and CONR^{6a};
R^{1b} is selected from the group consisting of halogen, CN, C₁-C₆-alkyl, C₁-C₆-haloalkyl, C₂-C₆-alkenyl, C₂-C₆-alkynyl, C₁-C₆-alkoxy, C₁-C₆-haloalkoxy, C₃-C₈-cycloalkyl, C₃-C₈-halocycloalkyl, S(O)ₙR^{6a} and C₃-C₈-cycloalkyl-C₁-C₆-alkyl; wherein each group of R^{1b} may optionally be substituted by one or more groups selected from the group consisting of halogen, R^{6a}, OR^{6a}, S(O)ₙR^{6a}, N(R^{6a})₂, COOR^{6a} and CONR^{6a};
R² is selected from the group consisting of hydrogen, halogen, CN, C₁-C₆-alkyl, C₁-C₆-haloalkyl, C₁-C₆-alkoxy, C₃-C₆-cycloalkyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
Z is Z-1 or Z-2;

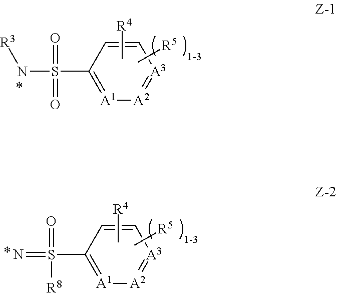

* represents the point of attachment to the carbonyl group attached to the triazole ring;
R³ is selected from the group consisting of hydrogen, C₁-C₆-alkyl, C₁-C₆-haloalkyl, C₁-C₆-alkoxy, C₃-C₆-cycloalkyl and C₃-C₈-cycloalkyl-C₁-C₆-alkyl; wherein each group of R³ may optionally be substituted with halogen;
A¹, A² and A³ represent C or N; wherein not more than two of A¹, A² and A³ represent N;
R⁴ is selected from the group consisting of hydrogen, halogen, CN, OR⁶, C₁-C₆-alkyl, C₁-C₆-haloalkyl, C₂-C₆-alkenyl, and C₂-C₆-alkynyl;
R⁵ is selected from the group consisting of hydrogen, halogen, CN, nitro, (C═O)—R⁶, O(C═O)—R⁶, OR⁶, N(R⁶)₂, CR⁶═NR⁶, COOR⁶, CON(R⁶)₂, (C═S)—N(R⁶)₂, S(O)ₙR⁶, OS(O)ₙR⁶, S(O)ₙN(R⁶)₂, —S(═O)₀₋₁R⁷(═N—R⁶), —N═S(═O)₀₋₁(R⁷)₂, C₁-C₆-alkyl, C₁-C₆-haloalkyl, C₂-C₆-alkenyl, C₂-C₆-alkynyl, C₃-C₈-cycloalkyl, C₃-C₈-halocycloalkyl, C₃-C₈-cycloalkyl-C₁-C₆-alkyl, phenyl, C₃-C₆-carbocyclyl and heterocyclic 3 to 6-membered ring; wherein each group of R⁵ may optionally be substituted by one or more groups selected from the group consisting of halogen, R^{6a}, OR^{6a}, S(O)ₙR^{6a}, N(R^{6a})₂, COOR^{6a} and CONR^{6a};
R⁶ is selected from the group consisting of hydrogen, hydroxy, C₁-C₆-alkyl, C₁-C₆-alkoxy, C₁-C₆-haloalkyl, C₂-C₆-alkenyl, C₂-C₆-alkynyl, C₃-C₈-cycloalkyl and C₃-C₈-cycloalkyl-C₁-C₆-alkyl;
R^{6a} is selected from the group consisting of hydrogen, C₁-C₆-alkyl, C₁-C₆-haloalkyl and C₃-C₈-cycloalkyl;
R⁷ and R⁸ are independently selected from the group consisting of hydrogen, C₁-C₆-alkyl, C₁-C₆-haloalkyl, C₂-C₆-alkenyl and C₃-C₈-cycloalkyl and C₃-C₈-cycloalkyl-C₁-C₆-alkyl; n is an integer selected from 0 to 2;
or salts, stereoisomers, polymorphs or N-oxides thereof; with the proviso that the following compounds are excluded from the definition of compounds of formula (I): 1H-1,2,3-Triazole-4-carboxamide, 1-(2-cyclopropylethyl)-N-[(2,3-difluorophenyl)sulfonyl], 1H-1,2,3-Triazole-4-carboxamide, N-[(4-bromo-2-chlorophenyl)sulfonyl]-1-(2-cyclopropylethyl), 1H-1,2,3-Triazole-4-carboxamide, 1-(2-cyclopropylethyl)-N-[[3-[(dimethylamino)carbonyl]phenyl]sulfonyl], 1H-1,2,3-Triazole-4-carboxamide, 1-cyclohexyl-N—[[3-(4-methyl-2-thiazolyl)phenyl]sulfonyl] and 1H-1,2,3-Triazole-4-carboxamide, 1-cyclopropyl-5-(difluoromethyl)-N-[[4-methyl-3-(trifluoromethyl) phenyl]sulfonyl].

In one embodiment, the present invention provides a compound of formula (Ia),

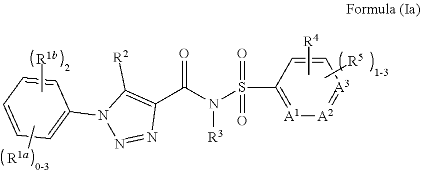

wherein, R^{1a}, R^{1b}, R², R³, R⁴, R⁵, A¹, A² and A³ are as defined above.

In another embodiment, the present invention provides a compound of formula (Ib),

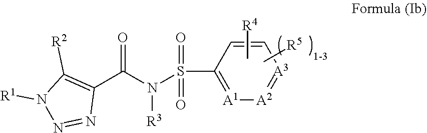

wherein, R¹ is selected from C₁-C₆-haloalkyl, C₂-C₆-alkenyl, C₂-C₆-alkynyl, C₃-C₈-cycloalkyl, C₃-C₈-halocycloalkyl and C₃-C₈-cycloalkyl-C₁-C₆-alkyl; wherein each group of R¹ may optionally be substituted with halogen or C₁-C₆-haloalkyl; preferably R¹ is C₁-C₆-haloalkyl, C₃-C₈-cycloalkyl and C₃-C₈-cycloalkyl-C₁-C₆-alkyl which may optionally be substituted with halogen or C₁-C₆-haloalkyl; R², R³, R⁴, R⁵, A¹, A² and A³ are as defined above.

In a preferred embodiment, A¹, A² and A³ represent carbon atoms which may optionally be substituted with one or more R⁵.

In a further preferred embodiment, the present invention provides a compound of formula (Ia-1),

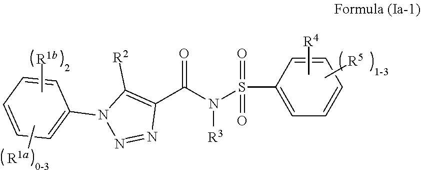

wherein, R^{1a}, R^{1b}, R², R³, R⁴ and R⁵ are as defined above.

In another preferred embodiment, the present invention provides a compound of formula (Ia-2),

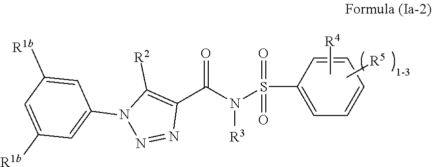

wherein, R^{1b}, R², R³, R⁴ and R⁵ are as defined above.

In another preferred embodiment, the present invention provides a compound of formula (Ia-3),

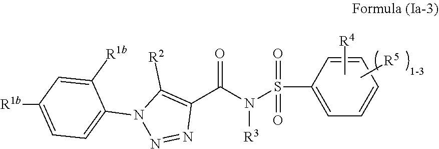

wherein, R^{1b}, R², R³, R⁴ and R⁵ are as defined above.

In another preferred embodiment, the present invention provides a compound of formula (Ib-1),

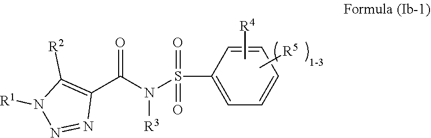

wherein, R¹, R², R³, R⁴ and R⁵ are as defined above.

In one embodiment, the present invention provides a compound of formula (Ic),

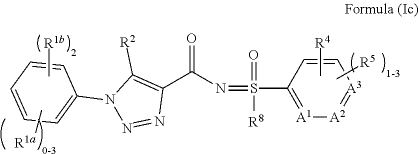

wherein, R^{1a}, R^{1b}, R², R⁴, R⁵, R⁸, A¹, A² and A³ are as defined above.

In another embodiment, the present invention provides a compound of formula (Id),

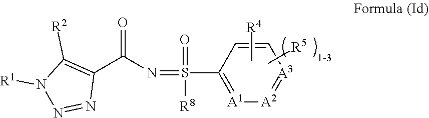

wherein, R¹, R², R⁴, R⁵, R⁸, A¹, A² and A³ are as defined above.

In one preferred embodiment, A¹, A² and A³ represent carbon atoms which may optionally be substituted with one or more R⁵.

In another preferred embodiment, the present invention provides a compound of formula (Ic-1),

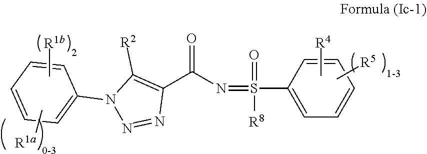

wherein, R^{1a}, R^{1b}, R², R⁴, R⁵ and R⁸ are as defined above.

In yet another preferred embodiment, the present invention provides a compound of formula (Ic-2),

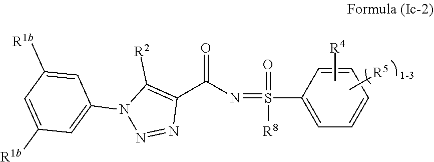

wherein, R^{1b}, R², R⁴, R⁵ and R⁸ are as defined above.

In yet another preferred embodiment, the present invention provides a compound of formula (Ic-3),

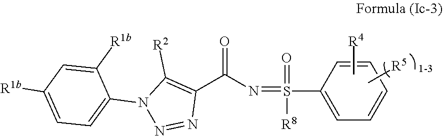

wherein, R^{1b}, R², R⁴, R⁵ and R⁸ are as defined above.

In still another preferred embodiment, the present invention provides a compound of formula (Id-1),

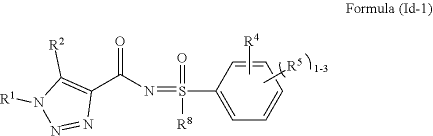

wherein, R¹, R², R⁴, R⁵ and R⁸ are as defined above.

The following list provides definitions, including preferred definitions, for substituents R¹, Ria R^{1b}, R², Z, A¹, A², A³, R³, R⁴, R⁵, R⁶, R^{6a}, R⁷ and R⁸ with reference to the compounds of formula (I) of the present invention. For any one of these substituents, any of the definitions given below may be combined with any definition of any other substituent given below or elsewhere in this document.

In one embodiment of the present invention, R¹ is selected from the group consisting of C₁-C₄-haloalkyl, C₂-C₄-alkenyl, C₂-C₄-alkynyl, C₃-C₆-cycloalkyl, C₃-C₆-halocycloalkyl and C₃-C₆-cycloalkyl-C₁-C₄-alkyl; wherein each group of R¹ may optionally be substituted by one or more groups selected from the group consisting of halogen, CN, R^{6a}, OR^{6a}, S(O)ₙR^{6a}, N(R^{6a})₂ and COOR^{6a}.

In another embodiment of the present invention, R¹ is selected from the group consisting of C₁-C₄-haloalkyl, C₂-C₄-alkenyl, C₂-C₄-alkynyl, C₃-C₆-cycloalkyl, C₃-C₆-halocycloalkyl and C₃-C₆-cycloalkyl-C₁-C₄-alkyl; wherein each group of R¹ may optionally be substituted by one or more groups selected from the group consisting of halogen, CN and R^{6a}.

In yet another embodiment of the present invention, R¹ is selected from the group consisting of C₁-C₄-haloalkyl, C₂-C₄-alkenyl, C₂-C₄-alkynyl, C₃-C₆-cycloalkyl, C₃-C₆-halocycloalkyl and C₃-C₆-cycloalkyl-C₁-C₄-alkyl; wherein each group of R¹ may optionally be substituted by one or more groups selected from the group consisting of halogen, CN and R^{6a}.

In yet another embodiment of the present invention, R¹ is selected from the group consisting of C₁-C₄-haloalkyl, C₂-C₄-alkenyl, C₂-C₄-alkynyl, C₃-C₆-cycloalkyl, C₃-C₆-halocycloalkyl and C₃-C₆-cycloalkyl-C₁-C₄-alkyl.

In one embodiment of the present invention, R¹ is:

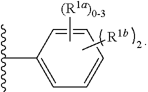

In a preferred embodiment of the present invention, R¹ is:

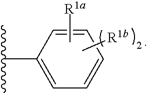

In a preferred embodiment of the present invention, R¹ is:

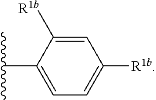

In another preferred embodiment of the present invention, R¹ is:

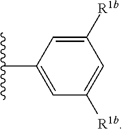

In one embodiment of the present invention, R^{1a} is selected from the group consisting of hydrogen, halogen, CN, OR^{6a}, C₁-C₄-alkyl and C₁-C₄-haloalkyl; wherein each group of R^{1a} may optionally be substituted by one or more groups selected from the group consisting of halogen, CN, R^{6a}, OR^{6a}, S(O)ₙR^{6a}, N(R^{6a})₂, COOR^{6a} and CONR^{6a}.

In another embodiment of the present invention, R^{1a} is selected from the group consisting of hydrogen, halogen, CN, OR^{6a}, C₁-C₄-alkyl and C₁-C₄-haloalkyl; wherein each group of R^{1a} may optionally be substituted by one or more groups selected from the group consisting of halogen, CN and R^{6a}.

In a preferred embodiment of the present invention, R^{1a} is selected from the group consisting of hydrogen, halogen, CN, methoxy, ethoxy, propoxy, butyloxy, cyclopropyloxy, cyclobutyloxy, cyclopentyloxy, fluoromethoxy, difluoromethoxy, difluoroethoxy, fluoroethoxy, trifluoromethoxy, trifluoroethoxy, methyl, ethyl, n-propyl, isopropyl, n-butyl, isobutyl, sec-butyl, tert-butyl, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl, 2-chloroethyl and pentafluoroethyl.

In one embodiment of the present invention, R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl, S(O)ₐR^{6a} and C₃-C₆-cycloalkyl-C₁-C₄-alkyl; wherein each group of R^{1b} may optionally be substituted by one or more groups selected from the group consisting of halogen, R^{6a}, OR^{6a}, S(O)ₙR^{6a}, N(R^{6a})₂, COOR^{6a} and CONR^{6a}.

In a preferred embodiment of the present invention, R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a}.

In a more preferred embodiment of the present invention, R^{1b} is selected from the group consisting of halogen, CN, methyl, ethyl, n-propyl, isopropyl, n-butyl, isobutyl, sec-butyl, tert-butyl, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl, 2-chloroethyl, pentafluoroethyl, methoxy, ethoxy, propoxy, butyloxy, fluoromethoxy, difluoromethoxy, difluoroethoxy, fluoroethoxy, trifluoromethoxy, trifluoroethoxy, cyclopropyl, cyclobutyl, cyclopentyl, cyclopropyl methyl, cyclopropyl ethyl, cyclobutyl methyl, cyclobutyl ethyl and cyclopentyl methyl.

In one embodiment of the present invention, R^{6a} is selected from the group consisting of hydrogen, C₁-C₄-alkyl, C₁-C₄-haloalkyl and C₃-C₆-cycloalkyl.

In another embodiment of the present invention, R^{6a} is selected from the group consisting of hydrogen, methyl, ethyl, n-propyl, isopropyl, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl, 2-chloroethyl, pentafluoroethyl, cyclopropyl, cyclobutyl and cyclopentyl.

In one embodiment of the present invention, OR^{6a} is selected from the group consisting of hydroxy, C₁-C₄-alkoxy, C₁-C₄-haloalkoxy and C₃-C₆-cycloalkyloxy.

In a preferred embodiment of the present invention, OR^{6a} is selected from the group consisting of hydroxy, methoxy, ethoxy, propoxy, butyloxy, allyloxy, propargyloxy, cyclopropyloxy, cyclobutyloxy, cyclopentyloxy, cyclopropylmethoxy, cyclopropylethoxy, cyclobutylmethoxy, fluoromethoxy, difluoromethoxy, fluoroethoxy, trifluoromethoxy and trifluoroethoxy.

In one embodiment of the present invention, —S(O)ₙR^{6a} is selected from the group consisting of —SR^{6a}, —S(O)R^{6a} and —S(O)₂R^{6a}.

In another embodiment of the present invention, —SR^{6a} is selected from the group consisting of methylthio, ethylthio, propylthio, isopropylthio, n-butylthio, isobutylthio, sec-butylthio and tert-butylthio, preferably methylthio or ethylthio.

In yet another embodiment of the present invention, —S(O)R^{6a} is selected from the group consisting of methylsulfinyl, ethylsulfinyl, propylsulfinyl, isopropylsulfinyl, n-butylsulfinyl, isobutylsulfinyl, sec-butylsulfinyl and tert-butylsulfinyl, preferably methylsulfinyl or ethylsulfinyl.

In yet another embodiment of the present invention, —S(O)₂R^{6a} is selected from the group consisting of methylsulfonyl, ethylsulfonyl, propylsulfonyl, isopropylsulfonyl, n-butylsulfonyl, isobutylsulfonyl, sec-butylsulfonyl and tert-butylsulfonyl, preferably methylsulfonyl or ethylsulfonyl.

In one embodiment of the present invention, —N(R^{6a})₂ is selected from the group consisting of amino, dimethylamino, diethylamino, diisopropylamino and dipropylamino.

In one embodiment of the present invention, —COOR^{6a} is selected from the group consisting of —COOH, —COOMe, —COOEt, —COOPr and —COOiPr.

In one embodiment of the present invention, —CONR^{6a} is selected from the group consisting of —CONH₂, —CONHMe, —CON(Me)₂, —CONHEt and —CON(Et)₂.

In one embodiment of the present invention, R² is selected from the group consisting of hydrogen, halogen, CN, C₁-C₄-alkyl, C₁-C₄-haloalkyl, C₁-C₄-alkoxy, C₃-C₆-cycloalkyl and phenyl; wherein said phenyl ring may optionally be substituted by one or more groups selected from the group consisting of halogen, CN, methyl, ethyl, propyl, isopropyl, methoxy, ethoxy, difluoromethyl and trifluoromethyl.

In another embodiment of the present invention, R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, methyl, ethyl, propyl, isopropyl, methoxy, ethoxy, difluoromethyl and trifluoromethyl.

In a preferred embodiment of the present invention, R²is selected from the group consisting of hydrogen, halogen, CN, C₁-C₄-alkyl, C₁-C₄-haloalkyl, C₁-C₄-alkoxy, C₃-C₆-cycloalkyl and phenyl.

In another preferred embodiment of the present invention, R² is selected from the group consisting of hydrogen, methyl, ethyl, propyl, isopropyl, methoxy, ethoxy, cyclopropyl, fluoromethyl, difluoromethyl, trifluoromethyl, chloromethyl, dichloromethyl and trichloromethyl.

In a more preferred embodiment of the present invention, R² is selected from the group consisting of hydrogen, methyl, ethyl, cyclopropyl and trifluoromethyl.

In the most preferred embodiment of the present invention, R² is methyl.

In one embodiment of the present invention, Z is Z-1 or Z-2 wherein, A¹, A² and A³ represents C:

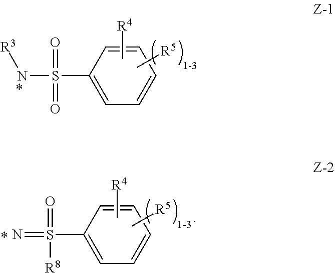

In another embodiment of the present invention, Z is Z-1 or Z-2 wherein, A¹, A² and A³ represents C, R³ represent H and R⁸ represent methyl:

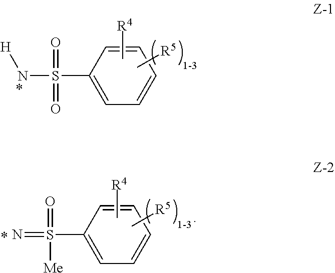

In a preferred embodiment of the present invention, Z is Z-1.

In one embodiment of the present invention, R³ is selected from the group consisting of hydrogen, C₁-C₄-alkyl, C₁-C₄-haloalkyl, C₁-C₄-alkoxy, C₃-C₆-cycloalkyl and C₃-C₈-cycloalkyl-C₁-C₄-alkyl; wherein each group of R³ may optionally be substituted with halogen.

In another embodiment of the present invention, R³ is selected from the group consisting of hydrogen, C₁-C₄-alkyl, C₁-C₄-haloalkyl and C₃-C₈-cycloalkyl-C₁-C₄-alkyl.

In a preferred embodiment of the present invention, R³ is selected from the group consisting of hydrogen, methyl, ethyl, cyclopropyl methyl, cyclopropyl ethyl.

In a more preferred embodiment of the present invention, R³ is hydrogen.

In one embodiment of the present invention, R⁴ is selected from the group consisting of hydrogen, halogen, CN, OR⁶, C₁-C₆-alkyl and C₁-C₆-haloalkyl.

In another embodiment of the present invention, R⁴ is selected from the group consisting of hydrogen, halogen, CN, OR⁶, C₁-C₄-alkyl and C₁-C₄-haloalkyl.

In a preferred embodiment of the present invention, R⁴ is selected from the group consisting of hydrogen, halogen, CN, methoxy, ethoxy, propoxy, butyloxy, cyclopropyloxy, cyclobutyloxy, cyclopentyloxy, fluoromethoxy, difluoromethoxy, difluoroethoxy, fluoroethoxy, trifluoromethoxy, trifluoroethoxy, methyl, ethyl, n-propyl, isopropyl, n-butyl, isobutyl, sec-butyl, tert-butyl, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl and 2-chloroethyl, pentafluoroethyl.

In a more preferred embodiment of the present invention, R⁴ is selected from the group consisting of hydrogen, halogen, CN, methoxy, ethoxy, propoxy, difluoromethoxy, difluoroethoxy, trifluoromethoxy, trifluoroethoxy, methyl, ethyl, n-propyl, isopropyl, difluoromethyl, trifluoromethyl and chloromethyl.

In one embodiment of the present invention, R⁵ is selected from the group consisting of hydrogen, halogen, CN, nitro, (C═O)—R⁶, OR⁶, N(R⁶)₂, CR⁶═NR⁶, COOR⁶, CON(R⁶)₂, S(O)ₙR⁶, S(O)ₐN(R⁶)₂, —S(═O)₀₋₁R⁷(═N—R⁶), —N═S(═O)₀₋₁(R⁷)₂, C₁-C₆-alkyl, C₁-C₆-haloalkyl, C₂-C₆-alkenyl, C₂-C₆-alkynyl, C₃-C₈-cycloalkyl, C₃-C₈-halocycloalkyl, C₃-C₈-cycloalkyl-C₁-C₆-alkyl, phenyl, C₃-C₆-carbocyclyl and a heterocyclic 3 to 6-membered ring; wherein each group of R⁵ may optionally be substituted by one or more groups selected from the group consisting of halogen, R^{6a}, OR^{6a}, S(O)ₙR^{6a}, N(R^{6a})₂, COOR^{6a} and CONR^{6a}.

In another embodiment of the present invention, R⁵ is selected from the group consisting of hydrogen, halogen, CN, nitro, (C═O)—R⁶, OR⁶, N(R⁶)₂, S(O)ₙR⁶, S(O)ₐN(R⁶)₂, —S(═O)₀₋₁R⁷(═N—R⁶), —N═S(═O)₀₋₁(R⁷)₂, C₁-C₄-alkyl, C₁-C₄-haloalkyl, C₂-C₄-alkenyl, C₂-C₄-alkynyl, C₃-C₆-cycloalkyl, C₃-C₆-halocycloalkyl, C₃-C₆-cycloalkyl-C₁-C₄-alkyl, phenyl, C₃-C₆-carbocyclyl and a heterocyclic 3 to 6-membered ring; wherein each group of R⁵ may optionally be substituted by one or more groups selected from the group consisting of halogen and R^{6a}.

In yet another embodiment of the present invention, R⁵ is selected from the group consisting of hydrogen, halogen, CN, nitro, OR⁶, C₁-C₄-alkyl, C₁-C₄-haloalkyl, C₃-C₆-cycloalkyl, C₃-C₆-cycloalkyl-C₁-C₄-alkyl and C₃-C₆-carbocyclyl; wherein each group of R⁵ may optionally be substituted by one or more groups selected from the group consisting of halogen and R^{6a}.

In a preferred embodiment of the present invention, R⁵ is selected from the group consisting of hydrogen, halogen, CN, OR⁶, C₁-C₄-alkyl and C₁-C₄-haloalkyl.

In a more preferred embodiment of the present invention, R⁵ is selected from the group consisting of hydrogen, halogen, CN, methoxy, ethoxy, propoxy, difluoromethoxy, difluoroethoxy, trifluoromethoxy, trifluoroethoxy, methyl, ethyl, n-propyl, isopropyl, difluoromethyl, trifluoromethyl and chloromethyl.

In one embodiment of the present invention, R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl, C₃-C₆-cycloalkyl and C₃-C₆-cycloalkyl-C₁-C₄-alkyl.

In another embodiment of the present invention, R⁶ is selected from the group consisting of hydrogen, hydroxy, methyl, ethyl, n-propyl, isopropyl, methoxy, ethoxy, propoxy, 1-methylethoxy, butoxy, 1-methylpropoxy, 2-methylpropoxy, 1,1-dimethylethoxy, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl, 2-chloroethyl, pentafluoroethyl, cyclopropyl, cyclobutyl, cyclopentyl, cyclopropyl methyl, cyclopropyl ethyl, cyclobutyl methyl and cyclobutyl ethyl.

In a preferred embodiment of the present invention, R⁶ is selected from the group consisting of hydrogen, hydroxy, methyl, ethyl, n-propyl, isopropyl, methoxy, ethoxy, propoxy, 1-methylethoxy, butoxy, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl, 2-chloroethyl, pentafluoroethyl, cyclopropyl, cyclobutyl and cyclopentyl.

In one embodiment of the present invention, OR⁶ is selected from hydroxy, C₁-C₆-alkoxy, C₁-C₆-haloalkoxy, C₃-C₆-cycloalkyloxy or —O—C₁-C₄-alkyl-C₃-C₆-cycloalkyl.

In another embodiment of the present invention, OR⁶ is selected from hydroxy, C₁-C₆-alkoxy or C₁-C₆-haloalkoxy.

In one embodiment of the present invention, R⁷ is selected from the group consisting of hydrogen, C₁-C₄-alkyl, C₁-C₄-haloalkyl, C₂-C₄-alkenyl and C₃-C₆-cycloalkyl and C₃-C₆-cycloalkyl-C₁-C₄-alkyl.

In another embodiment of the present invention, R⁷ is selected from the group consisting of hydrogen, methyl, ethyl, n-propyl, isopropyl, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl, 2-chloroethyl, pentafluoroethyl, cyclopropyl, cyclobutyl, cyclopentyl, cyclopropyl methyl, cyclopropyl ethyl, cyclobutyl methyl and cyclobutyl ethyl.

In a preferred embodiment of the present invention, R⁷ is selected from the group consisting of hydrogen, methyl, ethyl, n-propyl, isopropyl, fluoromethyl, difluoromethyl, trifluoromethyl, chloromethyl, dichloromethyl, cyclopropyl, cyclobutyl, cyclopentyl, cyclopropyl methyl, cyclopropyl ethyl, cyclobutyl methyl and cyclobutyl ethyl.

In one embodiment of the present invention, R⁸ is selected from the group consisting of hydrogen, C₁-C₄-alkyl, C₁-C₄-haloalkyl, C₂-C₄-alkenyl and C₃-C₆-cycloalkyl and C₃-C₆-cycloalkyl-C₁-C₄-alkyl.

In another embodiment of the present invention, R⁸ is selected from the group consisting of hydrogen, methyl, ethyl, n-propyl, isopropyl, fluoromethyl, difluoromethyl, trifluoromethyl, chloromethyl, dichloromethyl, trichloromethyl, 2,2,2-trifluoroethyl, 2,2-difluoroethyl, 1,1-difluoroethyl, 1,1,2,2-tetrafluoroethyl, 2-fluoroethyl, 2-chloroethyl, pentafluoroethyl, cyclopropyl, cyclobutyl, cyclopentyl, cyclopropyl methyl, cyclopropyl ethyl, cyclobutyl methyl and cyclobutyl ethyl.

In a preferred embodiment of the present invention, R⁸ is selected from the group consisting of hydrogen, methyl, ethyl, n-propyl, isopropyl, fluoromethyl, difluoromethyl, trifluoromethyl, chloromethyl, dichloromethyl, cyclopropyl, cyclobutyl, cyclopentyl, cyclopropyl methyl, cyclopropyl ethyl, cyclobutyl methyl and cyclobutyl ethyl.

In one embodiment of the present invention, n is an integer selected from 0 to 2.

In one preferred embodiment of the present invention, the present invention provides a compound of formula (Ia),

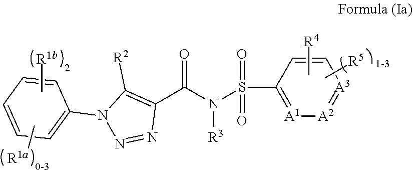

wherein,
R^{1a} is selected from the group consisting of hydrogen, halogen, CN, OR^{6a}, C₁-C₄-alkyl and C₁-C₄-haloalkyl; wherein each group of R^{1a} may optionally be substituted by one or more groups selected from the group consisting of halogen, CN and R^{6a};
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
R³ is selected from the group consisting of hydrogen, C₁-C₄-alkyl, C₁-C₄-haloalkyl and C₃-C₈-cycloalkyl-C₁-C₄-alkyl;
A¹, A² and A³ represent C or N; wherein not more than two of A¹, A² and A³ represent N;
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, (C═O)—R⁶, OR⁶, N(R⁶)₂, S(O)ₙR⁶, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl, C₃-C₆-carbocyclyl and a heterocyclic 3 to 6-membered ring; wherein each group of R⁵ may optionally be substituted by one or more groups selected from the group consisting of halogen, R^{6a};
R^{6a} is selected from the group consisting of hydrogen, C₁-C₄-alkyl, C₁-C₄-haloalkyl and C₃-C₆-cycloalkyl;
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl and C₃-C₆-cycloalkyl and C₃-C₆-cycloalkyl-C₁-C₄-alkyl;
n is an integer selected from 0 to 2;
or salts, stereoisomers, polymorphs or N-oxides thereof.

In another preferred embodiment of the present invention, the present invention provides a compound of formula (Ia-1),

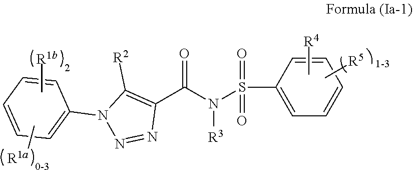

wherein,
R^{1a} is selected from the group consisting of hydrogen, halogen, CN, OR^{6a}, C₁-C₄-alkyl and C₁-C₄-haloalkyl; wherein each group of R^{1a} may optionally be substituted by one or more groups selected from the group consisting of halogen, CN and R^{6a};
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
R³ is selected from the group consisting of hydrogen, C₁-C₄-alkyl, C₁-C₄-haloalkyl and C₃-C₈-cycloalkyl-C₁-C₄-alkyl;
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, (C═O)—R⁶, OR⁶, N(R⁶)₂, S(O)ₙR⁶, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl, C₃-C₆-carbocyclyl and a heterocyclic 3 to 6-membered ring;
wherein each group of R⁵ may optionally be substituted by one or more groups selected from the group consisting of halogen, R^{6a};
R^{6a} is selected from the group consisting of hydrogen, C₁-C₄-alkyl, C₁-C₄-haloalkyl and C₃-C₆-cycloalkyl;
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl and C₃-C₆-cycloalkyl and C₃-C₆-cycloalkyl-C₁-C₄-alkyl;
n is an integer selected from 0 to 2;
or salts, stereoisomers, polymorphs or N-oxides thereof.

In yet another preferred embodiment of the present invention, the present invention provides a compound of formula (Ia-2),

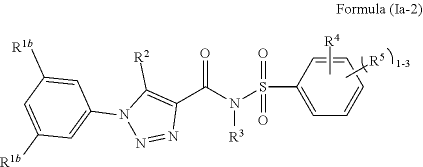

wherein,
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
R³ is selected from the group consisting of hydrogen, C₁-C₄-alkyl, C₁-C₄-haloalkyl and C₃-C₈-cycloalkyl-C₁-C₄-alkyl;
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, (C═O)—R⁶, OR⁶, N(R⁶)₂, S(O)ₐR⁶, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl, C₃-C₆-carbocyclyl and a heterocyclic 3 to 6-membered ring;
wherein each group of R⁵ may optionally be substituted by one or more groups selected from the group consisting of halogen, R^{6a};
R^{6a} is selected from the group consisting of hydrogen, C₁-C₄-alkyl, C₁-C₄-haloalkyl and C₃-C₆-cycloalkyl;
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl and C₃-C₆-cycloalkyl and C₃-C₆-cycloalkyl-C₁-C₄-alkyl;
n is an integer selected from 0 to 2;
or salts, stereoisomers, polymorphs or N-oxides thereof.

In yet another preferred embodiment of the present invention, the present invention provides a compound of formula (Ia-3),

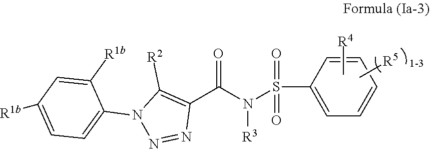

wherein,
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
R³ is selected from the group consisting of hydrogen, C₁-C₄-alkyl, C₁-C₄-haloalkyl and C₃-C₈-cycloalkyl-C₁-C₄-alkyl;
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, (C═O)—R⁶, OR⁶, N(R⁶)₂, S(O)ₙR⁶, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl, C₃-C₆-carbocyclyl and a heterocyclic 3 to 6-membered ring;
wherein each group of R⁵ may optionally be substituted by one or more groups selected from the group consisting of halogen, R^{6a};
R^{6a} is selected from the group consisting of hydrogen, C₁-C₄-alkyl, C₁-C₄-haloalkyl and C₃-C₆-cycloalkyl;
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl and C₃-C₆-cycloalkyl and C₃-C₆-cycloalkyl-C₁-C₄-alkyl;
n is an integer selected from 0 to 2;
or salts, stereoisomers, polymorphs or N-oxides thereof.

In one embodiment of the present invention, R^{1a} is selected from the group consisting of hydrogen, halogen, CN, OR^{6a}, C₁-C₄-alkyl and C₁-C₄-haloalkyl; wherein each group of Ria may optionally be substituted by one or more groups selected from the group consisting of halogen, CN and R^{6a};
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, OR⁶, C₁-C₄-alkyl, C₁-C₄-haloalkyl, C₃-C₆-cycloalkyl, C₃-C₆-cycloalkyl-C₁-C₄-alkyl and C₃-C₆-carbocyclyl; wherein each group of R⁵ may optionally be substituted by one or more groups selected from the group consisting of halogen and R^{6a};
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl, C₃-C₆-cycloalkyl and C₃-C₆-cycloalkyl-C₁-C₄-alkyl;
R^{6a} is selected from the group consisting of hydrogen, C₁-C₄-alkyl, C₁-C₄-haloalkyl and C₃-C₆-cycloalkyl.

In one preferred embodiment of the present invention, the phenyl ring attached to the sulfonamide group is di-substituted.

In another preferred embodiment of the present invention, R⁴ and R⁵ are independently selected from halogen, OR⁶, C₁-C₆-alkyl or C₁-C₆-haloalkyl, wherein OR⁶ represents C₁-C₆-alkoxy or C₁-C₆-haloalkoxy.

In one embodiment of the present invention, the C₃-C₆-cycloalkyl group is preferably cyclopropyl.

The compounds of the present invention as defined by formula (I), listed in tables 1-19 and/or listed in table (A) can be prepared as shown in the following schemes, in which, unless otherwise stated, the definition of each variable is as defined above in the detailed description of the invention.

### Sulfonamides—General Schemes:

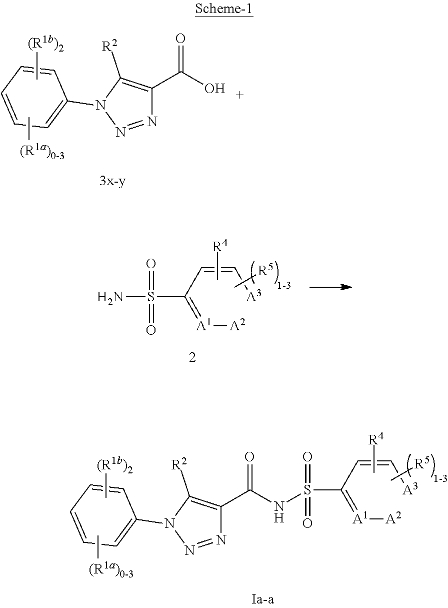

As shown in scheme 1, a compound of formula Ia-a can be prepared from a compound of formula 3x-y by the reaction with a compound of formula 2 in the presence of a suitable reagent such as a coupling reagent for e.g. 1-ethyl-3-(3-dimethylaminopropyl)carbodiimide, (1-[bis(dimethylamino)methylene]-1H-1,2,3-triazolo[4,5-b]pyridinium 3-oxide hexafluorophosphate (HATU); or halogenating reagent for e.g. phosphorous oxychloride, thionyl chloride, phosphorous pentachloride or oxalyl chloride, in the presence of a suitable organic or inorganic base such as 4-dimethylaminopyridine, N,N-diisopropylethylamine, trimethylamine, sodium or potassium hydride, and in the presence of a suitable polar or non polar solvent such as dichloromethane, tetrahydrofuran, N,N-dimethylformamide or tert-butyl alcohol or a mixture thereof.

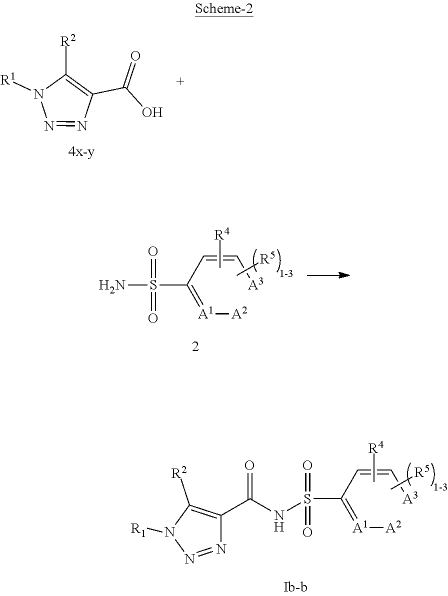

As shown in scheme 2, a compound of formula Ib-b can be prepared from a compound of formula 4x-y by the reaction with a compound of formula 2 in the presence of a suitable reagent such as a coupling reagent for e.g. 1-ethyl-3-(3-dimethylaminopropyl)carbodiimide or (1-[bis(dimethylamino)methylene]-1H-1,2,3-triazolo[4,5-b]pyridinium 3-oxide hexafluorophosphate (HATU); or halogenating reagent for e.g. phosphorous oxychloride, thionyl chloride, phosphorous pentachloride or oxalyl chloride, in the presence of a suitable organic or inorganic base such as 4-dimethylaminopyridine, N,N-diisopropylethylamine, trimethylamine, sodium or potassium hydride, and in the presence of a suitable polar or non polar solvent such as dichloromethane, tetrahydrofuran, N,N-dimethylformamide or tert-butyl alcohol or a mixture thereof.

Sulfonamide derivatives of formula 2 are commercially available or may be synthesized by following an analogous procedure as disclosed in WO2007023186 or WO2007144579.

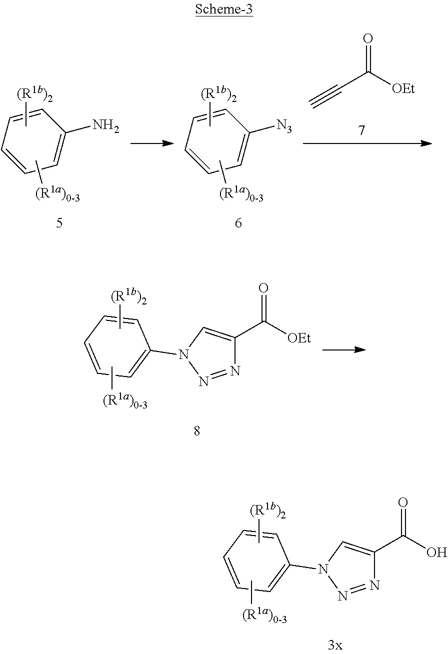

As shown in scheme 3, a compound of formula 5 can be converted into a compound of formula 6 through a Sandmayer reaction using suitable diazotizing agents (e.g. methyl nitrite, t-butyl nitrite, i-amyl nitrite, sodium nitrite, etc.) followed by the reaction with an appropriate reagent such as trimethylsilyl-azide, sodium azide, in the presence of a suitable solvent such as methyl tert-butyl ether (MTBE) or acetonitrile at 0° C. to 25° C. In the next step, the compound of formula 6 can be converted into a compound of formula 8 following a Click reaction using an appropriate reagent such as ethyl propiolate of formula 7, L-ascorbic acid and cupric sulfate, in the presence of a suitable solvent such as dimethylsulfoxide. Further, the compounds of formula 8 can be converted into a triazole carboxylic acid of formula 3x using a suitable base such as lithium hydroxide or sodium hydroxide, in the presence of a suitable solvent such as water, tetrahydrofuran, methanol or ethanol or a mixture thereof.

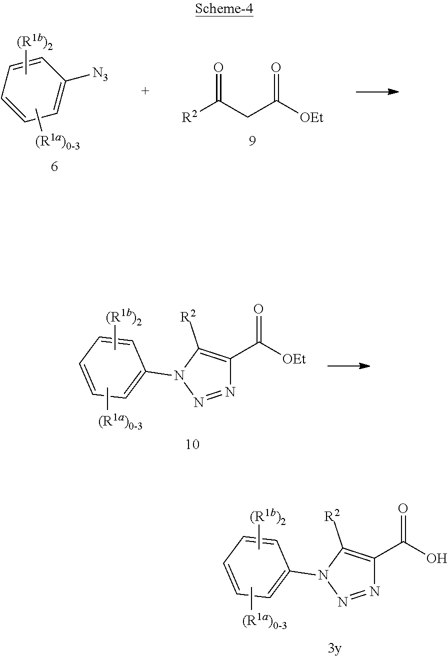

As shown in scheme 4, a compound of formula 10 can be prepared by cyclocondensation of the compound of formula 6 with a 3-keto-esters of formula 9 such as ethyl acetoacetate or ethyltrifluoroacetoacetate, in the presence of a suitable organic or inorganic base such as piperidine, diethylamine or potassium carbonate, in the presence of a suitable solvent such as methyl tert-butyl ether (MTBE), dimethylsulfoxide or water or a mixture thereof. The resulting compound of formula 10 can then be converted into the triazole carboxylic acid of formula 3y by using a suitable base such as lithium hydroxide or sodium hydroxide, in the presence of a suitable solvent such as water, tetrahydrofuran, methanol or ethanol or a mixture thereof.

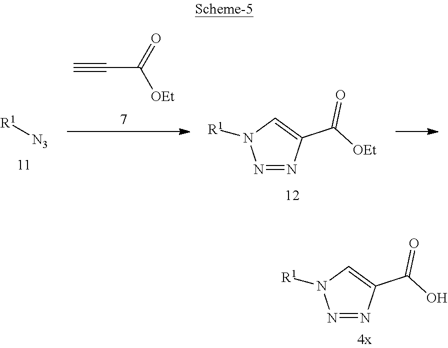

As shown in scheme 5, a compound of formula 12 can be prepared by reacting a compound of formula 11 by a (3+2) cycloaddition reaction with ethyl propiolate (formula 7) in the presence of an appropriate reagent such as L-ascorbic acid sodium salt and cupric sulfate in the presence of a suitable solvent such as dimethyl sulfoxide. A compound of formula 4× can then be prepared by hydrolyzing the compounds of formula 12 by using a suitable base such as lithium hydroxide or sodium hydroxide, in the presence of a suitable solvent such as water, tetrahydrofuran, methanol or ethanol or a mixture thereof.

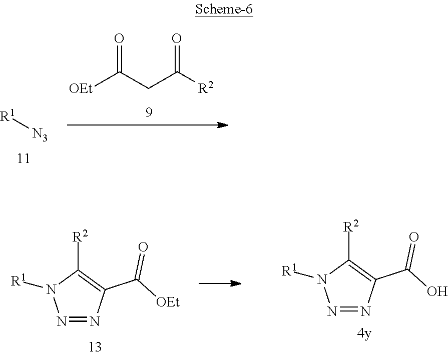

As shown in scheme 6, a compound of formula 13 can also be prepared by reacting the compound of formula 11 with the 3-keto-esters of formula 9, such as ethyl acetoacetate or ethyl trifluoroacetoacetate, in the presence of a suitable organic or inorganic base such as piperidine, potassium carbonate or sodium carbonate, in the presence of a suitable solvent such as dimethylsulfoxide or water or a mixture thereof. A compound of formula 4y can then be prepared by hydrolyzing the compound of formula 13 by using an ester hydrolyzing reagent such as lithium hydroxide, sodium hydroxide, in the presence of a suitable solvent such as water, tetrahydrofuran, methanol or ethanol or a mixture thereof.

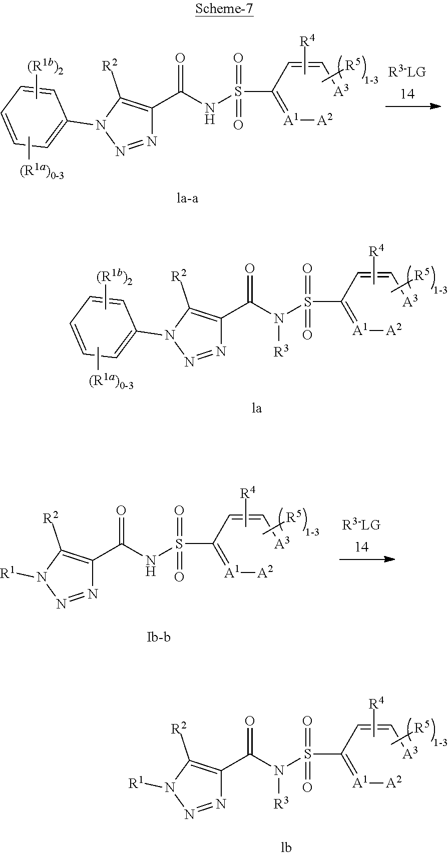

As shown in scheme 7, a compound of formula Ia and Ib can be prepared by an alkylation of the compounds of formula Ia-a and Ib-b by reacting them with a compound of formula 14 in the presence of a suitable base such as sodium hydride, sodium tert-butoxide and potassium tert-butoxide, in the presence of a suitable solvent such as N,N-dimethylformamide, dimethyl sulfoxide or acetonitrile or a mixture thereof.

### Sulfoximine—General Schemes:

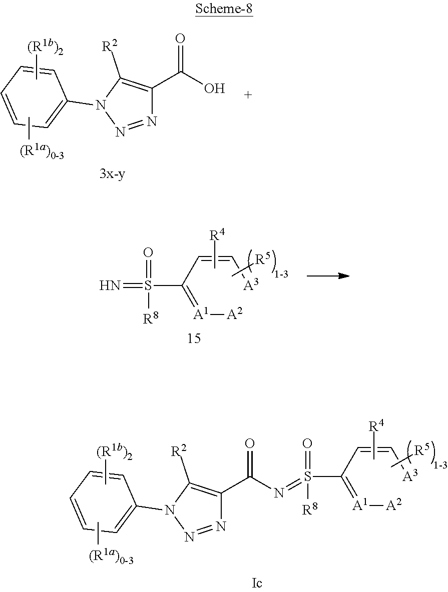

As shown in scheme 8, a compound of formula Ic can be prepared by coupling the compound of formula 3x-y with a compound of formula 15, in the presence of suitable coupling reagent such as 1-ethyl-3-(3-dimethylaminopropyl)carbodiimide or (1-[bis(dimethylamino)methylene]-1H-1,2,3-triazolo[4,5-b]pyridinium 3-oxide hexafluorophosphate (HATU), in the presence of a suitable base such as 4-dimethylaminopyridine, N,N-diisopropylethylamine or trimethylamine, in the presence of a suitable solvent such as dichloromethane, N,N-dimethylformamide or tert-butyl alcohol or a mixture thereof.

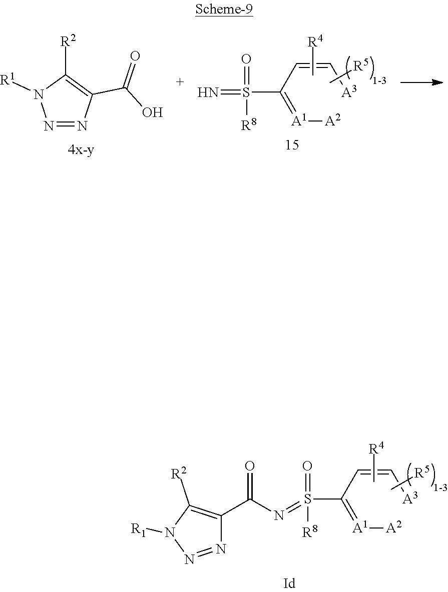

As shown in scheme 9, a compound of formula Id can be prepared by the coupling of the compound of formula 4x-y with the compound of formula 15 in the presence of a suitable coupling reagents such as 1-ethyl-3-(3-dimethylaminopropyl)carbodiimide or (1-[bis(dimethylamino)methylene]-1H-1,2,3-triazolo[4,5-b]pyridinium 3-oxide hexafluorophosphate (HATU), in the presence of suitable base such as 4-dimethylaminopyridine, N,N-diisopropylethylamine, trimethylamine, in the presence of a suitable solvent such as dichloromethane, N,N-dimethylformamide or tert-butyl alcohol or a mixture thereof.

Sulfoximines derivatives of formula 15 can be synthesized by following an analogous procedure as disclosed in WO2019150219.

As shown above the compound of formula 3x-y includes the compound of formula 3x and 3y.

The compound of formula 4x-y includes the compound of formula 4x and 4y.

Any of the compounds according to this invention can exist in one or more optical, geometric, or chiral isomer forms depending on the number of asymmetric centers in the compound. The invention thus relates equally to all the optical isomers and to their racemic or scalemic mixtures (the term “scalemic” denotes a mixture of enantiomers in different proportions), and to the mixtures of all possible stereoisomers, in all proportions. The diastereoisomers and/or the optical isomers can be separated according to the methods which are known per se by a person ordinary skilled in the art.

Any of the compounds according to the invention can also exist in one or more geometric isomer forms depending on the number of double bonds in the compound. The invention thus relates equally to all geometric isomers and to all possible mixtures, in all proportions. The geometric isomers can be separated according to general methods, which are known per se by a person ordinary skilled in the art.

Any of the compounds according to the invention, can also exist in one or more amorphic or isomorphic or polymorphic forms, depending on their preparation, purification storage and various other influencing factors. The invention thus relates all the possible amorphic, isomorphic and polymorphic forms, in all proportions. The amorphic, isomorphic and polymorphic forms can be prepared and/or separated and/or purified according to general methods, which are known per se by a person ordinary skilled in the art.

For carrying out chemical conversions, there is a large number of suitable known standard methods, such as alkylation, halogenation, acylation, amidation, oximation, oxidation and reduction. The choice of the preparation methods which are suitable in specific reaction steps are depending on the properties (e.g., the reactivity) of the substituents in the involved intermediates. These reactions can be conveniently performed mostly in a solvent. They may conveniently be performed at various temperatures, usually under an inert atmosphere. The reactants can be reacted in the presence of an appropriate base. Examples of suitable bases, in a non-limiting way, are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, N,N-diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent.

The reactions according to scheme-1 to scheme-9 are advantageously carried out in a temperature range from approximately −20° C. to approximately +50° C., preferably from approximately 0° C. to approximately 30° C.

A compound of formula (I) can be converted in a manner known per se into another compound of formula (I) by replacing one or more substituents of the starting compound of formula (I) in the customary manner by (an)other substituent(s) according to the invention. Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step. Salts of compounds of formula (I) can be prepared in a manner known per se. Thus, for example, acid addition salts of compounds of formula (I) are obtained by treating with a suitable acid or a suitable ion exchanger reagent and salts with bases are obtained by the treatment with a suitable base or with a suitable ion exchanger reagent. A salt is chosen depending on its tolerances for the compound's use, such as agricultural or physiological tolerance. Salts of compounds of formula (I) can be converted in the customary manner into the free compounds of formula (I), acid addition salts, for example, by the treatment with a suitable basic compound or with a suitable ion exchanger reagent and salts with bases, for example, by treating with a suitable acid or with a suitable ion exchanger reagent. Salts of the compounds of formula (I) can be converted in a manner known per se into other salts of compounds of formula (I), acid addition salts, for example, into other acid addition salts, for example by treating of a salt of an inorganic acid such as hydrochloride with a suitable metal salt such as a sodium, barium or silver salt, or by the treatment of an acid, for example with silver acetate, in a suitable solvent in which an inorganic salt which forms, for example silver chloride, is insoluble and thus precipitates from the reaction mixture.

In one embodiment, the present invention provides a method for the synthesis of a compound of formula (I), comprising the steps selected from a) to d):
a) reacting a compound of formula (11) with a compound of formula (9), in the presence of a suitable base and a suitable solvent, to obtain a compound of formula (13), which on further reaction with a hydrolyzing reagent in the presence of a suitable solvent provides a compound of formula (4y):

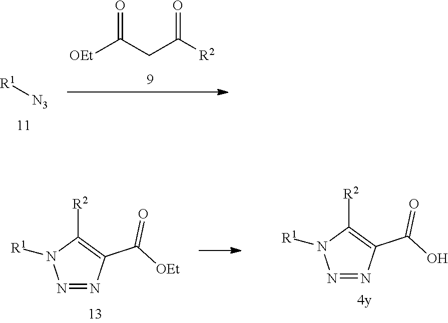

b) reacting a compound of formula (4y) with a compound of formula (2) in the presence of a suitable reagent, a suitable solvent and a suitable base to obtain a compound of formula (Ib-b):

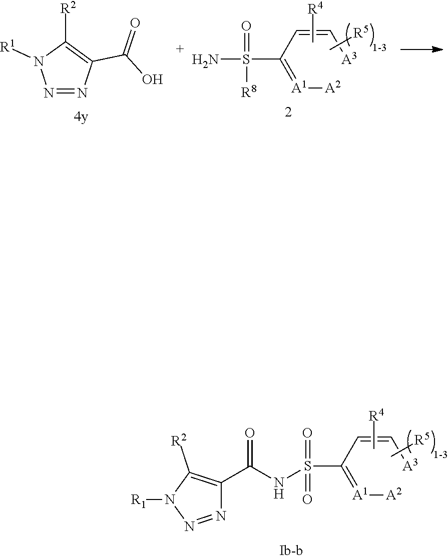

c) a compound of formula (Ib) is obtained by reacting a compound of formula (Ib-b) with a compound of formula (14), in the presence of a suitable solvent and a suitable base:

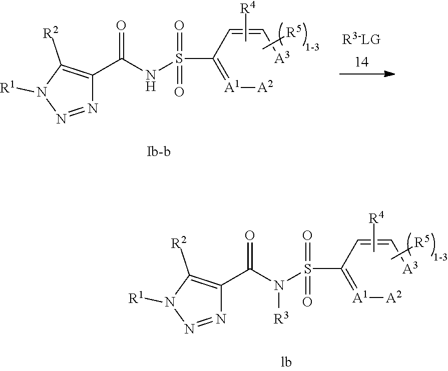

d) a compound of formula (Id) is obtained by reacting a compound of formula (4y) with a compound of formula (15) in the presence of a suitable coupling reagent, a suitable solvent and a suitable base:

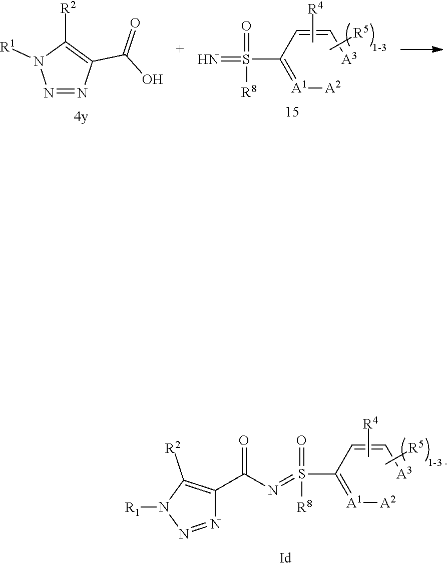

In another embodiment, compounds of formula (I) of the present invention are also read as including salts thereof. Exemplary salts include, but are not limited to hydrochloride, hydrobromide, hydroiodide, acetate, trifluoroacetate, and trifluoromethane sulfonate.

In yet another embodiment the present invention provides the compounds of formula (I) as mentioned in the Tables 1 to 19 which can be prepared according to the methods described above and the experimental procedures described below for the compounds given in table-A. The examples which follow are intended to illustrate the invention and show the preferred compounds of formula (I), in the form of a compound of formula (I-1).

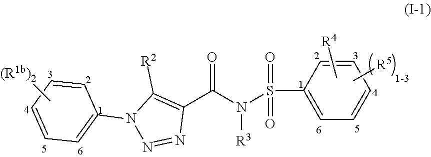

Table Y describes the variables R², R³, R⁴ and R⁵ of the compounds of formula (I-1).

TABLE Y

| Comp. | | | | |
|---|---|---|---|---|
| No. | R² | R³ | R⁴ | R⁵ |
| Y. 1 | CH₃ | H | H | 2-F |
| Y. 2 | CH₃ | H | H | 3-Cl |
| Y. 3 | CH₃ | H | H | 4-Cl |
| Y. 4 | CH₃ | H | H | 3-CH₃ |
| Y. 5 | CH₃ | H | H | 4-OCH₃ |
| Y. 6 | CH₃ | H | H | 2-CHF₂ |
| Y. 7 | CH₃ | H | H | 3-CHF₂ |
| Y. 8 | CH₃ | H | H | 2-CF₃ |
| Y. 9 | CH₃ | H | H | 4-OCF₃ |
| Y. 10 | CH₃ | H | H | 4-OCH₂CF₃ |
| Y. 11 | CH₃ | H | H | 2-CN |
| Y. 12 | CH₃ | H | H | 2-CyPr |
| Y. 13 | CH₃ | H | H | 4-N(CH₃)₂ |
| Y. 14 | CH₃ | H | H | 3-NO₂ |
| Y. 15 | CH₃ | H | H | 2,3-di-F |
| Y. 16 | CH₃ | H | H | 2,4-di-F |
| Y. 17 | CH₃ | H | H | 2,5-di-F |
| Y. 18 | CH₃ | H | H | 3,4-di-F |
| Y. 19 | CH₃ | H | H | 3,5-di-F |
| Y. 20 | CH₃ | H | H | 3,5-di-CH₃ |
| Y. 21 | CH₃ | H | H | 3,4-di-CH₃ |
| Y. 22 | CH₃ | H | H | 2,3-di-Cl |
| Y. 23 | CH₃ | H | H | 2,4-di-Cl |
| Y. 24 | CH₃ | H | H | 2,5-di-Cl |
| Y. 25 | CH₃ | H | H | 2,6-di-Cl |
| Y. 26 | CH₃ | H | H | 3,4-di-Cl |
| Y. 27 | CH₃ | H | H | 3,6-di-Cl |
| Y. 28 | CH₃ | H | H | 2,3-di-CH₃ |
| Y. 29 | CH₃ | H | H | 2,4-di-CH₃ |
| Y. 30 | CH₃ | H | H | 2,5-di-CH₃ |
| Y. 31 | CH₃ | H | H | 2,6-di-CH₃ |
| Y. 32 | CH₃ | H | H | 2,3-di-CF₃ |
| Y. 33 | CH₃ | H | H | 2,4-di-CF₃ |
| Y. 34 | CH₃ | H | H | 2,5-di-CF₃ |
| Y. 35 | CH₃ | H | H | 2,6-di-CF₃ |
| Y. 36 | CH₃ | H | H | 3,4-di-CF₃ |
| Y. 37 | CH₃ | H | H | 3,5-di-CF₃ |
| Y. 38 | CH₃ | H | H | 3,6-di-CF₃ |
| Y. 39 | CH₃ | H | H | 2,3-di-i-Pr |
| Y. 40 | CH₃ | H | H | 2,5-di-i-Pr |
| Y. 41 | CH₃ | H | H | 2-F, 3-Cl |
| Y. 42 | CH₃ | H | H | 2-F, 4-Cl |
| Y. 43 | CH₃ | H | H | 2-F, 5-Cl |
| Y. 44 | CH₃ | H | H | 3-F, 4-Cl |
| Y. 45 | CH₃ | H | H | 3-F, 5-Cl |
| Y. 46 | CH₃ | H | H | 2-Cl, 3-F |
| Y. 47 | CH₃ | H | H | 2-Cl, 4-F |
| Y. 48 | CH₃ | H | H | 2-Cl, 5-F |
| Y. 49 | CH₃ | H | H | 3-Cl, 4-F |
| Y. 50 | CH₃ | H | H | 2-F, 4-Br |
| Y. 51 | CH₃ | H | H | 2-F, 5-Br |
| Y. 52 | CH₃ | H | H | 3-F, 4-Br |
| Y. 53 | CH₃ | H | H | 2-Cl, 4-Br |
| Y. 54 | CH₃ | H | H | 2-Cl, 5-Br |
| Y. 55 | CH₃ | H | H | 2-Cl, 4-CH₃ |
| Y. 56 | CH₃ | H | H | 2-Cl, 5-CH₃ |
| Y. 57 | CH₃ | H | H | 3-Cl, 4-Br |
| Y. 58 | CH₃ | H | H | 2-Br, 4-F |
| Y. 59 | CH₃ | H | H | 2-Br, 3-CF₃ |
| Y. 60 | CH₃ | H | H | 2-Br, 4-CF₃ |
| Y. 61 | CH₃ | H | H | 2-Br, 5-CF₃ |
| Y. 62 | CH₃ | H | H | 2-Br, 6-CF₃ |
| Y. 63 | CH₃ | H | H | 3-Br, 4-CF₃ |
| Y. 64 | CH₃ | H | H | 3-Br, 5-CF₃ |
| Y. 65 | CH₃ | H | H | 3-Br, 6-CF₃ |
| Y. 66 | CH₃ | H | H | 3-Br, 4-F |
| Y. 67 | CH₃ | H | H | 2-Br, 5-Cl |
| Y. 68 | CH₃ | H | H | 3-Br, 4-Cl |
| Y. 69 | CH₃ | H | H | 2-F, 3-CH₃ |
| Y. 70 | CH₃ | H | H | 2-F, 4-CH₃ |
| Y. 71 | CH₃ | H | H | 2-F, 5-CH₃ |
| Y. 72 | CH₃ | H | H | 2-F, 6-CH₃ |
| Y. 73 | CH₃ | H | H | 2-Cl, 3-CH₃ |
| Y. 74 | CH₃ | H | H | 2-CH₃, 3-Cl |
| Y. 75 | CH₃ | H | H | 2-CH₃, 4-Cl |
| Y. 76 | CH₃ | H | H | 2-CH₃, 5-Cl |
| Y. 77 | CH₃ | H | H | 2-F, 5-CH₃ |
| Y. 78 | CH₃ | H | H | 2-F, 6-CH₃ |
| Y. 79 | CH₃ | H | H | 2-Br, 5-CH₃ |
| Y. 80 | CH₃ | H | H | 2-CH₃, 3-Br |
| Y. 81 | CH₃ | H | H | 3-CH₃, 5-Br |
| Y. 82 | CH₃ | H | H | 2-CH₃, 4-Br |
| Y. 83 | CH₃ | H | H | 2-CH₃, 3-F |
| Y. 84 | CH₃ | H | H | 2-CH₃, 4-F |
| Y. 85 | CH₃ | H | H | 2-CH₃, 5-F |
| Y. 86 | CH₃ | H | H | 3-CN, 4-F |
| Y. 87 | CH₃ | H | H | 3-CN, 4-Cl |
| Y. 88 | CH₃ | H | H | 2-F, 5-CN |
| Y. 89 | CH₃ | H | H | 2-Cl, 5-CN |
| Y. 90 | CH₃ | H | H | 2-F, 4-CN |
| Y. 91 | CH | H | H | 2-Cl, 4-CN |
| Y. 92 | CH₃ | H | H | 3-F, 4-CN |
| Y. 93 | CH₃ | H | H | 3-Cl, 4-CN |
| Y. 94 | CH₃ | H | H | 3-Cl, 5-CN |
| Y. 95 | CH₃ | H | H | 2-Br, 5-CN |
| Y. 96 | CH₃ | H | H | 2-CN, 3-Br |
| Y. 97 | CH₃ | H | H | 3-CN, 5-Br |
| Y. 98 | CH₃ | H | H | 2-CN, 4-Br |
| Y. 99 | CH₃ | H | H | 3-Br, 4-CN |
| Y. 100 | CH₃ | H | H | 2-Br, 4-CN |
| Y. 101 | CH₃ | H | H | 2-Br, 4-CF₃ |
| Y. 102 | CH₃ | H | H | 2-Br, 5-CF₃ |
| Y. 103 | CH₃ | H | H | 2-Br, 6-CF₃ |
| Y. 104 | CH₃ | H | H | 2-F, 3-CF₃ |
| Y. 105 | CH₃ | H | H | 2-F, 4-CF₃ |
| Y. 106 | CH₃ | H | H | 2-F, 5-CF₃ |
| Y. 107 | CH₃ | H | H | 2-F, 6-CF₃ |
| Y. 108 | CH₃ | H | H | 3-F, 4-CF₃ |
| Y. 109 | CH₃ | H | H | 4-F, 3-CF₃ |
| Y. 110 | CH₃ | H | H | 2-Cl, 3-CF₃ |
| Y. 111 | CH₃ | H | H | 2-Cl, 4-CF₃ |
| Y. 112 | CH | H | H | 3-CF₃, 5-Br |
| Y. 113 | CH₃ | H | H | 3-CF₃, 4-Br |
| Y. 114 | CH₃ | H | H | 3-CF₃, 5-CH₃ |
| Y. 115 | CH₃ | H | H | 2-CH₃, 4-CF₃ |
| Y. 116 | CH₃ | H | H | 2-CH₃, 5-CF₃ |
| Y. 117 | CH₃ | H | H | 3-CF₃, 4-CH₃ |
| Y. 118 | CH₃ | H | H | 3-CF₃, 5-CN |
| Y. 119 | CH₃ | H | H | 3-CN, 4-CF₃ |
| Y. 120 | CH₃ | H | H | 2-OCHF₂, 4-Cl |
| Y. 121 | CH₃ | H | H | 2-OCHF₂, 4-F |
| Y. 122 | CH₃ | H | H | 2-Cl, 4-OCF₃ |
| Y. 123 | CH₃ | H | H | 2-Cl, 5-CF₃ |
| Y. 124 | CH₃ | H | H | 3-Cl, 5-CF₃ |
| Y. 125 | CH₃ | H | H | 4-Cl, 3-CF₃ |
| Y. 126 | CH₃ | H | H | 2-F, 4-SCH₃ |
| Y. 127 | CH₃ | H | H | 2-Cl, 5-SO₂CH₃ |
| Y. 128 | CH₃ | H | H | 2-OCH₃, 3-CH₃ |
| Y. 129 | CH₃ | H | H | 2-OCH₃, 4-CH₃ |
| Y. 130 | CH₃ | H | H | 2-OCH₃, 5-CH₃ |
| Y. 131 | CH₃ | H | H | 2-OCH₃, 3-Cl |
| Y. 132 | CH₃ | H | H | 2-OCH₃, 4-Cl |
| Y. 133 | CH₃ | H | H | 2-OCH₃, 5-Cl |
| Y. 134 | CH₃ | H | H | 2-F, 3-OCH₃ |
| Y. 135 | CH₃ | H | H | 2-Cl, 3-OCH₃ |
| Y. 136 | CH₃ | H | H | 2-Cl, 4-OCH₃ |
| Y. 137 | CH₃ | H | H | 3-OCH₃, 4-Br |
| Y. 138 | CH₃ | H | H | 3-Br, 5-OCH₃ |
| Y. 139 | CH₃ | H | H | 2,4-di-F, 5-Cl |
| Y. 140 | CH₃ | H | H | 2,3-di-F, 5-Cl |
| Y. 141 | CH₃ | H | H | 2-Cl, 4,5-di-F |
| Y. 142 | CH₃ | H | H | 3,5-di-Cl, 4-F |
| Y. 143 | CH₃ | H | H | 3,4-di-F, 5-CF₃ |
| Y. 144 | CH₃ | H | H | 3,5-di-Cl, 4-CH₃ |
| Y. 145 | CH₃ | H | H | 3,5-di-CH₃, 4-F |
| Y. 146 | CH₃ | H | H | 2,5-di-CH₃, 3-Cl |
| Y. 147 | CF₃ | H | H | 2-F |
| Y. 148 | CF₃ | H | H | 3-Cl |
| Y. 149 | CF₃ | H | H | 4-Cl |
| Y. 150 | CF₃ | H | H | 3-CH₃ |
| Y. 151 | CF₃ | H | H | 4-OCH₃ |
| Y. 152 | CF₃ | H | H | 2-CHF₂ |
| Y. 153 | CF₃ | H | H | 3-CHF₂ |
| Y. 154 | CF₃ | H | H | 2-CF₃ |
| Y. 155 | CF₃ | H | H | 4-OCF₃ |
| Y. 156 | CF₃ | H | H | 4-OCH₂CF₃ |
| Y. 157 | CF₃ | H | H | 2-CN |
| Y. 158 | CF₃ | H | H | 2-CyPr |
| Y. 159 | CF₃ | H | H | 4-N(CH₃)₂ |
| Y. 160 | CF₃ | H | H | 3-NO₂ |
| Y. 161 | CF₃ | H | H | 2,3-di-F |
| Y. 162 | CF₃ | H | H | 2,4-di-F |
| Y. 163 | CF₃ | H | H | 2,5-di-F |
| Y. 164 | CF₃ | H | H | 3,4-di-F |
| Y. 165 | CF₃ | H | H | 3,5-di-F |
| Y. 166 | CF₃ | H | H | 3,5-di-CH₃ |
| Y. 167 | CF₃ | H | H | 3,4-di-CH₃ |
| Y. 168 | CF₃ | H | H | 2,3-di-Cl |
| Y. 169 | CF₃ | H | H | 2,4-di-Cl |
| Y. 170 | CF₃ | H | H | 2,5-di-Cl |
| Y. 171 | CF₃ | H | H | 2,6-di-Cl |
| Y. 172 | CF₃ | H | H | 3,4-di-Cl |
| Y. 173 | CF₃ | H | H | 3,6-di-Cl |
| Y. 174 | CF₃ | H | H | 2,3-di-CH₃ |
| Y. 175 | CF₃ | H | H | 2,4-di-CH₃ |
| Y. 176 | CF₃ | H | H | 2,5-di-CH₃ |
| Y. 177 | CF₃ | H | H | 2,6-di-CH₃ |
| Y. 178 | CF₃ | H | H | 2,3-di-CF₃ |
| Y. 179 | CF₃ | H | H | 2,4-di-CF₃ |
| Y. 180 | CF₃ | H | H | 2,5-di-CF₃ |
| Y. 181 | CF₃ | H | H | 2,6-di-CF₃ |
| Y. 182 | CF₃ | H | H | 3,4-di-CF₃ |
| Y. 183 | CF₃ | H | H | 3,5-di-CF₃ |
| Y. 184 | CF₃ | H | H | 3,6-di-CF₃ |
| Y. 185 | CF₃ | H | H | 2,3-di-i-Pr |
| Y. 186 | CF₃ | H | H | 2,5-di-i-Pr |
| Y. 187 | CF₃ | H | H | 2-F, 3-Cl |
| Y. 188 | CF₃ | H | H | 2-F, 4-Cl |
| Y. 189 | CF₃ | H | H | 2-F, 5-Cl |
| Y. 190 | CF₃ | H | H | 3-F, 4-Cl |
| Y. 191 | CF₃ | H | H | 3-F, 5-Cl |
| Y. 192 | CF₃ | H | H | 2-Cl, 3-F |
| Y. 193 | CF₃ | H | H | 2-Cl, 4-F |
| Y. 194 | CF₃ | H | H | 2-Cl, 5-F |
| Y. 195 | CF₃ | H | H | 3-Cl, 4-F |
| Y. 196 | CF₃ | H | H | 2-F, 4-Br |
| Y. 197 | CF₃ | H | H | 2-F, 5-Br |
| Y. 198 | CF₃ | H | H | 3-F, 4-Br |
| Y. 199 | CF₃ | H | H | 2-Cl, 4-Br |
| Y. 200 | CF₃ | H | H | 2-Cl, 5-Br |
| Y. 201 | CF₃ | H | H | 2-Cl, 4-CH₃ |
| Y. 202 | CF₃ | H | H | 2-Cl, 5-CH₃ |
| Y. 203 | CF₃ | H | H | 3-Cl, 4-Br |
| Y. 204 | CF₃ | H | H | 2-Br, 4-F |
| Y. 205 | CF₃ | H | H | 2-Br, 3-CF₃ |
| Y. 206 | CF₃ | H | H | 2-Br, 4-CF₃ |
| Y. 207 | CF₃ | H | H | 2-Br, 5-CF₃ |
| Y. 208 | CF₃ | H | H | 2-Br, 6-CF₃ |
| Y. 209 | CF₃ | H | H | 3-Br, 4-CF₃ |
| Y. 210 | CF₃ | H | H | 3-Br, 5-CF₃ |
| Y. 211 | CF₃ | H | H | 3-Br, 6-CF₃ |
| Y. 212 | CF₃ | H | H | 3-Br, 4-F |
| Y. 213 | CF₃ | H | H | 2-Br, 5-Cl |
| Y. 214 | CF₃ | H | H | 3-Br, 4-Cl |
| Y. 215 | CF₃ | H | H | 2-F, 3-CH₃ |
| Y. 216 | CF₃ | H | H | 2-F, 4-CH₃ |
| Y. 217 | CF₃ | H | H | 2-F, 5-CH₃ |
| Y. 218 | CF₃ | H | H | 2-F, 6-CH₃ |
| Y. 219 | CF₃ | H | H | 2-Cl, 3-CH₃ |
| Y. 220 | CF₃ | H | H | 2-CH₃, 3-Cl |
| Y. 221 | CF₃ | H | H | 2-CH₃, 4-Cl |
| Y. 222 | CF₃ | H | H | 2-CH₃, 5-Cl |
| Y. 223 | CF₃ | H | H | 2-F, 5-CH₃ |
| Y. 224 | CF₃ | H | H | 2-F, 6-CH₃ |
| Y. 225 | CF₃ | H | H | 2-Br, 5-CH₃ |
| Y. 226 | CF₃ | H | H | 2-CH₃, 3-Br |
| Y. 227 | CF₃ | H | H | 3-CH₃, 5-Br |
| Y. 228 | CF₃ | H | H | 2-CH₃, 4-Br |
| Y. 229 | CF₃ | H | H | 2-CH₃, 3-F |
| Y. 230 | CF₃ | H | H | 2-CH₃, 4-F |
| Y. 231 | CF₃ | H | H | 2-CH₃, 5-F |
| Y. 232 | CF₃ | H | H | 3-CN, 4-F |
| Y. 233 | CF₃ | H | H | 3-CN, 4-Cl |
| Y. 234 | CF₃ | H | H | 2-F, 5-CN |
| Y. 235 | CF₃ | H | H | 2-Cl, 5-CN |
| Y. 236 | CF₃ | H | H | 2-F, 4-CN |
| Y. 237 | CF₃ | H | H | 2-Cl, 4-CN |
| Y. 238 | CF₃ | H | H | 3-F, 4-CN |
| Y. 239 | CF₃ | H | H | 3-Cl, 4-CN |
| Y. 240 | CF₃ | H | H | 3-Cl, 5-CN |
| Y. 241 | CF₃ | H | H | 2-Br, 5-CN |
| Y. 242 | CF₃ | H | H | 2-CN, 3-Br |
| Y. 243 | CF₃ | H | H | 3-CN, 5-Br |
| Y. 244 | CF₃ | H | H | 2-CN, 4-Br |
| Y. 245 | CF₃ | H | H | 3-Br, 4-CN |
| Y. 246 | CF₃ | H | H | 2-Br, 4-CN |
| Y. 247 | CF₃ | H | H | 2-Br, 4-CF₃ |
| Y. 248 | CF₃ | H | H | 2-Br, 5-CF₃ |
| Y. 249 | CF₃ | H | H | 2-Br, 6-CF₃ |
| Y. 250 | CF₃ | H | H | 2-F, 3-CF₃ |
| Y. 251 | CF₃ | H | H | 2-F, 4-CF₃ |
| Y. 252 | CF₃ | H | H | 2-F, 5-CF₃ |
| Y. 253 | CF₃ | H | H | 2-F, 6-CF₃ |
| Y. 254 | CF₃ | H | H | 3-F, 4-CF₃ |
| Y. 255 | CF₃ | H | H | 4-F, 3-CF₃ |
| Y. 256 | CF₃ | H | H | 2-Cl, 3-CF₃ |
| Y. 257 | CF₃ | H | H | 2-Cl, 4-CF₃ |
| Y. 258 | CF₃ | H | H | 3-CF₃, 5-Br |
| Y. 259 | CF₃ | H | H | 3-CF₃, 4-Br |
| Y. 260 | CF₃ | H | H | 3-CF₃, 5-CH₃ |
| Y. 261 | CF₃ | H | H | 2-CH₃, 4-CF₃ |
| Y. 262 | CF₃ | H | H | 2-CH₃, 5-CF₃ |
| Y. 263 | CF₃ | H | H | 3-CF₃, 4-CH₃ |
| Y. 264 | CF₃ | H | H | 3-CF₃, 5-CN |
| Y. 265 | CF₃ | H | H | 3-CN, 4-CF₃ |
| Y. 266 | CF₃ | H | H | 2-OCHF₂, 4-Cl |
| Y. 267 | CF₃ | H | H | 2-OCHF₂, 4-F |
| Y. 268 | CF₃ | H | H | 2-Cl, 4-OCF₃ |
| Y. 269 | CF₃ | H | H | 2-Cl, 5-CF₃ |
| Y. 270 | CF | H | H | 3-Cl, 5-CF₃ |
| Y. 271 | CF₃ | H | H | 4-Cl, 3-CF₃ |
| Y. 272 | CF₃ | H | H | 2-F, 4-SCH₃ |
| Y. 273 | CF₃ | H | H | 2-Cl, 5-SO₂CH₃ |
| Y. 274 | CF₃ | H | H | 2-OCH₃, 3-CH₃ |
| Y. 275 | CF₃ | H | H | 2-OCH₃, 4-CH₃ |
| Y. 276 | CF₃ | H | H | 2-OCH₃, 5-CH₃ |
| Y. 277 | CF₃ | H | H | 2-OCH₃, 3-Cl |
| Y. 278 | CF₃ | H | H | 2-OCH₃, 4-Cl |
| Y. 279 | CF₃ | H | H | 2-OCH₃, 5-Cl |
| Y. 280 | CF₃ | H | H | 2-F, 3-OCH₃ |
| Y. 281 | CF₃ | H | H | 2-Cl, 3-OCH₃ |
| Y. 282 | CF₃ | H | H | 2-Cl, 4-OCH₃ |
| Y. 283 | CF₃ | H | H | 3-OCH₃, 4-Br |
| Y. 284 | CF₃ | H | H | 3-Br, 5-OCH₃ |
| Y. 285 | CF₃ | H | H | 2,4-di-F, 5-Cl |
| Y. 286 | CF₃ | H | H | 2,3-di-F, 5-Cl |
| Y. 287 | CF₃ | H | H | 2-Cl, 4,5-di-F |
| Y. 288 | CF₃ | H | H | 3,5-di-Cl, 4-F |
| Y. 289 | CF₃ | H | H | 3,4-di-F, 5-CF₃ |
| Y. 290 | CF₃ | H | H | 3,5-di-Cl, 4-CH₃ |
| Y. 291 | CF₃ | H | H | 3,5-di-CH₃, 4-F |
| Y. 292 | CF₃ | H | H | 2,5-di-CH₃, 3-Cl |
| Y. 293 | CyPr | H | H | 2-F |
| Y. 294 | CyPr | H | H | 3-Cl |
| Y. 295 | CyPr | H | H | 4-Cl |
| Y. 296 | CyPr | H | H | 3-CH₃ |
| Y. 297 | CyPr | H | H | 4-OCH₃ |
| Y. 298 | CyPr | H | H | 2-CHF₂ |
| Y. 299 | CyPr | H | H | 3-CHF₂ |
| Y. 300 | CyPr | H | H | 2-CF₃ |
| Y. 301 | CyPr | H | H | 4-OCF₃ |
| Y. 302 | CyPr | H | H | 4-OCH₂CF₃ |
| Y. 303 | CyPr | H | H | 2-CN |
| Y. 304 | CyPr | H | H | 2-CyPr |
| Y. 305 | CyPr | H | H | 4-N(CH₃)₂ |
| Y. 306 | CyPr | H | H | 3-NO₂ |
| Y. 307 | CyPr | H | H | 2,3-di-F |
| Y. 308 | CyPr | H | H | 2,4-di-F |
| Y. 309 | CyPr | H | H | 2,5-di-F |
| Y. 310 | CyPr | H | H | 3,4-di-F |
| Y. 311 | CyPr | H | H | 3,5-di-F |
| Y. 312 | CyPr | H | H | 3,5-di-CH₃ |
| Y. 313 | CyPr | H | H | 3,4-di-CH₃ |
| Y. 314 | CyPr | H | H | 2,3-di-Cl |
| Y. 315 | CyPr | H | H | 2,4-di-Cl |
| Y. 316 | CyPr | H | H | 2,5-di-Cl |
| Y. 317 | CyPr | H | H | 2,6-di-Cl |
| Y. 318 | CyPr | H | H | 3,4-di-Cl |
| Y. 319 | CyPr | H | H | 3,6-di-Cl |
| Y. 320 | CyPr | H | H | 2,3-di-CH₃ |
| Y. 321 | CyPr | H | H | 2,4-di-CH₃ |
| Y. 322 | CyPr | H | H | 2,5-di-CH₃ |
| Y. 323 | CyPr | H | H | 2,6-di-CH₃ |
| Y. 324 | CyPr | H | H | 2,3-di-CF₃ |
| Y. 325 | CyPr | H | H | 2,4-di-CF₃ |
| Y. 326 | CyPr | H | H | 2,5-di-CF₃ |
| Y. 327 | CyPr | H | H | 2,6-di-CF₃ |
| Y. 328 | CyPr | H | H | 3,4-di-CF₃ |
| Y. 329 | CyPr | H | H | 3,5-di-CF₃ |
| Y. 330 | CyPr | H | H | 3,6-di-CF₃ |
| Y. 331 | CyPr | H | H | 2,3-di-i-Pr |
| Y. 332 | CyPr | H | H | 2,5-di-i-Pr |
| Y. 333 | CyPr | H | H | 2-F, 3-Cl |
| Y. 334 | CyPr | H | H | 2-F, 4-Cl |
| Y. 335 | CyPr | H | H | 2-F, 5-Cl |
| Y. 336 | CyPr | H | H | 3-F, 4-Cl |
| Y. 337 | CyPr | H | H | 3-F,5-Cl |
| Y. 338 | CyPr | H | H | 2-Cl, 3-F |
| Y. 339 | CyPr | H | H | 2-Cl, 4-F |
| Y. 340 | CyPr | H | H | 2-Cl, 5-F |
| Y. 341 | CyPr | H | H | 3-Cl, 4-F |
| Y. 342 | CyPr | H | H | 2-F, 4-Br |
| Y. 343 | CyPr | H | H | 2-F, 5-Br |
| Y. 344 | CyPr | H | H | 3-F, 4-Br |
| Y. 345 | CyPr | H | H | 2-Cl, 4-Br |
| Y. 346 | CyPr | H | H | 2-Cl, 5-Br |
| Y. 347 | CyPr | H | H | 2-Cl, 4-CH₃ |
| Y. 348 | CyPr | H | H | 2-Cl, 5-CH₃ |
| Y. 349 | CyPr | H | H | 3-Cl, 4-Br |
| Y. 350 | CyPr | H | H | 2-Br, 4-F |
| Y. 351 | CyPr | H | H | 2-Br, 3-CF₃ |
| Y. 352 | CyPr | H | H | 2-Br, 4-CF₃ |
| Y. 353 | CyPr | H | H | 2-Br, 5-CF₃ |
| Y. 354 | CyPr | H | H | 2-Br, 6-CF₃ |
| Y. 355 | CyPr | H | H | 3-Br, 4-CF₃ |
| Y. 356 | CyPr | H | H | 3-Br, 5-CF₃ |
| Y. 357 | CyPr | H | H | 3-Br, 6-CF₃ |
| Y. 358 | CyPr | H | H | 3-Br, 4-F |
| Y. 359 | CyPr | H | H | 2-Br, 5-Cl |
| Y. 360 | CyPr | H | H | 3-Br, 4-Cl |
| Y. 361 | CyPr | H | H | 2-F, 3-CH₃ |
| Y. 362 | CyPr | H | H | 2-F, 4-CH₃ |
| Y. 363 | CyPr | H | H | 2-F, 5-CH₃ |
| Y. 364 | CyPr | H | H | 2-F, 6-CH₃ |
| Y. 365 | CyPr | H | H | 2-Cl, 3-CH₃ |
| Y. 366 | CyPr | H | H | 2-CH₃, 3-Cl |
| Y. 367 | CyPr | H | H | 2-CH₃, 4-Cl |
| Y. 368 | CyPr | H | H | 2-CH₃, 5-Cl |
| Y. 369 | CyPr | H | H | 2-F, 5-CH₃ |
| Y. 370 | CyPr | H | H | 2-F, 6-CH₃ |
| Y. 371 | CyPr | H | H | 2-Br, 5-CH₃ |
| Y. 372 | CyPr | H | H | 2-CH₃, 3-Br |
| Y. 373 | CyPr | H | H | 3-CH₃, 5-Br |
| Y. 374 | CyPr | H | H | 2-CH₃, 4-Br |
| Y. 375 | CyPr | H | H | 2-CH₃, 3-F |
| Y. 376 | CyPr | H | H | 2-CH₃, 4-F |
| Y. 377 | CyPr | H | H | 2-CH₃, 5-F |
| Y. 378 | CyPr | H | H | 3-CN, 4-F |
| Y. 379 | CyPr | H | H | 3-CN, 4-Cl |
| Y. 380 | CyPr | H | H | 2-F, 5-CN |
| Y. 381 | CyPr | H | H | 2-Cl, 5-CN |
| Y. 382 | CyPr | H | H | 2-F, 4-CN |
| Y. 383 | CyPr | H | H | 2-Cl, 4-CN |
| Y. 384 | CyPr | H | H | 3-F, 4-CN |
| Y. 385 | CyPr | H | H | 3-Cl, 4-CN |
| Y. 386 | CyPr | H | H | 3-Cl, 5-CN |
| Y. 387 | CyPr | H | H | 2-Br, 5-CN |
| Y. 388 | CyPr | H | H | 2-CN, 3-Br |
| Y. 389 | CyPr | H | H | 3-CN, 5-Br |
| Y. 390 | CyPr | H | H | 2-CN, 4-Br |
| Y. 391 | CyPr | H | H | 3-Br, 4-CN |
| Y. 392 | CyPr | H | H | 2-Br, 4-CN |
| Y. 393 | CyPr | H | H | 2-Br, 4-CF₃ |
| Y. 394 | CyPr | H | H | 2-Br, 5-CF₃ |
| Y. 395 | CyPr | H | H | 2-Br, 6-CF₃ |
| Y. 396 | CyPr | H | H | 2-F, 3-CF₃ |
| Y. 397 | CyPr | H | H | 2-F, 4-CF₃ |
| Y. 398 | CyPr | H | H | 2-F, 5-CF₃ |
| Y. 399 | CyPr | H | H | 2-F, 6-CF₃ |
| Y. 400 | CyPr | H | H | 3-F, 4-CF₃ |
| Y. 401 | CyPr | H | H | 4-F, 3-CF₃ |
| Y. 402 | CyPr | H | H | 2-Cl, 3-CF₃ |
| Y. 403 | CyPr | H | H | 2-Cl, 4-CF₃ |
| Y. 404 | CyPr | H | H | 3-CF₃, 5-Br |
| Y. 405 | CyPr | H | H | 3-CF₃, 4-Br |
| Y. 406 | CyPr | H | H | 3-CF₃, 5-CH₃ |
| Y. 407 | CyPr | H | H | 2-CH₃, 4-CF₃ |
| Y. 408 | CyPr | H | H | 2-CH₃, 5-CF₃ |
| Y. 409 | CyPr | H | H | 3-CF₃, 4-CH₃ |
| Y. 410 | CyPr | H | H | 3-CF₃, 5-CN |
| Y. 411 | CyPr | H | H | 3-CN, 4-CF₃ |
| Y. 412 | CyPr | H | H | 2-OCHF₂, 4-Cl |
| Y. 413 | CyPr | H | H | 2-OCHF₂, 4-F |
| Y. 414 | CyPr | H | H | 2-Cl, 4-OCF₃ |
| Y. 415 | CyPr | H | H | 2-Cl, 5-CF₃ |
| Y. 416 | CyPr | H | H | 3-Cl, 5-CF₃ |
| Y. 417 | CyPr | H | H | 4-Cl, 3-CF₃ |
| Y. 418 | CyPr | H | H | 2-F, 4-SCH₃ |
| Y. 419 | CyPr | H | H | 2-Cl, 5-SO₂CH₃ |
| Y. 420 | CyPr | H | H | 2-OCH₃, 3-CH₃ |
| Y. 421 | CyPr | H | H | 2-OCH₃, 4-CH₃ |
| Y. 422 | CyPr | H | H | 2-OCH₃, 5-CH₃ |
| Y. 423 | CyPr | H | H | 2-OCH₃, 3-Cl |
| Y. 424 | CyPr | H | H | 2-OCH₃, 4-Cl |
| Y. 425 | CyPr | H | H | 2-OCH₃, 5-Cl |
| Y. 426 | CyPr | H | H | 2-F, 3-OCH₃ |
| Y. 427 | CyPr | H | H | 2-Cl, 3-OCH₃ |
| Y. 428 | CyPr | H | H | 2-Cl, 4-OCH₃ |
| Y. 429 | CyPr | H | H | 3-OCH₃, 4-Br |
| Y. 430 | CyPr | H | H | 3-Br, 5-OCH₃ |
| Y. 431 | CyPr | H | H | 2,4-di-F, 5-Cl |
| Y. 432 | CyPr | H | H | 2,3-di-F, 5-Cl |
| Y. 433 | CyPr | H | H | 2-Cl, 4,5-di-F |
| Y. 434 | CyPr | H | H | 3,5-di-Cl, 4-F |
| Y. 435 | CyPr | H | H | 3,4-di-F, 5-CF₃ |
| Y. 436 | CyPr | H | H | 3,5-di-Cl, 4-CH₃ |
| Y. 437 | CyPr | H | H | 3,5-di-CH₃, 4-F |
| Y. 438 | CyPr | H | H | 2,5-di-CH₃, 3-Cl |

*CyPr = cyclopropyl

Each of the Tables 1-19, which follow the Table Y above, consist of 438 compounds of the formula (I-1) in which R², R³, R⁴ and R⁵ have the values given in each row in the Table Y, and R^{1b} is defined in the relevant Tables 1-19.

Thus compound 1.1 corresponds to a compound of formula (I-1) where R², R³, R⁴ and R⁵ are as defined in row 1 of Table Y and where R^{1b} is as defined in Table 1; compound 1.2 corresponds to a compound of formula (I-1) where R², R³, R⁴ and R⁵ are as defined in row 2 of Table Y and where Het is as defined in Table 1; and so on.

Table 1: This table discloses 438 compounds 1.1 to 1.438 of the formula, wherein R^{1b} is 2-Cl, 4-OCF₃; R², R³, R⁴ and R⁵ are as defined in Table Y. For example compound No. 1.1 has the following structure:

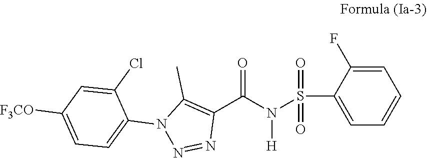 [SMILES: Cc1c(C(=O)NS(=O)(=O)c2ccccc2F)nnn1-c1ccc(OC(F)(F)F)cc1Cl]

Table 2: This table discloses 438 compounds 2.1 to 2.438 of the formula, wherein R^{1b} is 2-Cl, 4-Cl; R², R³, R⁴ and R⁵ are as defined in Table Y.

Table 3: This table discloses 438 compounds 3.1 to 3.438 of the formula, wherein R^{1b} is 2-CF₃, 4-Cl; R², R³, R⁴ and R⁵ are as defined in Table Y.

Table 4: This table discloses 438 compounds 4.1 to 4.438 of the formula, wherein R^{1b} is 2-Cl, 4-CF₃; R², R³, R⁴ and R⁵ are as defined in Table Y.

Table 5: This table discloses 438 compounds 5.1 to 5.438 of the formula, wherein R^{1b} is 2-Cl, 4-CN; R², R³, R⁴ and R⁵ are as defined in Table Y.

Table 6: This table discloses 438 compounds 6.1 to 6.438 of the formula, wherein R^{1b} is 2-Cl, 4-F; R², R³, R⁴ and R⁵ are as defined in Table Y.

Table 7: This table discloses 438 compounds 7.1 to 7.438 of the formula, wherein R^{1b} is 2-Cl, 4-OCH₃; R², R³, R⁴ and R⁵ are as defined in Table Y.

Table 8: This table discloses 438 compounds 8.1 to 8.438 of the formula, wherein R^{1b} is 2-F, 4-CN; R², R³, R⁴ and R⁵ are as defined in Table Y.

Table 9: This table discloses 438 compounds 9.1 to 9.438 of the formula, wherein R^{1b} is 2-F, 4-Cl; R², R³, R⁴ and R⁵ are as defined in Table Y.

Table 10: This table discloses 438 compounds 10.1 to 10.438 of the formula, wherein R^{1b} is 2-F, 4-F; R², R³, R⁴ and R⁵ are as defined in Table Y.

Table 11: This table discloses 438 compounds 11.1 to 11.438 of the formula, wherein R^{1b} is 2-Cl, 4-CH₃; R², R³, R⁴ and R⁵ are as defined in Table Y.

Table 12: This table discloses 438 compounds 12.1 to 12.438 of the formula, wherein R^{1b} is 2-F, 4-CH₃; R², R³, R⁴ and R⁵ are as defined in Table Y.

Table 13: This table discloses 438 compounds 13.1 to 13.438 of the formula, wherein R^{1b} is 2-CF₃, 4-Cl; R², R³, R⁴ and R⁵ are as defined in Table Y.

Table 14: This table discloses 438 compounds 14.1 to 14.438 of the formula, wherein R^{1b} is 2-Cl, 4-OCHF₂; R², R³, R⁴ and R⁵ are as defined in Table Y.

Table 15: This table discloses 438 compounds 15.1 to 15.438 of the formula, wherein R^{1b} is 3-Cl, 5-Cl; R², R³, R⁴ and R⁵ are as defined in Table Y.

Table 16: This table discloses 438 compounds 16.1 to 16.438 of the formula, wherein R^{1b} is 3-Cl, 5-OCF₃; R², R³, R⁴ and R⁵ are as defined in Table Y.

Table 17: This table discloses 438 compounds 17.1 to 17.438 of the formula, wherein R^{1b} is 3-CF₃, 5-Cl; R², R³, R⁴ and R⁵ are as defined in Table Y.

Table 18: This table discloses 438 compounds 18.1 to 18.438 of the formula, wherein R^{1b} is 3-CF₃, 5-CF₃; R², R³, R⁴ and R⁵ are as defined in Table Y.

Table 19: This table discloses 438 compounds 19.1 to 19.438 of the formula, wherein R^{1b} is 3-Cl, 5-Cl; R², R³, R⁴ and R⁵ are as defined in Table Y.

In one embodiment, the present invention provides the use of compounds of formula (I), including stereoisomers, salts, polymorphs or N-oxides thereof or compositions or combinations thereof for controlling or preventing agricultural crops and/or horticultural crops against insects, nematodes or mites.

In a preferred embodiment, the present invention provides the use of compounds of formula (I), including salts, stereoisomers, polymorphs or N-oxides thereof or compositions or combinations thereof for controlling or preventing agricultural crops and/or horticultural crops against nematodes.

The respective agricultural crops are selected from cereals, corn, rice, soybean and other leguminous plants, fruits and fruit trees, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers, other vegetables or ornamentals.

The compounds according to the invention can be used for controlling or destroying pests such as nematodes which occur in particular on plants, especially on useful plants and ornamentals in agriculture and in horticulture, or on organs, such as fruits, flowers, foliage, stalks, tubers, seeds or roots, of such plants. In some cases even plant organs which are formed at a later point in time remain protected against these pests.

The compounds of formula (I) according to the invention are preventively and/or curatively valuable active ingredients in the field of pest control, even at low rates of application, which can be used against pesticide resistant nematodes. The compounds of formula (I) according to the invention have a very favorable biocidal spectrum and are well tolerated by warm-blooded species, fish, and plants. Accordingly, the present invention also makes available a pesticidal composition comprising compounds of the invention, such as formula (I). It has now been found that the compounds of formula (I), according to the invention, have, for practical purposes, a very advantageous spectrum of activities for protecting animals and useful plants against the attack and damage by nematodes. Accordingly, the present invention also makes available a nematocidal composition comprising compounds of the invention, such as formula (I). The compounds of the formula (I) can possess the potent microbicidal activity and can be used for the control of unwanted nematodes, in agricultural or horticultural crop protection.

The compounds of formula (I) can be used as nematicides for protecting crops, for example, for the control of Tylenchida, Rhabditida, Dorylaimida, and Tryplonchida.

The compounds of formula (I) can be used for controlling or preventing the infection of agricultural crops or horticultural crops with phytopathogenic nematodes.

The compounds of formula (I) can be used for protecting crops, wherein the agricultural crops are cereals, corn, rice, soybean and other leguminous plants, fruits and fruit trees, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and other vegetables and ornamentals.

The compounds of formula (I) are especially useful for the control of nematodes. Thus, in a further aspect, the invention also relates to a method of controlling the damage to plant and parts thereof by plant parasitic nematodes (Endoparasitic, Semiendoparasitic and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Eelonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

Particularly, the nematode species Meloidogyne spp., Heterodera spp., Rotylenchus spp., Pratylenchus spp. and Radopholus spp. can be controlled with the compounds of the invention.

In one embodiment, the present invention provides a composition for controlling or preventing phytopathogenic nematodes comprising a compound of formula (I), stereoisomers, salts, polymorphs, or N-oxides thereof and one or more inert carriers.

In another embodiment, the composition may additionally comprise one or more active compatible compounds selected from fungicides, insecticides, nematicides, acaricides, biopesticides, herbicides, plant growth regulators, antibiotics, nutrients or fertilizers.

The concentration of the compound of formula (I) ranges from 1 to 90% by weight with respect to the total weight of the composition, preferably from 5 to 50% by weight with respect to the total weight of the composition.

The present invention further relates to a composition for controlling unwanted nematodes comprising at least one of the compounds of the formula (I) and one or more inert carrier. The inert carrier further comprises agriculturally suitable auxiliaries, solvents, diluents, surfactants and/or extenders and the like.

The present invention further relates to a composition for controlling unwanted nematodes, comprising at least one of the compounds of the formula (I) and/or one or more active compatible compounds selected from fungicides, bactericides, acaricides, insecticides, nematicides, herbicides, biopesticides, plant growth regulators, antibiotics, fertilizers and/or mixtures thereof.

Generally, a compound of the present invention is used in the form of a composition (e.g. formulation) containing a carrier. A compound of the invention and compositions thereof can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra-low volume (ulv) liquid, ultra-low volume (ulv) suspension, water dispersible granules or tablets, water dispersible powder for slurry treatment, water soluble granules or tablets, water soluble powder for seed treatment and wettable powder.

A formulation typically comprises a liquid or solid carrier and optionally one or more customary formulation auxiliaries, which may be solid or liquid auxiliaries, for example unepoxidized or epoxidized vegetable oils (for example epoxidized coconut oil, rapeseed oil or soya oil), antifoams, for example silicone oil, preservatives, clays, inorganic compounds, viscosity regulators, surfactant, binders and/or tackifiers. The composition may also further comprise a fertilizer, a micronutrient donor or other preparations which influence the growth of plants as well as comprising a combination containing the compound of the invention with one or more other biologically active agents, such as bactericides, fungicides, nematicides, plant activators, acaricides, and insecticides.

Accordingly, the present invention also makes available a composition comprising a compound of the invention and an agronomical carrier and optionally one or more customary formulation auxiliaries.

The compositions are prepared in a manner known per se, in the absence of auxiliaries for example by grinding, screening and/or compressing a solid compound of the present invention and, in the presence of at least one auxiliary for example, by intimately mixing and/or grinding the compound of the present invention with the auxiliary (auxiliaries). In the case of solid compounds of the invention, the grinding/milling of the compounds is to ensure a specific particle size. These processes of the preparation of the compositions and the use of the compounds of the invention for the preparation of these compositions are also a subject of the invention.

Examples of compositions for use in agriculture are emulsifiable concentrates, suspension concentrates, microemulsions, oil dispersibles, directly sprayable or dilutable solutions, spreadable pastes, dilute emulsions, soluble powders, dispersible powders, wettable powders, dusts, granules or encapsulations in polymeric substances, which comprise—at least—a compound according to the invention and the type of composition is to be selected to suit the intended aims and the prevailing circumstances.

Examples of suitable liquid carriers are unhydrogenated or partially hydrogenated aromatic hydrocarbons, preferably the fractions C₈ to C₁₂ of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils. Examples of solid carriers which are used for example for dusts and dispersible powders are, as a rule, ground natural minerals such as calcite, talc, kaolin, montmorillonite or attapulgite. To improve the physical properties, it is also possible to add highly disperse silicas or highly disperse absorbtive polymers. Suitable particulate adsorptive carriers for granules are porous types, such as pumice, brick grit, sepiolite or bentonite, and suitable non-sorptive carrier materials are calcite or sand. In addition, a large number of granulated materials of inorganic or organic nature can be used, in particular dolomite or comminuted plant residues. Suitable surface-active compounds are, depending on the type of the active ingredient to be formulated, non-ionic, cationic and/or anionic surfactants or surfactant mixtures which have good emulsifying, dispersing and wetting properties. The surfactants mentioned below are only to be considered as examples; a large number of further surfactants which are conventionally used in the art of formulation and suitable according to the invention are described in the relevant literature. Suitable non-ionic surfactants are, especially, polyglycol ether derivatives of aliphatic or (cyclo)aliphatic alcohols, of saturated or unsaturated fatty acids or of alkyl phenols which may contain approximately 3 to approximately glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediaminopolypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propylene glycol unit. Examples which may be mentioned are nonylphenoxypolyethoxyethanol, castor oil polyglycol ether, polypropylene glycol/polyethylene oxide adducts, tributylphenoxypolyethoxyethanol, polyethylene glycol or octylphenoxypolyethoxyethanol. Also suitable are fatty acid esters of polyoxyethylene sorbitan, such as polyoxyethylene sorbitan trioleate. The cationic surfactants are, especially, quarternary ammonium salts which generally have at least one alkyl radical of approximately 8 to approximately 22 Carbon atoms as substituents and as further substituents (unhalogenated or halogenated) lower alkyl or hydroxyalkyl or benzyl radicals. The salts are preferably in the form of halides, methylsulfates or ethylsulfates. Examples are stearyltrimethylammonium chloride and benzylbis(2-chloroethyl)ethylammonium bromide.

Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 Carbon atoms, such as the sodium or potassium salts of oleic or stearic acid, or of natural fatty acid mixtures which are obtainable for example from coconut or tall oil; mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 Carbon atoms, alkyl also to be understood as including the alkyl moiety of acyl radicals; examples which may be mentioned are the sodium or calcium salts of lignosulfonic acid, of the dodecylsulphuric ester or of a fatty alcohol sulfate mixture prepared from natural fatty acids. This group also includes the salts of the sulphuric esters and sulfonic acids of fatty alcohol/ethylene oxide adducts. The sulfonated benzimidazole derivatives preferably contain 2 sulfonyl groups and a fatty acid radical of approximately 8 to approximately 22 Carbon atoms. Examples of alkylarylsulfonates are the sodium, calcium or triethanolammonium salts of decylbenzenesulfonic acid, of dibutylnaphthalenesulfonic acid or of a naphthalenesulfonic acid/formaldehyde condensate.

Also possible are, furthermore, suitable phosphates, such as salts of the phosphoric ester of a p-nonylphenol/(4-14)ethylene oxide adduct, or phospholipids.

As a rule, the compositions comprise 0.1 to 99%, especially 0.1 to 95%, of a compound according to the present invention and 1 to 99.9%, especially 5 to 99.9%, of at least one solid or liquid carrier, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient.

Examples of foliar formulation types for pre-mix compositions are:

| | |
|---|---|
| GR: granules | EW: emulsions, oil in water |
| WP: wettable powders | ME: micro-emulsion |
| WG: water dispersible granules | SC: aqueous suspension |
| (powders) | concentrate |
| SG: water soluble granules | CS: aqueous capsule suspension |
| SL: soluble concentrates | OD: oil-based suspension |
| EC: emulsifiable concentrate | concentrate, and |
| | SE: aqueous suspo-emulsion. |

Whereas, examples of seed treatment formulation types for pre-mix compositions are:

| | |
|---|---|
| WS: wettable powders for seed | FS: suspension concentrates for seed |
| treatment slurry | treatment |
| LS: solution for seed treatment | WG: water dispersible granules, and |
| ES: emulsions for seed treatment | CS: aqueous capsule suspension. |

Examples of formulation types suitable for tank-mix compositions are solutions, dilute emulsions, suspensions, or a mixture thereof, and dusts.

As with the nature of the formulations, the methods of application, such as foliar, drench, spraying, atomizing, dusting, scattering, coating, or pouring, are chosen in accordance with the intended objectives and the prevailing circumstances.

The tank-mix compositions are generally prepared by diluting with a solvent (for example, water) the one or more pre-mix compositions containing different pesticides, and optionally further auxiliaries. Suitable carriers and adjuvants can be solid or liquid and are the substances ordinarily employed in formulation technology, e.g. natural or regenerated mineral substances, solvents, dispersants, wetting agents, tackifiers, thickeners, binders or fertilizers.

Generally, a tank-mix formulation for foliar or soil application comprises 0.1 to 20%, especially 0.1 to 15%, of the desired ingredients, and 99.9 to 80%, especially 99.9 to 85%, of a solid or liquid auxiliary (including, for example, a solvent such as water), where the auxiliaries can be a surfactant in an amount of 0 to 20%, especially 0.1 to 15%, based on the tank-mix formulation.

Typically, a pre-mix formulation for foliar application comprises 0.1 to 99.9%, especially 1 to 95%, of the desired ingredients, and 99.9 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation.

Normally, a tank-mix formulation for seed treatment application comprises 0.25 to 80%, especially 1 to 75%, of the desired ingredients, and 99.75 to 20%, especially 99 to 25%, of a solid or liquid auxiliary (including, for example, a solvent such as water), where the auxiliaries can be a surfactant in an amount of 0 to 40%, especially 0.5 to 30%, based on the tank-mix formulation.

Typically, a pre-mix formulation for seed treatment application comprises 0.5 to 99.9%, especially 1 to 95%, of the desired ingredients, and 99.5 to 0.1%, especially 99 to 5%, of a solid or liquid adjuvant (including, for example, a solvent such as water), where the auxiliaries can be a surfactant in an amount of 0 to 50%, especially 0.5 to 40%, based on the pre-mix formulation whereas commercial products will preferably be formulated as concentrates (e.g., pre-mix composition (formulation)), the end user will normally employ dilute formulations (e.g., tank mix composition).

Preferred seed treatment pre-mix formulations are aqueous suspension concentrates. The formulation can be applied to the seeds using conventional treating techniques and machines, such as fluidized bed techniques, the roller mill method, roto static seed treaters, and drum coaters. Other methods, such as spouted beds may also be useful. The seeds may be pre sized before coating. After coating, the seeds are typically dried and then transferred to a sizing machine for sizing. Such procedures are known in the art. The compounds of the present invention are particularly suited for use in soil and seed treatment applications.

In general, the pre-mix compositions of the invention contain 0.5 to 99.9% especially 1 to 95%, advantageously 1 to 50%, by mass of the desired ingredients, and 99.5 to 0.1%, especially 99 to 5%, by mass of a solid or liquid adjuvant (including, for example, a solvent such as water), where the auxiliaries (or adjuvant) can be a surfactant in an amount of 0 to 50%, especially 0.5 to 40%, by mass based on the mass of the pre-mix formulation.

A compound of the formula (I) in a preferred embodiment, independent of any other embodiments, is in the form of a plant propagation material treating (or protecting) composition, wherein said plant propagation material protecting compositions may comprise additionally a coloring agent. The plant propagation material protecting composition or mixture may also comprise at least one polymer from water-soluble and water-dispersible film-forming polymers that improve the adherence of the active ingredients to the treated plant propagation material, which polymer generally has an average molecular weight of at least 10,000 to about 100,000.

In an embodiment, the present invention provides a method of controlling or preventing infestation of useful plants by phytopathogenic nematodes in agricultural crops and/or horticultural crops, wherein the compound of formula (I) or salts, stereoisomers, polymorphs, or N-oxides thereof or composition or combination thereof, is applied to the plants, to parts thereof or a locus thereof.

In another embodiment, the present invention provides a method of controlling or preventing an infestation of useful plants by phytopathogenic nematodes in agricultural crops and/or horticultural crops, wherein the compound of formula (I) or salts, stereoisomers, polymorphs or N-oxides thereof or composition or combination thereof is applied to the seeds of plants.

In yet another embodiment, the present invention provides a method of controlling or preventing the infection with phytopathogenic nematodes in agricultural crops and/or horticultural crops using the compound of formula (I) or salts, stereoisomers, polymorphs or N-oxides thereof or composition or combination thereof comprising a step of applying an effective dosage of the compound or the composition or the combination, in amounts ranging from 1 g to 5 kg per hectare of agricultural crops and/or horticultural crops.

Examples of application methods for the compounds of the invention and compositions thereof, that is the method of controlling pests in the agriculture, are spraying, atomizing, dusting, brushing on, dressing, scattering or pouring which are to be selected to suit the intended aims of the prevailing circumstances.

One method of application in agriculture is the application to the foliage of the plants (foliar application), it being possible to select frequency and rate of application to match the danger of infestation with the pest in question. Alternatively, the active ingredient can reach the plants via the root system (systemic action), by applying the compound to the locus of the plants, for example by an application of a liquid composition of the compound into the soil (by drenching), or by applying a solid form of the compound in the form of granules to the soil (soil application).

In the case of paddy rice plants, such granules can be metered into the flooded paddy-field. The application of the compounds of the present invention to the soil is a preferred application method.

Typical rates of application per hectare are generally 1 to 2000 g of active ingredient per hectare, in particular 10 to 1000 g/ha, preferably 10 to 600 g/ha, such as 50 to 300 g/ha.

In one embodiment, the present invention provides a seed comprising the compound of formula (I) or salts, stereoisomers, polymorphs or N-oxides thereof or composition or combination thereof, wherein the amount of the compound of the formula (I) is ranging from 0.1 g to 10 kg per 100 kg of seed.

The compounds of the invention and compositions thereof are also suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest to be controlled and are generally between 1 to 200 g per 100 kg of seeds, preferably between 5 to 150 g per 100 kg of seeds, such as between 10 to 100 g per 100 kg of seeds. The application of the compounds of the present invention to seeds is a preferred application method.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seed.

The present invention also comprises seeds coated or treated with or containing a compound of formula (I). The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I) or a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound of formula (I), which is a preferred application method, can be carried out by any known methods, such as spraying or by dusting the seeds before sowing or during the sowing/planting of the seeds.

Suitable target plants are, in particular, cereals, such as wheat, barley, rye, oats, rice, maize or sorghum; beet, such as sugar or fodder beet; fruit, for example banana, pomaceous fruit, stone fruit or soft fruit, such as apples, pears, plums, peaches, almonds, cherries or berries, for example strawberries, raspberries or blackberries; leguminous plants, such as beans, lentils, peas or soya; oil plants, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family, latex plants and ornamentals (such as flowers, and lawn grass or turf). In an embodiment, the plant is selected from cereals, corn, soybean, rice, sugarcane, vegetables, and oil plants.

The term “plant” is to be understood as including also plants which have been so transformed by the use of recombinant DNA techniques that they are capable of synthesizing one or more selectively acting toxins, such as are known, for example, from toxin-producing bacteria, especially those of the genus Bacillus and also plants which have been selected or hybridized to preserve and/or attain a desired trait, such as insect, and/or nematode resistance. Toxins that can be expressed by such transgenic plants include, for example, insecticidal proteins from Bacillus cereus or Bacillus popilliae; or insecticidal proteins from Bacillus thuringiensis, such as 8-endotoxins, e.g. Cry1Ab, Cry1Ac, Cry1F, CrylFa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases. In the context of the present invention there are to be understood by 8-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins.

Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesizing such toxins are disclosed, for example, in EP-A-0 374 753, WO 93/07278, WO 95/34656, EP-A-0 427 529, EP-A-451 878 and WO 03/052073. The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367474, EP-A-0 401 979 and WO 90/13651. The toxin contained in the transgenic plants imparts to the plant's tolerance to harmful insects. Such insects can occur in any taxonomic group of insects but are especially commonly found in the beetles (Coleoptera), two-winged insects (Diptera) and butterflies (Lepidoptera). Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); HerculexI® (maize variety that expresses a Cry1Fa2 toxin and the enzyme phosphinothricine N-acetyltransferase(PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); Newleaf® (potato variety that expresses a Cry3A toxin); NatureGard®, 25Agrisure® GT Advantage (GA21 glyphosate-tolerant trait), Agrisure® CB Advantage (Bt11 corn borer (CB) trait) and Protecta®. Further examples of such transgenic plants are: i) Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubi/alis and Sesamia nonagrioides) by transgenic expression of a truncated Cry1Ab toxin. Bt11 maize also transgenically expresses the enzyme PAT to achieve tolerance to the herbicide glufosinate ammonium; ii) Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubi/alis and Sesamia nonagrioides) by transgenic expression of a Cry1Ab toxin. Bt176 maize also transgenically expresses the enzyme PAT to achieve tolerance to the herbicide glufosinate ammonium; iii) MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by the insertion of a cathepsin-G-protease recognition sequence. The preparation of such transgenic maize plants is described in WO 03/018810; iv) MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects; v) IPC 531 Cotton from Monsanto Europe S.A. 270-272 Avenue de Tervuren, B-1150 Brussels, Belgium, registration number C/ES/96/02; vi) 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium; vii) NK603×MON 810 Maize from Monsanto Europe S.A. 270-272 Avenue de Tervuren, B-1150 Brussels, Belgium, registration number C/GB/02/M3/03. Consists of conventionally breed hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

In one embodiment, the present invention provides a combination comprising a compound of formula (I), including its salts, stereoisomers, polymorphs or N-oxides thereof and one or more active compatible compound(s) selected from fungicides, insecticides, nematicides, acaricides, biopesticides, herbicides, plant growth regulators, antibiotics, nutrients or fertilizers.

Compounds of this invention are effective for controlling the nematodes of agronomic plants, both growing and harvested, when employed alone. They may also be used in combination with other biological active agents applied in agriculture, such as one or more chemical nematicides, biological nematicides, insecticides, acaricides, fungicides, biologics, bactericides, plant activator, molluscicide, and pheromones (whether chemical or biological). Mixing the compounds of the invention or the compositions thereof in the use form as pesticides with other pesticides frequently results in a broader pesticidal spectrum of action. For example, the formula (I) compounds of this invention may be used effectively in conjunction or combination with pyrethroids, neonicotinoids, macrolides, diamides, phosphates, carbamates, cyclodienes, formamidines, phenol tin compounds, chlorinated hydrocarbons, benzoylphenyl ureas, pyrroles and the like.

The activity of the compositions according to the invention can be broadened considerably and adapted to prevailing circumstances, by adding, for example, one or more insecticidally, acaricidally, nematicidally and/or fungicidally active agents. The combination of compounds of formula (I) with other insecticidally, acaricidally, nematicidally and/or fungicidally active agents including biologics may also have further surprising advantages. For example, better tolerance by plants, reduced phytotoxicity, pests can be controlled in their different development stages or better behavior during their production, for example during grinding or mixing, during their storage or during their use.

The known and reported active compounds such as fungicides, insecticides, nematicides, acaricides, biopesticides, herbicides, safeners, plant growth regulators, antibiotics, fertilizers and nutrients can be combined with at least one compound of formula (I) of the present invention. For example, fungicides, insecticides, nematicides, acaricides, biopesticides, herbicides, safeners, plant growth regulators, antibiotics, fertilizers, and nutrients disclosed and reported in WO2017076739 (A to 0) can be combined with the compound of formula (I) of the present invention. The present invention also relates to such combinations comprising compounds of the present invention and active compatible compounds reported in WO2017076739.

The active substances referred above, their preparation and their activity e.g. against harmful fungi/insects/nematodes is known (cf.: http://www.alanwood.net/pesticides/); these substances are mostly commercially available.

The mass ratio of any two ingredients in each combination is selected as to give the desired effect, for example, enhanced activity. In general, the mass ratio would vary depending on the specific ingredient and how many ingredients are present in the combination.

According to further embodiments of the binary combination and compositions, the weight ratio of the component 1) and the component 2) usually is in the range of from 1000:1 to 1:1, often in the range of from 100:1 to 1:1, regularly in the range of from 50:1 to 1:1.

According to a further embodiments of the binary combination and compositions, the weight ratio of the component 1) and the component 2) usually is in the range of from 1:1 to 1:1000, often in the range of from 1:1 to 1:100, regularly in the range of from 1:1 to 1:50.

The mixing ratios are understood to include, on the one hand, ratios by mass and also, on other hand, molar ratios.

The combinations of the present invention (i.e. those comprising a compound of the present invention and one or more other biological active agents) may be applied simultaneously or sequentially.

In the event, the ingredients of a combination are applied sequentially within a reasonable period of each other to attain the biological performance, such as within a few hours or days.

The order of applying the ingredients in the combination is not essential for working the present invention.

In the event ingredients of the combinations are applied simultaneously in the present invention, they may be applied as a composition containing the combination, in which case (A) the compound of formula (I) and the one or more other ingredients in the combinations can be obtained from separate formulation sources and mixed together (known as a tank-mix, ready-to-apply, spray broth, or slurry), or (B) the compound of formula (I) and the one or more other ingredients can be obtained as single formulation mixture source (known as a pre-mix, ready-mix, concentrate, or formulated product).

In an embodiment, independent of other embodiments, a compound according to the present invention is applied as a combination. Accordingly, the present invention also provides a composition comprising a compound according to the invention as herein described and one or more other biological active agents, and optionally one or more customary formulation auxiliaries, which may be in the form of a tank-mix or pre-mix composition.

The compounds of formula (I) are particularly useful for controlling and preventing helminth and nematode endo and ecto-parasitic infestations and infections in warm-blooded animals such as cattle, sheep, swine, camels, deer, horses, poultry, fish, rabbits, goats, mink, fox, chinchillas, dogs, and cats as well as humans. In the context of the control and prevention of an infestation and infections in warm-blooded animals, the compounds of the invention are especially useful for the control of helminths and nematodes. Examples for helminths are members of the class Trematoda, commonly known as flukes or flatworms, especially members of the genera Fasciola, Fascioloides, Paramphistomu, Dicrocoelium, Eurytrema, Ophisthorchis, Fasciolopsis, Echinostoma and Paragonimus. Nematodes which can be controlled by the compounds of formula (I) include the genera Haemonchus, Ostertagia, Cooperia, Oesphagastomu, Nematodirus, Dictyocaulus, Trichuris, Dirofilaria, Ancyclostoma, Ascaria and the like. For oral administration to warm-blooded animals, the compounds of the invention may be formulated as animal feeds, animal feed premixes, animal feed concentrates, pills, solutions, pastes, suspensions, drenches, gels, tablets, boluses and capsules. In addition, the compounds of the invention may be administered to the animals in their drinking water. For oral administration, the dosage form chosen should provide the animal with about 0.01 mg/kg to 10 g/kg of animal body weight per day of the compound of the invention. Alternatively, the compounds of the invention may be administered to animals parenterally, for example, by intraruminal, intramuscular, intravenous or subcutaneous injection. The compounds of the invention may be dispersed or dissolved in a physiologically acceptable carrier for a subcutaneous injection. Alternatively, the compounds of the invention may be formulated into an implant for subcutaneous administration. In addition, the compounds of the invention may be transdermally administered to animals. For parenteral administration, the dosage form chosen should provide the animal with about 0.01 mg/kg to 100 mg/kg of animal body weight per day of the compound of the invention.

The compounds of the invention may also be applied topically to the animals in the form of dips, dusts, powders, collars, medallions, sprays and pour-on formulations. For topical application, dips and sprays usually contain about 0.5 ppm to 5,000 ppm and preferably about 1 ppm to 3,000 ppm of the compound of the invention. In addition, the compounds of the invention may be formulated as ear tags for animals, particularly quadrupeds such as cattle and sheep.

In an embodiment, independent of any other embodiments, a compound of formula (I) is an anti-helminth compound.

In an embodiment, independent of any other embodiments, a compound of formula (I) is a pesticidal compound, preferably a nematicidal compound.

The compounds of the present invention not only control nematode pests effectively but also show positive crop response such as plant growth enhancement effects like enhanced crop vigor, enhanced root growth, enhanced tolerance to drought, high salt, high temperature, chill, frost or light radiation, improved flowering, efficient water & nutrient utilization (such as improved nitrogen assimilation), enhanced plant product quality, more number of productive tillers, enhanced resistance to insect pests and the like, which results in higher yields.

### CHEMISTRY EXAMPLES

The following examples set forth the manner and process of making compounds of the present invention without being a limitation thereof and include the best mode contemplated by the inventors for carrying out the invention.

### Example-1: 1-(3,5-dichlorophenyl)-N-((2,5-dichlorophenyl)sulfonyl)-1H-1,2,3-triazole-4-carboxamide

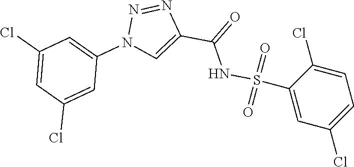 [SMILES: O=C(NS(=O)(=O)c1cc(Cl)ccc1Cl)c1cn(-c2cc(Cl)cc(Cl)c2)nn1]

### Step-1: Preparation of 1-azido-3,5-dichlorobenzene

To a stirred solution of 3,5-dichloroaniline (8 g, 49.4 mmol) in acetonitrile (80 mL), azidotrimethylsilane (9.87 mL, 74.1 mmol) and tert-butyl nitrite (8.81 mL, 74.1 mmol) were successively added at 0° C., and the resulting mixture was stirred for 3 h at the same temperature. After the completion of the reaction, the reaction mixture was concentrated under reduced pressure to obtain a crude product which was purified by column chromatography on silica gel to obtain 1-azido-3,5-dichlorobenzene (8 g, 42.6 mmol, 86% yield) as a pale brown solid; LCMS (M-2): 186.0.

### Step-2: Preparation of ethyl 1-(3,5-dichlorophenyl)-1H-1,2,3-triazole-4-carboxylate

To a stirred solution of 1-azido-3,5-dichlorobenzene (2 g, 8.93 mmol) in dimethyl sulfoxide (20 mL), ethyl propiolate (6.47 μl, 63.8 mmol) was added at 25° C., followed by the addition of an aq. solution of cupric sulfate (1.1 g, 4.3 mmol) and L-ascorbic acid sodium salt (1.7 g, 8.5 mmol). The reaction mixture was stirred at 50° C. for 12 h. After the completion of the reaction, the reaction mixture was cooled and poured into ice-cold water. The obtained solid was filtered, dried under reduced pressure and purified by column chromatography on silica gel to obtain ethyl 1-(3,5-dichlorophenyl)-1H-1,2,3-triazole-4-carboxylate (2.49 g, 8.7 mmol, 82% yield); (M−1): 285.45.

### Step-3: Preparation of 1-(3,5-dichlorophenyl)-1H-1,2,3-triazole-4-carboxylic acid

To a stirred solution of ethyl 1-(3,5-dichlorophenyl)-1H-1,2,3-triazole-4-carboxylate (9 g, 31.5 mmol) in a mixture of tetrahydrofuran (80 mL) and water (8 mL), LiOH (2.26 g, 94 mmol) was added portion wise at 25° C. The reaction mixture was stirred at 25° C. for 16 h. After the completion of the reaction, the reaction mixture was concentrated under reduced pressure. The resulting residue was neutralized by addition of 10% aq. HCl. The solid formed was filtered and dried under reduced pressure to obtain 1-(3,5-dichlorophenyl)-1H-1,2,3-triazole-4-carboxylic acid (8 g, 31.0 mmol, 99% yield); (M−1): 257.45.

### Step-4: Preparation of 1-(3,5-dichlorophenyl)-N-((2,5-dichlorophenyl)sulfonyl)-1H-1,2,3-triazole-4-carboxamide

To a stirred solution of 1-(3,5-dichlorophenyl)-1H-1,2,3-triazole-4-carboxylic acid (400 mg, 1.56 mmol) in N,N-dimethylformamide (5 mL), 1-(3-dimethylaminopropyl)-3-ethylcarbodiimide hydrochloride (594 mg, 3.10 mmol) and 1-hydroxy-1-H-benzotriazole (419 mg, 3.10 mmol) were added at 25° C. To the resulting mixture, 4-dimethylaminopyridine (189 mg, 1.55 mmol) was added. The reaction mixture was stirred for 10 min, after which 2,5-dichlorobenzenesulfonamide (421 mg, 1.90 mmol) was added. Then the reaction mixture was stirred at 25° C. for further 24 h. After the completion of the reaction, the reaction mixture was poured into ice-water (40 mL). The aqueous layer was extracted with ethyl acetate (2×50 mL), the combined organic layers were washed with water (30 mL) and brine (30 mL), dried over anhydrous sodium sulphate and concentrated under reduced pressure to obtain a crude product which was purified by column chromatography on silica gel to obtain 1-(3,5-dichlorophenyl)-N-((2,5-dichlorophenyl)sulfonyl)-1H-1,2,3-triazole-4-carboxamide (70 mg, 0.15 mmol, 10% yield); LCMS (M+1): 463.9. ¹H-NMR (400 MHz, DMSO-d₆): δ 9.29 (s, 1H), 8.11 (d, J=1.6 Hz, 2H), 8.03 (d, J=2.4 Hz, 1H), 7.77 (t, J=1.6 Hz, 1H), 7.62 (dd, J=2 Hz, 1H), 7.55 (d, J=8.4 Hz, 1H); LCMS (M+): 466.8.

### Example-2: N-((2-chlorophenyl)sulfonyl)-1-(3,5-dichlorophenyl)-5-methyl-1H-1,2,3-triazole-4-carboxamide

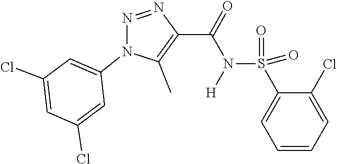 [SMILES: Cc1c(C(=O)NS(=O)(=O)c2ccccc2Cl)nnn1-c1cc(Cl)cc(Cl)c1]

### Step-1: Preparation of ethyl 1-(3,5-dichlorophenyl)-5-methyl-1H-1,2,3-triazole-4-carboxylate

To a stirred solution of ethyl 3-oxobutanoate (0.415 g, 3.19 mmol) in a mixture of dimethyl sulfoxide (9 mL) and water (1 mL), piperidine (0.053 mL, 0.532 mmol) was added at 25° C., followed by the addition of 1-azido-3,5-dichlorobenzene (0.50 g, 2.66 mmol). The resulting mixture was stirred at 25° C. for 24 h. After the completion of the reaction, the reaction mixture was poured into ice-cold water, the obtained solid was filtered and dried under reduced pressure to obtain ethyl 1-(3,5-dichlorophenyl)-5-methyl-1H-1,2,3-triazole-4-carboxylate (0.45 g, 1.50 mmol, 56% yield); (M+1): 301.8.

### Step-3: Preparation of 1-(3,5-dichlorophenyl)-5-methyl-1H-1,2,3-triazole-4-carboxylic acid

To a stirred solution of ethyl 1-(3,5-dichlorophenyl)-5-methyl-1H-1,2,3-triazole-4-carboxylate (0.123 g, 0.410 mmol) in a mixture of tetrahydrofuran (9 mL) and water (1 mL), lithium hydroxide monohydrate (0.052 g, 1.23 mmol) was added. The reaction mixture was stirred at 25° C. for further 16 h. After the completion of the reaction, the solvent was evaporated. The resultant residue was diluted with water and neutralized by the addition of a 10% HCl solution. The obtained solid was filtered and dried under reduced pressure to obtain 1-(3,5-dichlorophenyl)-5-methyl-1H-1,2,3-triazole-4-carboxylic acid (0.08 g, 0.29 mmol, 72% yield); (M+1): 270.9.

### Step-4: Preparation of N-((2-chlorophenyl)sulfonyl)-1-(3,5-dichlorophenyl)-5-methyl-1H-1,2,3-triazole-4-carboxamide

To a stirred solution of 1-(3,5-dichlorophenyl)-5-methyl-1H-1,2,3-triazole-4-carboxylic acid (500 mg, 1.84 mmol) in a mixture of dichloromethane (5 mL) and tert-butanol (5 mL), 1-ethyl-3-(3-dimethylaminopropyl) carbodiimide (528 mg, 2.76 mmol) and 4-dimethylaminopyridine (674 mg, 5.51 mmol) were added, and the resulting reaction mixture was stirred at 25° C. for 15 min. To this reaction mixture, 2-chlorobenzenesulfonamide (528 mg, 2.76 mmol) was added and the stirring was continued for further 24 h at the same temperature. After the completion of the reaction, the reaction mixture was diluted with dichloromethane (20 mL), the organic layer was washed with water (10 mL) and 10% aq. HCl solution (10 mL), dried over anhydrous sodium sulphate and concentrated under reduced pressure to obtain a crude product which was purified by column chromatography on silica gel to obtain N-((2-chlorophenyl)sulfonyl)-1-(3,5-dichlorophenyl)-5-methyl-1H-1,2,3-triazole-4-carboxamide (280 mg, 0.63 mmol, 34% yield). ¹H-NMR (400 MHz, DMSO-d₆) δ 8.16 (dd, J=7.9, 1.5 Hz, 1H), 7.93 (t, J=1.8 Hz, 1H), 7.82 (d, J=2.1 Hz, 2H), 7.71-7.57 (m, 3H), 2.41 (s, 3H); LCMS (M+1): 446.9.

### Example-3: Preparation of N-((2-chloro-5-methoxyphenyl)sulfonyl)-1-(3,5-dichlorophenyl)-5-methyl-1H-1,2,3-triazole-4-carboxamide

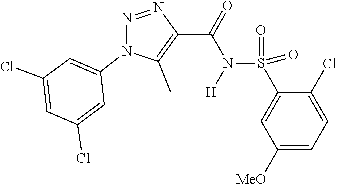 [SMILES: COc1ccc(Cl)c(S(=O)(=O)NC(=O)c2nnn(-c3cc(Cl)cc(Cl)c3)c2C)c1]

To a stirred solution of 1-(3,5-dichlorophenyl)-5-methyl-1H-1,2,3-triazole-4-carboxylic acid (200 mg, 0.74 mmol) in N,N-dimethylformamide (5 mL), 4-dimethylaminopyridine (44.9 mg, 0.34 mmol), N,N-diisopropylethylamine (0.39 mL, 2.20 mmol) and HATU (363 mg, 1.0 mmol) were added. 2-Chloro-5-methoxybenzenesulfonamide (163 mg, 0.74 mmol) was then added after 10 min. The reaction mixture was stirred at 25° C. for further 12 h. After the completion of the reaction, the reaction mixture was diluted with dichloromethane (15 mL) and neutralised by the addition of 10% aq. HCl. The organic layer was separated, washed with water (10 mL), dried over anhydrous sodium sulphate and concentrated under reduced pressure to obtain a crude product which was purified by column chromatography on silica gel to obtain N-((2-chloro-5-methoxyphenyl)sulfonyl)-1-(3,5-dichlorophenyl)-5-methyl-1H-1,2,3-triazole-4-carboxamide (300 mg, 0.63 mmol, 86% yield). ¹H NMR (400 MHz, DMSO-d₆): δ 7.85 (t, J=1.6 Mz, 1H), 7.78 (d, J=1.6 Mz, 2H), 7.55 (d, J=2.8 Mz, 1H), 7.29 (d, J=8.8 Mz, 1H), 6.97 (dd, J=2.8, 3.2 Mz, 1H), 3.78 (s, 3H), 2.55 (s, 3H); LCMS (M+1): 476.9.

### Example-4: N-((2-chloro-5-methoxyphenyl)sulfonyl)-1-(3,3,3-trifluoropropyl)-1H-1,2,3-triazole-4-carboxamide

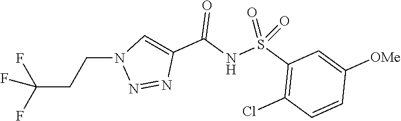 [SMILES: COc1ccc(Cl)c(S(=O)(=O)NC(=O)c2cn(CCC(F)(F)F)nn2)c1]

### Step-1: Preparation of ethyl 1-(3,3,3-trifluoropropyl)-1H-1,2,3-triazole-4-carboxylate

To a stirred solution of 1,1,1-trifluoro-3-iodopropane (2 g, 8.93 mmol) in dimethyl sulfoxide (20 mL), sodium azide (0.581 g, 8.93 mmol) was added, and the stirring was continued at 25° C. for 12 h. Water (10 mL) was added to the reaction mass, followed by the addition of ethyl propiolate (0.91 mL, 8.93 mmol), L-ascorbic acid sodium salt (0.531 g, 2.68 mmol) and the dropwise addition of an aq. solution of cupric sulfate (0.33 g, 1.34 mmol). The reaction mixture was allowed to stir at 25° C. for further 12 h. After the completion of the reaction, the reaction mixture was diluted with water (20 mL). The aqueous layer was extracted with ethyl acetate (2×20 mL), the combined organic layers were washed with a saturated aqueous brine solution (20 mL), dried over anhydrous sodium sulphate and concentrated under reduced pressure to obtain ethyl 1-(3,3,3-trifluoropropyl)-1H-1,2,3-triazole-4-carboxylate (1.8 g, 7.59 mmol, 85% yield) as a brown solid; (M+1): 238.0.

### Step-2: Preparation of 1-(3,3,3-trifluoropropyl)-1H-1,2,3-triazole-4-carboxylic acid

To a stirred solution of ethyl 1-(3,3,3-trifluoropropyl)-1H-1,2,3-triazole-4-carboxylate (1.50 g, 6.32 mmol) in a mixture of tetrahydrofuran (10 mL) and water (2 mL), lithium hydroxide (0.76 g, 31.6 mmol) was added and the reaction mixture was allowed to stir at 25° C. for 12 h. After the completion of the reaction, the solvent was evaporated. The residue obtained was diluted with water and acidified by the addition of 10% aq. HCl solution. The obtained solid was filtered, washed with water and dried under reduced pressure to obtain 1-(3,3,3-trifluoropropyl)-1H-1,2,3-triazole-4-carboxylic acid (1.1 g, 5.26 mmol, 83% yield) as a white solid; (M+1): 209.90.

### Step-3: Preparation of N-((2-chloro-5-methoxyphenyl)sulfonyl)-1-(3,3,3-trifluoropropyl)-1H-1,2,3-triazole-4-carboxamide

To a stirred solution of 4-dimethylaminopyridine (438 mg, 3.59 mmol) and 1-ethyl-3-(3-dimethylaminopropyl)carbodiimide (573 mg, 2.99 mmol) in a mixture of tert-butanol (5 mL) and dichloromethane (5 mL), 1-(3,3,3-trifluoropropyl)-1H-1,2,3-triazole-4-carboxylic acid (250 mg, 1.12 mmol) was added, and the resulting reaction mixture was stirred at 25° C. for 15 min. Subsequently, 2-chloro-5-methoxybenzenesulfonamide (244 mg, 1.10 mmol) was added to the reaction mixture and the stirring was continued at 25° C. for 12 h. After the completion of the reaction, the reaction mixture was diluted with water (10 mL). The aqueous layer was extracted with dichloromethane (2×10 mL), the combined organic layers were washed with aq. 10% HCl solution, dried over anhydrous sodium sulphate and concentrated under reduced pressure to obtain a crude product which was purified by column chromatography on silica gel to obtain N-((2-chloro-5-methoxyphenyl)sulfonyl)-1-(3,3,3-trifluoropropyl)-1H-1,2,3-triazole-4-carboxamide (270 mg, 0.65 mmol, 55% yield). ¹H NMR (400 MHz, DMSO-d₆): δ 8.85 (s, 1H), 7.60-7.55 (m, 2H), 7.28 (dd, J=2.8, 3.2 Mz, 1H), 4.73-4.67 (m, 2H), 3.84 (s, 3H), 3.06-2.95 (m, 2H); LCMS (M+1): 412.9.

### Example-5: 1-(3,5-dichlorobenzyl)-N-((2-fluorophenyl)sulfonyl)-1H-1,2,3-triazole-4-carboxamide

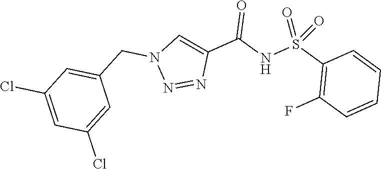 [SMILES: O=C(NS(=O)(=O)c1ccccc1F)c1cn(Cc2cc(Cl)cc(Cl)c2)nn1]

### Step-1: Preparation of ethyl 1-(3,5-dichlorobenzyl)-1H-1,2,3-triazole-4-carboxylate

To a stirred solution of 1-(bromomethyl)-3,5-dichlorobenzene (1 g, 4.17 mmol) in dimethyl sulfoxide (10 mL), sodium azide (0.27 g, 4.17 mmol) was added, and the resulting mixture was allowed to stir at 25° C. for 12 h. Water (10 mL) was added to the reaction mixture followed by the addition of L-ascorbic acid sodium salt (0.25 g, 1.250 mmol), ethyl propiolate (0.42 mL, 4.17 mmol) and an aq. solution of cupric sulfate (0.156 g, 0.63 mmol). The reaction mixture was stirred further at 25° C. for 12 h. After the completion of the reaction, the reaction mixture was diluted with water (10 mL). The aqueous layer was extracted with ethyl acetate (2×15 mL), dried over anhydrous sodium sulphate and concentrated under reduced pressure to obtain a crude product which was purified by column chromatography on silica gel to obtain ethyl 1-(3,5-dichlorobenzyl)-1H-1,2,3-triazole-4-carboxylate (0.96 g, 3.20 mmol, 77% yield) as a greenish liquid; (M−1): 299.95.

### Step-2: Preparation of 1-(3,5-dichlorobenzyl)-1H-1,2,3-triazole-4-carboxylic acid

To a stirred solution of ethyl 1-(3,5-dichlorobenzyl)-1H-1,2,3-triazole-4-carboxylate (1 g, 3.33 mmol) in a mixture of tetrahydrofuran (30 mL) and water (5 mL), lithium hydroxide (0.399 g, 16.66 mmol) was added, and the resulting reaction mixture was allowed to stir at 25° C. for 12 h. After the completion of the reaction, the solvent was evaporated under reduced pressure. The residue obtained was diluted with water and acidified by addition of aq. 10% HCl solution. The obtained solid was filtered, washed with water and dried under reduced pressure to obtain 1-(3,5-dichlorobenzyl)-1H-1,2,3-triazole-4-carboxylic acid (512 mg, 1.88 mmol, 56% yield) as a white solid; (M+1): 270.9.

### Step-3: Preparation of 1-(3,5-dichlorobenzyl)-N-((2-fluorophenyl)sulfonyl)-1H-1,2,3-triazole-4-carboxamide

To a stirred solution of 4-dimethylaminopyridine (337 mg, 2.76 mmol) and 1-ethyl-3-(3-dimethylaminopropyl)carbodiimide (440 mg, 2.297 mmol) in a mixture of tert-butanol (4 mL) and dichloromethane (4 mL), 1-(3,5-dichlorobenzyl)-1H-1,2,3-triazole-4-carboxylic acid (250 mg, 0.92 mmol) was added, and the resulting reaction mixture was stirred at 25° C. for 15 min followed by the addition of 2-fluorobenzenesulfonamide (148 mg, 0.86 mmol). The resulting reaction mixture was stirred at 25° C. for further 16 h. After the completion of the reaction, the reaction mixture was diluted with water (10 mL). The aqueous layer was extracted with dichloromethane (2×20 mL), the combined organic layers were washed with aq. 10% HCl solution, dried over anhydrous sodium sulphate and concentrated under reduced pressure to obtain a crude product which was purified by column chromatography on silica gel to obtain 1-(3,5-dichlorobenzyl)-N-((2-fluorophenyl)sulfonyl)-1H-1,2,3-triazole-4-carboxamide (110 mg, 0.256 mmol, 27.9% yield) as a white solid. ¹H NMR (400 MHz, DMSO-d₆): δ 8.85 (s, 1H), 8.0-7.96 (m, 1H), 7.79-7.73 (m, 1H), 7.63 (t, J=4.0 Mz, 1H), 7.46-7.41 (m, 4H), 5.68 (s, 2H); LCMS (M+1): 428.8.

### Example-6: N-((2-chlorophenyl)sulfonyl)-1-(3,5-dichlorobenzyl)-5-methyl-1H-1,2,3-triazole-4-carboxamide

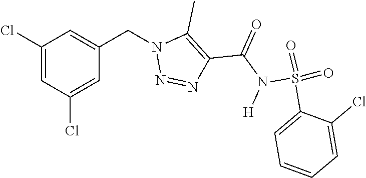 [SMILES: Cc1c(C(=O)NS(=O)(=O)c2ccccc2Cl)nnn1Cc1cc(Cl)cc(Cl)c1]

### Step-1: Preparation of 1-(azidomethyl)-3,5-dichlorobenzene

To a stirred solution of 1-(bromomethyl)-3,5-dichlorobenzene (2.5 g, 10.42 mmol) in dimethylsulfoxide (15 mL), sodium azide (0.813 g, 12.50 mmol) was added and the resulting reaction mixture was stirred at 25° C. for 18 h. After the completion of the reaction, the reaction mixture was diluted with ethyl acetate (30 mL), washed with water (30 mL) and brine solution (30 mL), dried over anhydrous sodium sulphate and concentrated under reduced pressure to obtain a crude product which was purified by column chromatography on silica gel to obtain 1-(azidomethyl)-3,5-dichlorobenzene (1.80 g, 8.91 mmol, 86% yield); (M−1): 200.9.

### Step-2: Preparation of ethyl 1-(3,5-dichlorobenzyl)-5-methyl-1H-1,2,3-triazole-4-carboxylate

To a stirred solution of ethyl 3-oxobutanoate (5.77 mL, 45.3 mmol) in dimethyl sulfoxide (50 mL), potassium carbonate (16.7 g, 121 mmol) was added followed by the addition of 1-(azidomethyl)-3,5-dichlorobenzene (6.10 g, 30.2 mmol). The resulting reaction mixture was stirred at 40° C. for 8 h. After the completion of the reaction, the reaction mixture was poured into ice-cold water. The obtained solid was filtered and dried under reduced pressure to obtain a crude product which was purified by column chromatography on silica gel to obtain ethyl 1-(3,5-dichlorobenzyl)-5-methyl-1H-1,2,3-triazole-4-carboxylate (7.8 g, 24.8 mmol, 82% yield); (M+1): 315.75.

### Step-3: Preparation of 1-(3,5-dichlorobenzyl)-5-methyl-1H-1,2,3-triazole-4-carboxylic acid

To a stirred solution of ethyl 1-(3,5-dichlorobenzyl)-5-methyl-1H-1,2,3-triazole-4-carboxylate (800 mg, 2.55 mmol) in a mixture of water (10 mL), tetrahydrofuran (30 mL) and ethanol (20 mL), lithium hydroxide hydrate (214 mg, 5.09 mmol) was added. The resulting solution was stirred at 25° C. for 6 h. After the completion of the reaction, the reaction mixture was concentrated under reduced pressure. The residue was diluted with water and acidified by the addition of aq.10% HCl solution. The obtained solid was filtered and dried to obtain 1-(3,5-dichlorobenzyl)-5-methyl-1H-1,2,3-triazole-4-carboxylic acid (324 mg, 1.13 mmol, 44.5% yield) as pale yellow solid; (M+1): 286.00.

### Step-4: Preparation of N-((2-chlorophenyl)sulfonyl)-1-(3,5-dichlorobenzyl)-5-methyl-1H-1,2,3-triazole-4-carboxamide

To a stirred solution of 4-dimethylaminopyridine (448 mg, 3.67 mmol) and 1-ethyl-3-(3-dimethylaminopropyl)carbodiimide (469 mg, 2.45 mmol) in a mixture of tert-butanol (5 mL) and dichloromethane (5 mL), 1-(3,5-dichlorobenzyl)-5-methyl-1H-1,2,3-triazole-4-carboxylic acid (350 mg, 1.22 mmol) was added and stirred for 15 min followed by the addition of 2-chlorobenzenesulfonamide (216 mg, 1.13 mmol). The resulting mixture was stirred at 25° C. for further 12 h. After the completion of the reaction, the reaction mixture was diluted with dichloromethane (10 mL), washed with aq. 10% HCl solution, dried over anhydrous sodium sulphate and concentrated under reduced pressure to obtain a crude product which was purified by column chromatography on silica gel to obtain N-((2-chlorophenyl)sulfonyl)-1-(3,5-dichlorobenzyl)-5-methyl-1H-1,2,3-triazole-4-carboxamide (386.7 mg, 0.84 mmol, 69% yield). ¹H-NMR (400 MHz, DMSO-d₆) δ 12.97 (s, NH), 8.14 (dd, J=7.8, 1.5 Hz, 1H), 7.71-7.57 (m, 4H), 7.30 (d, J=2.0 Hz, 2H), 5.63 (s, 2H), 2.38 (s, 3H); LCMS (M+1): 460.80.

### Example-7: N-((4-chlorophenyl)sulfonyl)-1-(2,4-dichlorobenzyl)-1H-1,2,3-triazole-4-carboxamide

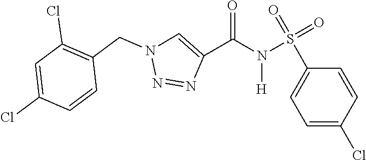 [SMILES: O=C(NS(=O)(=O)c1ccc(Cl)cc1)c1cn(Cc2ccc(Cl)cc2Cl)nn1]

### Step-1: Preparation of 1-(azidomethyl)-2,4-dichlorobenzene

To a stirred solution of 1-(bromomethyl)-2,4-dichlorobenzene (5.0 g, 20.84 mmol) in dimethyl sulfoxide (25 mL), sodium azide (2.17 g, 33.3 mmol) was added and the resulting mixture was allowed to stir at 25° C. for 18 h. After the completion of the reaction, the reaction mixture was diluted with ethyl acetate, washed with water, and then with brine. The organic layer was dried over anhydrous sodium sulphate, filtered and concentrated under reduced pressure to obtain a crude product. The obtained crude product was purified by column chromatography to afford ethyl 1-(2,4-dichlorobenzyl)-1H-1,2,3-triazole-4-carboxylate (4.8 g, 16.0 mmol, 77% yield); (M−1): 200.1.

### Step-2: Preparation of ethyl 1-(2,4-dichlorobenzyl)-1H-1,2,3-triazole-4-carboxylate

To a stirred solution of 1-(azidomethyl)-2,4-dichlorobenzene (2.70 g, 13.36 mmol) in dimethyl sulfoxide (15 mL), L-ascorbic acid sodium salt (0.32 g, 1.60 mmol), ethyl propiolate (1.36 mL, 13.36 mmol), and an aq. solution of copper(II) sulfate pentahydrate (0.334 g, 1.336 mmol) were added slowly. The resulting reaction mixture was stirred at 25° C. for 18 h. After the completion of the reaction, the reaction mixture was poured into ice-cold water. The obtained solid was filtered, dried under reduced pressure and purified by column chromatography on silica gel to obtain ethyl 1-(2,4-dichlorobenzyl)-1H-1,2,3-triazole-4-carboxylate (1.5 g, 5.0 mmol, 37% yield); (M+1): 299.90.

### Step-3: Preparation of 1-(2,4-dichlorobenzyl)-1H-1,2,3-triazole-4-carboxylic acid

To a stirred solution of ethyl 1-(2,4-dichlorobenzyl)-1H-1,2,3-triazole-4-carboxylate (1.40 g, 4.66 mmol) in a mixture of water (5 mL), tetrahydrofuran (15 mL) and ethanol (10 mL), lithium hydroxide hydrate (0.4 g, 9.33 mmol) was added, and the resulting mixture was stirred at 25° C. for 6 h. After completion of the reaction, the reaction mixture was concentrated under reduced pressure to obtain a crude product which was diluted with water and acidified by the addition of aq. 10% HCl solution. The solid obtained was filtered and dried to obtain 1-(2,4-dichlorobenzyl)-1H-1,2,3-triazole-4-carboxylic acid (1.2 g, 4.41 mmol, 95% yield); (M+1): 272.08.

### Step-4: Preparation of N-((4-chlorophenyl)sulfonyl)-1-(2,4-dichlorobenzyl)-1H-1,2,3-triazole-4-carboxamide

To a stirred solution of 4-dimethylaminopyridine (550 mg, 4.50 mmol) and 1-ethyl-3-(3-dimethylaminopropyl)carbodiimide (493 mg, 2.57 mmol) in a mixture of tert-butanol (5 mL) and dichloromethane (5 mL), 1-(2,4-dichlorobenzyl)-1H-1,2,3-triazole-4-carboxylic acid (350 mg, 1.3 mmol) was added and the reaction mixture was stirred for 15 min followed by the addition of 4-chlorobenzenesulfonamide (247 mg, 1.286 mmol). The reaction mixture was stirred further for 12 h at 25° C. After completion of the reaction, the reaction mixture was diluted with dichloromethane (10 mL), washed with aq. 10% HCl solution, dried over anhydrous sodium sulphate and concentrated under reduced pressure to obtain a crude product which was purified by column chromatography on silica gel to obtain N-((4-chlorophenyl)sulfonyl)-1-(2,4-dichlorobenzyl)-1H-1,2,3-triazole-4-carboxamide (333.4 mg, 0.75 mmol, 58.2% yield); ¹H-NMR (400 MHz, DMSO-d₆) δ 8.77 (s, 1H), 7.98-7.95 (m, 2H), 7.73-7.69 (m, 3H), 7.48 (dd, J=8.3, 2.2 Hz, 1H), 7.35 (d, J=8.3 Hz, 1H), 5.74 (s, 2H); LCMS (M+1): 446.80.

### Example-8: 1-(2,4-dichlorophenyl)-N-((2,5-dimethylphenyl)(methyl)(oxo)-λ⁶-sulfaneylidene)-5-methyl-1H-1,2,3-triazole-4-carboxamide

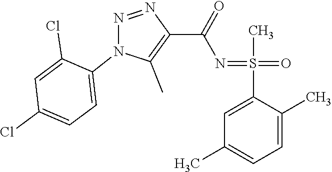 [SMILES: Cc1ccc(C)c(S(C)(=O)=NC(=O)c2nnn(-c3ccc(Cl)cc3Cl)c2C)c1]

### Step-1: Preparation of (2,5-dimethylphenyl)(methyl)sulfane

To a stirred solution of 2,5-dimethylbenzenethiol (500 mg, 3.62 mmol) in N,N-dimethylformamide (10 mL), potassium carbonate (1000 mg, 7.23 mmol) was added, and cooled to 0° C. Iodomethane (3.49 ml, 55.8 mmol) was added and the resulting reaction mixture was stirred at 25° C. for 24 h. After the completion of the reaction, the reaction mixture was quenched in ice cold water (20 mL). The aqueous layer was extracted with ethyl acetate (2×25 mL), the combined organic layers were washed with saturated brine solution (30 mL), dried over anhydrous sodium sulphate and concentrated under reduced pressure to obtain a crude product which was purified by column chromatography on silica gel to obtain the desired product (2,5-dimethylphenyl)(methyl)sulfane (400 mg, 2.63 mmol, 72.6% yield); (M+1) 152.2.

### Step-2: Preparation of (2,5-dimethylphenyl)(imino)(methyl)-λ⁶-sulfanone

To a stirred solution of (2,5-dimethylphenyl)(methyl)sulfane (200 mg, 1.314 mmol) in methanol (30 mL), ammonium carbamate (205 mg, 2.63 mmol) was added at 0° C. The reaction mixture was warmed to 25° C. and diacetoxy iodobenzene (846 mg, 2.63 mmol) was added portion wise at 25° C. over 15 min. The resulting reaction mixture was stirred at 25° C. for 16 h. After the completion of the reaction, the reaction mixture was evaporated under reduced pressure to obtain a crude product which was diluted with dichloromethane (50 mL). The organic layer was washed with water (100 mL) and saturated brine solution (50 mL), dried over anhydrous sodium sulphate and concentrated under reduced pressure to obtain a crude compound which was purified by column chromatography on silica gel to obtain (2,5-dimethylphenyl)(imino)(methyl)-λ⁶-sulfanone (144 mg, 0.788 mmol, 60% yield); (M+1) 183.2.

### Step-3: Preparation of 1-(2,4-dichlorophenyl)-N-((2,5-dimethylphenyl)(methyl)(oxo)-λ⁶-sulfaneylidene)-5-methyl-1H-1,2,3-triazole-4-carboxamide

To a stirred solution of 1-(2,4-dichlorophenyl)-5-methyl-1H-1,2,3-triazole-4-carboxylic acid (223 mg, 0.818 mmol) in dichloroethane (6 mL), hexafluorophosphate azabenzotriazole tetramethyl uronium (HATU) (622 mg, 1.637 mmol) and 4-dimethylaminopyridine (200 mg, 1.637 mmol) were added at 25° C. and the reaction mixture was stirred for 15 minutes followed by the addition of (2,5-dimethylphenyl)(imino)(methyl)-λ⁶-sulfanone (150 mg, 0.818 mmol). The resulting reaction mixture was stirred at 25° C. for further 16 h. After the completion of the reaction, the reaction mixture was diluted with water (20 mL) and the aqueous layer was extracted with dichloromethane (2×30 mL). The combined organic layers were separated, washed with aq. 10% HCl solution (15 L), dried over anhydrous sodium sulphate and concentrated under reduced pressure to obtain a crude product which was purified by column chromatography on silica gel to obtain 1-(2,4-dichlorophenyl)-N-((2,5-dimethylphenyl)(methyl)(oxo)-λ⁶-sulfaneylidene)-5-methyl-1H-1,2,3-triazole-4-carboxamide (215 mg, 0.492 mmol, 60.1% yield). ¹H-NMR (400 MHz, DMSO-d₆) δ 8.00 (d, J=2.1 Hz, 1H), 7.91 (d, J=8.9 Hz, 1H), 7.85 (s, 1H), 7.69 (dq, J=8.9, 1.2 Hz, 1H), 7.46 (d, J=7.9 Hz, 1H), 7.37 (d, J=7.9 Hz, 1H), 3.58 (s, 3H), 2.59 (s, 3H), 2.39 (s, 3H), 2.35 (s, 3H); LCMS(M+1): 487.2.

### Example 9

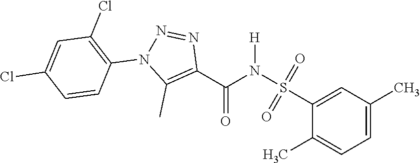 [SMILES: Cc1ccc(C)c(S(=O)(=O)NC(=O)c2nnn(-c3ccc(Cl)cc3Cl)c2C)c1]

### Step-1: Preparation of 1-azido-2,4-dichlorobenzene

To a stirred solution of 1.5N HCl (123 mL, 185 mmol) and 2,4-dichloroaniline (10 g, 61.7 mmol) in methyl tert-butyl ether (50 mL), sodium nitrite (4.90 g, 71.0 mmol), as a solution in water (25 mL), was added over 30 min. at 0-5° C. After the completion of the addition, the reaction mixture was stirred for 15 min at the same temperature followed by the addition of sodium azide (4.41 g, 67.9 mmol), as a solution in water (25 mL) over 30 min. After the completion of the addition, the reaction mixture was stirred for further 30 min at the same temperature. After the completion of the reaction, the organic layer was washed with water (60 mL) and sat. NaHCO₃ solution (60 mL), then diluted with DMSO (30 mL) and water (3 mL). The resulting mixture was concentrated under reduced pressure to remove methyl tert-butyl ether and used as such for the step 2; (M−1): 187.01.

### Step 2: Preparation of ethyl 1-(2,4-dichlorophenyl)-5-methyl-1H-1,2,3-triazole-4-carboxylate

To the crude solution of 1-azido-2,4-dichlorobenzene (11.6 g, 61.7 mmol) from step-1, ethyl-3-oxobutanoate (9.45 mL, 74.0 mmol) and piperidine (1.2 mL, 12.34 mmol) were added to the reaction mixture at 10-15° C. and the resulting mixture was stirred at 25° C. for 16 h. After the completion of the reaction, the reaction mixture was poured into ice cold water (60 mL). The precipitate obtained was filtered and the filter cake was washed with water followed by hexane. The solid was dried under reduced pressure to obtain ethyl 1-(2,4-dichlorophenyl)-5-methyl-1H-1,2,3-triazole-4-carboxylate (16.62 g, 55.4 mmol, 90% yield) as an off-white solid; M+1: 300.10.

### Step-3: Preparation of 1-(2,4-dichlorophenyl)-5-methyl-1H-1,2,3-triazole-4-carboxylic acid

1-(2,4-Dichlorophenyl)-5-methyl-1H-1,2,3-triazole-4-carboxylic acid (14.2 g, 52.2 mmol, 94% yield) was synthesized by following the same procedure as described in step-3 of example-2, using ethyl 1-(2,4-dichlorophenyl)-5-methyl-1H-1,2,3-triazole-4-carboxylate (16.6 g, 55.3 mmol); (M+1): 271.90.

### Step 4: Preparation of 2,5-dimethylbenzenesulfonyl chloride

To a stirred solution of p-xylene (11.03 mL, 89 mmol) in dichloromethane (50 mL), chlorosulfuric acid (17.87 ml, 268 mmol), as a solution in dichloromethane (50 ml), was added to the reaction mixture at 0-5° C. dropwise over 1 h and the resulting mixture was stirred further at 25° C. for 4 h. After the completion of the reaction, the reaction mixture was poured into ice water (150 mL). The aqueous layer was extracted with dichloromethane (3×150 mL), the combined organic layers were washed with saturated sodium bicarbonate solution (100 mL), brine (100 mL), dried over anhydrous sodium sulphate and concentrated under reduced pressure to obtain a crude product 2,5-dimethylbenzenesulfonyl chloride (17.8 g, 87 mmol, 97% yield) which was used in the next step without purification.

### Step 5: Preparation of 2,5-dimethylbenzenesulfonamide

To a stirred solution of 2,5-dimethylbenzenesulfonyl chloride (17 g, 83 mmol) in tetrahydrofuran (50 mL), ammonia (32 mL, 415 mmol) was added dropwise at 0-5° C. over 30 min and the resulting mixture was stirred further at 25° C. for 1 h. After the completion of the reaction, the reaction mixture was concentrated under reduced pressure to obtain a white solid which was filtered and the filter cake was washed with water, followed by hexane (50 mL) and dried under reduced pressure to obtain 2,5-dimethylbenzenesulfonamide (14.5 g, 78 mmol, 94% yield) as white solid; (M−1): 183.9.

### Step-6: 1-(2,4-dichlorophenyl)-N-((2,5-dimethylphenyl)sulfonyl)-5-methyl-1H-1,2,3-triazole-4-carboxamide

1-(2,4-dichlorophenyl)-N-((2,5-dimethylphenyl)sulfonyl)-5-methyl-1H-1,2,3-triazole-4-carboxamide was synthesized by following the procedure as described in step-4 of example-2, using 1-(2,4-dichlorophenyl)-5-methyl-1H-1,2,3-triazole-4-carboxylic acid and 2,5-dimethylbenzenesulfonamide as starting materials. ¹H-NMR (400 MHz, DMSO-d₆) δ 12.94 (s, 1H), 8.05 (d, J=1.8 Hz, 1H), 7.86 (d, J=0.9 Hz, 1H), 7.78-7.72 (m, 2H), 7.39 (d, J=7.6 Hz, 1H), 7.29 (d, J=7.6 Hz, 1H), 2.59 (s, 3H), 2.36 (s, 3H), 2.25 (s, 3H); LCMS (M+1): 440.0.

Table A: Representative compounds of the present disclosure were prepared according to the suitable methods as described in the respective schemes and examples.

TABLE A

| Comp No: | Compound Name | Analytical Data |
|---|---|---|
| 1 | 1-(3,5-dichlorophenyl)-N-((2- | ¹H-NMR (400 MHz, DMSO-d₆): δ 9.28 (bs, 1H), 8.28 (s, |
| | (trifluoromethyl)phenyl)sulfonyl)- | 1H), 8.14 (s, 2H), 7.85-7.71 (m, 3H), 7.70-7.63 (m, 1H); |
| | 1H-1,2,3-triazole-4-carboxamide | LCMS (M − 1): 464.8 |
| 2 | 1-(3,5-dichlorophenyl)-N-((2,5- | ¹H-NMR (400 MHz, DMSO-d₆): δ 9.29 (s, 1H), 8.11 (d, |
| | dichlorophenyl)sulfonyl)-1H-1,2,3- | J = 1.6 Hz, 2H), 8.03 (d, J = 2.4 Hz, 1H), 7.77 (t, J = 1.6 |
| | triazole-4-carboxamide | Hz, 1H), 7.62 (dd, J = 2 Hz, 1H), 7.55 (d, J = 8.4 Hz, 1H); |
| | | LCMS (M+): 466.8 |
| 3 | 1-(3,5-dichlorophenyl)-N-((2- | ¹H-NMR (400 MHz, DMSO-d₆): δ 12.65 (bs, 1H), 9.49 |
| | methoxyphenyl)sulfonyl)-1H-1,2,3- | (s, 1H), 8.06 (d, J = 1.8 Hz, 2H), 7.92 (dd, J = 7.9, 1.5 |
| | triazole-4-carboxamide | Hz, 1H), 7.84 (t, J = 1.8 Hz, 1H), 7.69-7.65 (m, 1H), 7.23 |
| | | (d, J = 7.9 Hz, 1H), 7.17-7.13 (m, 1H), 3.85 (s, 3H); |
| | | LCMS (M+): 427.0 |
| 4 | N-((2-chlorophenyl)sulfonyl)-1-(3,5- | ¹H-NMR (400 MHz, DMSO-d₆): δ 9.56 (s, 1H), 8.17 (d, |
| | dichlorophenyl)-1H-1,2,3-triazole-4- | J = 5.2 Hz, 1H), 8.09 (s, 2H), 7.83 (s, 1H), 7.71-7.61 (m, |
| | carboxamide | 3H); LCMS (M + 1): 432.8 |
| 5 | 1-(3,5-dichlorophenyl)-N-((2- | ¹H-NMR (400 MHz, DMSO-d₆): δ 12.58 (bs, 1H), 9.49 |
| | methoxy-5-methylphenyl)sulfonyl)- | (s, 1H), 8.06 (d, J = 1.5 Hz, 2H), 7.84 (t, J = 1.8 Hz, 1H), |
| | 1H-1,2,3-triazole-4-carboxamide | 7.71 (d, J = 2.0 Hz, 1H), 7.47 (dd, J = 8.7, 2.0 Hz, 1H), |
| | | 7.12 (d, J = 8.3 Hz, 1H), 3.85-3.76 (m, 3H), 2.33 (s, 3H); |
| | | LCMS (M − 1): 440.8 |
| 6 | N-((4-chlorophenyl)sulfonyl)-1-(3,5- | ¹H-NMR (400 MHz, DMSO-d₆): δ 9.50 (s, 1H), 8.08 (d, |
| | dichlorophenyl)-1H-1,2,3-triazole-4- | J = 1.8 Hz, 2H), 8.01 (d, J = 8.6 Hz, 2H), 7.83 (t, J = 1.7 |
| | carboxamide | Hz, 1H), 7.73 (d, J = 8.9 Hz, 2H); LCMS (M + 1): 432.7 |
| 7 | N-((2-chlorophenyl)sulfonyl)-1-(3,5- | ¹H-NMR (400 MHz, DMSO-d₆) δ 8.16 (dd, J = 7.9, 1.5 |
| | dichlorophenyl)-5-methyl-1H-1,2,3- | Hz, 1H), 7.93 (t, J = 1.8 Hz, 1H), 7.82 (d, J = 2.1 Hz, 2H), |
| | triazole-4-carboxamide | 7.71-7.57 (m, 3H), 2.41 (s, 3H); LCMS (M + 1): 446.9 |
| 8 | 1-(3,5-dichlorophenyl)-N-((2,5- | ¹H-NMR (400 MHz, DMSO-d₆) δ 8.08 (d, J = 2.4 Hz, |
| | dichlorophenyl)sulfonyl)-5-methyl- | 1H), 7.93 (t, J = 1.8 Hz, 1H), 7.82 (d, J = 1.7 Hz, 2H), |
| | 1H-1,2,3-triazole-4-carboxamide | 7.73 (dd, J = 32.3, 8.3 Hz, 2H), 2.44 (s, 3H), |
| | | (LCMS(M + 1): 380.9 |
| 9 | 1-(3,5-dichlorophenyl)-N- | ¹H-NMR (400 MHz, DMSO-d₆): δ 12.88 (bs, 1H), 9.50 |
| | (phenylsulfonyl)-1H-1,2,3-triazole- | (s, 1H), 8.07 (d, J = 1.5 Hz, 2H), 8.01 (d, J = 7.3 Hz, 2H), |
| | 4-carboxamide | 7.83 (t, J = 1.7 Hz, 1H), 7.72 (t, J = 7.3 Hz, 1H), 7.64 (t, |
| | | J = 7.5 Hz, 2H); LCMS (M − 1): 396.8 |
| 10 | 1-(3,5-dichlorophenyl)-5-methyl-N- | ¹H-NMR (400 MHz, DMSO-d₆): δ 12.69 (bs, 1H), 8.02- |
| | (phenylsulfonyl)-1H-1,2,3-triazole- | 8.00 (m, 2H), 7.93 (t, J = 1.8 Hz, 1H), 7.80 (d, J = 1.8 Hz, |
| | 4-carboxamide | 2H), 7.74-7.68 (m, 1H), 7.67-7.61 (m, 2H), 2.43 (s, 3H); |
| | | LCMS (M+): 410.9 |
| 11 | 1-(3,5-dichlorophenyl)-N-((2- | ¹H-NMR (400 MHz, DMSO-d₆): δ 12.39 (bs, 1H), 7.94 |
| | methoxyphenyl)sulfonyl)-5-methyl- | (t, J = 1.8 Hz, 1H), 7.90 (dd, J = 7.8, 1.7 Hz, 1H), 7.83 (d, |
| | 1H-1,2,3-triazole-4-carboxamide | J = 1.8 Hz, 2H), 7.68-7.64 (m, 1H), 7.23 (d, J = 8.6 Hz, |
| | | 1H), 7.14 (t, J = 7.3 Hz, 1H), 3.86 (s, 3H), 2.41 (s, 3H); |
| | | LCMS (M+): 440.9 |
| 12 | 1-(3,5-dichlorophenyl)-N-((3- | ¹H-NMR (400 MHz, DMSO-d₆): δ 12.62 (s, 1H), 7.94 (t, |
| | methoxyphenyl)sulfonyl)-5-methyl- | J = 1.8 Hz, 1H), 7.81 (d, J = 1.8 Hz, 2H), 7.60-7.51 (m, |
| | 1H-1,2,3-triazole-4-carboxamide | 3H), 7.29-7.26 (m, 1H), 3.83 (s, 3H), 2.43 (s, 3H); LCMS |
| | | (M+): 440.9 |
| 13 | N-((2-chloro-5- | ¹H-NMR (400 MHz, DMSO-d₆): δ 9.48 (s, 1H), 8.36 (d, |
| | (trifluoromethyl)phenyl)sulfonyl)-1- | J = 1.6 Hz, 1H), 8.12-8.05 (m, 3H), 7.91 (d, J = δ Hz, |
| | (3,5-dichlorophenyl)-1H-1,2,3- | 1H), 7.82 (t, J = 1.6 Hz, 1H); LCMS (M − 1): 498.7 |
| | triazole-4-carboxamide | |
| 14 | 1-(3,5-dichlorophenyl)-N-((4- | ¹H-NMR (400 MHz, DMSO-d₆): δ 12.79 (s, 1H), 9.49 (s, |
| | fluorophenyl)sulfonyl)-1H-1,2,3- | 1H), 8.09-8.06 (m, 4H), 7.88-7.83 (m, 1H), 7.48 (t, J = |
| | triazole-4-carboxamide | 8.7 Hz, 2H); LCMS (M + 1): 416.8 |
| 15 | N-((4-chlorophenyl)sulfonyl)-1-(3,5- | ¹H-NMR (400 MHz, DMSO-d₆): δ 12.82 (s, 1H), 7.98 (d, |
| | dichlorophenyl)-5-methyl-1H-1,2,3- | J = 8.9 Hz, 2H), 7.91 (t, J = 1.8 Hz, 1H), 7.78 (d, J = 1.8 |
| | triazole-4-carboxamide | Hz, 2H), 7.70 (d, J = 8.6 Hz, 2H), 2.41 (s, 3H); LCMS |
| | | (M + 1): 446.7 |
| 16 | 1-(3,5-dichlorophenyl)-N-((3,5- | ¹H-NMR (400 MHz, DMSO-d₆): δ 7.85 (t, J = 2 Hz, 1H), |
| | difluorophenyl)sulfonyl)-5-methyl- | 7.76 (d, J = 1.6 Hz, 2H), 7.51-7.48 (m, 1H), 7.45-7.41 (m, |
| | 1H-1,2,3-triazole-4-carboxamide | 2H), 7.35-7.28 (m, 1H), 2.52 (s, 3H); LCMS (M − 1): 446.8 |
| 17 | 1-(3,5-dichlorophenyl)-5-methyl-N- | ¹H-NMR (400 MHz, DMSO-d₆): δ 12.16 (s, 1H), 8.51- |
| | ((2- | 8.44 (m, 1H), 8.20-8.16 (m, 1H), 7.94 (t, J = 2 Hz, 1H), |
| | (trifluoromethoxy)phenyl)sulfonyl)- | 7.99-7.85 (m, 4H), 2.69 (s, 3H); LCMS (M − 1): 495.25 |
| | 1H-1,2,3-triazole-4-carboxamide | |
| 18 | 1-(3,5-dichlorophenyl)-N-((4- | ¹H-NMR (400 MHz, DMSO-d₆): δ 12.51 (s, 1H), 7.97- |
| | methoxyphenyl)sulfonyl)-5-methyl- | 7.93 (m, 3H), 7.81 (d, J = 1.8 Hz, 2H), 7.14 (d, J = 9.2 |
| | 1H-1,2,3-triazole-4-carboxamide | Hz, 2H), 3.84 (s, 3H), 2.43 (s, 3H); LCMS (M − 1): 440.8 |
| 19 | 1-(3,5-dichlorophenyl)-N-((2,4- | ¹H-NMR (400 MHz, DMSO-d₆): δ 12.87 (bs, 1H), 7.96- |
| | dimethylphenyl)sulfonyl)-5-methyl- | 7.89 (m, 2H), 7.81 (d, J = 1.6 Hz, 1H), 7.24 (d, J = 8.4 |
| | 1H-1,2,3-triazole-4-carboxamide | Hz, 1H), 7.20 (s, 1H), 2.59 (s, 3H), 2.40 (s, 3H), 2.33 (s, |
| | | 3H); LCMS (M − 1): 438.8 |
| 20 | 1-(3,5-dichlorophenyl)-N-((2,5- | ¹H-NMR (400 MHz, DMSO-d₆): δ 13.18 (s, 1H), 8.20 (d, |
| | dichlorophenyl)sulfonyl)-5- | J = 7.6 Hz, 1H), 7.97 (d, J = 2.4 Hz, 1H), 7.80 (t, J = 1.6 |
| | methoxy-1H-1,2,3-triazole-4- | Hz, 1H), 7.76 (d, J = 2 Hz, 2H), 7.49 (dd, J = 2.4 Hz, 1H), |
| | carboxamide | 7.44 (d, J = 8.4 Hz, 1H), 6.95 (d, J = 7.6 Hz, 1H) 4.16 (s, |
| | | 3H); LCMS (M+): 496.7 |
| 21 | N-((2-cyanophenyl)sulfonyl)-1-(3,5- | ¹H-NMR (400 MHz, DMSO-d₆): δ 11.76 (s, 1H), 8.53- |
| | dichlorophenyl)-5-methyl-1H-1,2,3- | 8.45 (m, 1H), 8.20-8.14 (m, 1H), 7.98 (t, J = 2 Hz, 1H), |
| | triazole-4-carboxamide | 7.94-7.89 (m, 4H), 2.63 (s, 3H); LCMS (M − 1): 435.7 |
| 22 | 1-(3,5-dichlorophenyl)-5- | ¹H-NMR (400 MHz, DMSO-d₆): δ 11.34 (s, 1H), 8.38 (s, |
| | (difluoromethyl)-N-(3- | 1H), 8.13 (d, J = 8.3 Hz, 1H), 8.02 (t, J = 1.8 Hz, 1H), |
| | (trifluoromethyl)phenyl)-1H-1,2,3- | 7.92 (d, J = 1.8 Hz, 2H), 7.86-7.60 (m, 2H), 7.51 (d, J = |
| | triazole-4-carboxamide | 7.6 Hz, 1H); LCMS (M − 2): 449.0 |
| 23 | N-((2-chlorophenyl)sulfonyl)-1-(2,4- | ¹H-NMR (400 MHz, DMSO-d₆): δ 8.17 (dd, J = 8.1, 1.4 |
| | dichlorophenyl)-5-methyl-1H-1,2,3- | Hz, 1H), 8.05 (d, J = 2.1 Hz, 1H), 7.78 (d, J = 8.3 Hz, |
| | triazole-4-carboxamide | 1H), 7.74 (dd, J = 8.6, 2.1 Hz, 1H), 7.70-7.66 (m, 2H), |
| | | 7.63-7.59 (m, 1H), 2.24 (s, 3H); LCMS (M+): 445.0 |
| 24 | 1-(3,5-dichlorophenyl)-5- | ¹H-NMR (400 MHz, DMSO-d₆): δ 11.29 (s, 1H), 8.04- |
| | (difluoromethyl)-N-(3- | 8.00 (m, 2H), 7.92 (d, J = 1.8 Hz, 2H), 7.90-7.87 (m, 1H), |
| | (trifluoromethoxy)phenyl)-1H-1,2,3- | 7.71 (t, J = 51.8 Hz, 1H), 7.51 (t, J = 8.3 Hz, 1H), 7.17- |
| | triazole-4-carboxamide | 7.12 (m, 1H); LCMS (M − 3): 464.8 |
| 25 | 1-(3,5-dichlorophenyl)-N-((4- | ¹H-NMR (400 MHz, DMSO-d₆): δ 12.76 (s, 1H), 8.11- |
| | fluorophenyl)sulfonyl)-5-methyl-1H- | 8.05 (m, 2H), 7.93 (t, J = 2 Hz, 1H), 7.80 (d, J = 2 Hz, |
| | 1,2,3-triazole-4-carboxamide | 2H), 7.52-7.45 (m, 2H), 2.43 (s, 3H); LCMS (M − 1): 428.8 |
| 26 | 1-(2,4-dichlorophenyl)-5-methyl-N- | ¹H-NMR (400 MHz, DMSO-d₆): δ 12.73 (s, 1H), 8.05- |
| | (phenylsulfonyl)-1H-1,2,3-triazole- | 8.01 (m, 3H), 7.77-7.69 (m, 3H), 7.64 (t, J = 7.6 Hz, 2H), |
| | 4-carboxamide | 2.26 (s, 3H); LCMS (M − 1): 410.7 |
| 27 | 1-(3,5-dichlorophenyl)-5- | ¹H-NMR (400 MHz, DMSO-d₆): δ 11.36 (s, 1H), 8.32 (s, |
| | (trifluoromethyl)-N-(3- | 1H), 8.12 (d, J = 8.6 Hz, 1H), 8.07 (t, J = 1.8 Hz, 1H), |
| | (trifluoromethyl)phenyl)-1H-1,2,3- | 8.03 (d, J = 1.8 Hz, 2H), 7.63 (t, J = 7.9 Hz, 1H), 7.52 (d, |
| | triazole-4-carboxamide | J = 7.9 Hz, 1H); LCMS (M − 2): 466.7 |
| 28 | 1-(2,4-dichlorophenyl)-N-((2,5- | ¹H-NMR (400 MHz, DMSO-d₆): δ 8.02 (dd, J = 6.6, 2.3 |
| | dichlorophenyl)sulfonyl)-5-methyl- | Hz, 2H), 7.76-7.69 (m, 2H), 7.60-7.47 (m, 2H), 2.33 (s, |
| | 1H-1,2,3-triazole-4-carboxamide | 3H); LCMS (M + 1): 480.6 |
| 29 | N-((3-chlorophenyl)sulfonyl)-1-(2,4- | ¹H-NMR (400 MHz, DMSO-d₆) δ 12.78 (bs, 1H), 8.05 |
| | dichlorophenyl)-5-methyl-1H-1,2,3- | (d, J = 1.5 Hz, 1H), 8.01-7.96 (m, 2H), 7.82-7.79 (m, 1H), |
| | triazole-4-carboxamide | 7.77-7.72 (m, 2H), 7.69 (t, J = 7.9 Hz, 1H), 2.28 (s, 3H); |
| | | LCMS (M − 1): 444.8 |
| 30 | N-((5-chloro-2- | ¹H-NMR (400 MHz, DMSO-d₆): δ 8.01-7.99 (m, 1H), |
| | methoxyphenyl)sulfonyl)-1-(3,5- | 7.93 (d, J = 1.8 Hz, 2H), 7.77 (d, J = 2.8 Hz, 1H), 7.46 |
| | dichlorophenyl)-5-(trifluoromethyl)- | (dd, J = 8.7, 2.9 Hz, 1H), 7.08 (d, J = 8.9 Hz, 1H), 3.76 |
| | 1H-1,2,3-triazole-4-carboxamide | (s, 3H); LCMS (M − 1): 528.7 |
| 31 | 1-(2,4-dichlorophenyl)-N-((4- | ¹H-NMR (400 MHz, DMSO-d₆): δ 12.51 (bs, 1H), 8.05 |
| | ethoxyphenyl)sulfonyl)-5-methyl- | (d, J = 2.4 Hz, 1H), 7.95-7.91 (m, 2H), 7.77-7.72 (m, 2H), |
| | 1H-1,2,3-triazole-4-carboxamide | 7.14-7.11 (m, 2H), 4.12 (q, J = 7.0 Hz, 2H), 2.27 (s, 3H), |
| | | 1.34 (t, J = 6.9 Hz, 3H); LCMS (M − 1): 454.7 |
| 32 | 1-(2,4-dichlorophenyl)-N-((2,4- | ¹H-NMR (400 MHz, DMSO-d₆): δ 8.16 (d, J = 8.6 Hz, |
| | dichlorophenyl)sulfonyl)-5-methyl- | 1H), 8.05 (d, J = 2.1 Hz, 1H), 7.89 (d, J = 2.1 Hz, 1H), |
| | 1H-1,2,3-triazole-4-carboxamide | 7.79-7.70 (m, 3H), 2.25 (s, 3H); LCMS (M + 1): 480.9 |
| 33 | N-((2-chloro-5- | ¹H-NMR (400 MHz, DMSO-d₆): δ 8.30 (d, J = 2.2 Hz, |
| | (trifluoromethyl)phenyl)sulfonyl)-1- | 1H), 8.02-7.99 (m, 1H), 7.80 (dd, J = 8.3, 2.0 Hz, 1H), |
| | (2,4-dichlorophenyl)-5-methyl-1H- | 7.73-7.68 (m, 3H), 2.34 (s, 3H); LCMS (M + 1): 514.7 |
| | 1,2,3-triazole-4-carboxamide | |
| 34 | 1-(3,5-dichlorophenyl)-N-((2,6- | ¹H-NMR (400 MHz, DMSO-d₆) δ 8.02-8.00 (m, 3H), |
| | dichlorophenyl)(methyl)(oxo)-λ⁶- | 7.76-7.74 (m, 2H), 7.68 (dd, J = 9.3, 6.6 Hz, 1H), 3.82 (s, |
| | sulfaneylidene)-5-(trifluoromethyl)- | 3H), LCMS(M+): 532.7 |
| | 1H-1,2,3-triazole-4-carboxamide | |
| 35 | 1-(3,5-dichlorophenyl)-N- | ¹H-NMR (400 MHz, DMSO-d₆): δ 8.04-7.99 (m, 3H), |
| | (phenylsulfonyl)-5-(trifluoromethyl)- | 7.94 (d, J = 1.8 Hz, 2H), 7.74-7.70 (m, 1H), 7.67-7.63 (m, |
| | 1H-1,2,3-triazole-4-carboxamide | 2H); LCMS (M − 1): 464.6 |
| 36 | 1-(3,5-dichlorophenyl)-N-((2- | ¹H-NMR (400 MHz, DMSO-d₆) δ 8.06-7.98 (m, 4H), |
| | fluorophenyl)(methyl)(oxo)-26- | 7.90-7.84(m, 1H), 7.60-7.54(m, 2H), 3.70 (s, 3H); |
| | sulfaneylidene)-5-(trifluoromethyl)- | LCMS (M+): 480.60 |
| | 1H-1,2,3-triazole-4-carboxamide | |
| 37 | 1-(4-chloro-2- | ¹H-NMR (400 MHz, DMSO-d₆): δ 8.21 (d, J = 2.1 Hz, |
| | (trifluoromethyl)phenyl)-N-((2- | 1H), 8.17 (dd, J = 7.8, 1.4 Hz, 1H), 8.09 (dd, J = 8.6, 2.4 |
| | chlorophenyl)sulfonyl)-5-methyl- | Hz, 1H), 7.87 (d, J = 8.6 Hz, 1H), 7.73-7.66 (m, 2H), |
| | 1H-1,2,3-triazole-4-carboxamide | 7.64-7.59 (m, 1H), 2.23 (s, 3H); LCMS (M + 1): 478.9 |
| 38 | 1-(2,4-dichlorophenyl)-5-methyl-N- | ¹H-NMR (400 MHz, DMSO-d₆) δ 8.04 (d, J = 2.0Hz, |
| | (methyl(oxo)(m-tolyl)-λ⁶- | 1H), 7.87(bs, 1H), 7.85-7.82(m, 1H), 7.77-7.71 (m, 2H), |
| | sulfaneylidene)-1H-1,2,3-triazole-4- | 7.61-7.57(m, 2H), 3.60 (s, 3H), 2.43 (s, 3H), 2.35 (s, 3H); |
| | carboxamide | LCMS (M+): 422.90 |
| 39 | 1-(4-chloro-2- | ¹H-NMR (400 MHz, CDCl3): δ 9.61 (bs, 1H), 8.16-8.10 |
| | (trifluoromethyl)phenyl)-N-((4- | (m, 2H), 7.90 (d, J = 2.4 Hz, 1H), 7.77 (dd, J = 8.3, 2.1 |
| | chlorophenyl)sulfonyl)-5-methyl- | Hz, 1H), 7.58-7.52 (m, 2H), 7.30 (d, J = 8.3 Hz, 1H), 2.35 |
| | 1H-1,2,3-triazole-4-carboxamide | (d, J = 14.7 Hz, 3H); LCMS (M + 1): 478.7 |
| 40 | 1-(4-chloro-2- | ¹H-NMR (400 MHz, CDC13): δ 8.35-8.33 (m, 1H), 7.89 |
| | (trifluoromethyl)phenyl)-N-((2- | (d, J = 2.4 Hz, 1H), 7.75 (dd, J = 8.6, 2.1 Hz, 1H), 7.57- |
| | chlorophenyl)sulfonyl)-N,5- | 7.47 (m, 3H), 7.29 (d, J = 8.6 Hz, 1H), 4.00 (s, 3H), 2.27 |
| | dimethyl-1H-1,2,3-triazole-4- | (s, 3H); LCMS (M + 1): 492.9 |
| | carboxamide | |
| 41 | 1-(2,4-dichlorophenyl)-5-methyl-N- | ¹H-NMR (400 MHz, DMSO-d6) δ 9.26-9.25 (m, 1H), |
| | (methyl(oxo)(5- | 8.67(dd, J = 1.6, 8.0 Hz, 1H), 8.47(d, J = 8.4Hz, 1H), 8.03 |
| | (trifluoromethyl)pyridin-2-yl)-λ⁶- | (d, J = 1.6 Hz, 1H), 7.77-7.71 (m, 2H), 3.60 (s, 3H), 2.32 |
| | sulfaneylidene)-1H-1,2,3-triazole-4- | (s, 3H); LCMS (M + 1): 479.70 |
| | carboxamide | |
| 42 | 1-(2,4-dichlorophenyl)-5-methyl-N- | ¹H-NMR (400 MHz, DMSO-d₆): δ 8.14 (dd, J = 8.1, 1.7 |
| | ((2- | Hz, 1H), 8.05 (d, J = 2.2 Hz, 1H), 7.86-7.82 (m, 1H), 7.78 |
| | (trifluoromethoxy)phenyl)sulfonyl)- | (d, J = 8.3 Hz, 1H), 7.74 (dd, J = 8.6, 2.2 Hz, 1H), 7.65- |
| | 1H-1,2,3-triazole-4-carboxamide | 7.59 (m, 2H), 2.25 (s, 3H); LCMS (M + 1): 494.9 |
| 43 | 1-(2,4-dichlorophenyl)-N-((4- | ¹H-NMR (400 MHz, DMSO-d₆): δ 8.09-8.04 (m, 3H), |
| | fluorophenyl)sulfonyl)-5-methyl-1H- | 7.77-7.72 (m, 2H), 7.47 (t, J = 8.8 Hz, 2H), 2.26 (s, 3H); |
| | 1,2,3-triazole-4-carboxamide | LCMS (M + 1): 430.6 |
| 44 | 1-(2,4-dichlorophenyl)-N-((3,5- | ¹H-NMR (400 MHz, DMSO-d₆): δ 8.00 (q, J = 0.8 Hz, |
| | difluorophenyl)sulfonyl)-5-methyl- | 1H), 7.72-7.67 (m, 2H), 7.49-7.44 (m, 2H), 7.34 (tt, J = |
| | 1H-1,2,3-triazole-4-carboxamide | 9.2, 2.4 Hz, 1H), 2.32 (s, 3H); LCMS (M + 1): 446.7 |
| 45 | 1-(2,4-dichlorophenyl)-5- | ¹H-NMR (400 MHz, DMSO-d₆): δ 8.01-7.99 (m, 2H), |
| | (difluoromethyl)-N-((2- | 7.82-7.80 (m, 1H), 7.70 (dd, J = 8.6, 2.4 Hz, 1H), 7.58- |
| | (trifluoromethoxy)phenyl)sulfonyl)- | 7.52 (m, 2H), 7.44 (td, J = 7.6, 1.0 Hz, 1H), 7.34 (dd, J = |
| | 1H-1,2,3-triazole-4-carboxamide | 6.7, 1.3 Hz, 1H); LCMS (M + 1): 530.8 |
| 46 | 1-(2,4-dichlorophenyl)-5- | ¹H-NMR (400 MHz, DMSO-d₆): δ 8.06 (dd, J = 6.4, 2.2 |
| | (difluoromethyl)-N-((2- | Hz, 1H), 7.89 (dd, J = 7.8, 1.7 Hz, 1H), 7.85 (d, J = 8.6 |
| | methoxyphenyl)sulfonyl)-1H-1,2,3- | Hz, 1H), 7.74 (td, J = 8.3, 2.2 Hz, 1H), 7.62 (t, J = 8.2 |
| | triazole-4-carboxamide | Hz, 2H), 7.20 (d, J = 8.1 Hz, 1H), 7.11 (t, J = 7.7 Hz, 1H), |
| | | 3.86 (s, 3H); LCMS (M + 1): 476.8 |
| 47 | 1-(2,4-dichlorophenyl)-5- | ¹H-NMR (400 MHz, DMSO-d₆): δ 8.01 (d, J = 2.2 Hz, |
| | (difluoromethyl)-N-((2- | 1H), 7.86-7.77 (m, 3H), 7.70 (dd, J = 8.4, 2.3 Hz, 1H), |
| | fluorophenyl)sulfonyl)-1H-1,2,3- | 7.51-7.46 (m, 1H), 7.25 (td, J = 7.6, 1.2 Hz, 1H), 7.22- |
| | triazole-4-carboxamide | 7.17 (m, 1H); LCMS (M + Na): 486.9 |
| 48 | 1-(2,4-dichlorophenyl)-5- | ¹H NMR (400 MHz, DMSO-d₆): δ 8.07-7.99 (m, 3H), |
| | (difluoromethyl)-N-((4- | 7.82 (d, J = 8.6 Hz, 1H), 7.76-7.71 (m, 1H), 7.45-7.38 (m, |
| | fluorophenyl)sulfonyl)-1H-1,2,3- | 3H); LCMS (M − 1): 462.8 |
| | triazole-4-carboxamide | |
| 49 | N-((3-chlorophenyl)sulfonyl)-1-(2,4- | ¹H NMR (400 MHz, DMSO-d₆): d 8.00 (d, J = 2.4 Mz, |
| | dichlorophenyl)-5-(difluoromethyl)- | 1H), 7.97-7.67 (m, 5H), 7.54-7.43 (m, 1H), 7.46 (t, J = 8 |
| | 1H-1,2,3-triazole-4-carboxamide | Mz, 1H); LCMS (M − 1): 480.6 |
| 50 | 1-(2,4-dichlorophenyl)-5- | ¹H NMR (400 MHz, DMSO-d₆): d 8.01 (d, J = 2 Mz, 1H), |
| | (difluoromethyl)-N-((2,5- | 7.90-7.67 (m, 4H), 7.13 (d, J = δ Mz, 1H), 7.07 (d, J = |
| | dimethylphenyl)sulfonyl)-1H-1,2,3- | 7.6 Mz, 1H), 2.49 (s, 3H), 2.31 (s, 3H); LCMS |
| | triazole-4-carboxamide | (M + NH4⁺): 492.0 |
| 51 | 1-(2,4-dichlorophenyl)-N-((2,5- | ¹H NMR (400 MHz, DMSO-d₆): d 12.94 (bs, 1H), 8.05 |
| | dimethylphenyl)sulfonyl)-5-methyl- | (d, J = 1.6 Mz, 1H), 7.85 (s, 1H), 7.79-7.72 (m, 2H), 7.39 |
| | 1H-1,2,3-triazole-4-carboxamide | (d, J = 7.6 Mz, 1H), 7.28 (d, J = 7.6 Mz, 1H), 2.59 (s, |
| | | 3H), 2.36 (s, 3H), 2.24 (s, 3H); LCMS (M + 1): 440.0 |
| 52 | 1-(4-chloro-2- | ¹H-NMR (400 MHz, CHLOROFORM-D) δ 9.96 (s, 1H), |
| | (trifluoromethyl)phenyl)-N-((2,6- | 7.91 (d, J = 2.1 Hz, 1H), 7.78 (dd, J = 8.6, 2.1 Hz, 1H), |
| | difluorophenyl)sulfonyl)-5- | 7.67-7.60 (m, 1H), 7.40 (d, J = 8.6 Hz, 1H), 7.13-7.08 (m, |
| | (trifluoromethyl)-1H-1,2,3-triazole- | 2H), LCMS(M+): 534.95 |
| | 4-carboxamide | |
| 53 | N-((2-chloro-5- | ¹H NMR (400 MHz, DMSO-d₆): d 7.85 (t, J = 1.6 Mz, |
| | methoxyphenyl)sulfonyl)-1-(3,5- | 1H), 7.78 (d, J = 1.6 Mz, 2H), 7.55 (d, J = 2.8 Mz, 1H), |
| | dichlorophenyl)-5-methyl-1H-1,2,3- | 7.29 (d, J = 8.8 Mz, 1H), 6.97 (dd, J = 2.8, 3.2 Mz, 1H), |
| | triazole-4-carboxamide | 3.78 (s, 3H), 2.55 (s, 3H); LCMS (M + 1): 476.9 |
| 54 | N-((2-chloro-5- | ¹H-NMR (400 MHz, DMSO-d₆): 7.80-8.03 (2H), 7.39- |
| | methoxyphenyl)sulfonyl)-1-(3,5- | 7.60 (1H), 7.15-7.34 (1H), 6.81-7.00 (2H), 3.64-3.88 |
| | dichlorophenyl)-5-(trifluoromethyl)- | (3H); LCMS (M + 1): 530.1 |
| | 1H-1,2,3-triazole-4-carboxamide | |
| 55 | N-((2-chloro-5- | ¹H NMR (400 MHz, DMSO-d₆): d 8.22-7.90 (m, 2H), |
| | methoxyphenyl)sulfonyl)-1-(3,5- | 7.83 (d, J = 2 Mz, 2H), 7.54 (d, J = 3.2 Mz, 1H), 7.32 (d, |
| | dichlorophenyl)-5-(difluoromethyl)- | J = 8.4 Mz, 1H), 7.00 (dd, J = 3.2, 3.2 Mz, 1H), 3.80 (s, |
| | 1H-1,2,3-triazole-4-carboxamide | 3H); LCMS (M + 1): 510.8 |
| 56 | N-((2-chloro-5- | ¹H NMR (400 MHz, DMSO-d₆): d 8.02-7.99 (m, 1H), |
| | methoxyphenyl)sulfonyl)-1-(2,4- | 7.73-7.67 (m, 2H), 7.55 (d, J = 3.2 Mz, 1H), 7.29 (d, J = |
| | dichlorophenyl)-5-methyl-1H-1,2,3- | 8.8 Mz, 1H), 6.97 (dd, J = 3.2, 3.2 Mz, 1H), 3.80 (s, 3H), |
| | triazole-4-carboxamide | 2.33 (s, 3H); LCMS (M + 1): 476.8 |
| 57 | 1-(4-chloro-2- | 1H NMR (400 MHz, DMSO-d₆): d 8.21 (d, J = 2 Mz, 1H), |
| | (trifluoromethyl)phenyl)-N-((2- | 8.08 (dd, J = 2, 2.4 Mz, 1H), 8.03 (d, J = 8.8 Mz, 1H), |
| | chloro-5-methoxyphenyl)sulfonyl)- | 7.55 (d, J = 3.2 Mz, 1H), 7.38 (d, J = 8.4 Mz, 1H), 7.05 |
| | 5-(trifluoromethyl)-1H-1,2,3- | (dd, J = 3.2, 3.2 Mz, 1H), 3.80 (s, 3H); LCMS (M + 1): |
| | triazole-4-carboxamide | 562.8 |
| 58 | N-((2-chloro-5- | ¹H NMR (400 MHz, DMSO-d₆): d 9.03 (bs, 1H), 8.01 (d, |
| | methoxyphenyl)sulfonyl)-1-(2,4- | J = 2.4 Mz, 1H), 7.78 (d, J = 8.4 Mz, 1H), 7.69 (dd, J = |
| | dichlorophenyl)-1H-1,2,3-triazole-4- | 2, 2.4 Mz, 1H), 7.60 (d, J = 3.2 Mz, 1H), 7.49 (d, J = 8.4 |
| | carboxamide | Mz, 1H), 7.19 (d, J = 8.8 Mz, 1H), 3.83 (s, 3H); LCMS |
| | | (M + 1): 460.8 |
| 59 | N-((2-chlorophenyl)sulfonyl)-1-(2,4- | ¹H NMR (400 MHz, DMSO-d₆): d 8.58 (s, 1H), 8.04-7.99 |
| | dichlorophenyl)-1H-1,2,3-triazole-4- | (m, 1H), 7.96 (d, J = 2 Mz, 1H), 7.72 (d, J = 8.4 Mz, 1H), |
| | carboxamide | 7.65 (dd, J = 2, 2.4 Mz, 1H), 7.42-7.35 (m, 3H); LCMS |
| | | (M − 1): 430.7 |
| 60 | 1-(2,4-dichlorophenyl)-N-((3,5- | ¹H NMR (400 MHz, DMSO-d₆): d 8.62 (s, 1H), 7.98 (d, |
| | difluorophenyl)sulfonyl)-1H-1,2,3- | J = 2 Mz, 1H), 7.72 (d, J = 8.4 Mz, 1H), 7.67 (dd, J = 2.4, |
| | triazole-4-carboxamide | 2 Mz, 1H), 7.50-7.44 (m, 2H), 7.39-7.31 (m, 1H); LCMS |
| | | (M + 1): 432.8 |
| 61 | 1-(4-chloro-2- | ¹H-NMR (400 MHz, CHLOROFORM-D) δ 9.88 (s, 1H), |
| | (trifluoromethyl)phenyl)-N-((2- | 7.91 (d, J = 2.2 Hz, 1H), 7.86 (d, J = 3.2 Hz, 1H), 7.77 |
| | chloro-5-methoxyphenyl)sulfonyl)- | (dd, J = 8.4, 2.1 Hz, 1H), 7.42 (d, J = 8.8 Hz, 1H), 7.32 |
| | 5-methyl-1H-1,2,3-triazole-4- | (d, J = 8.6 Hz, 1H), 7.10 (dd, J = 8.8, 3.2 Hz, 1H), 3.90 |
| | carboxamide | (s, 3H), 2.35 (s, 3H); LCMS (M + 1): 508.9 |
| 62 | 1-(2,4-dichlorophenyl)-N-((2,5- | ¹H NMR (400 MHz, DMSO-d₆): d 8.69 (bs, 1H), 8.02- |
| | dichlorophenyl)sulfonyl)-1H-1,2,3- | 7.97 (m, 2H), 7.73 (d, J = 8.4 Mz, 1H), 7.66 (dd, J = 2, |
| | triazole-4-carboxamide | 2.4 Mz, 1H), 7.56-7.45 (m, 2H); LCMS (M + 1): 466.8 |
| 63 | 1-(4-chloro-2- | 1H NMR (400 MHz, DMSO-d₆): d 9.08 (s, 1H), 8.14 (d, |
| | (trifluoromethyl)phenyl)-N-((2- | J = 2.4 Mz, 1H), 8.0 (dd, J = 2, 2.4 Mz, 1H), 7.79 (d, J = |
| | chloro-5-methoxyphenyl)sulfonyl)- | 8.4 Mz, 1H), 7.59 (d, J = 3.2 Mz, 1H), 7.54 (d, J = 8.8 |
| | 1H-1,2,3-triazole-4-carboxamide | Mz, 1H), 7.25 (dd, J = 3.2, 2.8 Mz, 1H), 3.82 (s, 3H); |
| | | LCMS (M + 1): 494.9 |
| 64 | 1-(2,4-dichlorophenyl)-N-((2,4- | ¹H NMR (400 MHz, DMSO-d₆): d 8.04-7.77 (m, 4H), |
| | dichlorophenyl)sulfonyl)-5- | 7.70 (dd, J = 2, 2.4 Mz, 1H), 7.60 (d, J = 2 Mz, 1H), 7.52 |
| | (difluoromethyl)-1H-1,2,3-triazole- | (dd, J = 2, 2.4 Mz, 1H); LCMS (M − 1): 514.6 |
| | 4-carboxamide | |
| 65 | 1-(2,4-dichlorophenyl)-N-((2,5- | ¹H NMR (400 MHz, DMSO-d₆): d 8.02-7.73 (m, 4H), |
| | dichlorophenyl)sulfonyl)-5- | 7.70 (dd, J = 2, 2.4 Mz, 1H), 7.55 (dd, J = 2.4, 2.4 Mz, |
| | (difluoromethyl)-1H-1,2,3-triazole- | 1H), 7.50 (d, J = 8.4 Mz, 1H); LCMS (M + Na): 539.0 |
| | 4-carboxamide | |
| 66 | N-((2-chloro-5- | ¹H NMR (400 MHz, DMSO-d₆): d 8.70 (bs, 1H), 8.29 (d, |
| | (trifluoromethyl)phenyl)sulfonyl)-1- | J = 2 Mz, 1H), 7.97 (d, J = 2 Mz, 1H), 7.83 (d, J = 7.6 |
| | (2,4-dichlorophenyl)-1H-1,2,3- | Mz, 1H), 7.75-7.64 (m, 3H); LCMS (M + 1): 500.8 |
| | triazole-4-carboxamide | |
| 67 | N-((2-chloro-5- | 1H NMR (400 MHz, DMSO-d₆): d 8.01 (d, J = 2.4 Mz, |
| | methoxyphenyl)sulfonyl)-1-(2,4- | 1H), 7.82 (d, J = 8.8 Mz, 1H), 7.7 (dd, J = 2, 2 Mz, 1H), |
| | dichlorophenyl)-5-(difluoromethyl)- | 7.56 (d, J = 3.2 Mz, 1H), 7.33 (d, J = 8.8 Mz, 1H), 7.01 |
| | 1H-1,2,3-triazole-4-carboxamide | (dd, J = 3.2, 3.2 Mz, 1H), 3.79 (s, 3H), 2.84 (s, 1H); |
| | | LCMS (M + 1): 510.7 |
| 68 | N-((2-cyanophenyl)sulfonyl)-1-(2,4- | ¹H NMR (400 MHz, DMSO-d₆): d 11.81 (bs, 1H), 9.49 |
| | dichlorophenyl)-1H-1,2,3-triazole-4- | (s, 1H), 8.49-8.46 (m, 1H), 8.19-8.15 (m, 1H), 8.05 (dd, |
| | carboxamide | J = 2.4, 2.4 Mz, 1H), 7.95-7.90 (m, 2H), 7.84 (dd, J = 8.4, |
| | | 8.8 Mz, 1H), 7.75 (dd, J = 2.4, 2.4 Mz, 1H); LCMS |
| | | (M + 1): 421.6 |
| 69 | N-((2-chlorophenyl)sulfonyl)-1-(2,4- | ¹H NMR (400 MHz, DMSO-d₆): d 8.05-8.01 (m, 2H), |
| | dichlorophenyl)-5-(difluoromethyl)- | 7.96 (s, 1H), 7.81 (d, J = 12 Mz, 1H), 7.70 (dd, J = 2, 2 |
| | 1H-1,2,3-triazole-4-carboxamide | Mz, 1H), 7.44-7.38 (m, 3H); LCMS (M + 1): 482.9 |
| 70 | 1-(2,4-dichlorophenyl)-5- | ¹H NMR (400 MHz, DMSO-d₆): d 8.01 (d, J = 2.4 Mz, |
| | (difluoromethyl)-N-((3,5- | 1H), 7.81-7.63 (m, 3H), 7.50-7.45 (m, 2H), 7.40-7.34 (m, |
| | difluorophenyl)sulfonyl)-1H-1,2,3- | 1H); LCMS (M − 1): 480.9 |
| | triazole-4-carboxamide | |
| 71 | 1-(4-chloro-2- | ¹H NMR (400 MHz, DMSO-d₆): d 9.18 (s, 1H), 8.18 (d, |
| | (trifluoromethyl)phenyl)-N-((2- | J = 2.4 Mz, 1H), 8.08-7.99 (m, 2H), 7.86 (d, J = 8.4 Mz, |
| | fluorophenyl)sulfonyl)-1H-1,2,3- | 1H), 7.81-7.76 (m, 1H), 7.49-7.44 (m, 2H), 5.74 (s, 1H); |
| | triazole-4-carboxamide | LCMS (M + 1): 448.9 |
| 72 | 1-(2-chloro-4- | ¹H-NMR (400 MHz, DMSO-d₆) δ 8.35 (d, J = 1.7 Hz, |
| | (trifluoromethyl)phenyl)-N-((2- | 1H), 8.20 (d, J = 8.1 Hz, 1H), 8.08-8.06 (m, 1H), 7.58 (d, |
| | chloro-5-methoxyphenyl)sulfonyl)- | J = 3.2 Hz, 1H), 7.45 (d, J = 8.8 Hz, 1H), 7.13 (dd, J = |
| | 5-(trifluoromethyl)-1H-1,2,3- | 8.8, 2.9 Hz, 1H), 3.82 (s, 3H); LCMS (M + 1): 563.25 |
| | triazole-4-carboxamide | |
| 73 | 1-(2-chloro-4- | ¹H-NMR (400 MHz, DMSO-d₆) δ 8.36 (d, J = 1.5 Hz, |
| | (trifluoromethyl)phenyl)-N-((2,5- | 1H), 8.20 (d, J = 8.1 Hz, 1H), 8.09-8.05 (m, 2H), 7.69 |
| | dichlorophenyl)sulfonyl)-5- | (dd, J = 8.4, 2.6 Hz, 1H), 7.63 (d, J = 8.6 Hz, 1H); LCMS |
| | (trifluoromethyl)-1H-1,2,3-triazole- | (M + 1): 567.6 |
| | 4-carboxamide | |
| 74 | 1-(2-chloro-4- | ¹H-NMR (400 MHz, DMSO-d₆) δ 8.33 (d, J = 1.7 Hz, |
| | (trifluoromethyl)phenyl)-N-((2- | 1H), 8.16 (d, J = 8.1 Hz, 1H), 8.05-8.01 (m, 2H), 7.44- |
| | chlorophenyl)sulfonyl)-5- | 7.38 (m, 3H); LCMS (M + 1): 533.2 |
| | (trifluoromethyl)-1H-1,2,3-triazole- | |
| | 4-carboxamide | |
| 75 | 1-(2-chloro-4- | ¹H-NMR (400 MHz, DMSO-d₆) δ 8.32 (d, J = 1.5 Hz, |
| | (trifluoromethyl)phenyl)-N-((4- | 1H), 8.14 (d, J = 8.3 Hz, 1H), 8.04 (dd, J = 8.4, 1.3 Hz, |
| | chlorophenyl)sulfonyl)-5- | 1H), 7.83 (dt, J = 9.0, 2.3 Hz, 2H), 7.50 (dt, J = 9.0, 2.3 |
| | (trifluoromethyl)-1H-1,2,3-triazole- | Hz, 2H); LCMS (M + 1): 533.2 |
| | 4-carboxamide | |
| 76 | 1-(2-chloro-4- | ¹H-NMR (400 MHz, DMSO-d₆) δ 8.33 (d, J = 1.5 Hz, |
| | (trifluoromethyl)phenyl)-N-((2- | 1H), 8.27 (d, J = 2.0 Hz, 1H), 8.18 (d, J = 8.1 Hz, 1H), |
| | chloro-5- | 8.06-8.03 (m, 1H), 7.82 (dd, J = 7.7, 2.3 Hz, 1H), 7.71 (d, |
| | (trifluoromethyl)phenyl)sulfonyl)-5- | J = 7.8 Hz, 1H); LCMS (M + 1): 601.2 |
| | (trifluoromethyl)-1H-1,2,3-triazole- | |
| | 4-carboxamide | |
| 77 | N-((2-chloro-5- | ¹H NMR (400 MHz, DMSO-d₆): d 8.87 (s, 1H), 7.63-7.54 |
| | methoxyphenyl)sulfonyl)-1-(3,5- | (m, 3H), 7.45 (d, J = 2 Hz, 2H), 7.28 (dd, J = 3.2, 3.2 Mz, |
| | dichlorobenzyl)-1H-1,2,3-triazole-4- | 1H), 5.68 (s, 1H), 3.83 (s, 3H); LCMS (M + 1): 477.0 |
| | carboxamide | |
| 78 | N-((2-chloro-5- | ¹H NMR (400 MHz, DMSO-d₆): d 8.85 (s, 1H), 7.60-7.55 |
| | methoxyphenyl)sulfonyl)-1-(3,3,3- | (m, 2H), 7.28 (dd, J = 2.8, 3.2 Mz, 1H), 4.73-4.67 (m, |
| | trifluoropropyl)-1H-1,2,3-triazole-4- | 2H), 3.84 (s, 3H), 3.06-2.95 (m, 2H); LCMS (M + 1): |
| | carboxamide | 412.9 |
| 79 | N-((5-chloropyridin-2-yl)sulfonyl)- | ¹H NMR (400 MHz, DMSO-d₆): d 8.83 (s, 1H), 8.80 (d, |
| | 1-(3,3,3-trifluoropropyl)-1H-1,2,3- | J = 1.6 Mz, 1H), 8.28 (dd, J = 2.4, 2.8 Mz, 1H), 8.15 (d, |
| | triazole-4-carboxamide | J = 8.8 Mz, 1H), 4.71 (t, J = 6.4 Mz, 2H), 3.06-2.94 (m, |
| | | 2H); LCMS (M + 1): 383.8 |
| 80 | N-((3,5-difluorophenyl)sulfonyl)-1- | ¹H NMR (400 MHz, DMSO-d₆): d 8.83 (s, 1H), 7.72-7.62 |
| | (3,3,3-trifluoropropyl)-1H-1,2,3- | (m, 3H), 4.70 (t, J = δ Mz, 2H), 3.06-2.96 (m, 2H); LCMS |
| | triazole-4-carboxamide | (M + 1): 384.9 |
| 81 | 1-(3,5-dichlorobenzyl)-N-((2- | ¹H NMR (400 MHz, DMSO-d₆): d 8.85 (s, 1H), 8.0-7.96 |
| | fluorophenyl)sulfonyl)-1H-1,2,3- | (m, 1H), 7.79-7.73 (m, 1H), 7.63 (t, J = 4.0 Mz, 1H), |
| | triazole-4-carboxamide | 7.46-7.41 (m, 4H), 5.68 (s, 2H); LCMS (M + 1): 428.8 |
| 82 | N-((2-chloro-5- | ¹H-NMR (400 MHz, DMSO-d₆): δ 8.85 (s, 1H), 7.61 (d, |
| | methoxyphenyl)sulfonyl)-1- | J = 2.9 Hz, 1H), 7.57 (d, J = 8.8 Hz, 1H), 7.29 (dd, J = |
| | (cyclopropylmethyl)-1H-1,2,3- | 8.8, 3.2 Hz, 1H), 4.29 (d, J = 7.6 Hz, 2H), 3.85 (s, 3H), |
| | triazole-4-carboxamide | 1.31-1.25 (m, 1H), 0.60-0.55 (m, 2H), 0.45-0.41 (m, 2H); |
| | | LCMS (M + 1): 392.9 |
| 83 | N-((2-chloro-5- | ¹H-NMR (400 MHz, DMSO-d₆): δ 8.79 (s, 1H), 7.60 (d, |
| | methoxyphenyl)sulfonyl)-1-propyl- | J = 3.2 Hz, 1H), 7.58-7.55 (m, 1H), 7.28 (dd, J = 8.9, 3.1 |
| | 1H-1,2,3-triazole-4-carboxamide | Hz, 1H), 4.39-4.33 (m, 2H), 3.84 (s, 3H), 1.89-1.78 (m, |
| | | 2H), 0.84 (dd, J = 15.3, 8.0 Hz, 3H); LCMS (M+): 358.8 |
| 84 | 1-(2-chloro-4- | ¹H-NMR (400 MHz, DMSO-d₆) δ 8.37 (d, J = 1.5 Hz, |
| | (trifluoromethyl)phenyl)-5-methyl- | 1H), 8.19 (d, J = 8.3 Hz, 1H), 8.09 (dd, J = 8.4, 1.4 Hz, |
| | N-(phenylsulfonyl)-1H-1,2,3- | 1H), 8.01-7.99 (m, 2H), 7.71 (d, J = 7.3 Hz, 1H), 7.64 (t, |
| | triazole-4-carboxamide | J = 7.5 Hz, 2H); LCMS (M + 1): 498.78 |
| 85 | 1-(2-chloro-4- | ¹H-NMR (400 MHz, DMSO-d₆) δ 8.38 (d, J = 1.5 Hz, |
| | (trifluoromethyl)phenyl)-N-tosyl-5- | 1H), 8.20 (d, J = 8.3 Hz, 1H), 8.10 (dd, J = 8.3, 1.5 Hz, |
| | (trifluoromethyl)-1H-1,2,3-triazole- | 1H), 7.89 (d, J = 8.3 Hz, 2H), 7.45 (d, J = 8.1 Hz, 2H), |
| | 4-carboxamide | 2.40 (s, 3H); LCMS (M + 1): 512.8 |
| 86 | N-((2-chlorophenyl)sulfonyl)-1-(3,5- | ¹H-NMR (400 MHz, DMSO-d₆) δ 12.97 (s, NH), 8.14 |
| | dichlorobenzyl)-5-methyl-1H-1,2,3- | (dd, J = 7.8, 1.5 Hz, 1H), 7.71-7.57 (m, 4H), 7.30 (d, J = |
| | triazole-4-carboxamide | 2.0 Hz, 2H), 5.63 (s, 2H), 2.38 (s, 3H); LCMS (M + 1): |
| | | 460.80 |
| 87 | N-((2-chlorophenyl)sulfonyl)-1-(2,4- | ¹H-NMR (400 MHz, DMSO-d₆) δ 8.79 (s, 1H), 8.15 (dd, |
| | dichlorobenzyl)-1H-1,2,3-triazole-4- | J = 7.9, 1.6 Hz, 1H), 7.73 (d, J = 2.1 Hz, 1H), 7.70-7.58 |
| | carboxamide | (m, 3H), 7.50 (dd, J = 8.3, 2.2 Hz, 1H), 7.40 (d, J = 8.3 |
| | | Hz, 1H), 5.75 (d, J = 3.7 Hz, 2H); LCMS (M + 1): 446.75 |
| 88 | N-((4-chlorophenyl)sulfonyl)-1-(2,4- | ¹H-NMR (400 MHz, DMSO-d₆) δ 8.77 (s, 1H), 7.98-7.95 |
| | dichlorobenzyl)-1H-1,2,3-triazole-4- | (m, 2H), 7.73-7.69 (m, 3H), 7.48 (dd, J = 8.3, 2.2 Hz, 1H), |
| | carboxamide | 7.35 (d, J = 8.3 Hz, 1H), 5.74 (s, 2H); LCMS (M + 1): |
| | | 446.80 |
| 89 | N-((4-chlorophenyl)sulfonyl)-1-(3,5- | ¹H-NMR (400 MHz, DMSO-d₆) δ 7.99 (dt, J = 9.1, 2.3 |
| | dichlorobenzyl)-5-methyl-1H-1,2,3- | Hz, 2H), 7.71 (dt, J = 9.3, 2.3 Hz, 2H), 7.59 (t, J = 2.0 Hz, |
| | triazole-4-carboxamide | 1H), 7.27 (d, J = 2.0 Hz, 2H), 5.63 (s, 2H), 2.40 (s, 3H); |
| | | LCMS (M + 1): 458.9 |
| 90 | 1-(3,5-bis(trifluoromethyl)phenyl)- | ¹H-NMR (400 MHz, CHLOROFORM-D) δ 8.11 (s, 1H), |
| | N-((3,5-difluorophenyl)sulfonyl)-5- | 7.95 (s, 2H), 7.73 (d, J = 3.7 Hz, 2H), 7.12 (m, 1H), 2.67 |
| | methyl-1H-1,2,3-triazole-4- | (s, 3H); LCMS (M − 1): 512.90 |
| | carboxamide | |
| 91 | 1-(2-chloro-4- | ¹H-NMR (400 MHz, CHLOROFORM-D) δ 9.91 (s, 1H), |
| | (trifluoromethyl)phenyl)-N-((2- | 8.23-8.19 (m, 1H), 7.92 (d, J = 2.0 Hz, 1H), 7.80-7.78 (m, |
| | fluorophenyl)sulfonyl)-5- | 1H), 7.73-7.67 (m, 1H), 7.61 (d, J = 7.8 Hz, 1H), 7.42- |
| | (trifluoromethyl)-1H-1,2,3-triazole- | 7.38 (m, 1H); LCMS(M − 1): 514.85 |
| | 4-carboxamide | |
| 92 | 1-(2-chloro-4- | ¹H-NMR (400 MHz, CHLOROFORM-D) δ 7.93 (s, 1H), |
| | (trifluoromethyl)phenyl)-5- | 7.80 (d, J = 8.3 Hz, 1H), 7.62 (d, J = 8.1 Hz, 1H), 6.86 (t, |
| | (trifluoromethyl)-N-((2,4,6- | J = 8.4 Hz, 2H); LCMS(M + 1): 574.80 |
| | trifluorophenyl)sulfonyl)-1H-1,2,3- | |
| | triazole-4-carboxamide | |
| 93 | N-((5-chloropyridin-2-yl)sulfonyl)- | ¹H-NMR (400 MHz, DMSO-d₆) δ 8.80 (d, J = 1.7 Hz, |
| | 1-(cyclobutylmethyl)-1H-1,2,3- | 1H), 8.72 (s, 1H), 8.28 (dd, J = 8.3, 2.4 Hz, 1H), 8.16 (d, |
| | triazole-4-carboxamide | J = 8.3 Hz, 1H), 4.44 (d, J = 7.3 Hz, 2H), 2.80-2.73 (m, |
| | | 1H), 1.99-1.94 (m, 2H), 1.88-1.71 (m, 4H), LCMS(M+): |
| | | 355.9 |
| 94 | 1-(cyclobutylmethyl)-N-((2- | ¹H-NMR (400 MHz, DMSO-d₆) δ 8.74 (s, 1H), 8.00-7.96 |
| | fluorophenyl)sulfonyl)-1H-1,2,3- | (m, 1H), 7.80-7.74 (m, 1H), 7.47-7.42 (m, 2H), 4.44 (d, |
| | triazole-4-carboxamide | J = 7.3 Hz, 2H), 2.80-2.72 (m, 1H), 2.00-1.94 (m, 2H), |
| | | 1.90-1.70 (m, 4H), LCMS(M+): 339.0 |
| 95 | 1-(3-chloro-5- | ¹H-NMR (400 MHz, DMSO-d₆) δ 8.19 (s, 1H), 8.16-8.15 |
| | (trifluoromethyl)phenyl)-N-((3,5- | (m, 1H), 8.07 (s, 1H), 7.70-7.65 (m, 3H), 2.45 (s, 3H); |
| | difluorophenyl)sulfonyl)-5-methyl- | LCMS (M + 1): 480.85 |
| | 1H-1,2,3-triazole-4-carboxamide | |
| 96 | 1-(3-chloro-5- | ¹H-NMR (400 MHz, DMSO-d₆) δ 8.2 (s, 1H), 8.16 (t, J = |
| | (trifluoromethyl)phenyl)-N-((2- | 1.6 Hz, 1H), 8.08 (s, 1H), 8.00 (td, J = 8, 1.6 Hz, 1H), |
| | fluorophenyl)sulfonyl)-5-methyl-1H- | 7.79-7.74 (m, 1H), 7.47-7.43 (m, 2H), 2.42 (s, 3H); |
| | 1,2,3-triazole-4-carboxamide | LCMS (M + 1): 462.90 |
| 97 | 1-(3-chloro-5- | ¹H-NMR (400 MHz, DMSO-d₆) δ 8.20-8.17 (m, 2H), |
| | (trifluoromethyl)phenyl)-5-methyl- | 8.09 (s, 1H), 7.47 (t, J = 7.2 Hz, 2H), 2.45 (s, 3H); LCMS |
| | N-((2,4,6-trifluorophenyl)sulfonyl)- | (M + 1): 498.85 |
| | 1H-1,2,3-triazole-4-carboxamide | |
| 98 | N-((2-chloro-5- | ¹H-NMR (400 MHz, DMSO-d₆) δ 8.21 (s, 1H), 7.69 (d, |
| | methoxyphenyl)sulfonyl)-1-(2,4- | J = 2.2 Hz, 1H), 7.51 (d, J = 3.2 Hz, 1H), 7.46 (dd, J = 8.3, |
| | dichlorobenzyl)-1H-1,2,3-triazole-4- | 2.0 Hz, 1H), 7.25 (dd, J = 15.8, 8.4 Hz, 2H), 6.95 (dd, J = |
| | carboxamide | 8.7, 3.1 Hz, 1H), 5.66 (s, 2H), 3.77 (s, 3H); LCMS (M + 1): |
| | | 476.75 |
| 99 | 1-(3,5-dichlorobenzyl)-N-((2,4,6- | ¹H-NMR (400 MHz, DMSO-d₆) δ 8.23 (s, 1H), 7.69 (d, |
| | trifluorophenyl)sulfonyl)-1H-1,2,3- | J = 2.2 Hz, 1H), 7.45 (dd, J = 8.3, 2.2 Hz, 1H), 7.23 (d, J = |
| | triazole-4-carboxamide | 8.3 Hz, 1H), 7.08 (dd, J = 9.3, 8.3 Hz, 2H), 5.66 (s, 2H); |
| | | LCMS (M+): 464.90 |
| 100 | N-((2-chloro-5- | ¹H-NMR (400 MHz, DMSO-d₆) δ 7.58-7.55 (m, 2H), |
| | methoxyphenyl)sulfonyl)-1- | 7.26 (dd, J = 8.9, 3.1 Hz, 1H), 4.02-3.98 (m, 1H), 3.84 (s, |
| | cyclopropyl-5-(trifluoromethyl)-1H- | 3H), 1.34-1.27 (m, 2H), 1.21 (td, J = 7.4, 5.7 Hz, 2H); |
| | 1,2,3-triazole-4-carboxamide | LCMS (M+): 424.80 |
| 101 | 1-(3,5-dichlorobenzyl)-5-methyl-N- | ¹H-NMR (400 MHz, DMSO-d₆) δ 7.60 (t, J = 2.0 Hz, 1H), |
| | ((2,4,6-trifluorophenyl)sulfonyl)-1H- | 7.43 (t, J = 9.4 Hz, 2H), 7.29 (d, J = 1.7 Hz, 2H), 5.63 (s, |
| | 1,2,3-triazole-4-carboxamide | 2H), 2.41 (s, 3H); LCMS (M+): 478.85 |
| 102 | 1-(3,5-dichlorobenzyl)-N-((2,5- | ¹H-NMR (400 MHz, DMSO-d₆) δ 8.07 (d, J = 2.7 Hz, |
| | dichlorophenyl)sulfonyl)-5-methyl- | 1H), 7.77 (dd, J = 8.6, 2.4 Hz, 1H), 7.68 (d, J = 8.6 Hz, |
| | 1H-1,2,3-triazole-4-carboxamide | 1H), 7.60 (t, J = 1.8 Hz, 1H), 7.29 (d, J = 2.0 Hz, 2H), |
| | | 5.63 (d, J = 5.9 Hz, 2H), 2.40 (s, 3H); LCMS (M+): |
| | | 494.75 |
| 103 | 1-(3,5-dichlorobenzyl)-N-((3,5- | ¹H-NMR (400 MHz, DMSO-d₆) δ 7.57 (t, J = 2.0 Hz, 1H), |
| | difluorophenyl)sulfonyl)-5-methyl- | 7.44-7.40 (m, 2H), 7.34-7.29 (m, 1H), 7.17 (d, J = 1.7 Hz, |
| | 1H-1,2,3-triazole-4-carboxamide | 2H), 5.55 (s, 2H), 2.43 (s, 3H); LCMS(M + 1): 462.65 |
| 104 | N-((2-chloro-5- | ¹H-NMR (400 MHz, DMSO-d₆) δ 8.29 (d, J = 2.0 Hz, |
| | (trifluoromethyl)phenyl)sulfonyl)-1- | 1H), 8.00 (dd, J = 8.3, 2.0 Hz, 1H), 7.86 (d, J = 8.3 Hz, |
| | cyclopropyl-5-(trifluoromethyl)-1H- | 1H), 3.97 (t, J = 3.4 Hz, 1H), 1.32-1.28 (m, 2H), 1.24- |
| | 1,2,3-triazole-4-carboxamide | 1.17 (m, 2H); LCMS(M − 2): 460.85 |
| 105 | 1-(3,5-dichlorobenzyl)-N-((2- | ¹H-NMR (400 MHz, DMSO-d₆) δ 13.02 (s, 1H), 7.97 (td, |
| | fluorophenyl)sulfonyl)-5-methyl-1H- | J = 7.6, 1.6 Hz, 1H), 7.78-7.73 (m, 1H), 7.60 (t, J = 1.8 |
| | 1,2,3-triazole-4-carboxamide | Hz, 1H), 7.46-7.41 (m, 2H), 7.29 (d, J = 2.0 Hz, 2H), 5.63 |
| | | (s, 2H), 2.39 (s, 3H); LCMS(M − 1): 442.85 |
| 106 | 1-(2,4-dichlorobenzyl)-N-((2,5- | ¹H-NMR (400 MHz, DMSO-d₆) δ 8.49-8.92 (1H), 8.10 |
| | dichlorophenyl)sulfonyl)-1H-1,2,3- | (d, J = 2.4 Hz, 1H), 7.81 (dd, J = 8.6, 2.4 Hz, 1H), 7.75 |
| | triazole-4-carboxamide | (d, J = 2.0 Hz, 1H), 7.71 (d, J = 8.6 Hz, 1H), 7.52 (dd, |
| | | J = 8.3, 2.0 Hz, 1H), 7.41 (d, J = 8.3 Hz, 1H), 5.76 (d, J = |
| | | 8.3 Hz, 2H); LCMS(M): 480.80 |
| 107 | N-((2-chloro-5- | ¹H-NMR (400 MHz, DMSO-d₆) δ 8.78 (s, 1H), 8.34 (d, |
| | (trifluoromethyl)phenyl)sulfonyl)-1- | J = 2.2 Hz, 1H), 8.10 (dd, J = 8.3, 2.0 Hz, 1H), 7.92 (d, J = |
| | (2,4-dichlorobenzyl)-1H-1,2,3- | 8.3 Hz, 1H), 7.71 (dd, J = 6.1, 2.2 Hz, 1H), 7.50 (dd, J = |
| | triazole-4-carboxamide | 8.3, 2.2 Hz, 1H), 7.39 (d, J = 8.3 Hz, 1H), 5.74 (d, J = 7.1 |
| | | Hz, 2H); LCMS(M + 1): 514.85 |
| 108 | 1-(3,5-bis(trifluoromethyl)phenyl)- | ¹H-NMR (400 MHz, DMSO-d₆) δ 13.32 (bs, 1H), 8.46 |
| | N-((2-chloro-5- | (d, J = 9.5 Hz, 3H), 7.62 (d, J = 3.2 Hz, 1H), 7.58 (d, J = |
| | methoxyphenyl)sulfonyl)-5-methyl- | 9.0 Hz, 1H), 7.29 (dd, J = 8.9, 3.1 Hz, 1H), 3.86 (s, 3H), |
| | 1H-1,2,3-triazole-4-carboxamide | 2.44 (s, 3H); LCMS (M + 1): 542.90 |
| 109 | 1-(3-chloro-5- | ¹H-NMR (400 MHz, DMSO-d₆) δ 13.28 (bs, 1H), 8.20- |
| | (trifluoromethyl)phenyl)-N-((2- | 8.19 (m, 2H), 8.10 (s, 1H), 7.62 (d, J = 2.9 Hz, 1H), 7.58 |
| | chloro-5-methoxyphenyl)sulfonyl)- | (d, J = 8.8 Hz, 1H), 7.28 (dd, J = 8.9, 3.1 Hz, 1H), 3.85 |
| | 5-methyl-1H-1,2,3-triazole-4- | (s, 3H), 2.43 (s, 3H); LCMS (M + 1): 508.90 |
| | carboxamide | |
| 110 | 1-cyclopropyl-N-((2,5- | ¹H-NMR (400 MHz, CHLOROFORM-D) δ 9.86 (s, 1H), |
| | dichlorophenyl)sulfonyl)-5- | 8.33 (d, J = 2.4 Hz, 1H), 7.55-7.52 (m, 1H), 7.45 (d, J = |
| | (trifluoromethyl)-1H-1,2,3-triazole- | 8.3 Hz, 1H), 3.84-3.80 (m, 1H), 1.52-1.48 (m, 2H), 1.36- |
| | 4-carboxamide | 1.31 (m, 2H); LCMS (M − 2): 426.95 |
| 111 | N-((2-chloro-5- | ¹H-NMR (400 MHz, CHLOROFORM-D) δ 9.88 (s, 1H), |
| | methoxyphenyl)sulfonyl)-1-(2,6- | 7.86 (d, J = 2.9 Hz, 1H), 7.64-7.57 (m, 1H), 7.42 (d, J = |
| | difluorophenyl)-5-methyl-1H-1,2,3- | 8.8 Hz, 1H), 7.19 (dd, J = 8.7, 7.2 Hz, 2H), 7.09 (dd, J = |
| | triazole-4-carboxamide | 8.8, 3.2 Hz, 1H), 3.90 (s, 3H), 2.40-2.45 (3H); |
| | | LCMS(M+): 442.90 |
| 112 | N-((2,5-dichlorophenyl)sulfonyl)-1- | ¹H-NMR (400 MHz, CHLOROFORM-D) δ 8.36 (d, J = |
| | (2,6-difluorophenyl)-5-methyl-1H- | 2.4 Hz, 1H), 7.65-7.53 (m, 2H), 7.48 (d, J = 8.6 Hz, 1H), |
| | 1,2,3-triazole-4-carboxamide | 7.19 (dd, J = 8.6, 7.3 Hz, 2H), 2.44 (s, 3H); LCMS(M+): |
| | | 446.90 |
| 113 | N-((2-chloro-5- | ¹H-NMR (400 MHz, CHLOROFORM-D) δ 9.73-10.16 |
| | (trifluoromethyl)phenyl)sulfonyl)-1- | (1H), 8.64 (d, J = 2.2 Hz, 1H), 7.84-7.82 (m, 1H), 7.69 |
| | (2,6-difluorophenyl)-5-methyl-1H- | (d, J = 8.1 Hz, 1H), 7.64-7.59 (m, 1H), 7.20 (dd, J = 8.6, |
| | 1,2,3-triazole-4-carboxamide | 7.1 Hz, 2H), 2.43 (s, 3H); LCMS(M+): 480.90 |
| 114 | N-((2-chlorophenyl)sulfonyl)-1-(2,6- | ¹H-NMR (400 MHz, CHLOROFORM-D) δ 9.92 (s, 1H), |
| | difluorophenyl)-5-methyl-1H-1,2,3- | 8.38 (dd, J = 7.7, 1.6 Hz, 1H), 7.64-7.49 (m, 4H), 7.21- |
| | triazole-4-carboxamide | 7.16 (m, 2H), 2.42 (s, 3H); LCMS(M+): 413.20 |
| 115 | 1-(2,6-difluorophenyl)-N-((2- | ¹H-NMR (400 MHz, CHLOROFORM-D) δ 9.83 (s, 1H), |
| | fluorophenyl)sulfonyl)-5-methyl-1H- | 8.22-8.17 (m, 1H), 7.69-7.57 (m, 2H), 7.39-7.35 (m, 1H), |
| | 1,2,3-triazole-4-carboxamide | 7.26-7.26 (1H), 7.19-7.17 (m, 2H), 2.43 (s, 3H); |
| | | LCMS(M + 1): 397.25 |
| 116 | 1-(2,6-difluorophenyl)-5-methyl-N- | ¹H-NMR (400 MHz, CHLOROFORM-D) δ 9.93 (s, 1H), |
| | ((2,4,6-trifluorophenyl)sulfonyl)-1H- | 7.66-7.58 (m, 1H), 7.20 (dd, J = 8.6, 7.3 Hz, 2H), 6.88- |
| | 1,2,3-triazole-4-carboxamide | 6.82 (m, 2H), 2.46 (s, 3H); LCMS(M + 1): 433.35 |
| 117 | N-((5-chloropyridin-2-yl)sulfonyl)- | ¹H-NMR (400 MHz, CHLOROFORM-D) δ 9.77 (s, 1H), |
| | 1-(2,6-difluorophenyl)-5-methyl-1H- | 8.69 (dd, J = 2.3, 0.6 Hz, 1H), 8.29 (dd, J = 8.3, 0.7 Hz, |
| | 1,2,3-triazole-4-carboxamide | 1H), 7.96 (dd, J = 8.3, 2.4 Hz, 1H), 7.65-7.57 (m, 1H), |
| | | 7.19 (dd, J = 8.6, 7.1 Hz, 2H), 2.43 (s, 3H); |
| | | LCMS(M + 1): 414.15 |
| 118 | N-((2-chloro-5- | ¹H-NMR (400 MHz, CHLOROFORM-D) δ 10.02 (s, |
| | methoxyphenyl)sulfonyl)-1-(3,5- | 1H), 7.85 (d, J = 3.2 Hz, 1H), 7.47 (d, J = 5.6 Hz, 2H), |
| | dichloro-4-fluorophenyl)-5-methyl- | 7.39 (s, 1H), 7.09 (dd, J = 8.8, 3.2 Hz, 1H), 3.90 (s, 3H), |
| | 1H-1,2,3-triazole-4-carboxamide | 2.56 (s, 3H); LCMS(M + 1): 494.80 |
| 119 | 1-(3,5-dichloro-4-fluorophenyl)-N- | ¹H-NMR (400 MHz, CHLOROFORM-D) δ 8.34 (d, J = |
| | ((2,5-dichlorophenyl)sulfonyl)-5- | 2.4 Hz, 1H), 7.54 (dd, J = 8.6, 2.4 Hz, 1H), 7.48-7.42 (m, |
| | methyl-1H-1,2,3-triazole-4- | 3H), 2.56 (d, J = 2.7 Hz, 3H); LCMS(M+): 498.80 |
| | carboxamide | |
| 120 | N-((2-chloro-5- | ¹H-NMR (400 MHz, DMSO-d₆) δ 8.35 (d, J = 2.2 Hz, |
| | (trifluoromethyl)phenyl)sulfonyl)-1- | 1H), 8.09-8.07 (m, 1H), 8.03 (d, J = 6.1 Hz, 2H), 7.92 (d, |
| | (3,5-dichloro-4-fluorophenyl)-5- | J = 8.6 Hz, 1H), 2.41 (s, 3H); LCMS(M + 1): 532.75 |
| | methyl-1H-1,2,3-triazole-4- | |
| | carboxamide | |
| 121 | 1-(3,5-dichloro-4-fluorophenyl)-5- | ¹H-NMR (400 MHz, DMSO-d₆) δ 8.35 (d, J = 7.6 Hz, |
| | methyl-N-((2- | 1H), 8.04-7.88 (m, 5H), 2.41 (s, 3H); LCMS(M+): 496.90 |
| | (trifluoromethyl)phenyl)sulfonyl)- | |
| | 1H-1,2,3-triazole-4-carboxamide | |
| 122 | N-((2-chlorophenyl)sulfonyl)-1-(2,6- | ¹H-NMR (400 MHz, CHLOROFORM-D) δ 9.97 (s, 1H), |
| | difluorophenyl)-5-(trifluoromethyl)- | 8.39 (dd, J = 7.8, 1.5 Hz, 1H), 7.69-7.50 (m, 4H), 7.22- |
| | 1H-1,2,3-triazole-4-carboxamide | 7.18 (m, 2H); LCMS(M − 1): 465.00 |
| 123 | 1-(2,6-difluorophenyl)-N-((2- | ¹H-NMR (400 MHz, CHLOROFORM-D) δ 9.90 (s, 1H), |
| | fluorophenyl)sulfonyl)-5- | 8.22-8.18 (m, 1H), 7.71-7.62 (m, 2H), 7.38 (td, J = 7.7, |
| | (trifluoromethyl)-1H-1,2,3-triazole- | 0.9 Hz, 1H), 7.24 (dd, J = 8.6, 1.0 Hz, 1H), 7.22-7.17 (m, |
| | 4-carboxamide | 2H); LCMS(M − 1): 449.05 |
| 124 | N-((2-chloro-5- | ¹H-NMR (400 MHz, CHLOROFORM-D) δ 7.85 (d, J = |
| | methoxyphenyl)sulfonyl)-5-methyl- | 3.2 Hz, 1H), 7.54-7.52 (m, 2H), 7.40 (d, J = 8.6 Hz, 1H), |
| | 1-(3,4,5-trichlorophenyl)-1H-1,2,3- | 7.09 (dd, J = 8.8, 2.9 Hz, 1H), 3.91 (s, 3H), 2.58 (s, 3H); |
| | triazole-4-carboxamide | LCMS(M+): 510.75 |
| 125 | N-((2,5-dichlorophenyl)sulfonyl)-5- | ¹H-NMR (400 MHz, CHLOROFORM-D) δ 9.84 (s, 1H), |
| | methyl-1-(3,4,5-trichlorophenyl)- | 8.35 (d, J = 2.4 Hz, 1H), 7.56-7.52 (m, 3H), 7.46 (d, J = |
| | 1H-1,2,3-triazole-4-carboxamide | 8.6 Hz, 1H), 2.59 (s, 3H); LCMS(M+): 514.80 |
| 126 | N-((2-chlorophenyl)sulfonyl)-5- | ¹H-NMR (400 MHz, CHLOROFORM-D) δ 9.85 (s, 1H), |
| | methyl-1-(3,4,5-trichlorophenyl)- | 8.38-8.36 (m, 1H), 7.61-7.57 (m, 1H), 7.55-7.50 (m, 4H), |
| | 1H-1,2,3-triazole-4-carboxamide | 2.58 (d, J = 7.1 Hz, 3H); LCMS(M+): 480.75 |
| 127 | N-((2-fluorophenyl)sulfonyl)-5- | ¹H-NMR (400 MHz, CHLOROFORM-D) δ 9.67-9.96 |
| | methyl-1-(3,4,5-trichlorophenyl)- | (1H), 8.19 (td, J = 7.6, 1.5 Hz, 1H), 7.69-7.64 (m, 1H), |
| | 1H-1,2,3-triazole-4-carboxamide | 7.52 (d, J = 3.4 Hz, 2H), 7.38 (td, J = 7.8, 1.1 Hz, 1H), |
| | | 7.25-7.20 (m, 1H), 2.58 (s, 3H); LCMS(M + 1): 464.65 |
| 128 | 1-(3,5-bis(trifluoromethyl)phenyl)- | ¹H-NMR (400 MHz, DMSO-d₆) δ 9.68 (s, 1H), 8.65 (s, |
| | N-((2-chloro-5- | 2H), 8.34 (s, 1H), 7.63 (d, J = 3.2 Hz, 1H), 7.56 (d, J = |
| | methoxyphenyl)sulfonyl)-1H-1,2,3- | 8.8 Hz, 1H), 7.28 (dd, J = 8.8, 2.9 Hz, 1H), 3.86 (s, 3H); |
| | triazole-4-carboxamide | LCMS (M + 1): 528.90 |
| 129 | 5-methyl-1-(3,4,5-trichlorophenyl)- | ¹H-NMR (400 MHz, CHLOROFORM-D) δ 9.60-9.90 |
| | N-((2- | (1H), 8.61 (d, J = 7.3 Hz, 1H), 7.92-7.89 (m, 1H), 7.85- |
| | (trifluoromethyl)phenyl)sulfonyl)- | 7.77 (m, 2H), 7.52 (s, 2H), 2.57 (s, 3H); LCMS(M+): |
| | 1H-1,2,3-triazole-4-carboxamide | 514.80 |
| 130 | 1-(3,5-dichloro-4-fluorophenyl)-5- | ¹H-NMR (400 MHz, CHLOROFORM-D) δ 9.87 (s, 1H), |
| | methyl-N-((2,4,5- | 8.43 (s, 1H), 7.64 (s, 1H), 7.48 (s, 1H), 7.46 (s, 1H), 2.59- |
| | trichlorophenyl)sulfonyl)-1H-1,2,3- | 2.57 (m, 3H); LCMS(M+): 532.75 |
| | triazole-4-carboxamide | |
| 131 | 1-(2-chloro-4- | ¹H-NMR (400 MHz, CHLOROFORM-D) δ 9.97 (s, 0H), |
| | (trifluoromethyl)phenyl)-N-((2,4,5- | 8.45 (s, 1H), 7.93 (s, 1H), 7.81-7.79 (m, 1H), 7.67 (s, 1H), |
| | trichlorophenyl)sulfonyl)-5- | 7.62 (d, J = 8.1 Hz, 1H); LCMS(M − 1): 600.80 |
| | (trifluoromethyl)-1H-1,2,3-triazole- | |
| | 4-carboxamide | |
| 132 | 1-(2-chloro-4- | ¹H-NMR (400 MHz, CHLOROFORM-D) δ 9.48-9.97 |
| | (trifluoromethoxy)phenyl)-5-methyl- | (1H), 8.63 (d, J = 7.3 Hz, 1H), 7.92-7.90 (m, 1H), 7.85- |
| | N-((2- | 7.77 (m, 2H), 7.51-7.45 (m, 2H), 7.37-7.35 (m, 1H), 2.41 |
| | (trifluoromethyl)phenyl)sulfonyl)- | (s, 3H); LCMS(M): 529.00 |
| | 1H-1,2,3-triazole-4-carboxamide | |
| 133 | 1-(2-chloro-4- | ¹H-NMR (400 MHz, CHLOROFORM-D) δ 9.89 (d, J = |
| | (trifluoromethoxy)phenyl)-N-((2- | 8.3 Hz, 1H), 7.86 (d, J = 3.1 Hz, 1H), 7.51 (d, J = 1.8 Hz, |
| | chloro-5-methoxyphenyl)sulfonyl)- | 1H), 7.48 (d, J = 8.6 Hz, 1H), 7.41 (t, J = 4.4 Hz, 1H), |
| | 5-methyl-1H-1,2,3-triazole-4- | 7.38-7.34 (m, 1H), 7.09 (dd, J = 8.7, 3.2 Hz, 1H), 3.90 (s, |
| | carboxamide | 3H), 2.38-2.43 (s, 3H); LCMS(M+): 526.10 |
| 134 | 1-(2-chloro-4- | ¹H-NMR (400 MHz, CHLOROFORM-D) δ 8.36 (d, J = |
| | (trifluoromethoxy)phenyl)-N-((2,5- | 2.8 Hz, 1H), 7.55 (dd, J = 8.6, 2.4 Hz, 1H), 7.51 (dd, J = |
| | dichlorophenyl)sulfonyl)-5-methyl- | 5.5, 2.1 Hz, 1H), 7.49 (s, 1H), 7.47 (s, 1H), 7.37 (dt, J = |
| | 1H-1,2,3-triazole-4-carboxamide | 7.7, 1.2 Hz, 1H), 2.42 (s, 3H); LCMS(M+): 530.30 |
| 135 | 1-(2-chloro-4- | ¹H-NMR (400 MHz, CHLOROFORM-D) δ 9.81-10.03 |
| | (trifluoromethoxy)phenyl)-N-((2- | (0H), 8.63 (d, J = 1.5 Hz, 1H), 7.83 (dd, J = 8.4, 2.0 Hz, |
| | chloro-5- | 1H), 7.69 (d, J = 8.3 Hz, 1H), 7.52 (d, J = 1.8 Hz, 1H), |
| | (trifluoromethyl)phenyl)sulfonyl)-5- | 7.48 (d, J = 8.9 Hz, 1H), 7.37 (d, J = 8.6 Hz, 1H), 2.41 (s, |
| | methyl-1H-1,2,3-triazole-4- | 3H); LCMS (M): 562.95 |
| | carboxamide | |
| 136 | 1-(2-chloro-4- | ¹H-NMR (400 MHz, CHLOROFORM-D) δ 9.85 (s, 1H), |
| | (trifluoromethoxy)phenyl)-5-methyl- | 8.45 (s, 1H), 7.65 (s, 1H), 7.52 (d, J = 1.5 Hz, 1H), 7.48 |
| | N-((2,4,5-trichlorophenyl)sulfonyl)- | (d, J = 8.9 Hz, 1H), 7.37 (dq, J = 8.7, 1.2 Hz, 1H), 2.42 |
| | 1H-1,2,3-triazole-4-carboxamide | (s, 3H); LCMS(M+): 564.70 |
| 137 | N-((2-chloro-5- | ¹H-NMR (400 MHz, CHLOROFORM-D) δ 7.82 (d, J = |
| | methoxyphenyl)sulfonyl)-1- | 3.1 Hz, 1H), 7.36 (d, J = 8.6 Hz, 1H), 7.04 (dd, J = 8.9, |
| | cyclopentyl-5-(trifluoromethyl)-1H- | 2.8 Hz, 1H), 3.88 (s, 3H), 3.32 (m, J = 7.3 Hz, 1H), 2.24 |
| | 1,2,3-triazole-4-carboxamide | (dd, J = 12.5, 5.8 Hz, 4H), 2.06-1.97 (m, 2H), 1.83-1.73 |
| | | (m, 2H); LCMS(M+): 452.65 |
| 138 | 1-(2-chloro-4- | ¹H-NMR (400 MHz, CHLOROFORM-D) δ 8.22 (td, J = |
| | (trifluoromethoxy)phenyl)-N-((2- | 7.5, 1.5 Hz, 1H), 7.73-7.67 (m, 1H), 7.48-7.43 (m, 3H), |
| | fluorophenyl)(methyl)(oxo)-λ⁶- | 7.34-7.28 (m, 2H), 3.61 (s, 3H), 2.42 (s, 3H); LCMS(M): |
| | sulfaneylidene)-5-methyl-1H-1,2,3- | 476.60 |
| | triazole-4-carboxamide | |
| 139 | 1-(2-chloro-4- | ¹H-NMR (400 MHz, CHLOROFORM-D) δ 8.15 (q, J = |
| | (trifluoromethoxy)phenyl)-N-((4- | 4.6 Hz, 2H), 7.48 (d, J = 8.6 Hz, 2H), 7.31 (t, J = 8.4 Hz, |
| | fluorophenyl)(methyl)(oxo)-λ⁶- | 3H), 3.48 (s, 3H), 2.44 (s, 3H); LCMS(M+): 476.60 |
| | sulfaneylidene)-5-methyl-1H-1,2,3- | |
| | triazole-4-carboxamide | |
| 140 | 1-(2-chloro-4- | ¹H-NMR (400 MHz, CHLOROFORM-D) δ 8.13 (dd, J = |
| | (trifluoromethoxy)phenyl)-N-((3,5- | 6.7, 2.1 Hz, 1H), 7.55 (dd, J = 6.7, 2.1 Hz, 2H), 7.51-7.45 |
| | difluorophenyl)sulfonyl)-5-methyl- | (m, 2H), 7.37 (d, J = 7.8 Hz, 1H), 2.44 (d, J = 4.6 Hz, 3H); |
| | 1H-1,2,3-triazole-4-carboxamide | LCMS(M+): 496.55 |
| 141 | 1-(2-chloro-4- | ¹H-NMR (400 MHz, CHLOROFORM-D) δ 9.75 (s, 1H), |
| | (trifluoromethoxy)phenyl)-N-((5- | 8.69-8.68 (m, 1H), 8.30-8.28 (m, 1H), 7.97 (dd, J = 8.3, |
| | chloropyridin-2-yl)sulfonyl)-5- | 2.4 Hz, 1H), 7.51 (d, J = 1.8 Hz, 1H), 7.47 (d, J = 8.6 Hz, |
| | methyl-1H-1,2,3-triazole-4- | 1H), 7.36 (dq, J = 8.8, 1.2 Hz, 1H), 2.41 (s, 3H); |
| | carboxamide | LCMS(M+): 495.50 |
| 142 | 1-(2-chloro-4- | ¹H-NMR (400 MHz, CHLOROFORM-D) δ 8.09 (t, J = |
| | (trifluoromethoxy)phenyl)-N-((3- | 2.0 Hz, 1H), 8.00 (dt, J = 8.0, 1.4 Hz, 1H), 7.67 (dq, J = |
| | chlorophenyl)(methyl)(oxo)-λ⁶- | 8.1, 1.0 Hz, 1H), 7.58 (t, J = 7.9 Hz, 1H), 7.50-7.48 (m, |
| | sulfaneylidene)-5-methyl-1H-1,2,3- | 2H), 7.34 (dt, J = 8.7, 1.1 Hz, 1H), 3.47 (s, 3H), 2.43 (s, |
| | triazole-4-carboxamide | 3H); LCMS(M): 492.55 |
| 143 | 1-(2-chloro-4- | ¹H-NMR (400 MHz, CHLOROFORM-D) δ 8.20 (td, J = |
| | (trifluoromethoxy)phenyl)-N-((2- | 7.6, 1.5 Hz, 1H), 7.68-7.65 (m, 1H), 7.51 (d, J = 1.7 Hz, |
| | fluorophenyl)sulfonyl)-5-methyl-1H- | 1H), 7.47 (d, J = 8.8 Hz, 1H), 7.40-7.35 (m, 2H), 7.23 (d, |
| | 1,2,3-triazole-4-carboxamide | J = 8.8 Hz, 1H), 2.41 (s, 3H); LCMS(M): 478.95 |
| 144 | 1-(2-chloro-4- | ¹H-NMR (400 MHz, CHLOROFORM-D) δ 8.38 (dd, J = |
| | (trifluoromethoxy)phenyl)-N-((2- | 7.8, 1.5 Hz, 1H), 7.59-7.46 (m, 5H), 7.36 (d, J = 8.8 Hz, |
| | chlorophenyl)sulfonyl)-5-methyl- | 1H), 2.40 (s, 3H); LCMS(M+): 494.95 |
| | 1H-1,2,3-triazole-4-carboxamide | |
| 145 | 1-(2-chloro-4- | ¹H-NMR (400 MHz, CHLOROFORM-D) δ 8.13 (dd, J = |
| | (trifluoromethoxy)phenyl)-N-((4- | 6.7, 2.1 Hz, 2H), 7.55 (dd, J = 6.7, 2.1 Hz, 2H), 7.51-7.45 |
| | chlorophenyl)sulfonyl)-5-methyl- | (m, 2H), 7.37 (d, J = 7.8 Hz, 1H), 2.44 (d, J = 4.6 Hz, 3H); |
| | 1H-1,2,3-triazole-4-carboxamide | LCMS(M+): 494.95 |
| 146 | 1-(2-chloro-4- | ¹H-NMR (400 MHz, DMSO-d₆) δ 8.22 (d, J = 5.9 Hz, |
| | (trifluoromethoxy)phenyl)-5-methyl- | 2H), 8.03 (t, J = 6.8 Hz, 3H), 7.89 (d, J = 8.1 Hz, 1H), |
| | N-((4- | 7.70 (d, J = 7.6 Hz, 1H), 2.29 (s, 3H); LCMS(M − 1): |
| | (trifluoromethyl)phenyl)sulfonyl)- | 526.95 |
| | 1H-1,2,3-triazole-4-carboxamide | |
| 147 | 5-methyl-1-(3,4,5-trichlorophenyl)- | ¹H-NMR (400 MHz, DMSO-d₆) δ 8.21 (d, J = 8.3 Hz, |
| | N-((4- | 2H), 8.05 (s, 1H), 8.04 (s, 1H), 8.03 (d, J = 8.1 Hz, 2H), |
| | (trifluoromethyl)phenyl)sulfonyl)- | 2.44 (s, 3H); LCMS(M+): 512.75 |
| | 1H-1,2,3-triazole-4-carboxamide | |
| 148 | 1-(3,5-dichloro-4-fluorophenyl)-5- | ¹H-NMR (400 MHz, DMSO-d₆) δ 12.98 (s, 1H), 8.21 (d, |
| | methyl-N-((4- | J = 8.3 Hz, 2H), 8.05 (d, J = 8.6 Hz, 2H), 8.01 (d, J = 6.1 |
| | (trifluoromethyl)phenyl)sulfonyl)- | Hz, 1H), 8.01 (d, J = 6.1 Hz, 2H), 2.42 (s, 3H); LCMS(M − |
| | 1H-1,2,3-triazole-4-carboxamide | 2): 495.00 |
| 149 | 1-(2,6-difluorophenyl)-5- | ¹H-NMR (400 MHz, DMSO-d₆) δ 8.11 (d, J = 8.1 Hz, |
| | (trifluoromethyl)-N-((4- | 2H), 7.94-7.86 (m, 3H), 7.57 (dd, J = 8.7, 7.9 Hz, 2H); |
| | (trifluoromethyl)phenyl)sulfonyl)- | LCMS(M − 1): 498.95 |
| | 1H-1,2,3-triazole-4-carboxamide | |
| 150 | 1-(2-chloro-4- | ¹H-NMR (400 MHz, CHLOROFORM-D) δ 9.82 (s, 1H), |
| | (trifluoromethoxy)phenyl)-N-((2- | 8.50 (s, 1H), 7.86 (d, J = 3.1 Hz, 1H), 7.69 (d, J = 8.9 Hz, |
| | chloro-5-methoxyphenyl)sulfonyl)- | 1H), 7.52-7.50 (m, 1H), 7.41 (dd, J = 5.0, 3.5 Hz, 1H), |
| | 1H-1,2,3-triazole-4-carboxamide | 7.36 (dq, J = 8.8, 1.2 Hz, 1H), 7.10 (dd, J = 8.9, 3.1 Hz, |
| | | 1H), 3.89 (d, J = 15.0 Hz, 3H); LCMS(M − 1): 510.80 |
| 151 | 1-(2-chloro-4- | ¹H-NMR (400 MHz, CHLOROFORM-D) δ 9.81 (d, J = |
| | (trifluoromethoxy)phenyl)-N-((2,5- | 35.5 Hz, 1H), 8.52 (s, 1H), 8.36 (d, J = 2.4 Hz, 1H), 7.69 |
| | dichlorophenyl)sulfonyl)-1H-1,2,3- | (d, J = 8.9 Hz, 1H), 7.55 (dd, J = 8.4, 2.6 Hz, 1H), 7.52- |
| | triazole-4-carboxamide | 7.50 (m, 1H), 7.47 (d, J = 8.6 Hz, 1H), 7.38-7.35 (m, 1H); |
| | | LCMS(M+): 514.75 |
| 152 | 1-(2-chloro-4- | ¹H-NMR (400 MHz, CHLOROFORM-D) δ 8.64 (d, J = |
| | (trifluoromethoxy)phenyl)-N-((2- | 1.8 Hz, 1H), 8.51 (s, 1H), 7.83 (dd, J = 8.3, 1.8 Hz, 1H), |
| | chloro-5- | 7.69 (dd, J = 8.9, 2.8 Hz, 2H), 7.52-7.50 (m, 1H), 7.37 |
| | (trifluoromethyl)phenyl)sulfonyl)- | (dq, J = 8.9, 1.1 Hz, 1H); LCMS(M+): 548.75 |
| | 1H-1,2,3-triazole-4-carboxamide | |
| 153 | 1-(2-chloro-4- | ¹H-NMR (400 MHz, CHLOROFORM-D) δ 8.52 (s, 1H), |
| | (trifluoromethoxy)phenyl)-N-((2,4,5- | 8.45 (s, 1H), 7.69 (dd, J = 8.9, 2.1 Hz, 1H), 7.64 (s, 1H), |
| | trichlorophenyl)sulfonyl)-1H-1,2,3- | 7.52-7.51 (m, 1H), 7.39-7.36 (m, 1H); LCMS(M+): |
| | triazole-4-carboxamide | 550.70 |
| 154 | 1-(2-chloro-4- | ¹H-NMR (400 MHz, CHLOROFORM-D) δ 9.61 (s, 1H), |
| | (trifluoromethoxy)phenyl)-N-((2- | 8.65-8.63 (m, 1H), 8.49 (s, 1H), 7.91 (dd, J = 7.2, 1.8 Hz, |
| | (trifluoromethyl)phenyl)sulfonyl)- | 1H), 7.85-7.78 (m, 1H), 7.69-7.66 (m, 1H), 7.49 (d, J = |
| | 1H-1,2,3-triazole-4-carboxamide | 2.0 Hz, 1H), 7.36 (dq, J = 8.8, 1.1 Hz, 1H); LCMS(M+): |
| | | 514.75 |
| 155 | 1-(2-chloro-4- | ¹H-NMR (400 MHz, DMSO-d₆) δ 9.20 (s, 1H), 8.16 (dd, |
| | (trifluoromethoxy)phenyl)-N-((3- | J = 6.8, 2.4 Hz, 1H), 8.03-7.99 (m, 2H), 7.91 (d, J = 8.8 |
| | chloro-4-fluorophenyl)sulfonyl)-1H- | Hz, 1H), 7.72-7.66 (m, 2H); LCMS(M+): 498.85 |
| | 1,2,3-triazole-4-carboxamide | |
| 156 | N-((5-chloro-2,4- | ¹H-NMR (400 MHz, DMSO-d₆) δ 9.11 (s, 1H), 8.11 (t, |
| | difluorophenyl)sulfonyl)-1-(2- | J = 7.6 Hz, 1H), 7.98 (d, J = 2.0 Hz, 1H), 7.91 (d, J = 8.8 |
| | chloro-4-(trifluoromethoxy)phenyl)- | Hz, 1H), 7.79 (t, J = 9.5 Hz, 1H), 7.66 (dd, J = 8.9, 1.6 |
| | 1H-1,2,3-triazole-4-carboxamide | Hz, 1H); LCMS(M+): 516.85 |
| 157 | 1-(2-chloro-4- | ¹H-NMR (400 MHz, DMSO-d₆) δ 9.17 (s, 1H), 7.99 (d, |
| | (trifluoromethoxy)phenyl)-N-((2,5- | J = 2.2 Hz, 1H), 7.92 (d, J = 8.8 Hz, 1H), 7.78-7.74 (m, |
| | difluorophenyl)sulfonyl)-1H-1,2,3- | 1H), 7.69-7.62 (m, 2H), 7.53 (dd, J = 9.5, 4.2 Hz, 1H); |
| | triazole-4-carboxamide | LCMS(M+): 482.90 |
| 158 | N-((5-chloro-2- | ¹H-NMR (400 MHz, DMSO-d₆) δ 9.03 (s, 1H), 7.97 (s, |
| | fluorophenyl)sulfonyl)-1-(2-chloro- | 1H), 7.91-7.89 (m, 2H), 7.73 (s, 1H), 7.67-7.64 (m, 1H), |
| | 4-(trifluoromethoxy)phenyl)-1H- | 7.44 (s, 1H); LCMS(M+): 498.95 |
| | 1,2,3-triazole-4-carboxamide | |
| 159 | N-((5-chloro-2- | ¹H-NMR (400 MHz, DMSO-d₆) δ 8.14-8.00 (m, 2H), |
| | fluorophenyl)sulfonyl)-1-(2-chloro- | 7.83-7.65 (m, 3H), 7.38 (t, J = 9.2 Hz, 1H); LCMS(M+): |
| | 4-(trifluoromethoxy)phenyl)-5- | 566.90 |
| | (trifluoromethyl)-1H-1,2,3-triazole- | |
| | 4-carboxamide | |
| 160 | N-((5-chloro-2,4- | ¹H-NMR (400 MHz, DMSO-d₆) δ 8.14-7.98 (m, 3H), |
| | difluorophenyl)sulfonyl)-1-(2- | 7.76-7.68 (m, 2H); LCMS(M+): 584.95 |
| | chloro-4-(trifluoromethoxy)phenyl)- | |
| | 5-(trifluoromethyl)-1H-1,2,3- | |
| | triazole-4-carboxamide | |
| 161 | 1-(2-chloro-4- | ¹H-NMR (400 MHz, DMSO-d₆) δ 8.10-7.94 (m, 4H), |
| | (trifluoromethoxy)phenyl)-N-((3- | 7.72-7.62 (m, 2H); LCMS(M+): 566.85 |
| | chloro-4-fluorophenyl)sulfonyl)-5- | |
| | (trifluoromethyl)-1H-1,2,3-triazole- | |
| | 4-carboxamide | |
| 162 | N-((3,5- | ¹H-NMR (400 MHz, DMSO-d₆) δ 8.40 (s, 2H), 8.35 (s, |
| | bis(trifluoromethyl)phenyl)sulfonyl)- | 1H), 8.07 (d, J = 8.8 Hz, 1H), 8.01 (d, J = 2.7 Hz, 1H), |
| | 1-(2-chloro-4- | 7.70 (dq, J = 8.8, 1.2 Hz, 1H); LCMS(M+): 649.25 |
| | (trifluoromethoxy)phenyl)-5- | |
| | (trifluoromethyl)-1H-1,2,3-triazole- | |
| | 4-carboxamide | |
| 163 | 1-(2-chloro-4- | ¹H-NMR (400 MHz, DMSO-d₆) δ 8.12 (d, J = 8.9 Hz, |
| | (trifluoromethoxy)phenyl)-N-((2- | 1H), 8.05 (d, J = 2.1 Hz, 1H), 7.73 (dq, J = 8.9, 1.2 Hz, |
| | chloro-5-methoxyphenyl)sulfonyl)- | 1H), 7.60 (d, J = 3.1 Hz, 1H), 7.54 (d, J = 8.6 Hz, 1H), |
| | 5-(trifluoromethyl)-1H-1,2,3- | 7.23 (dd, J = 8.7, 3.2 Hz, 1H), 3.84 (s, 3H); LCMS(M+): |
| | triazole-4-carboxamide | 578.95 |
| 164 | 1-(2-chloro-4- | ¹H-NMR (400 MHz, DMSO-d₆) δ 8.11 (d, J = 8.9 Hz, |
| | (trifluoromethoxy)phenyl)-N-((2,5- | 1H), 8.05 (d, J = 2.4 Hz, 1H), 8.04 (d, J = 2.1 Hz, 1H), |
| | dichlorophenyl)sulfonyl)-5- | 7.74-7.69 (m, 2H), 7.64 (d, J = 8.6 Hz, 1H); LCMS(M − |
| | (trifluoromethyl)-1H-1,2,3-triazole- | 1): 582.70 |
| | 4-carboxamide | |
| 165 | 1-(2-chloro-4- | ¹H-NMR (400 MHz, DMSO-d₆) δ 8.13-8.08 (m, 2H), |
| | (trifluoromethoxy)phenyl)-N-((2,4,5- | 8.01 (d, J = 2.4 Hz, 1H), 7.92 (s, 1H), 7.70 (d, J = 8.8 Hz, |
| | trichlorophenyl)sulfonyl)-5- | 1H); LCMS(M − 2): 616.35 |
| | (trifluoromethyl)-1H-1,2,3-triazole- | |
| | 4-carboxamide | |
| 166 | 1-(2-chloro-4- | ¹H-NMR (400 MHz, DMSO-d₆) δ 8.34 (d, J = 7.6 Hz, |
| | (trifluoromethoxy)phenyl)-5- | 1H), 8.10 (d, J = 8.9 Hz, 1H), 8.04 (d, J = 2.1 Hz, 1H), |
| | (trifluoromethyl)-N-((2- | 7.94 (d, J = 7.6 Hz, 1H), 7.91-7.87 (m, 1H), 7.83 (t, J = |
| | (trifluoromethyl)phenyl)sulfonyl)- | 7.3 Hz, 1H), 7.72 (dq, J = 8.9, 1.2 Hz, 1H); LCMS(M+): |
| | 1H-1,2,3-triazole-4-carboxamide | 582.70 |
| 167 | N-((5-chloro-2,4- | ¹H-NMR (400 MHz, DMSO-d₆) δ 8.00-7.96 (m, 2H), |
| | difluorophenyl)sulfonyl)-1-(2- | 7.86 (d, J = 8.8 Hz, 1H), 7.68-7.61 (m, 2H), 2.31 (s, 3H); |
| | chloro-4-(trifluoromethoxy)phenyl)- | LCMS(M − 2): 528.85 |
| | 5-methyl-1H-1,2,3-triazole-4- | |
| | carboxamide | |
| 168 | N-((5-chloro-2- | ¹H-NMR (400 MHz, DMSO-d₆) δ 7.98-7.93 (m, 2H), |
| | fluorophenyl)sulfonyl)-1-(2-chloro- | 7.86 (s, 1H), 7.66 (dq, J = 8.8, 1.2 Hz, 2H), 7.58 (t, J = |
| | 4-(trifluoromethoxy)phenyl)-5- | 9.4 Hz, 1H), 2.32 (s, 3H); LCMS(M − 1): 512.65 |
| | methyl-1H-1,2,3-triazole-4- | |
| | carboxamide | |
| 169 | 1-(2-chloro-4- | ¹H-NMR (400 MHz, DMSO-d₆) δ 8.01 (d, J = 2.0 Hz, |
| | (trifluoromethoxy)phenyl)-N-((2,5- | 1H), 7.91 (d, J = 8.8 Hz, 1H), 7.78-7.74 (m, 1H), 7.71- |
| | difluorophenyl)sulfonyl)-5-methyl- | 7.63 (m, 2H), 7.54 (td, J = 9.3, 4.2 Hz, 1H), 2.28 (s, 3H); |
| | 1H-1,2,3-triazole-4-carboxamide | LCMS(M+): 495.15 |
| 170 | 1-(2-chloro-4- | ¹H-NMR (400 MHz, DMSO-d₆) δ 8.14 (dd, J = 6.8, 2.2 |
| | (trifluoromethoxy)phenyl)-N-((3- | Hz, 1H), 8.02-7.98 (m, 2H), 7.88 (d, J = 8.8 Hz, 1H), |
| | chloro-4-fluorophenyl)sulfonyl)-5- | 7.70-7.65 (m, 2H), 2.31 (d, J = 6.6 Hz, 3H); LCMS(M+): |
| | methyl-1H-1,2,3-triazole-4- | 512.80 |
| | carboxamide | |
| 171 | 1-(2-chloro-4- | ¹H-NMR (400 MHz, DMSO-d₆) δ 8.30 (d, J = 2.4 Hz, |
| | (trifluoromethoxy)phenyl)-N-((2- | 1H), 8.09 (d, J = 8.8 Hz, 1H), 8.01 (d, J = 2.4 Hz, 1H), |
| | chloro-5- | 7.91 (dd, J = 8.3, 1.7 Hz, 1H), 7.79 (d, J = 8.3 Hz, 1H), |
| | (trifluoromethyl)phenyl)sulfonyl)-5- | 7.70 (dt, J = 7.7, 1.3 Hz, 1H); LCMS(M + 1): 614.70 |
| | (trifluoromethyl)-1H-1,2,3-triazole- | |
| | 4-carboxamide | |
| 172 | 1-(2-chloro-4- | ¹H-NMR (400 MHz, DMSO-d₆) δ 8.33 (d, J = 1.7 Hz, |
| | (trifluoromethyl)phenyl)-N-((2- | 1H), 8.05 (dd, J = 8.4, 1.6 Hz, 1H), 8.00 (d, J = 8.3 Hz, |
| | chloro-5-methoxyphenyl)sulfonyl)- | 1H), 7.62 (d, J = 2.9 Hz, 1H), 7.57 (d, J = 8.8 Hz, 1H), |
| | 5-methyl-1H-1,2,3-triazole-4- | 7.28 (dd, J = 8.8, 3.2 Hz, 1H), 3.85 (s, 3H), 2.29 (s, 3H); |
| | carboxamide | LCMS(M+): 508.95 |
| 173 | 1-(2-chloro-4- | ¹H-NMR (400 MHz, DMSO-d₆) δ 8.36 (d, J = 2.0 Hz, |
| | (trifluoromethyl)phenyl)-N-((2- | 1H), 8.32 (s, 1H), 8.07-8.03 (m, 2H), 7.99 (d, J = 8.1 Hz, |
| | chloro-5- | 1H), 7.91 (d, J = 8.3 Hz, 1H), 2.30 (s, 3H); LCMS(M − 2): |
| | (trifluoromethyl)phenyl)sulfonyl)-5- | 544.95 |
| | methyl-1H-1,2,3-triazole-4- | |
| | carboxamide | |
| 174 | 1-(2-chloro-4- | ¹H-NMR (400 MHz, DMSO-d₆) δ 8.32 (d, J = 1.5 Hz, |
| | (trifluoromethyl)phenyl)-N-((2,5- | 1H), 8.10 (d, J = 2.4 Hz, 1H), 8.04 (dd, J = 8.7, 1.6 Hz, |
| | dichlorophenyl)sulfonyl)-5-methyl- | 1H), 7.99 (d, J = 8.3 Hz, 1H), 7.77 (dd, J = 8.6, 2.4 Hz, |
| | 1H-1,2,3-triazole-4-carboxamide | 1H), 7.69 (d, J = 8.6 Hz, 1H), 2.30 (s, 3H); LCMS(M+): |
| | | 512.75 |
| 175 | 1-(2-chloro-4- | ¹H-NMR (400 MHz, DMSO-d₆) δ 8.38 (d, J = 7.6 Hz, |
| | (trifluoromethyl)phenyl)-5-methyl- | 1H), 8.33 (d, J = 1.2 Hz, 1H), 8.06-7.89 (m, 5H), 2.30 (d, |
| | N-((2- | J = 11.0 Hz, 3H), LCMS(M + 1): 512.90 |
| | (trifluoromethyl)phenyl)sulfonyl)- | |
| | 1H-1,2,3-triazole-4-carboxamide | |
| 176 | 1-(2-chloro-4- | ¹H-NMR (400 MHz, DMSO-d₆) δ 8.32 (d, J = 1.5 Hz, |
| | (trifluoromethyl)phenyl)-N-((2,4- | 1H), 8.15 (d, J = 8.8 Hz, 1H), 8.05 (dd, J = 8.6, 1.7 Hz, |
| | dichlorophenyl)sulfonyl)-5-methyl- | 1H), 7.99 (d, J = 8.3 Hz, 1H), 7.86 (s, 1H), 7.69 (dd, J = |
| | 1H-1,2,3-triazole-4-carboxamide | 8.6, 2.0 Hz, 1H), 2.29 (s, 3H); LCMS(M+): 512.85 |
| 177 | 1-(2-chloro-4- | ¹H-NMR (400 MHz, DMSO-d₆) δ 8.32 (d, J = 1.2 Hz, |
| | (trifluoromethyl)phenyl)-N-((2,5- | 1H), 8.05 (dd, J = 8.4, 1.3 Hz, 1H), 7.99 (d, J = 8.1 Hz, |
| | difluorophenyl)sulfonyl)-5-methyl- | 1H), 7.78-7.74 (m, 1H), 7.67-7.63 (m, 1H), 7.54 (td, J = |
| | 1H-1,2,3-triazole-4-carboxamide | 9.3, 4.2 Hz, 1H), 2.30 (s, 3H); LCMS(M − 1): 479.05 |
| 178 | N-((5-chloro-2- | ¹H-NMR (400 MHz, DMSO-d₆) δ 8.32 (d, J = 1.5 Hz, |
| | fluorophenyl)sulfonyl)-1-(2-chloro- | 1H), 8.05 (dd, J = 8.6, 1.7 Hz, 1H), 7.99 (d, J = 8.3 Hz, |
| | 4-(trifluoromethyl)phenyl)-5-methyl- | 1H), 7.94 (q, J = 2.9 Hz, 1H), 7.87-7.83 (m, 1H), 7.54 (t, |
| | 1H-1,2,3-triazole-4-carboxamide | J = 9.3 Hz, 1H), 2.30 (s, 3H); LCMS(M − 2): 494.90 |
| 179 | N-((3,5- | ¹H-NMR (400 MHz, DMSO-d₆) δ 8.51 (d, J = 9.8 Hz, |
| | bis(trifluoromethyl)phenyl)sulfonyl)- | 3H), 8.31 (d, J = 1.5 Hz, 1H), 8.04 (dd, J = 8.7, 1.6 Hz, |
| | 1-(2-chloro-4- | 1H), 7.95 (d, J = 8.1 Hz, 1H), 2.31 (s, 3H); LCMS(M − 1): |
| | (trifluoromethyl)phenyl)-5-methyl- | 578.95 |
| | 1H-1,2,3-triazole-4-carboxamide | |
| 180 | N-((5-chloro-2,4- | ¹H-NMR (400 MHz, DMSO-d₆) δ 8.32 (d, J = 1.5 Hz, |
| | difluorophenyl)sulfonyl)-1-(2- | 1H), 8.13 (t, J = 7.6 Hz, 1H), 8.05 (dd, J = 8.3, 1.5 Hz, |
| | chloro-4-(trifluoromethyl)phenyl)-5- | 1H), 7.99 (d, J = 8.3 Hz, 1H), 7.84 (t, J = 9.5 Hz, 1H), |
| | methyl-1H-1,2,3-triazole-4- | 2.30 (s, 3H); LCMS(M − 2): 512.90 |
| | carboxamide | |
| 181 | N-((4-chloro-2,5- | ¹H-NMR (400 MHz, DMSO-d₆) δ 13.12 (s, 0H), 8.32 (d, |
| | dimethylphenyl)sulfonyl)-1-(2- | J = 1.5 Hz, 1H), 8.05 (dd, J = 8.4, 1.3 Hz, 1H), 7.99 (d, |
| | chloro-4-(trifluoromethyl)phenyl)-5- | J = 8.1 Hz, 2H), 7.52 (s, 1H), 2.60 (s, 3H), 2.39 (s, 3H), |
| | methyl-1H-1,2,3-triazole-4- | 2.28 (s, 3H); LCMS(M+): 506.90 |
| | carboxamide | |
| 182 | 1-(4-chloro-2- | ¹H-NMR (400 MHz, DMSO-d₆) δ 8.21 (d, J = 2.1 Hz, |
| | (trifluoromethyl)phenyl)-N-((5- | 1H), 8.09 (dd, J = 8.6, 2.1 Hz, 1H), 7.94 (q, J = 3.0 Hz, |
| | chloro-2-fluorophenyl)sulfonyl)-5- | 1H), 7.87-7.83 (m, 2H), 7.54 (t, J = 9.3 Hz, 1H), 2.26 (s, |
| | methyl-1H-1,2,3-triazole-4- | 3H); LCMS(M+): 497.00 |
| | carboxamide | |
| 183 | N-((4-chloro-2,5- | ¹H-NMR (400 MHz, DMSO-d₆) δ 13.07 (s, 1H), 8.21 (d, |
| | dimethylphenyl)sulfonyl)-1-(4- | J = 2.4 Hz, 1H), 8.09 (dd, J = 8.3, 2.1 Hz, 1H), 8.00 (s, |
| | chloro-2-(trifluoromethyl)phenyl)-5- | 1H), 7.86 (d, J = 8.6 Hz, 1H), 7.52 (s, 1H), 2.59-2.55 (m, |
| | methyl-1H-1,2,3-triazole-4- | 3H), 2.39 (t, J = 15.6 Hz, 3H), 2.20 (s, 3H); LCMS(M − 1): |
| | carboxamide | 506.35 |
| 184 | N-((3,5- | ¹H-NMR (400 MHz, DMSO-d₆) δ 8.56 (s, 3H), 8.20 (d, |
| | bis(trifluoromethyl)phenyl)sulfonyl)- | J = 2.1 Hz, 1H), 8.08 (dd, J = 8.4, 2.3 Hz, 1H), 7.84 (d, J = |
| | 1-(4-chloro-2- | 8.6 Hz, 1H), 2.30-2.23 (m, 3H); LCMS(M − 1): 578.95 |
| | (trifluoromethyl)phenyl)-5-methyl- | |
| | 1H-1,2,3-triazole-4-carboxamide | |
| 185 | 1-(4-chloro-2- | ¹H-NMR (400 MHz, DMSO-d₆) δ 8.21 (d, J = 2.4 Hz, |
| | (trifluoromethyl)phenyl)-5-methyl- | 1H), 8.17-8.13 (m, 2H), 8.09 (dd, J = 8.4, 2.3 Hz, 1H), |
| | N-((4- | 7.87-7.83 (m, 1H), 7.65 (d, J = 7.9 Hz, 2H), 2.24 (d, J = |
| | (trifluoromethoxy)phenyl)sulfonyl)- | 14.4 Hz, 3H); LCMS(M+): 528.95 |
| | 1H-1,2,3-triazole-4-carboxamide | |
| 186 | 1-(2-chloro-4- | ¹H-NMR (400 MHz, DMSO-d₆) δ 13.12 (s, 0H), 8.27 (s, |
| | (trifluoromethyl)phenyl)-5-methyl- | 1H), 8.20 (d, J = 7.6 Hz, 2H), 7.39 (d, J = 7.8 Hz, 2H), |
| | N-((4- | 6.95 (d, J = 7.6 Hz, 2H), 2.34 (s, 3H); LCMS(M+): 528.85 |
| | (trifluoromethoxy)phenyl)sulfonyl)- | |
| | 1H-1,2,3-triazole-4-carboxamide | |
| 187 | 1-(2-chloro-4- | ¹H-NMR (400 MHz, DMSO-d₆) δ 8.36 (d, J = 1.7 Hz, |
| | (trifluoromethyl)phenyl)-N-((4- | 1H), 8.18 (d, J = 8.3 Hz, 1H), 8.10 (dd, J = 8.4, 6.5 Hz, |
| | (trifluoromethoxy)phenyl)sulfonyl)- | 3H), 7.61 (d, J = 8.1 Hz, 2H); LCMS(M+): 582.55 |
| | 5-(trifluoromethyl)-1H-1,2,3- | |
| | triazole-4-carboxamide | |
| 188 | 1-(2-chloro-4- | ¹H-NMR (400 MHz, DMSO-d₆) δ 8.12-8.08 (m, 3H), |
| | (trifluoromethoxy)phenyl)-N-((4- | 8.04 (d, J = 2.0 Hz, 1H), 7.74-7.72 (m, 1H), 7.62 (d, J = |
| | (trifluoromethoxy)phenyl)sulfonyl)- | 8.1 Hz, 2H); LCMS(M − 2): 596.55 |
| | 5-(trifluoromethyl)-1H-1,2,3- | |
| | triazole-4-carboxamide | |
| 189 | 1-(2,6-difluorophenyl)-N-((4- | ¹H-NMR (400 MHz, DMSO-d₆) δ 8.11-8.08 (m, 2H), |
| | (trifluoromethoxy)phenyl)sulfonyl)- | 7.95-7.88 (m, 1H), 7.62-7.57 (m, 4H); LCMS(M − 1): |
| | 5-(trifluoromethyl)-1H-1,2,3- | 514.90 |
| | triazole-4-carboxamide | |
| 190 | 1-(3,5-dichloro-4-fluorophenyl)-5- | ¹H-NMR (400 MHz, DMSO-d₆) δ 12.78 (s, 0H), 8.14 (dt, |
| | methyl-N-((4- | J = 9.5, 2.6 Hz, 2H), 8.01 (d, J = 6.1 Hz, 2H), 7.65 (d, J = |
| | (trifluoromethoxy)phenyl)sulfonyl)- | 8.1 Hz, 2H), 2.42 (s, 3H); LCMS(M+): 512.80 |
| | 1H-1,2,3-triazole-4-carboxamide | |
| 191 | 1-(4-chloro-2- | ¹H-NMR (400 MHz, DMSO-d₆) δ 8.24 (d, J = 2.0 Hz, |
| | (trifluoromethyl)phenyl)-N-((4- | 1H), 8.13-8.04 (m, 4H), 7.58 (d, J = 8.6 Hz, 2H); |
| | (trifluoromethoxy)phenyl)sulfonyl)- | LCMS(M − 1): 580.95 |
| | 5-(trifluoromethyl)-1H-1,2,3- | |
| | triazole-4-carboxamide | |
| 192 | N-((4-chloro-2,5- | ¹H-NMR (400 MHz, DMSO-d₆) δ 8.26 (d, J = 2.2 Hz, |
| | dimethylphenyl)sulfonyl)-1-(4- | 1H), 8.14-8.06 (m, 2H), 7.97 (s, 1H), 7.52 (s, 1H), 2.59 |
| | chloro-2-(trifluoromethyl)phenyl)-5- | (s, 3H), 2.38 (s, 3H); LCMS(M − 1): 560.60 |
| | (trifluoromethyl)-1H-1,2,3-triazole- | |
| | 4-carboxamide | |
| 193 | 1-(4-chloro-2- | ¹H-NMR (400 MHz, DMSO-d₆) δ 8.22 (d, J = 2.0 Hz, |
| | (trifluoromethyl)phenyl)-N-((2,4,5- | 1H), 8.13 (s, 1H), 8.11-8.04 (m, 2H), 7.93 (s, 1H); |
| | trichlorophenyl)sulfonyl)-5- | LCMS(M − 1): 600.80 |
| | (trifluoromethyl)-1H-1,2,3-triazole- | |
| | 4-carboxamide | |
| 194 | 1-(4-chloro-2- | ¹H-NMR (400 MHz, DMSO-d₆) δ 8.23 (d, J = 2.2 Hz, |
| | (trifluoromethyl)phenyl)-N-((5- | 1H), 8.11-8.03 (m, 2H), 7.82 (q, J = 2.9 Hz, 1H), 7.66- |
| | chloro-2-fluorophenyl)sulfonyl)-5- | 7.62 (m, 1H), 7.36 (t, J = 9.2 Hz, 1H); LCMS(M − 2): |
| | (trifluoromethyl)-1H-1,2,3-triazole- | 548.60 |
| | 4-carboxamide | |
| 195 | N-((3,5- | ¹H-NMR (400 MHz, DMSO-d₆) δ 8.40 (s, 2H), 8.35 (s, |
| | bis(trifluoromethyl)phenyl)sulfonyl)- | 1H), 8.22 (d, J = 2.0 Hz, 1H), 8.09 (dd, J = 8.6, 2.2 Hz, |
| | 1-(4-chloro-2- | 1H), 8.03 (d, J = 8.6 Hz, 1H); LCMS(M − 1): 632.80 |
| | (trifluoromethyl)phenyl)-5- | |
| | (trifluoromethyl)-1H-1,2,3-triazole- | |
| | 4-carboxamide | |
| 196 | 1-(2-chloro-4- | ¹H-NMR (400 MHz, DMSO-d₆) δ 8.32 (d, J = 1.5 Hz, |
| | (trifluoromethyl)phenyl)-N-((2- | 1H), 8.18 (dd, J = 7.8, 1.2 Hz, 1H), 8.06-8.04 (m, 1H), |
| | chlorophenyl)sulfonyl)-5-methyl- | 8.00 (d, J = 8.3 Hz, 1H), 7.72-7.66 (m, 2H), 7.63-7.59 (m, |
| | 1H-1,2,3-triazole-4-carboxamide | 1H), 2.28 (s, 3H); LCMS(M+): 478.55 |
| 197 | 1-(2-chloro-4- | ¹H-NMR (400 MHz, DMSO-d₆) δ 8.33 (d, J = 1.7 Hz, |
| | (trifluoromethyl)phenyl)-N-((2- | 1H), 8.06-7.98 (m, 3H), 7.80-7.75 (m, 1H), 7.48-7.44 (m, |
| | fluorophenyl)sulfonyl)-5-methyl-1H- | 2H), 2.28 (s, 3H); LCMS(M+): 462.50 |
| | 1,2,3-triazole-4-carboxamide | |
| 198 | 1-(2-chloro-4- | ¹H-NMR (400 MHz, DMSO-d₆) δ 8.33 (d, J = 1.7 Hz, |
| | (trifluoromethyl)phenyl)-N-((3,5- | 1H), 8.06-8.04 (m, 1H), 7.98 (d, J = 8.3 Hz, 1H), 7.73- |
| | difluorophenyl)sulfonyl)-5-methyl- | 7.67 (m, 3H), 2.32 (s, 3H); LCMS(M+): 480.55 |
| | 1H-1,2,3-triazole-4-carboxamide | |
| 199 | 1-(2-chloro-4- | ¹H-NMR (400 MHz, DMSO-d₆) δ 8.83 (d, J = 1.7 Hz, |
| | (trifluoromethyl)phenyl)-N-((5- | 1H), 8.33 (d, J = 1.5 Hz, 1H), 8.29 (dd, J = 8.4, 2.3 Hz, |
| | chloropyridin-2-yl)sulfonyl)-5- | 1H), 8.17 (d, J = 7.8 Hz, 1H), 8.05 (dd, J = 8.6, 1.7 Hz, |
| | methyl-1H-1,2,3-triazole-4- | 1H), 8.00 (d, J = 8.3 Hz, 1H), 2.27 (s, 3H); LCMS(M+): |
| | carboxamide | 479.50 |
| 200 | 1-(2-chloro-4- | ¹H-NMR (400 MHz, DMSO-d₆) δ 12.54-14.06 (0H), 8.15 |
| | (trifluoromethoxy)phenyl)-5-methyl- | (dd, J = 7.8, 1.7 Hz, 1H), 8.01 (d, J = 2.2 Hz, 1H), 7.91 |
| | N-((2- | (q, J = 4.4 Hz, 1H), 7.84 (td, J = 7.8, 1.5 Hz, 1H), 7.70 |
| | (trifluoromethoxy)phenyl)sulfonyl)- | (dq, J = 8.7, 1.2 Hz, 1H), 7.65-7.59 (m, 2H), 2.26 (s, 3H); |
| | 1H-1,2,3-triazole-4-carboxamide | LCMS(M+): 544.55 |
| 201 | N-((2-chloro-5- | ¹H-NMR (400 MHz, DMSO-d₆) δ 8.39 (d, J = 2.0 Hz, |
| | (trifluoromethyl)phenyl)sulfonyl)-1- | 1H), 8.16 (dd, J = 8.8, 2.0 Hz, 1H), 8.06-8.01 (m, 2H), |
| | (2,4-dichlorophenyl)-N,5-dimethyl- | 7.78-7.73 (m, 2H), 3.84 (s, 3H), 2.19 (s, 3H); LCMS |
| | 1H-1,2,3-triazole-4-carboxamide | (M + 1): 526.45 |
| 202 | N-((2-chloro-5- | ¹H-NMR (400 MHz, DMSO-d₆) δ 8.38 (d, J = 2.0 Hz, |
| | (trifluoromethyl)phenyl)sulfonyl)-N- | 1H), 8.15 (dd, J = 8.6, 2.0 Hz, 1H), 8.06 (d, J = 2.0 Hz, |
| | (cyclopropylmethyl)-1-(2,4- | 1H), 7.99 (d, J = 8.3 Hz, 1H), 7.81-7.73 (m, 2H), 4.51 (d, |
| | dichlorophenyl)-5-methyl-1H-1,2,3- | J = 7.1 Hz, 2H), 2.18 (d, J = 7.6 Hz, 3H), 1.27 (t, J = 7.7 |
| | triazole-4-carboxamide | Hz, 1H), 0.59-0.54 (m, 2H), 0.38 (q, J = 5.0 Hz, 2H); |
| | | LCMS (M + 1): 568.40 |
| 203 | N-((2-chloro-5- | ¹H-NMR (400 MHz, DMSO-d₆) δ 8.05 (d, J = 2.2 Hz, |
| | methoxyphenyl)sulfonyl)-5- | 1H), 7.85 (d, J = 8.6 Hz, 1H), 7.74 (dd, J = 8.4, 2.3 Hz, |
| | cyclopropyl-1-(2,4-dichlorophenyl)- | 1H), 7.62-7.59 (m, 2H), 7.30 (dd, J = 8.8, 3.2 Hz, 1H), |
| | 1H-1,2,3-triazole-4-carboxamide | 3.86 (s, 3H), 1.87-1.80 (m, 1H), 0.83-0.78 (m, 2H), 0.68- |
| | | 0.64 (m, 2H); LCMS (M + 1): 502.85 |
| 204 | N-((5-chloro-2- | ¹H-NMR (400 MHz, DMSO-d₆) δ 8.05 (d, J = 2.2 Hz, |
| | fluorophenyl)sulfonyl)-5- | 1H), 7.95 (q, J = 2.9 Hz, 1H), 7.89-7.83 (m, 2H), 7.74 |
| | cyclopropyl-1-(2,4-dichlorophenyl)- | (dd, J = 8.6, 2.2 Hz, 1H), 7.57 (t, J = 9.4 Hz, 1H), 1.88- |
| | 1H-1,2,3-triazole-4-carboxamide | 1.81 (m, 1H), 0.83-0.78 (m, 2H), 0.67-0.63 (m, 2H); |
| | | LCMS (M + 1): 490.80 |
| 205 | N-((5-chloro-2,4- | ¹H-NMR (400 MHz, DMSO-d₆) δ 8.15 (t, J = 7.6 Hz, 1H), |
| | difluorophenyl)sulfonyl)-5- | 8.05 (d, J = 2.4 Hz, 1H), 7.91-7.83 (m, 2H), 7.74 (dd, J = |
| | cyclopropyl-1-(2,4-dichlorophenyl)- | 8.6, 2.2 Hz, 1H), 1.89-1.82 (m, 1H), 0.84-0.75 (m, 2H), |
| | 1H-1,2,3-triazole-4-carboxamide | 0.69-0.65 (m, 2H); LCMS (M + 1): 508.80 |
| 206 | N-((2-chloro-5- | ¹H-NMR (400 MHz, DMSO-d₆) δ 8.35 (d, J = 1.8 Hz, |
| | (trifluoromethyl)phenyl)sulfonyl)-5- | 1H), 8.12 (dd, J = 8.4, 2.0 Hz, 1H), 8.05 (d, J = 2.4 Hz, |
| | cyclopropyl-1-(2,4-dichlorophenyl)- | 1H), 7.96 (d, J = 8.6 Hz, 1H), 7.85 (d, J = 8.6 Hz, 1H), |
| | 1H-1,2,3-triazole-4-carboxamide | 7.73 (dd, J = 8.4, 2.3 Hz, 1H), 1.87-1.83 (m, 1H), 0.81- |
| | | 0.76 (m, 2H), 0.65-0.61 (m, 2H); LCMS (M + 1): 540.85 |
| 207 | N-((2-chlorophenyl)sulfonyl)-1-(2,4- | ¹H-NMR (400 MHz, DMSO-d₆) δ 8.15 (d, J = 7.3 Hz, |
| | dichlorophenyl)-5-(trifluoromethyl)- | 1H), 8.09 (d, J = 2.2 Hz, 1H), 7.97 (d, J = 8.6 Hz, 1H), |
| | 1H-1,2,3-triazole-4-carboxamide | 7.77 (dd, J = 8.6, 2.2 Hz, 1H), 7.65 (t, J = 6.0 Hz, 2H), |
| | | 7.60-7.56 (m, 1H); LCMS(M + 1): 500.85 |
| 208 | 1-(2,4-dichlorophenyl)-N-((2- | ¹H-NMR (400 MHz, DMSO-d₆) δ 8.09 (d, J = 2.2 Hz, |
| | fluorophenyl)sulfonyl)-5- | 1H), 7.99-7.95 (m, 2H), 7.78-7.73 (m, 2H), 7.45-7.41 (m, |
| | (trifluoromethyl)-1H-1,2,3-triazole- | 2H); LCMS(M − 2): 480.85 |
| | 4-carboxamide | |
| 209 | 1-(2,4-dichlorophenyl)-5- | ¹H-NMR (400 MHz, DMSO-d₆) δ 8.35 (d, J = 8.1 Hz, |
| | (trifluoromethyl)-N-((2- | 1H), 8.08 (d, J = 2.2 Hz, 1H), 7.97-7.95 (m, 2H), 7.92- |
| | (trifluoromethyl)phenyl)sulfonyl)- | 7.89 (m, 1H), 7.85 (t, J = 7.5 Hz, 1H), 7.76 (dd, J = 8.7, |
| | 1H-1,2,3-triazole-4-carboxamide | 2.3 Hz, 1H); LCMS(M+): 532.80 |
| 210 | N-((2-chloro-5- | ¹H-NMR (400 MHz, DMSO-d₆) δ 8.30 (d, J = 2.2 Hz, |
| | (trifluoromethyl)phenyl)sulfonyl)-1- | 1H), 8.06 (d, J = 2.2 Hz, 1H), 7.95 (d, J = 8.6 Hz, 1H), |
| | (2,4-dichlorophenyl)-5- | 7.92 (dd, J = 8.8, 2.2 Hz, 1H), 7.79 (d, J = 8.1 Hz, 1H), |
| | (trifluoromethyl)-1H-1,2,3-triazole- | 7.74 (dd, J = 8.6, 2.2 Hz, 1H); LCMS(M+): 566.90 |
| | 4-carboxamide | |
| 211 | 1-(2,4-dichlorophenyl)-N-((2,4- | ¹H-NMR (400 MHz, DMSO-d₆) δ 8.12 (d, J = 8.6 Hz, |
| | dichlorophenyl)sulfonyl)-5- | 1H), 8.08 (d, J = 2.2 Hz, 1H), 7.97 (d, J = 8.6 Hz, 1H), |
| | (trifluoromethyl)-1H-1,2,3-triazole- | 7.83 (d, J = 2.0 Hz, 1H), 7.76 (dd, J = 8.6, 2.2 Hz, 1H), |
| | 4-carboxamide | 7.66 (dd, J = 8.6, 2.0 Hz, 1H); LCMS(M+): 534.75 |
| 212 | 1-(2,4-dichlorophenyl)-N-((2,5- | ¹H-NMR (400 MHz, DMSO-d₆) δ 8.07 (d, J = 2.1 Hz, |
| | difluorophenyl)sulfonyl)-5- | 1H), 7.95 (d, J = 8.6 Hz, 1H), 7.75 (dd, J = 8.6, 2.4 Hz, |
| | (trifluoromethyl)-1H-1,2,3-triazole- | 1H), 7.66-7.62 (m, 1H), 7.51-7.46 (m, 1H), 7.40 (td, J = |
| | 4-carboxamide | 9.2, 4.1 Hz, 1H); LCMS(M+): 500.80 |
| 213 | N-((5-chloro-2- | ¹H-NMR (400 MHz, DMSO-d₆) δ 8.07 (d, J = 2.1 Hz, |
| | fluorophenyl)sulfonyl)-1-(2,4- | 1H), 7.95 (d, J = 8.6 Hz, 1H), 7.84 (q, J = 3.0 Hz, 1H), |
| | dichlorophenyl)-5-(trifluoromethyl)- | 7.75 (dd, J = 8.6, 2.4 Hz, 1H), 7.68 (qd, J = 4.3, 2.8 Hz, |
| | 1H-1,2,3-triazole-4-carboxamide | 1H), 7.39 (t, J = 9.2 Hz, 1H); LCMS(M − 2): 514.95 |
| 214 | N-((3-chloro-4- | ¹H-NMR (400 MHz, DMSO-d₆) δ 8.11 (dd, J = 6.9, 2.3 |
| | fluorophenyl)sulfonyl)-1-(2,4- | Hz, 1H), 8.09 (d, J = 2.1 Hz, 1H), 8.00-7.97 (m, 1H), 7.95 |
| | dichlorophenyl)-5-(trifluoromethyl)- | (d, J = 8.6 Hz, 1H), 7.77 (dd, J = 8.6, 2.1 Hz, 1H), 7.68 (t, |
| | 1H-1,2,3-triazole-4-carboxamide | J = 8.9 Hz, 1H); LCMS(M − 2): 514.75 |
| 215 | N-((5-chloro-2,4- | ¹H-NMR (400 MHz, DMSO-d₆) δ 8.03 (d, J = 2.1 Hz, |
| | difluorophenyl)sulfonyl)-1-(2,4- | 1H), 7.95 (t, J = 7.8 Hz, 1H), 7.92 (d, J = 8.6 Hz, 1H), |
| | dichlorophenyl)-5-(trifluoromethyl)- | 7.72 (dd, J = 8.6, 2.4 Hz, 1H), 7.63 (t, J = 9.5 Hz, 1H); |
| | 1H-1,2,3-triazole-4-carboxamide | LCMS(M − 2): 534.90 |
| 216 | 1-(2,4-dichlorophenyl)-N-((4- | ¹H-NMR (400 MHz, DMSO-d₆) δ 8.11-8.08 (m, 3H), |
| | (trifluoromethoxy)phenyl)sulfonyl)- | 7.95 (d, J = 8.6 Hz, 1H), 7.77 (dd, J = 8.6, 2.1 Hz, 1H), |
| | 5-(trifluoromethyl)-1H-1,2,3- | 7.61 (d, J = 7.9 Hz, 2H); LCMS(M − 2): 546.90 |
| | triazole-4-carboxamide | |
| 217 | 1-(2-chloro-4- | ¹H-NMR (400 MHz, DMSO-d₆) δ 12.95 (s, 1H), 8.01 (d, |
| | (trifluoromethoxy)phenyl)-N-((2,5- | J = 2.4 Hz, 1H), 7.90 (d, J = 8.8 Hz, 1H), 7.86 (s, 1H), |
| | dimethylphenyl)sulfonyl)-5-methyl- | 7.70 (d, J = 8.8 Hz, 1H), 7.39 (d, J = 7.3 Hz, 1H), 7.29 (d, |
| | 1H-1,2,3-triazole-4-carboxamide | J = 7.8 Hz, 1H), 2.60 (s, 3H), 2.36 (s, 3H), 2.27 (d, J = 6.6 |
| | | Hz, 3H); LCMS(M + 1): 489.20 |
| 218 | 1-(2,4-dichlorophenyl)-N-((2,4- | ¹H-NMR (400 MHz, DMSO-d₆) δ 8.09 (d, J = 2.2 Hz, |
| | difluorophenyl)sulfonyl)-5- | 1H), 8.04 (td, J = 8.6, 6.4 Hz, 1H), 7.96 (d, J = 8.6 Hz, |
| | (trifluoromethyl)-1H-1,2,3-triazole- | 1H), 7.77 (dd, J = 8.6, 2.4 Hz, 1H), 7.54-7.49 (m, 1H), |
| | 4-carboxamide | 7.31 (td, J = 8.4, 2.3 Hz, 1H); LCMS(M+): 500.90 |
| 219 | 1-(2,4-dichlorophenyl)-N-((3,5- | ¹H-NMR (400 MHz, DMSO-d₆) δ 8.07 (d, J = 2.2 Hz, |
| | difluorophenyl)sulfonyl)-5- | 1H), 7.94 (d, J = 8.6 Hz, 1H), 7.75 (dd, J = 8.6, 2.2 Hz, |
| | (trifluoromethyl)-1H-1,2,3-triazole- | 1H), 7.55 (d, J = 6.6 Hz, 3H); LCMS(M − 2): 498.95 |
| | 4-carboxamide | |
| 220 | N-((3,5- | ¹H-NMR (400 MHz, DMSO-d₆) δ 8.39 (s, 2H), 8.33 (s, |
| | bis(trifluoromethyl)phenyl)sulfonyl)- | 1H), 8.05 (d, J = 2.2 Hz, 1H), 7.92 (d, J = 8.6 Hz, 1H), |
| | 1-(2,4-dichlorophenyl)-5- | 7.73 (dd, J = 8.6, 2.2 Hz, 1H); LCMS(M − 1): 598.85 |
| | (trifluoromethyl)-1H-1,2,3-triazole- | |
| | 4-carboxamide | |
| 221 | N-((4-chloro-2,5- | ¹H-NMR (400 MHz, DMSO-d₆) δ 8.09 (d, J = 2.2 Hz, |
| | dimethylphenyl)sulfonyl)-1-(2,4- | 1H), 7.97 (d, J = 8.6 Hz, 2H), 7.80-7.76 (m, 1H), 7.53 (s, |
| | dichlorophenyl)-5-(trifluoromethyl)- | 1H), 2.59 (s, 3H), 2.39 (s, 3H); LCMS(M+): 526.75 |
| | 1H-1,2,3-triazole-4-carboxamide | |
| 222 | 1-(2,4-dichlorophenyl)-N-((2,5- | ¹H-NMR (400 MHz, DMSO-d₆) δ 8.07 (d, J = 2.2 Hz, |
| | dichlorophenyl)sulfonyl)-5- | 1H), 8.04 (d, J = 2.6 Hz, 1H), 7.96 (d, J = 8.6 Hz, 1H), |
| | (trifluoromethyl)-1H-1,2,3-triazole- | 7.75 (dd, J = 8.6, 2.2 Hz, 1H), 7.67 (dd, J = 8.6, 2.7 Hz, |
| | 4-carboxamide | 1H), 7.61 (d, J = 8.6 Hz, 1H); LCMS(M+): 534.85 |
| 223 | N-((2-chloro-5- | ¹H-NMR (400 MHz, DMSO-d₆) δ 13.29 (s, 1H), 8.00 (d, |
| | methoxyphenyl)sulfonyl)-1-(2,5- | J = 2.2 Hz, 1H), 7.87-7.80 (m, 2H), 7.62 (d, J = 3.2 Hz, |
| | dichlorophenyl)-5-methyl-1H-1,2,3- | 1H), 7.59 (d, J = 8.8 Hz, 1H), 7.29 (dd, J = 8.8, 3.2 Hz, |
| | triazole-4-carboxamide | 1H), 3.84 (s, 3H), 2.30 (s, 3H); LCMS (M + 1): 476.45 |
| 224 | 1-(2,5-dichlorophenyl)-N-((2,5- | ¹H-NMR (400 MHz, DMSO-d₆) δ 8.10 (d, J = 2.4 Hz, |
| | dichlorophenyl)sulfonyl)-5-methyl- | 1H), 7.99 (d, J = 2.1 Hz, 1H), 7.87-7.70 (m, 4H), 2.30 (s, |
| | 1H-1,2,3-triazole-4-carboxamide | 3H); LCMS (M+): 480.80 |
| 225 | N-((2-chloro-5- | ¹H-NMR (400 MHz, CHLOROFORM-D) δ 9.98 (s, 1H), |
| | (trifluoromethyl)phenyl)sulfonyl)-1- | 8.63 (d, J = 2.0 Hz, 1H), 7.83 (dd, J = 8.4, 1.6 Hz, 1H), |
| | (2,5-dichlorophenyl)-5-methyl-1H- | 7.69 (d, J = 8.3 Hz, 1H), 7.57-7.55 (m, 2H), 7.44 (q, J = |
| | 1,2,3-triazole-4-carboxamide | 1.0 Hz, 1H), 2.41 (s, 3H); LCMS (M + 1); 514.80 |
| 226 | 1-(2,5-dichlorophenyl)-N-((2,4- | ¹H-NMR (400 MHz, DMSO-d₆) δ 8.04 (dd, J = 14.9, 8.6 |
| | difluorophenyl)sulfonyl)-5-methyl- | Hz, 1H), 7.97 (d, J = 2.2 Hz, 1H), 7.85 (d, J = 8.6 Hz, |
| | 1H-1,2,3-triazole-4-carboxamide | 1H), 7.81 (dd, J = 8.6, 2.4 Hz, 1H), 7.54-7.49 (m, 1H), |
| | | 7.34-7.30 (m, 1H), 2.28 (s, 3H); LCMS (M + 1): 446.90 |
| 227 | 1-(2-chloro-4- | ¹H-NMR (400 MHz, DMSO-d₆) δ 8.35 (d, J = 2.2 Hz, |
| | (trifluoromethoxy)phenyl)-N-((2- | 1H), 8.12-8.10 (m, 1H), 8.01-7.94 (m, 3H), 7.70-7.68 (m, |
| | chloro-5- | 1H), 1.87-1.83 (m, 1H), 0.80-0.77 (m, 2H), 0.65 (dd, J = |
| | (trifluoromethyl)phenyl)sulfonyl)-5- | 5.4, 2.2 Hz, 2H); LCMS(M+): 588.45 |
| | cyclopropyl-1H-1,2,3-triazole-4- | |
| | carboxamide | |
| 228 | 1-(2-chloro-4- | ¹H-NMR (400 MHz, DMSO-d₆) δ 8.36 (d, J = 7.8 Hz, |
| | (trifluoromethoxy)phenyl)-5- | 1H), 8.04-7.91 (m, 5H), 7.69 (dq, J = 8.8, 1.2 Hz, 1H), |
| | cyclopropyl-N-((2- | 1.85-1.78 (m, 1H), 0.83-0.78 (m, 2H), 0.71-0.67 (m, 2H); |
| | (trifluoromethyl)phenyl)sulfonyl)- | LCMS(M+): 554.75 |
| | 1H-1,2,3-triazole-4-carboxamide | |
| 229 | 1-(2-chloro-4- | ¹H-NMR (400 MHz, DMSO-d₆) δ 8.10 (d, J = 2.4 Hz, |
| | (trifluoromethoxy)phenyl)-5- | 1H), 8.01-7.97 (m, 2H), 7.81 (dd, J = 8.6, 2.4 Hz, 1H), |
| | cyclopropyl-N-((2,5- | 7.74-7.68 (m, 2H), 1.89-1.82 (m, 1H), 0.83-0.78 (m, 2H); |
| | dichlorophenyl)sulfonyl)-1H-1,2,3- | 0.69-0.65 (m, 2H), LCMS(M + 1): 556.35 |
| | triazole-4-carboxamide | |
| 230 | 1-(2-chloro-4- | ¹H-NMR (400 MHz, DMSO-d₆) δ 8.01-7.98 (m, 2H), |
| | (trifluoromethoxy)phenyl)-N-((2- | 7.69 (dq, J = 8.9, 1.2 Hz, 1H), 7.62-7.59 (m, 2H), 7.30 |
| | chloro-5-methoxyphenyl)sulfonyl)- | (dd, J = 8.9, 3.1 Hz, 1H), 3.86 (s, 3H), 1.85-1.80 (m, 1H), |
| | 5-cyclopropyl-1H-1,2,3-triazole-4- | 0.83-0.78 (m, 2H), 0.69-0.65 (m, 2H); LCMS(M+): |
| | carboxamide | 550.50 |
| 231 | N-((2-chloro-5- | ¹H-NMR (400 MHz, DMSO-d₆) δ 8.36 (d, J = 2.0 Hz, |
| | (trifluoromethyl)phenyl)sulfonyl)-1- | 1H), 8.10 (dd, J = 8.4, 1.8 Hz, 1H), 8.06 (d, J = 2.2 Hz, |
| | (2,4-dichlorophenyl)-5-ethyl-1H- | 1H), 7.94 (d, J = 8.3 Hz, 1H), 7.84 (d, J = 8.6 Hz, 1H), |
| | 1,2,3-triazole-4-carboxamide | 7.74 (dd, J = 8.6, 2.2 Hz, 1H), 2.66 (d, J = 7.8 Hz, 2H), |
| | | 0.87 (t, J = 7.6 Hz, 3H); LCMS (M + 1): 528.90 |
| 232 | 1-(2-chloro-4-cyanophenyl)-N-((2,4- | ¹H-NMR (400 MHz, DMSO-d₆) δ 8.49 (d, J = 1.5 Hz, |
| | dichlorophenyl)sulfonyl)-5-methyl- | 1H), 8.17-8.14 (m, 2H), 7.97 (d, J = 8.1 Hz, 1H), 7.90 (d, |
| | 1H-1,2,3-triazole-4-carboxamide | J = 2.2 Hz, 1H), 7.71 (dd, J = 8.6, 2.0 Hz, 1H), 2.27 (s, |
| | | 3H); LCMS (M + 1): 471.70 |
| 233 | 1-(2-chloro-4-cyanophenyl)-N-((2- | ¹H-NMR (400 MHz, DMSO-d₆) δ 8.49 (d, J = 1.5 Hz, |
| | fluorophenyl)sulfonyl)-5-methyl-1H- | 1H), 8.15 (dd, J = 8.1, 1.7 Hz, 1H), 8.03-7.96 (m, 2H), |
| | 1,2,3-triazole-4-carboxamide | 7.80-7.75 (m, 1H), 7.49-7.44 (m, 2H), 2.27 (s, 3H); |
| | | LCMS (M+): 419.90 |
| 234 | 1-(2,5-dichlorophenyl)-5-methyl-N- | ¹H-NMR (400 MHz, DMSO-d₆) δ 7.99 (d, J = 2.2 Hz, |
| | ((2,4,6-trifluorophenyl)sulfonyl)-1H- | 1H), 7.86 (d, J = 8.8 Hz, 1H), 7.81 (dd, J = 8.7, 2.3 Hz, |
| | 1,2,3-triazole-4-carboxamide | 1H), 7.46 (t, J = 9.4 Hz, 2H), 2.30 (s, 3H); LCMS (M+): |
| | | 464.75 |
| 235 | N-((2-chloro-5- | ¹H-NMR (400 MHz, DMSO-d₆) δ 8.07 (d, J = 2.2 Hz, |
| | methoxyphenyl)sulfonyl)-1-(2,4- | 1H), 7.86 (d, J = 8.6 Hz, 1H), 7.74 (dd, J = 8.6, 2.2 Hz, |
| | dichlorophenyl)-5-ethyl-1H-1,2,3- | 1H), 7.62-7.57 (m, 2H), 7.29 (dd, J = 8.8, 3.2 Hz, 1H), |
| | triazole-4-carboxamide | 3.85 (s, 3H), 2.65 (q, J = 7.4 Hz, 2H), 0.88 (t, J = 7.5 Hz, |
| | | 3H); LCMS (M + 1): 488.90 |
| 236 | 1-(2,4-dichlorophenyl)-N-((2,5- | ¹H-NMR (400 MHz, DMSO-d₆) δ 8.11 (d, J = 2.4 Hz, |
| | dichlorophenyl)sulfonyl)-5-ethyl- | 1H), 8.06 (d, J = 2.2 Hz, 1H), 7.85 (d, J = 8.6 Hz, 1H), |
| | 1H-1,2,3-triazole-4-carboxamide | 7.80 (dd, J = 8.6, 2.7 Hz, 1H), 7.76-7.71 (m, 2H), 2.66 (q, |
| | | J = 7.3 Hz, 2H), 0.91-0.86 (m, 3H); LCMS (M + 1): 494.90 |
| 237 | 1-(2,4-dichlorophenyl)-N-((2,5- | ¹H-NMR (400 MHz, DMSO-d₆) δ 8.06 (d, J = 2.2 Hz, |
| | difluorophenyl)sulfonyl)-5-ethyl-1H- | 1H), 7.84 (d, J = 8.6 Hz, 1H), 7.80-7.73 (m, 2H), 7.69- |
| | 1,2,3-triazole-4-carboxamide | 7.65 (m, 1H), 7.56 (td, J = 9.3, 4.2 Hz, 1H), 2.69-2.63 (m, |
| | | 2H), 0.89 (t, J = 7.6 Hz, 3H); LCMS (M + 1): 460.90 |
| 238 | 1-(2,4-dichlorophenyl)-5-ethyl-N- | ¹H-NMR (400 MHz, DMSO-d₆) δ 8.37 (d, J = 7.8 Hz, |
| | ((2- | 1H), 8.07 (d, J = 2.2 Hz, 1H), 8.03-8.01 (m, 1H), 7.98- |
| | (trifluoromethyl)phenyl)sulfonyl)- | 7.89 (m, 2H), 7.84 (d, J = 8.6 Hz, 1H), 7.74 (dd, J = 8.6, |
| | 1H-1,2,3-triazole-4-carboxamide | 2.2 Hz, 1H), 2.65 (q, J = 7.3 Hz, 2H), 0.93-0.86 (m, 3H); |
| | | LCMS (M + 1): 492.95 |
| 239 | 1-(2,4-dichlorophenyl)-5-ethyl-N- | ¹H-NMR (400 MHz, DMSO-d₆) δ 8.15 (dd, J = 7.8, 1.7 |
| | ((2- | Hz, 1H), 8.06 (d, J = 2.2 Hz, 1H), 7.86-7.82 (m, 2H), 7.74 |
| | (trifluoromethoxy)phenyl)sulfonyl)- | (dd, J = 8.6, 2.2 Hz, 1H), 7.66-7.59 (m, 2H), 2.66-2.61 |
| | 1H-1,2,3-triazole-4-carboxamide | (m, 2H), 0.87 (t, J = 7.6 Hz, 3H); LCMS (M + 1): 508.95 |
| 240 | 1-(2,4-dichlorophenyl)-N-((2-fluoro- | ¹H-NMR (400 MHz, DMSO-d₆) δ 8.18 (dd, J = 6.1, 2.1 |
| | 5-(trifluoromethyl)phenyl)sulfonyl)- | Hz, 1H), 8.05-8.02 (m, 2H), 7.75-7.70 (m, 2H), 7.59 (t, |
| | 5-methyl-1H-1,2,3-triazole-4- | J = 9.0 Hz, 1H), 2.29 (s, 3H); LCMS (M + 1): 498.70 |
| | carboxamide | |
| 241 | 1-(2,4-dichlorophenyl)-5-methyl-N- | ¹H-NMR (400 MHz, DMSO-d₆) δ 8.36 (d, J = 7.6 Hz, |
| | ((2- | 1H), 8.05 (d, J = 2.0 Hz, 1H), 8.01-7.87 (m, 3H), 7.78- |
| | (trifluoromethyl)phenyl)sulfonyl)- | 7.72 (m, 2H), 2.26 (s, 3H); LCMS (M + 1): 478.90 |
| | 1H-1,2,3-triazole-4-carboxamide | |
| 242 | 1-(2-chloro-4-fluorophenyl)-N-((2- | ¹H-NMR (400 MHz, DMSO-d₆) δ 7.90-7.80 (m, 2H), |
| | chloro-5-methoxyphenyl)sulfonyl)- | 7.62 (d, J = 2.9 Hz, 1H), 7.59-7.52 (m, 2H), 7.28 (dd, J = |
| | 5-methyl-1H-1,2,3-triazole-4- | 8.8, 3.2 Hz, 1H), 3.85 (s, 3H), 2.25 (s, 3H); LCMS(M+): |
| | carboxamide | 458.9 |
| 243 | 1-(2-chloro-4-fluorophenyl)-N-((2,5- | ¹H-NMR (400 MHz, DMSO-d₆) δ 8.09 (d, J = 2.4 Hz, |
| | dichlorophenyl)sulfonyl)-5-methyl- | 1H), 7.88 (dd, J = 8.6, 2.7 Hz, 1H), 7.84-7.76 (m, 2H), |
| | 1H-1,2,3-triazole-4-carboxamide | 7.70 (d, J = 8.6 Hz, 1H), 7.54 (td, J = 8.6, 2.9 Hz, 1H), |
| | | 2.26 (s, 3H); LCMS(M + 1): 464.9 |
| 244 | 1-(2-chloro-4-fluorophenyl)-5- | ¹H-NMR (400 MHz, DMSO-d₆) δ 8.15-8.12 (m, 1H), |
| | methyl-N-((2- | 7.90-7.80 (m, 3H), 7.65-7.51 (m, 3H), 2.24 (s, 3H); |
| | (trifluoromethoxy)phenyl)sulfonyl)- | LCMS(M + 1): 478.9 |
| | 1H-1,2,3-triazole-4-carboxamide | |
| 245 | 1-(2-chloro-4-fluorophenyl)-5- | ¹H-NMR (400 MHz, DMSO-d₆) δ 8.37 (d, J = 7.3 Hz, |
| | methyl-N-((2- | 1H), 8.02-7.87 (m, 4H), 7.82 (dd, J = 8.8, 5.6 Hz, 1H), |
| | (trifluoromethyl)phenyl)sulfonyl)- | 7.54 (td, J = 8.5, 2.9 Hz, 1H), 2.25 (s, 3H); LCMS(M + 1): |
| | 1H-1,2,3-triazole-4-carboxamide | 463.0 |
| 246 | 1-(2-chloro-4-fluorophenyl)-N-((2- | ¹H-NMR (400 MHz, DMSO-d₆) δ 8.01-7.98 (m, 1H), |
| | fluorophenyl)sulfonyl)-5-methyl-1H- | 7.88 (dd, J = 8.6, 2.7 Hz, 1H), 7.82 (dd, J = 8.8, 5.6 Hz, |
| | 1,2,3-triazole-4-carboxamide | 1H), 7.76 (d, J = 9.8 Hz, 1H), 7.54 (td, J = 8.5, 3.0 Hz, |
| | | 1H), 7.47-7.43 (m, 2H), 2.24 (s, 3H); (LCMS(M + 1): |
| | | 413.0 |
| 247 | 1-(2-chloro-4-fluorophenyl)-N-((2,4- | ¹H-NMR (400 MHz, DMSO-d₆) δ 8.15 (d, J = 8.8 Hz, |
| | dichlorophenyl)sulfonyl)-5-methyl- | 1H), 7.90-7.81 (m, 3H), 7.71-7.69 (m, 1H), 7.54 (td, J = |
| | 1H-1,2,3-triazole-4-carboxamide | 8.5, 2.8 Hz, 1H), 2.25 (s, 3H); LCMS(M + 1): 464.90 |
| 248 | 1-(2-chloro-4-fluorophenyl)-N-((2,4- | ¹H-NMR (400 MHz, DMSO-d₆) δ 8.07 (dd, J = 14.9, 8.6 |
| | difluorophenyl)sulfonyl)-5-methyl- | Hz, 1H), 7.90-7.80 (m, 2H), 7.58-7.52 (m, 2H), 7.37-7.33 |
| | 1H-1,2,3-triazole-4-carboxamide | (m, 1H), 2.25 (s, 3H); (LCMS(M + 1): 430.8 |
| 249 | 1-(2-chloro-4-fluorophenyl)-N-((3- | ¹H-NMR (400 MHz, DMSO-d₆) δ 8.17 (d, J = 6.8 Hz, |
| | chloro-4-fluorophenyl)sulfonyl)-5- | 1H), 8.04 (d, J = 8.8 Hz, 1H), 7.88 (dd, J = 8.4, 2.8 Hz, |
| | methyl-1H-1,2,3-triazole-4- | 1H), 7.81 (dd, J = 8.9, 5.5 Hz, 1H), 7.71 (t, J = 8.8 Hz, |
| | carboxamide | 1H), 7.55 (dd, J = 8.3, 2.9 Hz, 2H), 2.27 (s, 3H); |
| | | LCMS(M+): 446.8 |
| 250 | N-((5-chloro-2,4- | ¹H-NMR (400 MHz, DMSO-d₆) δ 8.12 (t, J = 7.7 Hz, 1H), |
| | difluorophenyl)sulfonyl)-1-(2- | 7.89-7.79 (m, 4H), 7.54 (td, J = 8.5, 2.7 Hz, 1H), 2.27 (s, |
| | chloro-4-fluorophenyl)-5-methyl- | 3H); LCMS(M+): 464.8 |
| | 1H-1,2,3-triazole-4-carboxamide | |
| 251 | N-((4-chloro-2,5- | ¹H-NMR (400 MHz, DMSO-d₆) δ 7.99 (s, 1H), 7.88 (dd, |
| | dimethylphenyl)sulfonyl)-1-(2- | J = 8.6, 2.7 Hz, 1H), 7.81 (dd, J = 8.8, 5.6 Hz, 1H), 7.56- |
| | chloro-4-fluorophenyl)-5-methyl- | 7.52 (m, 2H), 2.59 (s, 3H), 2.39 (s, 3H), 2.24 (s, 3H); |
| | 1H-1,2,3-triazole-4-carboxamide | LCMS(M+): 456.8 |
| 252 | 1-(2-chloro-4-fluorophenyl)-N-((2- | ¹H-NMR (400 MHz, DMSO-d₆) δ 8.37 (d, J = 2.1 Hz, |
| | chloro-5- | 1H), 8.10 (dd, J = 8.4, 2.0 Hz, 1H), 7.94 (d, J = 8.3 Hz, |
| | (trifluoromethyl)phenyl)sulfonyl)-5- | 1H), 7.88 (dd, J = 8.6, 2.8 Hz, 1H), 7.82 (dd, J = 9.0, 5.7 |
| | methyl-1H-1,2,3-triazole-4- | Hz, 1H), 7.54 (td, J = 8.5, 2.9 Hz, 1H), 2.28-2.21 (m, 3H); |
| | carboxamide | LCMS(M − 1): 495.9 |
| 253 | N-((2-chloro-5- | ¹H-NMR (400 MHz, DMSO-d₆) δ 8.35 (s, 1H), 8.07 (d, |
| | (trifluoromethyl)phenyl)sulfonyl)-1- | J = 7.8 Hz, 1H), 7.93-7.90 (m, 2H), 7.82 (d, J = 1.7 Hz, |
| | (3,5-dichlorophenyl)-5-methyl-1H- | 2H), 2.43 (s, 3H); LCMS(M + 1): 514.7 |
| | 1,2,3-triazole-4-carboxamide | |
| 254 | 1-(3,5-dichlorophenyl)-5-methyl-N- | ¹H-NMR (400 MHz, DMSO-d₆) δ 8.35 (d, J = 8.3 Hz, |
| | ((2- | 1H), 8.00-7.93 (m, 4H), 7.82 (d, J = 1.7 Hz, 2H), 2.43 (s, |
| | (trifluoromethyl)phenyl)sulfonyl)- | 3H), (LCMS(M+): 478.8 |
| | 1H-1,2,3-triazole-4-carboxamide | |
| 255 | 1-(2,5-dichlorophenyl)-N-((2- | ¹H-NMR (400 MHz, DMSO-d₆) δ 8.02-7.98 (m, 2H), |
| | fluorophenyl)sulfonyl)-5-methyl-1H- | 7.87-7.76 (m, 3H), 7.48-7.44 (m, 2H), 2.27 (s, 3H); |
| | 1,2,3-triazole-4-carboxamide | LCMS (M + 1): 430.70 |
| 256 | 1-(4-chloro-2- | ¹H-NMR (400 MHz, DMSO-d₆) δ 8.22 (d, J = 2.2 Hz, |
| | (trifluoromethyl)phenyl)-N-((2,5- | 1H), 8.11-8.07 (m, 2H), 7.93 (d, J = 8.6 Hz, 1H), 7.80 |
| | dichlorophenyl)sulfonyl)-5-ethyl- | (dd, J = 8.7, 2.6 Hz, 1H), 7.72 (d, J = 8.6 Hz, 1H), 2.67 |
| | 1H-1,2,3-triazole-4-carboxamide | (q, J = 7.4 Hz, 2H), 0.87 (t, J = 7.5 Hz, 3H); LCMS(M + 1): |
| | | 528.90 |
| 257 | 1-(4-chloro-2- | ¹H-NMR (400 MHz, DMSO-d₆) δ 8.35 (d, J = 2.0 Hz, |
| | (trifluoromethyl)phenyl)-N-((2- | 1H), 8.22 (d, J = 2.2 Hz, 1H), 8.10-8.07 (m, 2H), 7.95- |
| | chloro-5- | 7.89 (m, 2H), 2.68 (t, J = 7.2 Hz, 3H), 0.92-0.85 (m, 3H); |
| | (trifluoromethyl)phenyl)sulfonyl)-5- | LCMS(M+): 560.95 |
| | ethyl-1H-1,2,3-triazole-4- | |
| | carboxamide | |
| 258 | 1-(4-chloro-2- | ¹H-NMR (400 MHz, DMSO-d₆) δ 8.22 (d, J = 2.2 Hz, |
| | (trifluoromethyl)phenyl)-N-((2- | 1H), 8.16 (d, J = 7.8 Hz, 1H), 8.09-8.07 (m, 1H), 7.92 (d, |
| | chlorophenyl)sulfonyl)-5-ethyl-1H- | J = 8.6 Hz, 1H), 7.68 (d, J = 7.6 Hz, 2H), 7.60 (s, 1H), |
| | 1,2,3-triazole-4-carboxamide | 2.66 (s, 2H), 0.86 (t, J = 7.5 Hz, 3H); LCMS(M+): 492.95 |
| 259 | 1-(2,5-dichlorophenyl)-5-methyl-N- | ¹H-NMR (400 MHz, DMSO-d₆) δ 13.21 (s, 1H), 8.37 (d, |
| | ((2- | J = 7.6 Hz, 1H), 8.02 (dd, J = 7.7, 1.3 Hz, 1H), 7.99-7.95 |
| | (trifluoromethyl)phenyl)sulfonyl)- | (m, 2H), 7.92 (t, J = 7.3 Hz, 1H), 7.86 (d, J = 8.3 Hz, 1H), |
| | 1H-1,2,3-triazole-4-carboxamide | 7.81 (dd, J = 8.6, 2.4 Hz, 1H), 2.27 (s, 3H); LCMS(M + 1): |
| | | 480.65 |
| 260 | 1-(2-chloro-4-cyanophenyl)-N-((2- | ¹H-NMR (400 MHz, DMSO-d₆) δ 8.47 (d, J = 1.5 Hz, |
| | chloro-5-methoxyphenyl)sulfonyl)- | 1H), 8.17-8.12 (m, 1H), 7.95 (d, J = 8.1 Hz, 1H), 7.59 (d, |
| | 5-methyl-1H-1,2,3-triazole-4- | J = 3.2 Hz, 1H), 7.46 (s, 1H), 7.14 (s, 1H), 3.82 (s, 3H), |
| | carboxamide | 2.31 (s, 3H); LCMS (M+): 465.8 |
| 261 | N-((2-bromo-5- | ¹H-NMR (400 MHz, DMSO-d₆) δ 8.38 (d, J = 2.1 Hz, |
| | (trifluoromethyl)phenyl)sulfonyl)-1- | 1H), 8.12 (d, J = 8.3 Hz, 1H), 8.05 (d, J = 2.1 Hz, 1H), |
| | (2,4-dichlorophenyl)-5-methyl-1H- | 7.98 (dd, J = 8.4, 2.0 Hz, 1H), 7.79-7.76 (m, 1H), 7.73 |
| | 1,2,3-triazole-4-carboxamide | (dd, J = 8.6, 2.1 Hz, 1H), 2.26 (s, 3H); LCMS (M + 1): |
| | | 558.65 |
| 262 | N-((2-chloro-5- | ¹H-NMR (400 MHz, DMSO-d₆) δ 8.36 (d, J = 1.8 Hz, |
| | (trifluoromethyl)phenyl)sulfonyl)-1- | 1H), 8.12 (dd, J = 8.4, 2.0 Hz, 1H), 8.06 (d, J = 2.4 Hz, |
| | (2,4-dichlorophenyl)-5-isopropyl- | 1H), 7.96 (d, J = 8.3 Hz, 1H), 7.87 (d, J = 8.3 Hz, 1H), |
| | 1H-1,2,3-triazole-4-carboxamide | 7.74 (dd, J = 8.6, 2.4 Hz, 1H), 3.01-2.94 (m, 1H), 1.09 (d, |
| | | J = 7.0 Hz, 3H), 1.04 (d, J = 7.0 Hz, 3H); LCMS (M + 1): |
| | | 542.80 |
| 263 | 1-(2,5-dichlorophenyl)-5-methyl-N- | ¹H-NMR (400 MHz, DMSO-d₆) δ 13.34 (s, 1H), 8.14 (dd, |
| | ((2- | J = 7.9, 1.6 Hz, 1H), 7.99-7.97 (m, 1H), 7.88-7.80 (m, |
| | (trifluoromethoxy)phenyl)sulfonyl)- | 3H), 7.64-7.58 (m, 2H), 2.26 (s, 3H); LCMS (M+): |
| | 1H-1,2,3-triazole-4-carboxamide | 495.05 |
| 264 | N-((2-chlorophenyl)sulfonyl)-1-(2,5- | ¹H-NMR (400 MHz, DMSO-d₆) δ 8.18 (dd, J = 7.9, 1.3 |
| | dichlorophenyl)-5-methyl-1H-1,2,3- | Hz, 1H), 7.99 (d, J = 2.0 Hz, 1H), 7.87-7.80 (m, 2H), |
| | triazole-4-carboxamide | 7.73-7.66 (m, 2H), 7.64-7.60 (m, 1H), 2.26 (s, 3H); |
| | | LCMS (M + 1): 446.80 |
| 265 | 1-(2,5-dichlorophenyl)-N-((2,5- | ¹H-NMR (400 MHz, DMSO-d₆) δ 7.98 (d, J = 2.2 Hz, |
| | difluorophenyl)sulfonyl)-5-methyl- | 1H), 7.87-7.75 (m, 3H), 7.70-7.64 (m, 1H), 7.55 (td, J = |
| | 1H-1,2,3-triazole-4-carboxamide | 9.4, 4.1 Hz, 1H), 2.30 (s, 3H); LCMS (M+): 446.80 |
| 266 | N-((2-chloro-5- | ¹H-NMR (400 MHz, DMSO-d₆) δ 8.06 (d, J = 2.2 Hz, |
| | methoxyphenyl)sulfonyl)-1-(2,4- | 1H), 7.87 (d, J = 8.6 Hz, 1H), 7.74 (dd, J = 8.4, 2.3 Hz, |
| | dichlorophenyl)-5-isopropyl-1H- | 1H), 7.62-7.58 (m, 2H), 7.29 (dd, J = 8.9, 3.1 Hz, 1H), |
| | 1,2,3-triazole-4-carboxamide | 3.85 (s, 3H), 2.95 (t, J = 7.1 Hz, 1H), 1.08 (dd, J = 21.9, |
| | | 7.0 Hz, 6H); LCMS (M + 1): 504.85 |
| 267 | 1-(2,4-dichlorophenyl)-N-((2,5- | ¹H-NMR (400 MHz, DMSO-d₆) δ 8.11 (d, J = 2.4 Hz, |
| | dichlorophenyl)sulfonyl)-5- | 1H), 8.06 (d, J = 2.2 Hz, 1H), 7.87 (d, J = 8.6 Hz, 1H), |
| | isopropyl-1H-1,2,3-triazole-4- | 7.81 (dd, J = 8.6, 2.7 Hz, 1H), 7.74 (dd, J = 8.6, 2.4 Hz, |
| | carboxamide | 2H), 2.97 (t, J = 7.1 Hz, 1H), 1.08 (dd, J = 22.1, 7.0 Hz, |
| | | 6H); LCMS (M + 1): 508.80 |
| 268 | N-((4-chloro-2,5- | ¹H-NMR (400 MHz, DMSO-d₆) δ 9.18 (s, 1H), 8.00 (s, |
| | dimethylphenyl)sulfonyl)-1-(2- | 1H), 7.88-7.82 (m, 2H), 7.54-7.49 (m, 2H), 2.57 (s, 3H), |
| | chloro-4-fluorophenyl)-1H-1,2,3- | 2.39 (s, 3H); LCMS(M+): 442.9 |
| | triazole-4-carboxamide | |
| 269 | 1-(2-chloro-4-cyanophenyl)-N-((2,5- | ¹H-NMR (400 MHz, DMSO-d₆) δ 8.49 (d, J = 1.7 Hz, |
| | difluorophenyl)sulfonyl)-5-methyl- | 1H), 8.15 (dd, J = 8.1, 1.7 Hz, 1H), 7.96 (d, J = 8.1 Hz, |
| | 1H-1,2,3-triazole-4-carboxamide | 1H), 7.79-7.75 (m, 1H), 7.69-7.63 (m, 1H), 7.55 (td, J = |
| | | 9.3, 4.1 Hz, 1H), 2.32-2.28 (s, 3H); LCMS (M+): 437.85 |
| 270 | 1-(2,5-dichlorophenyl)-N-((2,4- | ¹H-NMR (400 MHz, DMSO-d₆) δ 8.16 (d, J = 8.6 Hz, |
| | dichlorophenyl)sulfonyl)-5-methyl- | 1H), 7.98 (q, J = 2.4 Hz, 1H), 7.90-7.80 (m, 3H), 7.71 |
| | 1H-1,2,3-triazole-4-carboxamide | (dd, J = 8.7, 2.1 Hz, 1H), 2.27 (s, 3H); LCMS (M+): |
| | | 480.80 |
| 271 | 1-(2-chloro-4-cyanophenyl)-5- | ¹H-NMR (400 MHz, DMSO-d₆) δ 8.49 (d, J = 1.7 Hz, |
| | methyl-N-((2- | 1H), 8.37 (d, J = 7.6 Hz, 1H), 8.16 (dd, J = 8.3, 1.7 Hz, |
| | (trifluoromethyl)phenyl)sulfonyl)- | 1H), 8.02-8.00 (m, 1H), 7.98-7.89 (m, 3H), 2.32-2.28 (s, |
| | 1H-1,2,3-triazole-4-carboxamide | 3H); LCMS (M+): 469.90 |
| 272 | 1-(2-chloro-4-cyanophenyl)-N-((2- | ¹H-NMR (400 MHz, DMSO-d₆) δ 8.49 (d, J = 1.5 Hz, |
| | chlorophenyl)sulfonyl)-5-methyl- | 1H), 8.16 (td, J = 8.4, 1.6 Hz, 2H), 7.97 (d, J = 8.3 Hz, |
| | 1H-1,2,3-triazole-4-carboxamide | 1H), 7.73-7.66 (m, 2H), 7.64-7.59 (m, 1H), 2.26 (s, 3H); |
| | | LCMS (M+): 435.85 |
| 273 | 1-(2-chloro-4-fluorophenyl)-N-((2,5- | ¹H-NMR (400 MHz, DMSO-d₆) δ 9.16 (s, 1H), 8.11 (d, |
| | dichlorophenyl)sulfonyl)-1H-1,2,3- | J = 2.7 Hz, 1H), 7.87-7.78 (m, 2H), 7.71 (d, J = 8.6 Hz, |
| | triazole-4-carboxamide | 1H), 7.54-7.49 (m, 1H); LCMS(M + 1): 450.7 |
| 274 | N-((3,5- | ¹H-NMR (400 MHz, CHLOROFORM-D) δ 9.80 (s, 1H), |
| | bis(trifluoromethyl)phenyl)sulfonyl)- | 8.66 (d, J = 0.5 Hz, 2H), 8.16 (s, 1H), 7.96 (d, J = 1.5 Hz, |
| | 1-(2-chloro-4-cyanophenyl)-5- | 1H), 7.82 (dd, J = 8.2, 1.8 Hz, 1H), 7.55 (d, J = 8.1 Hz, |
| | methyl-1H-1,2,3-triazole-4- | 1H), 2.46 (s, 3H); LCMS (M − 2): 535.85 |
| | carboxamide | |
| 275 | 1-(2-chloro-4-cyanophenyl)-5- | ¹H-NMR (400 MHz, DMSO-d₆) δ 12.84 (s, 1H), 8.49 (d, |
| | methyl-N-((4- | J = 1.5 Hz, 1H), 8.16-8.13 (m, 3H), 7.95 (d, J = 8.1 Hz, |
| | (trifluoromethoxy)phenyl)sulfonyl)- | 1H), 7.66 (d, J = 8.1 Hz, 2H), 2.29 (s, 3H); LCMS (M+): |
| | 1H-1,2,3-triazole-4-carboxamide | 485.40 |
| 276 | N-((3,5- | ¹H-NMR (400 MHz, DMSO-d₆) δ 8.54 (d, J = 3.7 Hz, |
| | bis(trifluoromethyl)phenyl)sulfonyl)- | 3H), 7.94 (d, J = 2.4 Hz, 1H), 7.85 (d, J = 8.9 Hz, 1H), |
| | 1-(2,5-dichlorophenyl)-5-methyl- | 7.80 (dd, J = 8.9, 2.4 Hz, 1H), 2.30 (s, 3H); LCMS (M − |
| | 1H-1,2,3-triazole-4-carboxamide | 3): 544.45 |
| 277 | 1-(2,5-dichlorophenyl)-5-methyl-N- | ¹H-NMR (400 MHz, DMSO-d₆) δ 8.16-8.14 (m, 2H), |
| | ((4- | 7.96 (d, J = 2.2 Hz, 1H), 7.87-7.80 (m, 2H), 7.65 (d, J = |
| | (trifluoromethoxy)phenyl)sulfonyl)- | 8.1 Hz, 2H), 2.29 (s, 3H); LCMS (M+): 494.90 |
| | 1H-1,2,3-triazole-4-carboxamide | |
| 278 | 1-(2,4-dichlorophenyl)-N-((2- | ¹H-NMR (400 MHz, DMSO-d₆) δ 8.05 (d, J = 2.2 Hz, |
| | fluorophenyl)sulfonyl)-5-methyl-1H- | 1H), 8.00 (td, J = 7.7, 1.6 Hz, 1H), 7.80-7.72 (m, 3H), |
| | 1,2,3-triazole-4-carboxamide | 7.49-7.44 (m, 2H), 2.25 (s, 3H); LCMS (M + 1): 428.35 |
| 279 | 1-(2,4-dichlorophenyl)-N-((2,4- | ¹H-NMR (400 MHz, DMSO-d₆) δ 8.11-8.05 (m, 2H), |
| | difluorophenyl)sulfonyl)-5-methyl- | 7.78 (d, J = 8.6 Hz, 1H), 7.74 (dd, J = 8.4, 2.1 Hz, 1H), |
| | 1H-1,2,3-triazole-4-carboxamide | 7.60-7.54 (m, 1H), 7.36 (td, J = 8.6, 2.0 Hz, 1H), 2.26 (s, |
| | | 3H); LCMS (M + 1): 446.30 |
| 280 | N-((4-chloro-2,5- | ¹H-NMR (400 MHz, DMSO-d₆) δ 13.11 (s, 1H), 8.06- |
| | dimethylphenyl)sulfonyl)-1-(2,4- | 8.05 (m, 1H), 8.00 (s, 1H), 7.78-7.76 (m, 1H), 7.74 (dd, |
| | dichlorophenyl)-5-methyl-1H-1,2,3- | J = 8.4, 2.1 Hz, 1H), 7.52 (s, 1H), 2.59 (s, 3H), 2.39 (s, 3H), |
| | triazole-4-carboxamide | 2.25 (s, 3H); LCMS (M + 1): 474.30 |
| 281 | 1-(2,4-dichlorophenyl)-5-methyl-N- | ¹H-NMR (400 MHz, DMSO-d₆) δ 8.06 (d, J = 2.2 Hz, |
| | ((2,4,6-trifluorophenyl)sulfonyl)-1H- | 1H), 7.79 (d, J = 8.3 Hz, 1H), 7.74 (dd, J = 8.4, 2.1 Hz, |
| | 1,2,3-triazole-4-carboxamide | 1H), 7.48 (t, J = 9.3 Hz, 2H), 2.28 (s, 3H); LCMS (M + 1): |
| | | 464.35 |
| 282 | 1-(2-chloro-4-fluorophenyl)-N-((3- | ¹H-NMR (400 MHz, DMSO-d₆) δ 9.18 (s, 1H), 8.16 (dd, |
| | chloro-4-fluorophenyl)sulfonyl)-1H- | J = 6.7, 2.3 Hz, 1H), 8.02 (qd, J = 4.4, 2.4 Hz, 1H), 7.87- |
| | 1,2,3-triazole-4-carboxamide | 7.81 (m, 2H), 7.70 (t, J = 8.9 Hz, 1H), 7.51 (td, J = 8.4, |
| | | 2.7 Hz, 1H); LCMS(M − 2): 430.3 |
| 283 | 1-(2-chloro-4-fluorophenyl)-N-((2- | ¹H-NMR (400 MHz, DMSO-d₆) δ 9.18 (s, 1H), 7.88-7.84 |
| | chloro-5-methoxyphenyl)sulfonyl)- | (m, 2H), 7.63 (d, J = 3.2 Hz, 1H), 7.57 (d, J = 8.8 Hz, 1H), |
| | 1H-1,2,3-triazole-4-carboxamide | 7.54-7.49 (m, 1H), 7.28 (dd, J = 8.8, 3.2 Hz, 1H), 3.85 (s, |
| | | 3H); LCMS(M − 1): 444.3 |
| 284 | 1-(2-chloro-4-fluorophenyl)-N-((2- | ¹H-NMR (400 MHz, DMSO-d₆) δ 9.18 (s, 1H), 8.16 (dd, |
| | (trifluoromethoxy)phenyl)sulfonyl)- | J = 7.9, 1.6 Hz, 1H), 7.88-7.82 (m, 3H), 7.66-7.59 (m, |
| | 1H-1,2,3-triazole-4-carboxamide | 2H), 7.54-7.49 (m, 1H); LCMS(M+): 464.3 |
| 285 | 1-(2-chloro-4-fluorophenyl)-N-((2- | ¹H-NMR (400 MHz, DMSO-d₆) δ 9.20 (s, 1H), 8.39 (d, |
| | (trifluoromethyl)phenyl)sulfonyl)- | J = 7.6 Hz, 1H), 8.03-7.91 (m, 2H), 7.88-7.83 (m, 2H), |
| | 1H-1,2,3-triazole-4-carboxamide | 7.54-7.49 (m, 1H); LCMS(M+): 448.3 |
| 286 | 1-(2-chloro-4-fluorophenyl)-N-((4- | ¹H-NMR (400 MHz, DMSO-d₆) δ 9.19 (s, 1H), 8.18-8.14 |
| | (trifluoromethoxy)phenyl)sulfonyl)- | (m, 2H), 7.89-7.82 (m, 2H), 7.66 (d, J = 8.1 Hz, 2H), |
| | 1H-1,2,3-triazole-4-carboxamide | 7.55-7.50 (m, 1H); LCMS(M + 1): 464.3 |
| 287 | N-((2-chloro-5- | ¹H-NMR (400 MHz, DMSO-d₆) δ 8.27 (d, J = 2.2 Hz, |
| | (trifluoromethyl)phenyl)sulfonyl)-1- | 1H), 8.07 (dd, J = 8.4, 1.8 Hz, 1H), 7.94 (d, J = 8.3 Hz, |
| | (2,4-dichlorophenyl)-5-phenyl-1H- | 1H), 7.90-7.86 (m, 2H), 7.65 (dd, J = 8.6, 2.2 Hz, 1H), |
| | 1,2,3-triazole-4-carboxamide | 7.38-7.28 (m, 3H), 7.24-7.21 (m, 2H); LCMS (M + 1): |
| | | 576.3 |
| 288 | N-((2-chloro-5- | ¹H-NMR (400 MHz, DMSO-d₆) δ 7.90 (d, J = 8.6 Hz, |
| | methoxyphenyl)sulfonyl)-1-(2,4- | 1H), 7.87 (d, J = 2.2 Hz, 1H), 7.65 (dd, J = 8.6, 2.4 Hz, |
| | dichlorophenyl)-5-phenyl-1H-1,2,3- | 1H), 7.57 (d, J = 8.8 Hz, 1H), 7.53 (d, J = 3.2 Hz, 1H), |
| | triazole-4-carboxamide | 7.39-7.29 (m, 3H), 7.27-7.22 (m, 3H), 3.80 (s, 3H); |
| | | LCMS (M + 1): 538.25 |
| 289 | 1-(2,4-dichlorophenyl)-N-((2,5- | ¹H-NMR (400 MHz, DMSO-d₆) δ 8.02 (d, J = 2.4 Hz, |
| | dichlorophenyl)sulfonyl)-5-phenyl- | 1H), 7.90-7.86 (m, 2H), 7.76 (dd, J = 8.7, 2.6 Hz, 1H), |
| | 1H-1,2,3-triazole-4-carboxamide | 7.71 (d, J = 8.6 Hz, 1H), 7.65 (dd, J = 8.6, 2.2 Hz, 1H), |
| | | 7.39-7.29 (m, 3H), 7.25-7.22 (m, 2H); LCMS (M + 1): |
| | | 542.85 |
| 290 | 1-(2,4-dichlorophenyl)-5-phenyl-N- | ¹H-NMR (400 MHz, DMSO-d₆) δ 8.31-8.29 (m, 1H), |
| | ((2- | 8.01-7.99 (m, 1H), 7.93-7.87 (m, 4H), 7.65 (dd, J = 8.6, |
| | (trifluoromethyl)phenyl)sulfonyl)- | 2.2 Hz, 1H), 7.39-7.29 (m, 3H), 7.24 (dt, J = 6.5, 1.7 Hz, |
| | 1H-1,2,3-triazole-4-carboxamide | 2H); LCMS (M + 1): 542.85 |
| 291 | 1-(2,4-dichlorophenyl)-5-phenyl-N- | ¹H-NMR (400 MHz, DMSO-d₆) δ 8.06 (dd, J = 8.1, 1.7 |
| | ((2- | Hz, 1H), 7.89-7.87 (m, 2H), 7.81 (td, J = 7.9, 1.5 Hz, 1H), |
| | (trifluoromethoxy)phenyl)sulfonyl)- | 7.65 (dd, J = 8.6, 2.4 Hz, 1H), 7.59-7.55 (m, 2H), 7.38- |
| | 1H-1,2,3-triazole-4-carboxamide | 7.29 (m, 3H), 7.22 (dt, J = 6.6, 1.7 Hz, 2H); LCMS |
| | | (M + 1): 556.95 |
| 292 | 1-(2,4-dichlorophenyl)-N-((2- | ¹H-NMR (400 MHz, DMSO-d₆) δ 7.94-7.86 (m, 3H), |
| | fluorophenyl)sulfonyl)-5-phenyl-1H- | 7.77-7.71 (m, 1H), 7.65 (dd, J = 8.6, 2.2 Hz, 1H), 7.47- |
| | 1,2,3-triazole-4-carboxamide | 7.29 (m, 5H), 7.22 (dd, J = 8.3, 1.5 Hz, 2H); LCMS |
| | | (M + 1): 490.95 |
| 293 | N-((2-chlorophenyl)sulfonyl)-1-(2,4- | ¹H-NMR (400 MHz, DMSO-d₆) δ 8.10-8.08 (m, 1H), |
| | dichlorophenyl)-5-phenyl-1H-1,2,3- | 7.90-7.86 (m, 2H), 7.68-7.63 (m, 3H), 7.57-7.53 (m, 1H), |
| | triazole-4-carboxamide | 7.38-7.28 (m, 3H), 7.22 (dt, J = 6.6, 1.5 Hz, 2H); LCMS |
| | | (M + 1): 508.70 |
| 294 | 1-(2-chloro-4-fluorophenyl)-N-((2- | ¹H-NMR (400 MHz, DMSO-d₆) δ 9.21 (s, 1H), 8.19 (dd, |
| | chlorophenyl)sulfonyl)-1H-1,2,3- | J = 7.9, 1.5 Hz, 1H), 7.88-7.84 (m, 2H), 7.74-7.61 (m, |
| | triazole-4-carboxamide | 3H), 7.52 (td, J = 8.6, 2.8 Hz, 1H); LCMS(M − 1): 414.3 |
| 295 | N-((3,5- | ¹H-NMR (400 MHz, DMSO-d₆) δ 9.08 (s, 1H), 8.50 (d, |
| | bis(trifluoromethyl)phenyl)sulfonyl)- | J = 14.7 Hz, 3H), 7.85 (dd, J = 8.6, 2.8 Hz, 1H), 7.80 (dd, |
| | 1-(2-chloro-4-fluorophenyl)-1H- | J = 8.9, 5.5 Hz, 1H), 7.52-7.47 (m, 1H); (LCMS(M+): |
| | 1,2,3-triazole-4-carboxamide | 516.7 |
| 296 | N-((5-chloro-2- | ¹H-NMR (400 MHz, DMSO-d₆) δ 9.14 (s, 1H), 7.94 (q, |
| | fluorophenyl)sulfonyl)-1-(2-chloro- | J = 3.0 Hz, 1H), 7.88-7.82 (m, 3H), 7.55-7.49 (m, 2H); |
| | 4-fluorophenyl)-1H-1,2,3-triazole-4- | LCMS(M+): 432.3 |
| | carboxamide | |
| 297 | 1-(2-chloro-4-fluorophenyl)-N-((2,4- | ¹H-NMR (400 MHz, DMSO-d₆) δ 9.15 (s, 1H), 8.07 (td, |
| | difluorophenyl)sulfonyl)-1H-1,2,3- | J = 8.6, 6.2 Hz, 1H), 7.88-7.81 (m, 2H), 7.61-7.48 (m, |
| | triazole-4-carboxamide | 3H), 7.38-7.33 (m, 1H); LCMS, M+): 416.3 |
| 298 | 1-(2-chloro-4-fluorophenyl)-N-((2,5- | ¹H-NMR (400 MHz, DMSO-d₆) δ 9.16 (s, 1H), 7.88-7.82 |
| | difluorophenyl)sulfonyl)-1H-1,2,3- | (m, 2H), 7.80-7.75 (m, 1H), 7.69-7.63 (m, 1H), 7.61-7.49 |
| | triazole-4-carboxamide | (m, 2H); LCMS(M+): 416.3 |
| 299 | 1-(2-chloro-4-fluorophenyl)-N-((2- | ¹H-NMR (400 MHz, DMSO-d₆) δ 9.20 (s, 1H), 8.02 (td, |
| | fluorophenyl)sulfonyl)-1H-1,2,3- | J = 7.6, 1.7 Hz, 1H), 7.88-7.83 (m, 2H), 7.82-7.76 (m, |
| | triazole-4-carboxamide | 1H), 7.53-7.45 (m, 3H); LCMS(M+): 398.4 |
| 300 | N-((2-chloro-5- | ¹H-NMR (400 MHz, CHLOROFORM-D) δ 9.99 (s, 1H), |
| | (trifluoromethyl)phenyl)sulfonyl)-1- | 8.64 (d, J = 2.1 Hz, 1H), 7.85-7.82 (m, 1H), 7.73-7.69 (m, |
| | (2,4-dichloro-6-methylphenyl)-5- | 1H), 7.48-7.47 (m, 1H), 7.35-7.34 (m, 1H), 2.33 (s, 3H), |
| | methyl-1H-1,2,3-triazole-4- | 2.00 (s, 3H); LCMS (M + 1): 528.85 |
| | carboxamide | |
| 301 | N-((2-bromo-5- | ¹H-NMR (400 MHz, CHLOROFORM-D) δ 10.02 (s, |
| | (trifluoromethyl)phenyl)sulfonyl)-1- | 1H), 8.66 (d, J = 2.0 Hz, 1H), 7.91 (d, J = 8.3 Hz, 1H), |
| | (2,4-dichloro-6-methylphenyl)-5- | 7.74-7.71 (m, 1H), 7.48 (dd, J = 2.2, 0.5 Hz, 1H), 7.35 |
| | methyl-1H-1,2,3-triazole-4- | (dd, J = 2.2, 0.5 Hz, 1H), 2.33 (s, 3H), 2.00 (s, 3H); |
| | carboxamide | LCMS (M + 1): 572.20 |
| 302 | 1-(4-chloro-2- | ¹H-NMR (400 MHz, DMSO-d₆) δ 8.22 (d, J = 2.1 Hz, |
| | (trifluoromethyl)phenyl)-N-((2- | 1H), 8.09 (dd, J = 8.4, 2.3 Hz, 1H), 7.93 (d, J = 8.6 Hz, |
| | chloro-5-methoxyphenyl)sulfonyl)- | 1H), 7.61 (d, J = 3.1 Hz, 1H), 7.58 (d, J = 8.9 Hz, 1H), |
| | 5-ethyl-1H-1,2,3-triazole-4- | 7.28 (dd, J = 8.7, 2.9 Hz, 1H), 3.85 (s, 3H), 2.69-2.66 (m, |
| | carboxamide | 2H), 0.87 (t, J = 7.5 Hz, 3H); LCMS(M+): 522.95 |
| 303 | 1-(4-chloro-2- | ¹H-NMR (400 MHz, DMSO-d₆) δ 8.36 (d, J = 7.9 Hz, |
| | (trifluoromethyl)phenyl)-5-ethyl-N- | 1H), 8.22 (d, J = 2.1 Hz, 1H), 8.08 (dd, J = 8.6, 2.1 Hz, |
| | ((2- | 1H), 8.01-7.88 (m, 4H), 2.72-2.65 (m, 2H), 0.87 (t, J = |
| | (trifluoromethyl)phenyl)sulfonyl)- | 7.5 Hz, 3H); LCMS(M+): 526.95 |
| | 1H-1,2,3-triazole-4-carboxamide | |
| 304 | 1-(4-chloro-2- | ¹H-NMR (400 MHz, DMSO-d₆) δ 8.19 (d, J = 2.4 Hz, |
| | (trifluoromethyl)phenyl)-5-ethyl-N- | 1H), 8.06 (dd, J = 8.6, 2.4 Hz, 1H), 8.00-7.96 (m, 1H), |
| | ((2-fluorophenyl)sulfonyl)-1H-1,2,3- | 7.89 (d, J = 8.6 Hz, 1H), 7.77-7.72 (m, 1H), 7.46-7.41 (m, |
| | triazole-4-carboxamide | 2H), 2.62 (q, J = 7.2 Hz, 2H), 0.82 (d, J = 7.6 Hz, 3H); |
| | | LCMS(M+): 477.10 |
| 305 | 1-(2-chloro-4- | ¹H-NMR (400 MHz, DMSO-d₆) δ 8.10 (d, J = 2.8 Hz, |
| | (trifluoromethoxy)phenyl)-N-((2,5- | 1H), 8.02 (d, J = 2.4 Hz, 1H), 7.99-7.96 (m, 1H), 7.79 |
| | dichlorophenyl)sulfonyl)-5-ethyl- | (dd, J = 8.6, 2.4 Hz, 1H), 7.72-7.68 (m, 2H), 2.71-2.66 |
| | 1H-1,2,3-triazole-4-carboxamide | (m, 2H), 0.89 (t, J = 7.5 Hz, 3H); LCMS(M+): 542.95 |
| 306 | 1-(2-chloro-4- | ¹H-NMR (400 MHz, DMSO-d₆) δ 8.37 (d, J = 7.6 Hz, |
| | (trifluoromethoxy)phenyl)-5-ethyl- | 1H), 8.03-7.88 (m, 5H), 7.70 (dd, J = 8.7, 1.4 Hz, 1H), |
| | N-((2- | 2.67 (q, J = 7.3 Hz, 2H), 0.81-0.96 (3H); LCMS(M+): |
| | (trifluoromethyl)phenyl)sulfonyl)- | 542.95 |
| | 1H-1,2,3-triazole-4-carboxamide | |
| 307 | 1-(2,4-dichloro-6-methylphenyl)-N- | ¹H-NMR (400 MHz, CHLOROFORM-D) δ 10.00 (s, |
| | ((2-fluoro-5- | 1H), 8.48 (dd, J = 6.4, 2.0 Hz, 1H), 7.95-7.91 (m, 1H), |
| | (trifluoromethyl)phenyl)sulfonyl)-5- | 7.47 (d, J = 1.7 Hz, 1H), 7.41-7.34 (m, 2H), 2.34 (s, 3H), |
| | methyl-1H-1,2,3-triazole-4- | 2.00 (s, 3H); LCMS (M + 1): 510.95 |
| | carboxamide | |
| 308 | 1-(2-chloro-4-methoxyphenyl)-5- | ¹H-NMR (400 MHz, DMSO-d₆) δ 8.37 (d, J = 7.6 Hz, |
| | methyl-N-((2- | 1H), 8.01 (d, J = 7.6 Hz, 1H), 7.98-7.94 (m, 1H), 7.91 (t, |
| | (trifluoromethyl)phenyl)sulfonyl)- | J = 7.6 Hz, 1H), 7.61 (dd, J = 8.7, 2.8 Hz, 1H), 7.38 (d, |
| | 1H-1,2,3-triazole-4-carboxamide | J = 2.9 Hz, 1H), 7.16 (dd, J = 8.9, 2.8 Hz, 1H), 3.88 (d, J = |
| | | 2.9 Hz, 3H), 2.23 (s, 3H); LCMS(M+): 474.90 |
| 309 | 1-(2-chloro-4-methoxyphenyl)-N- | ¹H-NMR (400 MHz, DMSO-d₆) δ 8.17 (dd, J = 7.9, 1.3 |
| | ((2-chlorophenyl)sulfonyl)-5- | Hz, 1H), 7.73-7.66 (m, 2H), 7.64-7.59 (m, 2H), 7.38 (d, J |
| | methyl-1H-1,2,3-triazole-4- | J = 2.7 Hz, 1H), 7.15 (dd, J = 8.8, 2.7 Hz, 1H), 3.88 (d, J |
| | carboxamide | 3.2 Hz, 3H), 2.22 (s, 3H); LCMS(M+): 440.80 |
| 310 | 1-(2-chloro-4-methoxyphenyl)-N- | ¹H-NMR (400 MHz, DMSO-d₆) δ 8.02-7.98 (m, 1H), |
| | ((2-fluorophenyl)sulfonyl)-5-methyl- | 7.77 (dd, J = 11.6, 7.5 Hz, 1H), 7.62 (d, J = 8.8 Hz, 1H), |
| | 1H-1,2,3-triazole-4-carboxamide | 7.48-7.43 (m, 2H), 7.38 (d, J = 2.9 Hz, 1H), 7.15 (dd, J = |
| | | 8.8, 2.7 Hz, 1H), 3.87 (s, 3H), 2.22 (s, 3H); LCMS(M+): |
| | | 424.80 |
| 311 | 1-(2-chloro-4- | ¹H-NMR (400 MHz, DMSO-d₆) δ 8.36 (d, J = 2.1 Hz, |
| | (trifluoromethoxy)phenyl)-N-((2- | 1H), 8.10 (dd, J = 8.6, 1.8 Hz, 1H), 8.02 (d, J = 2.1 Hz, |
| | chloro-5- | 1H), 7.99-7.97 (m, 1H), 7.95 (d, J = 8.6 Hz, 1H), 7.70 |
| | (trifluoromethyl)phenyl)sulfonyl)-5- | (dq, J = 8.8, 1.2 Hz, 1H), 2.72-2.65 (m, 2H), 0.88 (t, J = |
| | ethyl-1H-1,2,3-triazole-4- | 7.5 Hz, 3H); LCMS(M − 2): 574.75 |
| | carboxamide | |
| 312 | 1-(2-chloro-4-methoxyphenyl)-N- | ¹H-NMR (400 MHz, DMSO-d₆) δ 8.10 (d, J = 2.4 Hz, |
| | ((2,5-dichlorophenyl)sulfonyl)-5- | 1H), 7.80 (dd, J = 8.7, 2.6 Hz, 1H), 7.72 (d, J = 8.6 Hz, |
| | methyl-1H-1,2,3-triazole-4- | 1H), 7.63 (d, J = 8.8 Hz, 1H), 7.38 (d, J = 2.7 Hz, 1H), |
| | carboxamide | 7.16 (dd, J = 8.9, 2.8 Hz, 1H), 3.87 (s, 3H), 2.23 (s, 3H); |
| | | LCMS(M+): 476.25 |
| 313 | 1-(2-chloro-4-methoxyphenyl)-N- | ¹H-NMR (400 MHz, DMSO-d₆) δ 7.53-7.68 (3H), 7.35- |
| | ((2-chloro-5- | 7.42 (1H), 7.24-7.32 (1H), 7.12-7.20 (1H), 3.86-3.89 |
| | methoxyphenyl)sulfonyl)-5-methyl- | (3H), 3.83-3.86 (3H), 2.18-2.26 (3H),LCMS(M+): |
| | 1H-1,2,3-triazole-4-carboxamide | 470.40 |
| 314 | 1-(2-chloro-4-methoxyphenyl)-N- | ¹H-NMR (400 MHz, DMSO-d₆) δ 8.36 (d, J = 2.2 Hz, |
| | ((2-chloro-5- | 1H), 8.08 (d, J = 10.5 Hz, 1H), 7.93 (d, J = 8.3 Hz, 1H), |
| | (trifluoromethyl)phenyl)sulfonyl)-5- | 7.62 (d, J = 8.8 Hz, 1H), 7.37 (d, J = 2.7 Hz, 1H), 7.15 |
| | methyl-1H-1,2,3-triazole-4- | (dd, J = 8.9, 2.8 Hz, 1H), 3.87 (s, 4H), 2.23 (s, 3H); |
| | carboxamide | LCMS(M+): 508.30 |
| 315 | 1-(2-chloro-4-cyanophenyl)-N-((2- | ¹H-NMR (400 MHz, DMSO-d₆) δ 8.49 (d, J = 1.7 Hz, |
| | chloro-5- | 1H), 8.36 (d, J = 2.0 Hz, 1H), 8.15 (dd, J = 8.3, 1.7 Hz, |
| | (trifluoromethyl)phenyl)sulfonyl)-5- | 1H), 8.10 (dd, J = 8.6, 2.0 Hz, 1H), 7.95 (dd, J = 11.5, 8.3 |
| | methyl-1H-1,2,3-triazole-4- | Hz, 2H), 2.30 (s, 3H); LCMS (M − 1): 503.30 |
| | carboxamide | |
| 316 | 1-(2-chloro-4-cyanophenyl)-N-((2,5- | ¹H-NMR (400 MHz, DMSO-d₆) δ 8.50 (dd, J = 5.0, 1.7 |
| | dichlorophenyl)sulfonyl)-5-methyl- | Hz, 1H), 8.16 (qd, J = 4.0, 1.7 Hz, 1H), 8.11 (d, J = 2.4 |
| | 1H-1,2,3-triazole-4-carboxamide | Hz, 1H), 8.00-7.96 (m, 1H), 7.81 (dd, J = 8.7, 2.6 Hz, |
| | | 1H), 7.72 (d, J = 8.6 Hz, 1H), 2.30 (d, J = 12.8 Hz, 3H); |
| | | LCMS (M + 1): 471.20 |
| 317 | 1-(2,4-dichlorophenyl)-N-((2,4- | ¹H-NMR (400 MHz, DMSO-d₆) δ 8.09 (d, J = 8.6 Hz, |
| | dichlorophenyl)sulfonyl)-5-phenyl- | 1H), 7.92-7.88 (m, 3H), 7.66 (dt, J = 8.6, 1.1 Hz, 2H), |
| | 1H-1,2,3-triazole-4-carboxamide | 7.38-7.31 (m, 3H), 7.26-7.24 (m, 2H); LCMS (M + 1): |
| | | 542.25 |
| 318 | N-((2-chloro-5- | ¹H-NMR (400 MHz, CHLOROFORM-D) δ 9.95 (s, 1H), |
| | methoxyphenyl)sulfonyl)-1-(2,4- | 7.87 (d, J = 3.1 Hz, 1H), 7.47 (d, J = 2.1 Hz, 1H), 7.42 (d, |
| | dichloro-6-methylphenyl)-5-methyl- | J = 8.6 Hz, 1H), 7.34 (q, J = 1.0 Hz, 1H), 7.10 (dd, J = |
| | 1H-1,2,3-triazole-4-carboxamide | 8.7, 3.2 Hz, 1H), 3.91 (s, 3H), 2.33 (s, 3H), 2.00 (s, 3H); |
| | | LCMS (M + 1): 490.30 |
| 319 | N-((2-chlorophenyl)sulfonyl)-1-(2,4- | ¹H-NMR (400 MHz, CHLOROFORM-D) δ 9.94 (s, 1H), |
| | dichloro-6-methylphenyl)-5-methyl- | 8.39 (dd, J = 7.9, 1.5 Hz, 1H), 7.62-7.50 (m, 3H), 7.47 (d, |
| | 1H-1,2,3-triazole-4-carboxamide | J = 1.8 Hz, 1H), 7.34 (t, J = 1.1 Hz, 1H), 2.32 (s, 3H), |
| | | 2.00 (s, 3H); LCMS (M + 1): 460.25 |
| 320 | 1-(2,4-dichloro-6-methylphenyl)-N- | ¹H-NMR (400 MHz, CHLOROFORM-D) δ 9.95 (s, 1H), |
| | ((2,4-dichlorophenyl)sulfonyl)-5- | 8.31 (d, J = 8.6 Hz, 1H), 7.56 (d, J = 2.1 Hz, 1H), 7.52- |
| | methyl-1H-1,2,3-triazole-4- | 7.47 (m, 2H), 7.35 (q, J = 0.9 Hz, 1H), 2.32 (s, 3H), 2.00 |
| | carboxamide | (s, 3H); LCMS (M + 1): 494.80 |
| 321 | 1-(2,4-dichloro-6-methylphenyl)-5- | ¹H-NMR (400 MHz, CHLOROFORM-D) δ 9.83 (s, 1H), |
| | methyl-N-((2- | 8.64 (dd, J = 7.6, 1.5 Hz, 1H), 7.93-7.90 (m, 1H), 7.85- |
| | (trifluoromethyl)phenyl)sulfonyl)- | 7.77 (m, 2H), 7.47-7.46 (m, 1H), 7.34-7.33 (m, 1H), 2.32 |
| | 1H-1,2,3-triazole-4-carboxamide | (s, 3H), 1.98 (s, 3H); LCMS (M + 1): 494.70 |
| 322 | 1-(2,4-dichloro-6-methylphenyl)-5- | ¹H-NMR (400 MHz, CHLOROFORM-D) δ 9.88 (s, 1H), |
| | methyl-N-((2- | 8.34 (dd, J = 8.1, 1.7 Hz, 1H), 7.74-7.69 (m, 1H), 7.53- |
| | (trifluoromethoxy)phenyl)sulfonyl)- | 7.49 (m, 1H), 7.47-7.43 (m, 2H), 7.34 (dd, J = 2.3, 0.6 |
| | 1H-1,2,3-triazole-4-carboxamide | Hz, 1H), 2.32 (s, 3H), 1.99 (s, 3H); LCMS (M + 1): 510.70 |
| 323 | 1-(2,4-dichloro-6-methylphenyl)-N- | ¹H-NMR (400 MHz, CHLOROFORM-D) δ 9.90 (s, 1H), |
| | ((2-fluorophenyl)sulfonyl)-5-methyl- | 8.22-8.18 (m, 1H), 7.70-7.64 (m, 1H), 7.47 (dd, J = 2.3, |
| | 1H-1,2,3-triazole-4-carboxamide | 0.6 Hz, 1H), 7.38 (td, J = 7.8, 1.1 Hz, 1H), 7.34-7.34 (m, |
| | | 1H), 7.25-7.23 (m, 1H), 2.33 (s, 3H), 2.00 (s, 3H); LCMS |
| | | (M + 1): 442.90 |
| 324 | 1-(2,4-dichloro-6-methylphenyl)-5- | ¹H-NMR (400 MHz, CHLOROFORM-D) δ 9.72 (s, 1H), |
| | methyl-N-((4- | 8.26 (dt, J = 9.6, 2.4 Hz, 2H), 7.47 (q, J = 1.0 Hz, 1H), |
| | (trifluoromethoxy)phenyl)sulfonyl)- | 7.40 (dd, J = 9.0, 1.0 Hz, 2H), 7.34 (q, J = 1.0 Hz, 1H), |
| | 1H-1,2,3-triazole-4-carboxamide | 2.36 (s, 3H), 1.98 (s, 3H); LCMS (M + 1): 508.90 |
| 325 | 1-(2,4-dichloro-6-methylphenyl)-N- | ¹H-NMR (400 MHz, CHLOROFORM-D) δ 9.91 (s, 1H), |
| | ((2,4-difluorophenyl)sulfonyl)-5- | 8.23 (td, J = 8.5, 6.0 Hz, 1H), 7.47 (dd, J = 2.2, 0.5 Hz, |
| | methyl-1H-1,2,3-triazole-4- | 1H), 7.34 (q, J = 1.0 Hz, 1H), 7.12-7.07 (m, 1H), 7.02- |
| | carboxamide | 6.96 (m, 1H), 2.33 (s, 3H), 2.00 (s, 3H); LCMS (M + 1): |
| | | 460.90 |
| 326 | 1-(2,4-dichloro-6-methylphenyl)-N- | ¹H-NMR (400 MHz, CHLOROFORM-D) δ 9.93 (s, 1H), |
| | ((2,5-difluorophenyl)sulfonyl)-5- | 7.93-7.89 (m, 1H), 7.48 (dd, J = 2.2, 0.5 Hz, 1H), 7.38- |
| | methyl-1H-1,2,3-triazole-4- | 7.32 (m, 2H), 7.22 (dd, J = 9.0, 3.9 Hz, 1H), 2.34 (s, 3H), |
| | carboxamide | 2.00 (s, 3H); LCMS (M + 1): 460.90 |
| 327 | N-((3,5- | ¹H-NMR (400 MHz, CHLOROFORM-D) δ 9.94 (s, 1H), |
| | bis(trifluoromethyl)phenyl)sulfonyl)- | 8.67 (s, 2H), 8.16 (s, 1H), 7.48 (d, J = 2.1 Hz, 1H), 7.35 |
| | 1-(2,4-dichloro-6-methylphenyl)-5- | (t, J = 1.1 Hz, 1H), 2.37 (s, 3H), 1.99 (s, 3H); LCMS |
| | methyl-1H-1,2,3-triazole-4- | (M + 1): 560.95 |
| | carboxamide | |
| 328 | 1-(2-chloro-4-cyanophenyl)-N-((2,5- | ¹H-NMR (400 MHz, DMSO-d₆) δ 8.49 (d, J = 1.5 Hz, |
| | dimethylphenyl)sulfonyl)-5-methyl- | 1H), 8.15 (dd, J = 8.3, 1.7 Hz, 1H), 8.00-7.95 (m, 1H), |
| | 1H-1,2,3-triazole-4-carboxamide | 7.86 (s, 1H), 7.39 (d, J = 6.6 Hz, 1H), 7.29 (d, J = 7.8 Hz, |
| | | 1H), 2.59 (s, 3H), 2.36 (s, 3H), 2.26 (s, 3H); LCMS (M+): |
| | | 430.00 |
| 329 | N-((2,5- | ¹H-NMR (400 MHz, DMSO-d₆) δ 8.61 (s, 1H), 8.48 (d, |
| | bis(trifluoromethyl)phenyl)sulfonyl)- | J = 1.7 Hz, 1H), 8.28 (d, J = 8.6 Hz, 1H), 8.21 (d, J = 8.3 |
| | 1-(2-chloro-4-cyanophenyl)-5- | Hz, 1H), 8.14 (dd, J = 8.3, 1.7 Hz, 1H), 7.96 (d, J = 8.1 |
| | methyl-1H-1,2,3-triazole-4- | Hz, 1H), 2.29 (s, 3H); LCMS (M − 2): 535.30 |
| | carboxamide | |
| 330 | 1-(2-chloro-4-fluorophenyl)-N- | ¹H-NMR (400 MHz, DMSO-d₆) δ 9.17 (s, 1H), 7.88-7.83 |
| | ((2,4,6-trifluorophenyl)sulfonyl)-1H- | (m, 2H), 7.54-7.44 (m, 3H); LCMS(M+): 434.3 |
| | 1,2,3-triazole-4-carboxamide | |
| 331 | 1-(2-chloro-4-fluorophenyl)-N-((2- | ¹H-NMR (400 MHz, DMSO-d₆) δ 8.17 (dd, J = 7.9, 1.3 |
| | chlorophenyl)sulfonyl)-5-methyl- | Hz, 1H), 7.88 (dd, J = 8.6, 2.9 Hz, 1H), 7.83 (dd, J = 8.8, |
| | 1H-1,2,3-triazole-4-carboxamide | 5.6 Hz, 1H), 7.72-7.66 (m, 2H), 7.63-7.59 (m, 1H), 7.54 |
| | | (td, J = 8.5, 2.9 Hz, 1H), 2.24 (s, 3H); LCMS(M + 1): |
| | | 430.3 |
| 332 | 1-(2-chloro-4-fluorophenyl)-N-((2- | ¹H-NMR (400 MHz, DMSO-d₆) δ 9.18 (s, 1H), 8.37 (d, |
| | chloro-5- | J = 2.2 Hz, 1H), 8.11 (dd, J = 8.6, 2.0 Hz, 1H), 7.94 (d, J = |
| | (trifluoromethyl)phenyl)sulfonyl)- | 8.1 Hz, 1H), 7.87-7.83 (m, 2H), 7.54-7.49 (m, 1H); |
| | 1H-1,2,3-triazole-4-carboxamide | LCMS(M − 1): 482.3 |
| 333 | 1-(2-chloro-4-fluorophenyl)-N-((2,5- | ¹H-NMR (400 MHz, DMSO-d₆) δ 7.88 (dd, J = 8.6, 2.7 |
| | difluorophenyl)sulfonyl)-5-methyl- | Hz, 1H), 7.82 (dd, J = 8.8, 5.6 Hz, 1H), 7.78-7.74 (m, |
| | 1H-1,2,3-triazole-4-carboxamide | 1H), 7.68-7.62 (m, 1H), 7.56-7.51 (m, 2H), 2.26 (s, 3H); |
| | | LCMS(M+): 430.3 |
| 334 | 1-(2,5-dichlorophenyl)-N-((2,5- | ¹H-NMR (400 MHz, DMSO-d₆) δ 12.98 (s, 1H), 7.98 (d, |
| | dimethylphenyl)sulfonyl)-5-methyl- | J = 2.2 Hz, 1H), 7.87-7.80 (m, 3H), 7.39 (d, J = 7.6 Hz, |
| | 1H-1,2,3-triazole-4-carboxamide | 1H), 7.29 (d, J = 7.8 Hz, 1H), 2.59 (s, 3H), 2.36 (s, 3H), |
| | | 2.26 (s, 3H); LCMS (M − 1): 438.45 |
| 335 | 1-(2,5-dichlorophenyl)-5- | ¹H-NMR (400 MHz, DMSO-d₆) δ 8.36 (d, J = 7.6 Hz, |
| | (trifluoromethyl)-N-((2- | 1H), 8.21 (d, J = 1.5 Hz, 1H), 7.99-7.84 (m, 5H); LCMS |
| | (trifluoromethyl)phenyl)sulfonyl)- | (M − 3): 530.25 |
| | 1H-1,2,3-triazole-4-carboxamide | |
| 336 | 1-(2-chloro-4- | ¹H-NMR (400 MHz, DMSO-d₆) δ 8.02 (d, J = 2.2 Hz, |
| | (trifluoromethoxy)phenyl)-N-((2- | 1H), 7.99 (d, J = 8.8 Hz, 1H), 7.70 (d, J = 8.8 Hz, 1H), |
| | chloro-5-methoxyphenyl)sulfonyl)- | 7.62 (d, J = 3.2 Hz, 1H), 7.57 (d, J = 8.6 Hz, 1H), 7.27 |
| | 5-ethyl-1H-1,2,3-triazole-4- | (dd, J = 8.8, 3.2 Hz, 1H), 3.85 (s, 3H), 2.68-2.66 (m, 2H), |
| | carboxamide | 0.89 (t, J = 7.6 Hz, 3H), LCMS(M+): 538.35 |
| 337 | 1-(2-chloro-4- | ¹H-NMR (400 MHz, DMSO-d₆) δ 12.96 (s, 1H), 8.02 (d, |
| | (trifluoromethoxy)phenyl)-N-((2,5- | J = 2.0 Hz, 1H), 7.97 (d, J = 8.8 Hz, 1H), 7.85 (s, 1H), |
| | dimethylphenyl)sulfonyl)-5-ethyl- | 7.70 (d, J = 8.8 Hz, 1H), 7.39 (d, J = 7.6 Hz, 1H), 7.29 (d, |
| | 1H-1,2,3-triazole-4-carboxamide | J = 7.6 Hz, 1H), 2.66 (t, J = 7.6 Hz, 2H), 2.59 (s, 3H), |
| | | 2.37 (s, 3H), 0.89 (t, J = 7.5 Hz, 3H); LCMS(M+): 502.40 |
| 338 | 1-(2-chloro-4- | ¹H-NMR (400 MHz, DMSO-d₆) δ 8.17 (dd, J = 7.9, 1.3 |
| | (trifluoromethoxy)phenyl)-N-((2- | Hz, 1H), 8.02 (d, J = 2.2 Hz, 1H), 7.98 (d, J = 8.8 Hz, |
| | chlorophenyl)sulfonyl)-5-ethyl-1H- | 1H), 7.68 (t, J = 8.2 Hz, 3H), 7.63-7.59 (m, 1H), 2.65 (dd, |
| | 1,2,3-triazole-4-carboxamide | J = 14.4, 7.8 Hz, 2H), 0.88 (t, J = 7.6 Hz, 3H); |
| | | LCMS(M+): 508.30 |
| 339 | 1-(2-chloro-4- | ¹H-NMR (400 MHz, DMSO-d₆) δ 8.02-7.96 (m, 3H), |
| | (trifluoromethoxy)phenyl)-5-ethyl- | 7.77 (s, 1H), 7.70 (d, J = 8.8 Hz, 1H), 7.45 (t, J = 8.1 Hz, |
| | N-((2-fluorophenyl)sulfonyl)-1H- | 2H), 2.68-2.65 (m, 2H), 0.88 (t, J = 7.6 Hz, 3H); |
| | 1,2,3-triazole-4-carboxamide | LCMS(M+): 492.40 |
| 340 | N-((2-chloro-5- | ¹H-NMR (400 MHz, DMSO-d₆) δ 8.30 (d, J = 2.2 Hz, |
| | (trifluoromethyl)phenyl)sulfonyl)-1- | 1H), 8.21 (t, J = 1.1 Hz, 1H), 7.93 (dd, J = 8.3, 1.7 Hz, |
| | (2,5-dichlorophenyl)-5- | 1H), 7.87-7.79 (m, 3H); LCMS (M − 1): 566.10 |
| | (trifluoromethyl)-1H-1,2,3-triazole- | |
| | 4-carboxamide | |
| 341 | N-((2-chlorophenyl)sulfonyl)-1-(2,5- | ¹H-NMR (400 MHz, DMSO-d₆) δ 8.22 (dd, J = 2.1, 0.6 |
| | dichlorophenyl)-5-(trifluoromethyl)- | Hz, 1H), 8.17 (dd, J = 7.8, 1.0 Hz, 1H), 7.90-7.85 (m, |
| | 1H-1,2,3-triazole-4-carboxamide | 2H), 7.72-7.66 (m, 2H), 7.62-7.58 (m, 1H); LCMS (M − |
| | | 1): 498.70 |
| 342 | 1-(2,5-dichlorophenyl)-N-((2- | ¹H-NMR (400 MHz, DMSO-d₆) δ 8.20 (d, J = 1.5 Hz, |
| | fluorophenyl)sulfonyl)-5- | 1H), 8.00-7.96 (m, 1H), 7.90-7.85 (m, 2H), 7.76-7.71 (m, |
| | (trifluoromethyl)-1H-1,2,3-triazole- | 1H), 7.46-7.41 (m, 2H); LCMS (M − 3): 480.20 |
| | 4-carboxamide | |
| 343 | N-((2,5- | ¹H-NMR (400 MHz, DMSO-d₆) δ 8.54 (s, 1H), 8.20 (d, |
| | bis(trifluoromethyl)phenyl)sulfonyl)- | J = 1.7 Hz, 1H), 8.12-8.07 (m, 2H), 7.86-7.81 (m, 2H); |
| | 1-(2,5-dichlorophenyl)-5- | LCMS (M − 3): 598.3 |
| | (trifluoromethyl)-1H-1,2,3-triazole- | |
| | 4-carboxamide | |
| 344 | 1-(2,5-dichlorophenyl)-N-((2,5- | ¹H-NMR (400 MHz, DMSO-d₆) δ 8.21 (q, J = 0.9 Hz, |
| | dimethylphenyl)sulfonyl)-5- | 1H), 7.91-7.88 (m, 2H), 7.86 (d, J = 4.4 Hz, 1H), 7.41 (d, |
| | (trifluoromethyl)-1H-1,2,3-triazole- | J = 6.6 Hz, 1H), 7.32 (d, J = 7.8 Hz, 1H), 2.60 (s, 3H), |
| | 4-carboxamide | 2.37 (s, 3H); LCMS (M − 3): 490.30 |
| 345 | 1-(2,4-dichloro-6-methylphenyl)-N- | ¹H-NMR (400 MHz, CHLOROFORM-D) δ 9.98 (s, 1H), |
| | ((2,5-dichlorophenyl)sulfonyl)-5- | 8.36 (d, J = 2.4 Hz, 1H), 7.55 (dd, J = 8.6, 2.4 Hz, 1H), |
| | methyl-1H-1,2,3-triazole-4- | 7.49-7.47 (m, 2H), 7.35 (q, J = 1.0 Hz, 1H), 2.34 (s, 3H), |
| | carboxamide | 2.00 (s, 3H); LCMS (M + 1): 494.80 |
| 346 | N-((4-chloro-2,5- | ¹H-NMR (400 MHz, DMSO-d₆) δ 8.01 (dd, J = 8.9, 5.5 |
| | dimethylphenyl)sulfonyl)-1-(2- | Hz, 1H), 7.95 (s, 1H), 7.92 (dd, J = 8.6, 2.7 Hz, 1H), 7.59- |
| | chloro-4-fluorophenyl)-5- | 7.54 (m, 1H), 7.49 (s, 1H), 2.58 (s, 3H), 2.38 (s, 3H); |
| | (trifluoromethyl)-1H-1,2,3-triazole- | LCMS(M − 1): 510.8 |
| | 4-carboxamide | |
| 347 | 1-(2-chloro-4-fluorophenyl)-N-((2,5- | ¹H-NMR (400 MHz, DMSO-d₆) δ 8.03-7.99 (m, 2H), |
| | dichlorophenyl)sulfonyl)-5- | 7.90 (dd, J = 8.6, 2.7 Hz, 1H), 7.65 (dd, J = 8.6, 2.7 Hz, |
| | (trifluoromethyl)-1H-1,2,3-triazole- | 1H), 7.60 (d, J = 8.6 Hz, 1H), 7.57-7.52 (m, 1H); |
| | 4-carboxamide | LCMS(M − 1): 516.8 |
| 348 | 1-(2-chloro-4-fluorophenyl)-N-((2- | ¹H-NMR (400 MHz, DMSO-d₆) δ 8.12 (dd, J = 7.8, 1.7 |
| | (trifluoromethoxy)phenyl)sulfonyl)- | Hz, 1H), 8.02 (dd, J = 8.9, 5.5 Hz, 1H), 7.91 (dd, J = 8.4, |
| | 5-(trifluoromethyl)-1H-1,2,3- | 2.8 Hz, 1H), 7.81-7.77 (m, 1H), 7.61-7.58 (m, 1H), 7.57- |
| | triazole-4-carboxamide | 7.54 (m, 2H); LCMS(M+): 532.9 |
| 349 | 1-(2-chloro-4-fluorophenyl)-N-((2- | ¹H-NMR (400 MHz, DMSO-d₆) δ 8.03 (dd, J = 8.9, 5.5 |
| | chloro-5-methoxyphenyl)sulfonyl)- | Hz, 1H), 7.92 (dd, J = 8.6, 2.7 Hz, 1H), 7.59 (q, J = 3.2 |
| | 5-(trifluoromethyl)-1H-1,2,3- | Hz, 1H), 7.57-7.53 (m, 2H), 7.23 (dd, J = 8.9, 3.1 Hz, |
| | triazole-4-carboxamide | 1H), 3.84 (s, 3H); LCMS(M − 2): 511.0 |
| 350 | 1-(2-chloro-4-fluorophenyl)-5- | ¹H-NMR (400 MHz, DMSO-d₆) δ 8.34 (d, J = 7.6 Hz, |
| | (trifluoromethyl)-N-((2- | 1H), 8.01 (dd, J = 8.9, 5.5 Hz, 1H), 7.94 (d, J = 7.8 Hz, |
| | (trifluoromethyl)phenyl)sulfonyl)- | 1H), 7.92-7.88 (m, 2H), 7.83 (t, J = 7.6 Hz, 1H), 7.58- |
| | 1H-1,2,3-triazole-4-carboxamide | 7.53 (m, 1H); LCMS(M − 1): 515.0 |
| 351 | 1-(2-chloro-4-fluorophenyl)-N-((4- | ¹H-NMR (400 MHz, DMSO-d₆) δ 8.11-8.08 (m, 2H), |
| | (trifluoromethoxy)phenyl)sulfonyl)- | 8.00 (dd, J = 8.9, 5.5 Hz, 1H), 7.91 (dd, J = 8.4, 2.8 Hz, |
| | 5-(trifluoromethyl)-1H-1,2,3- | 1H), 7.62-7.53 (m, 3H); LCMS(M − 1): 531.0 |
| | triazole-4-carboxamide | |
| 352 | 1-(2-chloro-4-methoxyphenyl)-N- | ¹H-NMR (400 MHz, DMSO-d₆) δ 8.16 (d, J = 8.9 Hz, |
| | ((2,4-dichlorophenyl)sulfonyl)-5- | 1H), 7.90 (d, J = 1.8 Hz, 1H), 7.71 (dd, J = 8.6, 2.1 Hz, |
| | methyl-1H-1,2,3-triazole-4- | 1H), 7.63 (d, J = 8.9 Hz, 1H), 7.38 (d, J = 2.8 Hz, 1H), |
| | carboxamide | 7.16 (dd, J = 9.0, 2.6 Hz, 1H), 3.87 (s, 3H), 2.22 (s, |
| | | 3H); LCMS(M + 1): 476.25 |
| 353 | 1-(2-chloro-4-methoxyphenyl)-N- | ¹H-NMR (400 MHz, DMSO-d₆) δ 8.07 (td, J = 8.7, 6.3 |
| | ((2,4-difluorophenyl)sulfonyl)-5- | Hz, 1H), 7.62 (d, J = 8.8 Hz, 1H), 7.59-7.54 (m, 1H), |
| | methyl-1H-1,2,3-triazole-4- | 7.38-7.33 (m, 2H), 7.16 (dd, J = 8.8, 2.7 Hz, 1H), 3.87 (s, |
| | carboxamide | 3H), 2.23 (s, 3H); LCMS(M+): 442.40 |
| 354 | 1-(2-chloro-4-methoxyphenyl)-N- | ¹H-NMR (400 MHz, DMSO-d₆) δ 7.79-7.75 (m, 1H), |
| | ((2,5-difluorophenyl)sulfonyl)-5- | 7.70-7.61 (m, 2H), 7.55 (td, J = 9.4, 4.1 Hz, 1H), 7.38 (d, |
| | methyl-1H-1,2,3-triazole-4- | J = 2.7 Hz, 1H), 7.16 (dd, J = 8.9, 2.8 Hz, 1H), 3.88 (d, J |
| | carboxamide | = 7.6 Hz, 3H), 2.24 (s, 3H); LCMS(M+): 442.35 |
| 355 | 1-(2-chloro-4-methoxyphenyl)-5- | ¹H-NMR (400 MHz, DMSO-d₆) δ 7.63 (d, J = 8.8 Hz, |
| | methyl-N-((2,4,6- | 1H), 7.46 (t, J = 9.3 Hz, 2H), 7.38 (d, J = 2.7 Hz, 1H), |
| | trifluorophenyl)sulfonyl)-1H-1,2,3- | 7.16 (dd, J = 8.9, 2.8 Hz, 1H), 3.87 (s, 3H), 2.25 (s, 3H); |
| | triazole-4-carboxamide | LCMS(M+): 460.90 |
| 356 | N-((2-bromo-5- | ¹H-NMR (400 MHz, DMSO-d₆) δ 8.38 (d, J = 2.0 Hz, |
| | (trifluoromethyl)phenyl)sulfonyl)-1- | 1H), 8.12 (d, J = 8.3 Hz, 1H), 7.98 (dd, J = 8.2, 2.1 Hz, |
| | (2-chloro-4-methoxyphenyl)-5- | 1H), 7.62 (d, J = 8.8 Hz, 1H), 7.38 (d, J = 2.7 Hz, 1H), |
| | methyl-1H-1,2,3-triazole-4- | 7.15 (dd, J = 8.9, 2.8 Hz, 1H), 3.89 (d, J = 11.2 Hz, 3H), |
| | carboxamide | 2.23 (s, 2H); LCMS(M+): 554.85 |
| 357 | N-((3,5- | ¹H-NMR (400 MHz, DMSO-d₆) δ 8.56 (s, 3H), 7.59 (d, |
| | bis(trifluoromethyl)phenyl)sulfonyl)- | J = 8.8 Hz, 1H), 7.37 (d, J = 2.7 Hz, 1H), 7.15 (dd, J = 8.8, |
| | 1-(2-chloro-4-methoxyphenyl)-5- | 2.7 Hz, 1H), 3.87 (s, 3H), 2.25 (s, 3H); LCMS(M+): |
| | methyl-1H-1,2,3-triazole-4- | 542.95 |
| | carboxamide | |
| 358 | 1-(2-chloro-4-methoxyphenyl)-5- | ¹H-NMR (400 MHz, DMSO-d₆) δ 8.14 (dd, J = 7.8, 1.7 |
| | methyl-N-((2- | Hz, 1H), 7.84 (td, J = 7.9, 1.5 Hz, 1H), 7.65-7.59 (m, 3H), |
| | (trifluoromethoxy)phenyl)sulfonyl)- | 7.38 (d, J = 2.7 Hz, 1H), 7.15 (dd, J = 8.8, 2.7 Hz, 1H), |
| | 1H-1,2,3-triazole-4-carboxamide | 3.87 (s, 3H), 2.22 (s, 3H); LCMS(M + 1): 491.10 |
| 359 | 1-(2-chloro-4-methoxyphenyl)-5- | ¹H-NMR (400 MHz, DMSO-d₆) δ 12.83 (s, 0H), 8.16- |
| | methyl-N-((4- | 8.13 (m, 2H), 7.65 (d, J = 7.9 Hz, 2H), 7.60 (d, J = 8.9 |
| | (trifluoromethoxy)phenyl)sulfonyl)- | Hz, 1H), 7.38 (d, J = 2.8 Hz, 1H), 7.15 (dd, J = 8.9, 2.8 |
| | 1H-1,2,3-triazole-4-carboxamide | Hz, 1H), 3.87 (s, 3H), 2.24 (s, 3H); LCMS(M+): 490.35 |
| 360 | N-((3,5- | ¹H-NMR (400 MHz, DMSO-d₆) δ 8.40 (s, 2H), 8.36 (s, |
| | bis(trifluoromethyl)phenyl)sulfonyl)- | 1H), 7.97 (dd, J = 8.9, 5.5 Hz, 1H), 7.88 (dd, J = 8.6, 2.7 |
| | 1-(2-chloro-4-fluorophenyl)-5- | Hz, 1H), 7.56-7.51 (m, 1H); LCMS(M − 2): 582.2 |
| | (trifluoromethyl)-1H-1,2,3-triazole- | |
| | 4-carboxamide | |
| 361 | 1-(2-chloro-4-fluorophenyl)-N-((2,4- | ¹H-NMR (400 MHz, DMSO-d₆) δ 8.07-8.00 (m, 2H), |
| | difluorophenyl)sulfonyl)-5- | 7.91 (dd, J = 8.4, 2.8 Hz, 1H), 7.59-7.49 (m, 2H), 7.31 |
| | (trifluoromethyl)-1H-1,2,3-triazole- | (td, J = 8.6, 1.9 Hz, 1H); LCMS(M − 2): 482.2 |
| | 4-carboxamide | |
| 362 | 1-(2-chloro-4-fluorophenyl)-N-((2,5- | ¹H-NMR (400 MHz, DMSO-d₆) δ 8.00 (dd, J = 8.9, 5.5 |
| | difluorophenyl)sulfonyl)-5- | Hz, 1H), 7.90 (dd, J = 8.6, 2.7 Hz, 1H), 7.68-7.64 (m, |
| | (trifluoromethyl)-1H-1,2,3-triazole- | 1H), 7.58-7.50 (m, 2H), 7.44 (q, J = 4.5 Hz, 1H); |
| | 4-carboxamide | LCMS(M − 2): 482.2 |
| 363 | 1-(2-chloro-4-fluorophenyl)-N-((2- | ¹H-NMR (400 MHz, DMSO-d₆) δ 8.04-7.96 (m, 2H), |
| | fluorophenyl)sulfonyl)-5- | 7.92 (dd, J = 8.4, 2.8 Hz, 1H), 7.75 (d, J = 5.9 Hz, 1H), |
| | (trifluoromethyl)-1H-1,2,3-triazole- | 7.59-7.54 (m, 1H), 7.46-7.42 (m, 2H); LCMS(M+): 466.3 |
| | 4-carboxamide | |
| 364 | 1-(2-chloro-4-fluorophenyl)-N-((2- | ¹H-NMR (400 MHz, DMSO-d₆) δ 8.30 (d, J = 2.2 Hz, |
| | chloro-5- | 1H), 8.00 (dd, J = 8.9, 5.5 Hz, 1H), 7.93-7.88 (m, 2H), |
| | (trifluoromethyl)phenyl)sulfonyl)-5- | 7.80 (d, J = 8.1 Hz, 1H), 7.54 (td, J = 8.5, 2.9 Hz, 1H); |
| | (trifluoromethyl)-1H-1,2,3-triazole- | LCMS(M − 2): 549.3 |
| | 4-carboxamide | |
| 365 | 1-(2,5-dichlorophenyl)-N-((2,4- | ¹H-NMR (400 MHz, DMSO-d₆) δ 8.22 (q, J = 1.0 Hz, |
| | difluorophenyl)sulfonyl)-5- | 1H), 8.06 (td, J = 8.6, 6.4 Hz, 1H), 7.92-7.86 (m, 2H), |
| | (trifluoromethyl)-1H-1,2,3-triazole- | 7.57-7.51 (m, 1H), 7.36-7.31 (m, 1H); LCMS (M+): |
| | 4-carboxamide | 500.95 |
| 366 | 1-(2,5-dichlorophenyl)-5- | ¹H-NMR (400 MHz, CHLOROFORM-D) δ 7.63-7.57 |
| | (trifluoromethyl)-N-((2,4,6- | (m, 2H), 7.48 (d, J = 1.7 Hz, 1H), 6.86 (t, J = 8.4 Hz, 2H); |
| | trifluorophenyl)sulfonyl)-1H-1,2,3- | LCMS (M+): 516.20 |
| | triazole-4-carboxamide | |
| 367 | N-((2-chloro-5- | ¹H-NMR (400 MHz, CHLOROFORM-D) δ 10.02 (d, J = |
| | (trifluoromethyl)phenyl)sulfonyl)-5- | 8.8 Hz, 1H), 8.64 (d, J = 2.2 Hz, 1H), 7.84-7.82 (m, 1H), |
| | methyl-1-(2,4,6-trichlorophenyl)- | 7.70 (d, J = 8.3 Hz, 1H), 7.58 (t, J = 3.8 Hz, 2H), 2.36 (s, |
| | 1H-1,2,3-triazole-4-carboxamide | 3H); LCMS (M − 1): 546.80 |
| 368 | N-((2,5-dichlorophenyl)sulfonyl)-5- | ¹H-NMR (400 MHz, CHLOROFORM-D) δ 9.97 (s, 1H), |
| | methyl-1-(2,4,6-trichlorophenyl)- | 8.36 (d, J = 2.4 Hz, 1H), 7.58 (s, 2H), 7.55 (dd, J = 8.4, |
| | 1H-1,2,3-triazole-4-carboxamide | 2.6 Hz, 1H), 7.48 (t, J = 4.3 Hz, 1H), 2.37 (s, 3H); LCMS |
| | | (M + 1): 516.70 |
| 369 | N-((2-bromo-5- | ¹H-NMR (400 MHz, CHLOROFORM-D) δ 10.03 (s, |
| | (trifluoromethyl)phenyl)sulfonyl)-5- | 1H), 8.66 (d, J = 2.0 Hz, 1H), 7.91 (d, J = 8.1 Hz, 1H), |
| | methyl-1-(2,4,6-trichlorophenyl)- | 7.74-7.71 (m, 1H), 7.58 (s, 2H), 2.36 (s, 3H); LCMS |
| | 1H-1,2,3-triazole-4-carboxamide | (M + 1): 592.75 |
| 370 | 1-(2,5-dichlorophenyl)-N-((2- | ¹H-NMR (400 MHz, DMSO-d₆) δ 8.22 (d, J = 1.2 Hz, |
| | (trifluoromethoxy)phenyl)sulfonyl)- | 1H), 8.14 (dd, J = 7.9, 1.5 Hz, 1H), 7.90-7.80 (m, 3H), |
| | 5-(trifluoromethyl)-1H-1,2,3- | 7.64-7.57 (m, 2H); LCMS (M − 3): 546.55 |
| | triazole-4-carboxamide | |
| 371 | 1-(2,5-dichlorophenyl)-N-((2,5- | ¹H-NMR (400 MHz, DMSO-d₆) δ 8.21 (d, J = 1.8 Hz, |
| | dichlorophenyl)sulfonyl)-5- | 1H), 8.06 (d, J = 2.4 Hz, 1H), 7.89-7.83 (m, 2H), 7.71 |
| | (trifluoromethyl)-1H-1,2,3-triazole- | (dd, J = 8.6, 2.4 Hz, 1H), 7.65 (d, J = 8.6 Hz, 1H); LCMS |
| | 4-carboxamide | (M − 2): 532.70 |
| 372 | N-((2-bromo-5- | ¹H-NMR (400 MHz, DMSO-d₆) δ 8.37 (d, J = 2.1 Hz, |
| | (trifluoromethyl)phenyl)sulfonyl)-1- | 1H), 8.10 (d, J = 8.6 Hz, 1H), 7.95 (dd, J = 8.4, 2.0 Hz, |
| | (2-chloro-4-fluorophenyl)-5-ethyl- | 1H), 7.90-7.86 (m, 2H), 7.54 (td, J = 8.6, 2.8 Hz, 1H), |
| | 1H-1,2,3-triazole-4-carboxamide | 2.68-2.65 (m, 2H), 0.88 (t, J = 7.5 Hz, 3H); LCMS(M + 1): |
| | | 556.8 |
| 373 | N-((3,5- | ¹H-NMR (400 MHz, DMSO-d₆) δ 8.55 (s, 3H), 7.90-7.83 |
| | bis(trifluoromethyl)phenyl)sulfonyl)- | (m, 2H), 7.54 (td, J = 8.5, 2.9 Hz, 1H), 2.72-2.65 (m, 2H), |
| | 1-(2-chloro-4-fluorophenyl)-5-ethyl- | 0.89 (t, J = 7.6 Hz, 3H); LCMS(M + 1): 545.0 |
| | 1H-1,2,3-triazole-4-carboxamide | |
| 374 | 1-(2-chloro-4-fluorophenyl)-N-((2,5- | ¹H-NMR (400 MHz, DMSO-d₆) δ 7.91-7.87 (m, 2H), |
| | difluorophenyl)sulfonyl)-5-ethyl-1H- | 7.80-7.64 (m, 2H), 7.59-7.52 (m, 2H), 2.65 (d, J = 6.6 Hz, |
| | 1,2,3-triazole-4-carboxamide | 2H), 0.89 (t, J = 7.5 Hz, 3H); LCMS(M+): 444.8 |
| 375 | 1-(2-chloro-4-fluorophenyl)-N-((2,5- | ¹H-NMR (400 MHz, DMSO-d₆) δ 8.10 (d, J = 2.8 Hz, |
| | dichlorophenyl)sulfonyl)-5-ethyl- | 1H), 7.91-7.87 (m, 2H), 7.79 (dd, J = 8.6, 2.4 Hz, 1H), |
| | 1H-1,2,3-triazole-4-carboxamide | 7.71 (d, J = 8.6 Hz, 1H), 7.54 (td, J = 8.4, 2.8 Hz, 1H), |
| | | 2.66 (q, J = 1.8 Hz, 2H), 0.89 (t, J = 7.6 Hz, 3H); |
| | | LCMS(M + 1): 478.8 |
| 376 | 1-(2-chloro-4-fluorophenyl)-5-ethyl- | ¹H-NMR (400 MHz, DMSO-d₆) δ 8.14 (dd, J = 7.9, 1.6 |
| | N-((2- | Hz, 1H), 7.89 (dt, J = 8.6, 2.6 Hz, 2H), 7.83 (s, 1H), 7.65- |
| | (trifluoromethoxy)phenyl)sulfonyl)- | 7.52 (m, 3H), 2.65 (s, 2H), 0.87 (t, J = 7.5 Hz, 3H); |
| | 1H-1,2,3-triazole-4-carboxamide | LCMS(M+): 492.9 |
| 377 | 1-(2-chloro-4-fluorophenyl)-N-((2- | ¹H-NMR (400 MHz, DMSO-d₆) δ 8.17 (dd, J = 8.1, 1.4 |
| | chlorophenyl)sulfonyl)-5-ethyl-1H- | Hz, 1H), 7.91-7.88 (m, 2H), 7.69 (td, J = 8.3, 6.4 Hz, 2H), |
| | 1,2,3-triazole-4-carboxamide | 7.63-7.59 (m, 1H), 7.54 (td, J = 8.6, 2.8 Hz, 1H), 2.66- |
| | | 2.63 (m, 2H), 0.87 (t, J = 7.5 Hz, 3H); LCMS(M − 1): 442.8 |
| 378 | 1-(2-chloro-4-fluorophenyl)-5-ethyl- | ¹H-NMR (400 MHz, DMSO-d₆) δ 8.59 (s, 1H), 8.23 (dd, |
| | N-((2-fluorophenyl)sulfonyl)-1H- | J = 20.6, 8.7 Hz, 2H), 7.89-7.85 (m, 2H), 7.53 (td, J = 8.4, |
| | 1,2,3-triazole-4-carboxamide | 2.8 Hz, 1H), 2.69-2.64 (m, 2H), 0.88 (t, J = 7.5 Hz, 3H); |
| | | LCMS(M + 1): 443.1 |
| 379 | 1-(2-chloro-4-fluorophenyl)-N-((2- | ¹H-NMR (400 MHz, DMSO-d₆) δ 7.92-7.88 (m, 2H), |
| | chloro-5-methoxyphenyl)sulfonyl)- | 7.62-7.52 (m, 3H), 7.28 (dd, J = 8.8, 3.2 Hz, 1H), 3.85 (s, |
| | 5-ethyl-1H-1,2,3-triazole-4- | 3H), 2.65 (d, J = 7.3 Hz, 2H), 0.90-0.85 (m, 3H); |
| | carboxamide | LCMS(M − 1): 472.9 |
| 380 | 1-(2-chloro-4-fluorophenyl)-5-ethyl- | ¹H-NMR (400 MHz, DMSO-d₆) δ 7.90-7.87 (m, 2H), |
| | N-((2,4,6-trifluorophenyl)sulfonyl)- | 7.54 (td, J = 8.4, 2.9 Hz, 1H), 7.43 (t, J = 9.3 Hz, 2H), |
| | 1H-1,2,3-triazole-4-carboxamide | 2.69-2.64 (m, 3H), 0.90 (t, J = 7.5 Hz, 3H); LCMS(M+): |
| | | 462.8 |
| 381 | 1-(2-chloro-4-fluorophenyl)-N-((2- | ¹H-NMR (400 MHz, DMSO-d₆) δ 8.36 (d, J = 1.8 Hz, |
| | chloro-5- | 1H), 8.09 (dd, J = 8.4, 2.0 Hz, 1H), 7.94 (d, J = 8.3 Hz, |
| | (trifluoromethyl)phenyl)sulfonyl)-5- | 1H), 7.90-7.87 (m, 2H), 7.54 (td, J = 8.6, 2.8 Hz, 1H), |
| | ethyl-1H-1,2,3-triazole-4- | 2.66 (t, J = 1.8 Hz, 2H), 0.88 (t, J = 7.6 Hz, 3H); |
| | carboxamide | LCMS(M − 1): 510.9 |
| 382 | 1-(2-chloro-4-fluorophenyl)-N-((2,4- | ¹H-NMR (400 MHz, DMSO-d₆) δ 8.07 (td, J = 8.6, 6.4 |
| | difluorophenyl)sulfonyl)-5-ethyl-1H- | Hz, 1H), 7.91-7.86 (m, 2H), 7.55 (qd, J = 8.5, 2.8 Hz, |
| | 1,2,3-triazole-4-carboxamide | 2H), 7.35 (td, J = 8.4, 2.1 Hz, 1H), 2.66-2.64 (m, 2H), |
| | | 0.89 (t, J = 7.5 Hz, 3H); LCMS(M+): 444.8 |
| 383 | 1-(2-chloro-4-fluorophenyl)-5-ethyl- | ¹H-NMR (400 MHz, DMSO-d₆) δ 8.37 (d, J = 7.6 Hz, |
| | N-((2- | 1H), 8.01 (d, J = 7.6 Hz, 1H), 7.95 (d, J = 7.6 Hz, 1H), |
| | (trifluoromethyl)phenyl)sulfonyl)- | 7.90 (td, J = 8.6, 5.7 Hz, 3H), 7.54 (td, J = 8.5, 3.0 Hz, |
| | 1H-1,2,3-triazole-4-carboxamide | 1H), 2.66-2.64 (m, 2H), 0.88 (t, J = 7.6 Hz, 3H); |
| | | LCMS(M+): 476.9 |
| 384 | 1-(2-chloro-4-fluorophenyl)-N-((2,5- | ¹H-NMR (400 MHz, DMSO-d₆) δ 7.86 (d, J = 8.3 Hz, |
| | dimethylphenyl)sulfonyl)-5-ethyl- | 2H), 7.52 (d, J = 7.9 Hz, 1H), 7.30-7.18 (m, 1H), 2.77- |
| | 1H-1,2,3-triazole-4-carboxamide | 2.64 (m, 2H), 2.54 (d, J = 7.0 Hz, 3H), 2.33-2.31 (m, 3H), |
| | | 0.91-0.84 (m, 3H); LCMS(M + 1): 437.0 |
| 385 | N-((2,5- | ¹H-NMR (400 MHz, DMSO-d₆) δ 8.00 (td, J = 7.6, 1.4 |
| | bis(trifluoromethyl)phenyl)sulfonyl)- | Hz, 1H), 7.90-7.86 (m, 2H), 7.79-7.74 (m, 1H), 7.54 (td, |
| | 1-(2-chloro-4-fluorophenyl)-5-ethyl- | J = 8.4, 2.8 Hz, 1H), 7.48-7.43 (m, 2H), 2.66-2.63 (m, |
| | 1H-1,2,3-triazole-4-carboxamide | 2H), 0.88 (t, J = 7.5 Hz, 3H); LCMS(M − 2): 542.3 |
| 386 | 1-(2-chloro-4-fluorophenyl)-N-((2,4- | ¹H-NMR (400 MHz, DMSO-d₆) δ 8.16 (d, J = 8.9 Hz, |
| | dichlorophenyl)sulfonyl)-5-ethyl- | 1H), 7.91-7.86 (m, 3H), 7.71 (dd, J = 8.6, 1.8 Hz, 1H), |
| | 1H-1,2,3-triazole-4-carboxamide | 7.54 (td, J = 8.6, 2.8 Hz, 1H), 2.66-2.64 (m, 2H), 0.88 (t, |
| | | J = 7.5 Hz, 3H), LCMS(M + 1): 478.8 |
| 387 | 1-(2-chloro-4-cyanophenyl)-N-((2- | ¹H-NMR (400 MHz, DMSO-d₆) δ 8.49 (d, J = 1.0 Hz, |
| | chloro-5- | 1H), 8.30 (d, J = 2.3 Hz, 1H), 8.16 (d, J = 1.0 Hz, 2H), |
| | (trifluoromethyl)phenyl)sulfonyl)-5- | 7.93 (dd, J = 8.3, 1.7 Hz, 1H), 7.80 (d, J = 8.3 Hz, 1H); |
| | (trifluoromethyl)-1H-1,2,3-triazole- | LCMS (M − 3): 555.50 |
| | 4-carboxamide | |
| 388 | 1-(2-chloro-4-cyanophenyl)-N-((2,5- | ¹H-NMR (400 MHz, DMSO-d₆) δ 8.50 (t, J = 1.1 Hz, 1H), |
| | dichlorophenyl)sulfonyl)-5- | 8.17 (d, J = 1.0 Hz, 2H), 8.04 (d, J = 2.4 Hz, 1H), 7.66 |
| | (trifluoromethyl)-1H-1,2,3-triazole- | (dd, J = 8.4, 2.6 Hz, 1H), 7.61 (d, J = 8.3 Hz, 1H); LCMS |
| | 4-carboxamide | (M − 3): 523.40 |
| 389 | 1-(2-chloro-4-cyanophenyl)-N-((2,5- | ¹H-NMR (400 MHz, DMSO-d₆) δ 8.52 (q, J = 0.7 Hz, |
| | dimethylphenyl)sulfonyl)-5- | 1H), 8.21-8.16 (m, 2H), 7.85 (s, 1H), 7.41 (d, J = 7.8 Hz, |
| | (trifluoromethyl)-1H-1,2,3-triazole- | 1H), 7.31 (d, J = 7.8 Hz, 1H), 2.59 (d, J = 12.0 Hz, 3H), |
| | 4-carboxamide | 2.37 (s, 3H); LCMS (M − 2): 581.50 |
| 390 | N-((2,5- | ¹H-NMR (400 MHz, DMSO-d₆) δ 8.54 (s, 1H), 8.48 (t, |
| | bis(trifluoromethyl)phenyl)sulfonyl)- | J = 1.0 Hz, 1H), 8.16 (d, J = 0.7 Hz, 2H), 8.12-8.07 (m, |
| | 1-(2-chloro-4-cyanophenyl)-5- | 2H); LCMS (M − 2): 589.60 |
| | (trifluoromethyl)-1H-1,2,3-triazole- | |
| | 4-carboxamide | |
| 391 | 1-(2-chloro-4-cyanophenyl)-N-((2,5- | ¹H-NMR (400 MHz, DMSO-d₆) δ 8.50 (q, J = 0.7 Hz, |
| | difluorophenyl)sulfonyl)-5- | 1H), 8.19-8.15 (m, 2H), 7.69-7.65 (m, 1H), 7.56-7.50 (m, |
| | (trifluoromethyl)-1H-1,2,3-triazole- | 1H), 7.43 (td, J = 9.2, 4.1 Hz, 1H); LCMS (M − 2): 489.45 |
| | 4-carboxamide | |
| 392 | 1-(2-chloro-4-cyanophenyl)-N-((2- | ¹H-NMR (400 MHz, DMSO-d₆) δ 8.52 (t, J = 1.1 Hz, 1H), |
| | chlorophenyl)sulfonyl)-5- | 8.18 (d, J = 1.2 Hz, 2H), 8.16-8.14 (m, 1H), 7.66-7.63 (m, |
| | (trifluoromethyl)-1H-1,2,3-triazole- | 2H), 7.60-7.56 (m, 1H); LCMS (M − 3): 487.45 |
| | 4-carboxamide | |
| 393 | 1-(2-chloro-4-cyanophenyl)-N-((2- | ¹H-NMR (400 MHz, DMSO-d₆) δ 8.52 (q, J = 0.7 Hz, |
| | fluorophenyl)sulfonyl)-5- | 1H), 8.20-8.15 (m, 2H), 8.00-7.95 (m, 1H), 7.75-7.70 (m, |
| | (trifluoromethyl)-1H-1,2,3-triazole- | 1H), 7.44-7.40 (m, 2H); LCMS (M − 3): 471.45 |
| | 4-carboxamide | |
| 394 | 1-(2-chloro-4-cyanophenyl)-5- | ¹H-NMR (400 MHz, DMSO-d₆) δ 8.51 (q, J = 0.7 Hz, |
| | (trifluoromethyl)-N-((2- | 1H), 8.34 (d, J = 7.6 Hz, 1H), 8.17 (dd, J = 9.7, 8.2 Hz, |
| | (trifluoromethyl)phenyl)sulfonyl)- | 2H), 7.95-7.81 (m, 3H); LCMS (M − 2): 521.55 |
| | 1H-1,2,3-triazole-4-carboxamide | |
| 395 | 1-(2-chloro-4-cyanophenyl)-N-((2- | ¹H-NMR (400 MHz, DMSO-d₆) δ 8.51 (t, J = 1.1 Hz, 1H), |
| | (trifluoromethoxy)phenyl)sulfonyl)- | 8.18-8.16 (m, 2H), 8.11 (dd, J = 7.9, 1.5 Hz, 1H), 7.78 |
| | 5-(trifluoromethyl)-1H-1,2,3- | (td, J = 7.9, 1.5 Hz, 1H), 7.60-7.56 (m, 1H), 7.54 (d, J = |
| | triazole-4-carboxamide | 8.3 Hz, 1H); LCMS (M − 2): 537.50 |
| 396 | 1-(2-chloro-4-cyanophenyl)-N-(o- | ¹H-NMR (400 MHz, DMSO-d₆) δ 8.52 (d, J = 0.9 Hz, |
| | tolylsulfonyl)-5-(trifluoromethyl)- | 1H), 8.20-8.15 (m, 2H), 8.04 (d, J = 7.6 Hz, 1H), 7.59 (t, |
| | 1H-1,2,3-triazole-4-carboxamide | J = 7.3 Hz, 1H), 7.44 (q, J = 7.7 Hz, 2H), 2.65 (s, 3H); |
| | | LCMS (M+): 469.40 |
| 397 | 1-(2-chloro-4-cyanophenyl)-5- | ¹H-NMR (400 MHz, DMSO-d₆) δ 8.43-8.48 (1H), 8.10- |
| | (trifluoromethyl)-N-((2,4,6- | 8.15 (2H), 7.17-7.28 (2H); LCMS (M+): 509.60 |
| | trifluorophenyl)sulfonyl)-1H-1,2,3- | |
| | triazole-4-carboxamide | |
| 398 | 1-(2-chloro-4-methoxyphenyl)-N- | ¹H-NMR (400 MHz, DMSO-d₆) δ 7.79 (d, J = 8.9 Hz, |
| | ((2-chloro-5- | 1H), 7.60 (d, J = 3.1 Hz, 1H), 7.54-7.52 (m, 1H), 7.39 (d, |
| | methoxyphenyl)sulfonyl)-5- | J = 2.4 Hz, 1H), 7.23-7.21 (m, 1H), 7.16 (dd, J = 8.9, 2.8 |
| | (trifluoromethyl)-1H-1,2,3-triazole- | Hz, 1H), 3.88 (s, 3H), 3.84 (s, 3H); LCMS(M+): 524.85 |
| | 4-carboxamide | |
| 399 | 1-(2-chloro-4-methoxyphenyl)-N- | ¹H-NMR (400 MHz, DMSO-d₆) δ 8.30 (d, J = 2.1 Hz, |
| | ((2-chloro-5- | 1H), 7.95 (dd, J = 8.3, 2.1 Hz, 1H), 7.82 (d, J = 8.3 Hz, |
| | (trifluoromethyl)phenyl)sulfonyl)-5- | 1H), 7.77 (d, J = 8.9 Hz, 1H), 7.37 (d, J = 2.4 Hz, 1H), |
| | (trifluoromethyl)-1H-1,2,3-triazole- | 7.14 (dd, J = 8.9, 2.8 Hz, 1H), 3.88 (s, 3H); LCMS(M − 2): |
| | 4-carboxamide | 560.85 |
| 400 | 1-(2-chloro-4-methoxyphenyl)-N- | ¹H-NMR (400 MHz, DMSO-d₆) δ 8.06 (d, J = 2.8 Hz, |
| | ((2,5-dichlorophenyl)sulfonyl)-5- | 1H), 7.79 (d, J = 8.9 Hz, 1H), 7.72 (dd, J = 8.6, 2.8 Hz, |
| | (trifluoromethyl)-1H-1,2,3-triazole- | 1H), 7.66 (d, J = 8.6 Hz, 1H), 7.39 (d, J = 2.8 Hz, 1H), |
| | 4-carboxamide | 7.16 (dd, J = 8.9, 2.8 Hz, 1H), 3.89 (d, J = 8.6 Hz, 3H); |
| | | LCMS(M + 1): 530.80 |
| 401 | 1-(2-chloro-4-methoxyphenyl)-N- | ¹H-NMR (400 MHz, DMSO-d₆) δ 7.99 (td, J = 7.7, 1.6 |
| | ((2-fluorophenyl)sulfonyl)-5- | Hz, 1H), 7.80-7.73 (m, 2H), 7.48-7.42 (m, 2H), 7.39 (d, |
| | (trifluoromethyl)-1H-1,2,3-triazole- | J = 2.8 Hz, 1H), 7.17 (dd, J = 9.2, 2.8 Hz, 1H), 3.88 (s, 3H); |
| | 4-carboxamide | LCMS(M + 1): 476.90 |
| 402 | 1-(2-chloro-4-methoxyphenyl)-5- | ¹H-NMR (400 MHz, DMSO-d₆) δ 8.35 (d, J = 7.6 Hz, |
| | (trifluoromethyl)-N-((2- | 1H), 7.96 (d, J = 7.6 Hz, 1H), 7.93-7.89 (m, 1H), 7.85 (t, |
| | (trifluoromethyl)phenyl)sulfonyl)- | J = 7.5 Hz, 1H), 7.78 (d, J = 8.9 Hz, 1H), 7.33-7.40 (1H), |
| | 1H-1,2,3-triazole-4-carboxamide | 7.15 (d, J = 2.8 Hz, 1H), 3.88 (s, 3H); LCMS(M+): 528.90 |
| 403 | 1-(2-chloro-4-methoxyphenyl)-N- | ¹H-NMR (400 MHz, DMSO-d₆) δ 8.17-8.14 (m, 1H), |
| | ((2-chlorophenyl)sulfonyl)-5- | 7.80 (d, J = 8.9 Hz, 1H), 7.70-7.65 (m, 2H), 7.61-7.57 (m, |
| | (trifluoromethyl)-1H-1,2,3-triazole- | 1H), 7.39 (d, J = 2.8 Hz, 1H), 7.16 (dd, J = 8.9, 2.8 Hz, |
| | 4-carboxamide | 1H), 3.88 (s, 3H); LCMS(M+): 495.05 |
| 404 | 1-(2-chloro-4-methoxyphenyl)-N- | ¹H-NMR (400 MHz, DMSO-d₆) δ 8.14 (dd, J = 7.8, 1.7 |
| | ((2- | Hz, 1H), 7.85-7.79 (m, 2H), 7.64-7.57 (m, 2H), 7.42-7.36 |
| | (trifluoromethoxy)phenyl)sulfonyl)- | (m, 1H), 7.16 (dd, J = 8.9, 2.8 Hz, 1H), 3.90 (d, J = 15.0 |
| | 5-(trifluoromethyl)-1H-1,2,3- | Hz, 3H); LCMS(M+): 544.95 |
| | triazole-4-carboxamide | |
| 405 | 1-(2-chloro-4-methoxyphenyl)-N- | ¹H-NMR (400 MHz, DMSO-d₆) δ 7.77 (d, J = 8.9 Hz, |
| | ((2,5-difluorophenyl)sulfonyl)-5- | 1H), 7.69-7.65 (m, 1H), 7.56-7.50 (m, 1H), 7.44 (td, J = |
| | (trifluoromethyl)-1H-1,2,3-triazole- | 9.2, 4.2 Hz, 1H), 7.38 (d, J = 2.4 Hz, 1H), 7.15 (dd, J = |
| | 4-carboxamide | 8.9, 2.8 Hz, 1H), 3.88 (s, 3H); LCMS(M+): 496.90 |
| 406 | 1-(2-chloro-4-methoxyphenyl)-N- | ¹H-NMR (400 MHz, DMSO-d₆) δ 8.04 (td, J = 8.6, 6.2 |
| | ((2,4-difluorophenyl)sulfonyl)-5- | Hz, 1H), 7.78 (d, J = 8.9 Hz, 1H), 7.56-7.50 (m, 1H), 7.39 |
| | (trifluoromethyl)-1H-1,2,3-triazole- | (d, J = 2.8 Hz, 1H), 7.32 (td, J = 8.4, 2.1 Hz, 1H), 7.16 |
| | 4-carboxamide | (dd, J = 8.9, 2.8 Hz, 1H), 3.88 (s, 3H); LCMS(M+): |
| | | 496.85 |
| 407 | 1-(2-chloro-4-methoxyphenyl)-5- | ¹H-NMR (400 MHz, DMSO-d₆) δ 7.78 (d, J = 8.9 Hz, |
| | (trifluoromethyl)-N-((2,4,6- | 1H), 7.38-7.32 (m, 3H), 7.16 (dd, J = 8.9, 2.8 Hz, 1H), |
| | trifluorophenyl)sulfonyl)-1H-1,2,3- | 3.89 (d, J = 6.1 Hz, 3H); LCMS(M+): 514.55 |
| | triazole-4-carboxamide | |
| 408 | N-((3,5- | ¹H-NMR (400 MHz, DMSO-d₆) δ 8.38 (d, J = 17.4 Hz, |
| | bis(trifluoromethyl)phenyl)sulfonyl)- | 3H), 7.73 (d, J = 8.9 Hz, 1H), 7.36 (d, J = 2.8 Hz, 1H), |
| | 1-(2-chloro-4-methoxyphenyl)-5- | 7.13 (dd, J = 9.2, 2.8 Hz, 1H), 3.88 (d, J = 5.2 Hz, 3H); |
| | (trifluoromethyl)-1H-1,2,3-triazole- | LCMS(M − 2): 593.85 |
| | 4-carboxamide | |
| 409 | N-((2-chloro-5- | ¹H-NMR (400 MHz, DMSO-d₆) δ 8.36 (d, J = 2.2 Hz, |
| | (trifluoromethyl)phenyl)sulfonyl)-1- | 1H), 8.32 (dd, J = 9.9, 1.3 Hz, 1H), 8.10-8.08 (m, 1H), |
| | (4-cyano-2-fluorophenyl)-5-methyl- | 8.03-7.92 (m, 3H), 2.35 (s, 3H); LCMS (M+): 487.90 |
| | 1H-1,2,3-triazole-4-carboxamide | |
| 410 | 1-(4-cyano-2-fluorophenyl)-N-((2,5- | ¹H-NMR (400 MHz, DMSO-d₆) δ 8.32 (dd, J = 9.9, 1.3 |
| | dichlorophenyl)sulfonyl)-5-methyl- | Hz, 1H), 8.10 (d, J = 2.4 Hz, 1H), 8.02 (dd, J = 8.4, 1.6 |
| | 1H-1,2,3-triazole-4-carboxamide | Hz, 1H), 7.99-7.95 (m, 1H), 7.79 (dd, J = 8.6, 2.7 Hz, |
| | | 1H), 7.71 (d, J = 8.6 Hz, 1H), 2.36 (s, 3H); LCMS (M+): |
| | | 453.80 |
| 411 | 1-(4-cyano-2-fluorophenyl)-N-((2,5- | ¹H-NMR (400 MHz, DMSO-d₆) δ 12.90 (s, 1H), 8.31 (dd, |
| | dimethylphenyl)sulfonyl)-5-methyl- | J = 9.9, 1.7 Hz, 1H), 8.03-7.94 (m, 2H), 7.86 (s, 1H), 7.39 |
| | 1H-1,2,3-triazole-4-carboxamide | (d, J = 6.4 Hz, 1H), 7.28 (d, J = 7.9 Hz, 1H), 2.61 (s, 3H), |
| | | 2.38 (s, 3H), 2.36 (s, 3H) LCMS (M+): 413.95 |
| 412 | N-((2,5- | ¹H-NMR (400 MHz, DMSO-d₆) δ 8.62 (s, 1H), 8.32 (dd, |
| | bis(trifluoromethyl)phenyl)sulfonyl)- | J = 10.1, 1.5 Hz, 2H), 8.25 (d, J = 8.3 Hz, 1H), 8.02 (dd, |
| | 1-(4-cyano-2-fluorophenyl)-5- | J = 8.4, 1.7 Hz, 1H), 7.97 (t, J = 7.8 Hz, 1H), 2.36 (s, 3H) |
| | methyl-1H-1,2,3-triazole-4- | LCMS (M+): 521.90 |
| | carboxamide | |
| 413 | 1-(4-cyano-2-fluorophenyl)-N-((2,4- | ¹H-NMR (400 MHz, DMSO-d₆) δ 8.32 (dd, J = 10.1, 1.5 |
| | difluorophenyl)sulfonyl)-5-methyl- | Hz, 1H), 8.09-8.04 (m, 1H), 8.02 (dd, J = 8.3, 1.2 Hz, |
| | 1H-1,2,3-triazole-4-carboxamide | 1H), 7.96 (t, J = 7.6 Hz, 1H), 7.55 (s, 1H), 7.35 (d, J = 2.4 |
| | | Hz, 1H), 2.35 (s, 3H); LCMS (M+) = 421.85 |
| 414 | N-((2-chlorophenyl)sulfonyl)-1-(4- | ¹H-NMR (400 MHz, DMSO-d₆) δ 8.32 (dd, J = 10.1, 1.5 |
| | cyano-2-fluorophenyl)-5-methyl-1H- | Hz, 1H), 8.17 (dd, J = 7.8, 1.4 Hz, 1H), 8.02 (dd, J = 8.4, |
| | 1,2,3-triazole-4-carboxamide | 1.7 Hz, 1H), 7.99-7.96 (m, 1H), 7.71-7.66 (m, 2H), 7.63- |
| | | 7.59 (m, 1H), 2.33 (s, 3H); LCMS (M+) = 419.80 |
| 415 | 1-(4-cyano-2-fluorophenyl)-N-((2- | ¹H-NMR (400 MHz, DMSO-d₆) δ 8.32 (dd, J = 9.9, 1.4 |
| | fluorophenyl)sulfonyl)-5-methyl-1H- | Hz, 1H), 8.03-7.94 (m, 3H), 7.78-7.75 (m, 1H), 7.48-7.43 |
| | 1,2,3-triazole-4-carboxamide | (m, 2H), 2.34 (s, 3H) LCMS (M+) = 403.90 |
| 416 | 1-(4-cyano-2-fluorophenyl)-5- | ¹H-NMR (400 MHz, DMSO-d₆) δ 8.38-8.31 (m, 2H), |
| | methyl-N-((2- | 8.04-7.89 (m, 5H), 2.34 (s, 3H); LCMS (M+) = 453.90 |
| | (trifluoromethyl)phenyl)sulfonyl)- | |
| | 1H-1,2,3-triazole-4-carboxamide | |
| 417 | 1-(4-cyano-2-fluorophenyl)-5- | ¹H-NMR (400 MHz, DMSO-d₆) δ 8.32 (dd, J = 10.0, 1.5 |
| | methyl-N-((2- | Hz, 1H), 8.14 (dd, J = 7.9, 1.6 Hz, 1H), 8.04-7.96 (m, |
| | (trifluoromethoxy)phenyl)sulfonyl)- | 2H), 7.84 (td, J = 7.9, 1.5 Hz, 1H), 7.65-7.59 (m, 2H), |
| | 1H-1,2,3-triazole-4-carboxamide | 2.34 (s, 3H); LCMS (M+) = 469.90 |
| 418 | N-((3,5- | ¹H-NMR (400 MHz, DMSO-d₆) δ 8.54 (s, 3H), 8.32 (dd, |
| | bis(trifluoromethyl)phenyl)sulfonyl)- | J = 10.0, 1.5 Hz, 1H), 8.01 (dd, J = 8.3, 1.2 Hz, 1H), 7.96- |
| | 1-(4-cyano-2-fluorophenyl)-5- | 7.92 (m, 1H), 2.37 (s, 3H); LCMS (M − 2): 519.55 |
| | methyl-1H-1,2,3-triazole-4- | |
| | carboxamide | |
| 419 | 1-(4-cyano-2-fluorophenyl)-5- | ¹H-NMR (400 MHz, DMSO-d₆) δ 8.33 (dd, J = 10.0, 1.5 |
| | methyl-N-((2,4,6- | Hz, 1H), 8.04-7.96 (m, 2H), 7.45 (t, J = 9.3 Hz, 2H), 2.37 |
| | trifluorophenyl)sulfonyl)-1H-1,2,3- | (s, 3H); LCMS (M − 2): 439.50 |
| | triazole-4-carboxamide | |
| 420 | N-((2-chloro-5- | ¹H-NMR (400 MHz, CHLOROFORM-D) δ 9.95 (s, 1H), |
| | methoxyphenyl)sulfonyl)-5-methyl- | 7.86 (d, J = 3.1 Hz, 1H), 7.58 (s, 2H), 7.42 (d, J = 8.9 Hz, |
| | 1-(2,4,6-trichlorophenyl)-1H-1,2,3- | 1H), 7.10 (dd, J = 8.9, 3.1 Hz, 1H), 3.90 (s, 3H), 2.36 (s, |
| | triazole-4-carboxamide | 3H); LCMS (M + 1): 510.40 |
| 421 | N-((2,5-dimethylphenyl)sulfonyl)-5- | ¹H-NMR (400 MHz, CHLOROFORM-D) δ 9.73 (s, 1H), |
| | methyl-1-(2,4,6-trichlorophenyl)- | 8.10 (d, J = 1.2 Hz, 1H), 7.58 (t, J = 3.7 Hz, 2H), 7.34 (dd, |
| | 1H-1,2,3-triazole-4-carboxamide | J = 7.6, 1.2 Hz, 1H), 7.22 (t, J = 3.8 Hz, 1H), 2.71 (s, 3H), |
| | | 2.43 (s, 3H), 2.36 (s, 3H); LCMS (M + 1): 474.55 |
| 422 | N-((2-fluorophenyl)sulfonyl)-5- | ¹H-NMR (400 MHz, CHLOROFORM-D) δ 9.89 (s, 1H), |
| | methyl-1-(2,4,6-trichlorophenyl)- | 8.22-8.18 (m, 1H), 7.70-7.64 (m, 1H), 7.58 (d, J = 3.7 Hz, |
| | 1H-1,2,3-triazole-4-carboxamide | 2H), 7.38 (td, J = 7.7, 1.0 Hz, 1H), 7.24 (dd, J = 8.7, 1.1 |
| | | Hz, 1H), 2.36 (s, 3H); LCMS (M − 1): 462.35 |
| 423 | N-((2-chlorophenyl)sulfonyl)-5- | ¹H-NMR (400 MHz, CHLOROFORM-D) δ 9.95 (s, 1H), |
| | methyl-1-(2,4,6-trichlorophenyl)- | 8.38 (dd, J = 7.6, 1.5 Hz, 1H), 7.61-7.50 (m, 5H), 2.35 (s, |
| | 1H-1,2,3-triazole-4-carboxamide | 3H); LCMS (M + 1): 480.45 |
| 424 | 5-methyl-1-(2,4,6-trichlorophenyl)- | ¹H-NMR (400 MHz, CHLOROFORM-D) δ 9.88 (s, 1H), |
| | N-((2- | 8.34 (dd, J = 7.9, 1.8 Hz, 1H), 7.73-7.69 (m, 1H), 7.58 (t, |
| | (trifluoromethoxy)phenyl)sulfonyl)- | J = 3.8 Hz, 2H), 7.53-7.49 (m, 1H), 7.44 (dt, J = 8.4, 1.4 |
| | 1H-1,2,3-triazole-4-carboxamide | Hz, 1H), 2.35 (s, 3H); LCMS (M + 1): 530.40 |
| 425 | 5-methyl-1-(2,4,6-trichlorophenyl)- | ¹H-NMR (400 MHz, CHLOROFORM-D) δ 9.75 (s, 1H), |
| | N-((4- | 8.26 (dt, J = 9.6, 2.4 Hz, 2H), 7.58 (s, 2H), 7.40 (dd, J = |
| | (trifluoromethoxy)phenyl)sulfonyl)- | 8.9, 0.9 Hz, 2H), 2.39 (s, 3H); LCMS (M + 1): 530.50 |
| | 1H-1,2,3-triazole-4-carboxamide | |
| 426 | N-((2,4-difluorophenyl)sulfonyl)-5- | ¹H-NMR (400 MHz, CHLOROFORM-D) δ 9.91 (s, 1H), |
| | methyl-1-(2,4,6-trichlorophenyl)- | 8.26-8.20 (m, 1H), 7.58 (s, 2H), 7.12-7.07 (m, 1H), 7.01- |
| | 1H-1,2,3-triazole-4-carboxamide | 6.96 (m, 1H), 2.36 (s, 3H); LCMS (M + 1): 482.45 |
| 427 | N-((5-chloro-2,4- | ¹H-NMR (400 MHz, CHLOROFORM-D) δ 9.95 (s, 1H), |
| | difluorophenyl)sulfonyl)-5-methyl- | 8.28 (t, J = 7.3 Hz, 1H), 7.59 (t, J = 3.7 Hz, 2H), 7.09 (dd, |
| | 1-(2,4,6-trichlorophenyl)-1H-1,2,3- | J = 9.2, 8.6 Hz, 1H), 2.38 (s, 3H); LCMS (M + 1): 516.40 |
| | triazole-4-carboxamide | |
| 428 | N-((5-chloro-2- | ¹H-NMR (400 MHz, CHLOROFORM-D) δ 9.93 (s, 1H), |
| | fluorophenyl)sulfonyl)-5-methyl-1- | 8.17 (q, J = 2.9 Hz, 1H), 7.63-7.59 (m, 1H), 7.58 (d, J = |
| | (2,4,6-trichlorophenyl)-1H-1,2,3- | 3.7 Hz, 2H), 7.20 (t, J = 9.0 Hz, 1H), 2.38 (s, 3H); LCMS |
| | triazole-4-carboxamide | (M + 1): 498.45 |
| 429 | N-((3,5- | ¹H-NMR (400 MHz, CHLOROFORM-D) δ 9.93 (s, 1H), |
| | bis(trifluoromethyl)phenyl)sulfonyl)- | 8.66 (d, J = 7.9 Hz, 2H), 8.16 (s, 1H), 7.60 (s, 2H), 2.38 |
| | 5-methyl-1-(2,4,6-trichlorophenyl)- | (s, 3H); LCMS (M + 1): 582.50 |
| | 1H-1,2,3-triazole-4-carboxamide | |
| 430 | N-((2,5- | ¹H-NMR (400 MHz, CHLOROFORM-D) δ 9.89 (s, 1H), |
| | bis(trifluoromethyl)phenyl)sulfonyl)- | 8.89 (s, 1H), 8.08-8.03 (m, 2H), 7.52 (s, 2H), 2.36 (s, 3H); |
| | 5-methyl-1-(2,4,6-trichlorophenyl)- | LCMS (M + 1): 582.45 |
| | 1H-1,2,3-triazole-4-carboxamide | |
| 431 | 1-(4-chloro-2-fluorophenyl)-N-((2,5- | ¹H-NMR (400 MHz, CHLOROFORM-D) δ 9.96 (s, 1H), |
| | dichlorophenyl)sulfonyl)-5-methyl- | 8.35 (d, J = 2.4 Hz, 1H), 7.55-7.52 (m, 1H), 7.48-7.38 (m, |
| | 1H-1,2,3-triazole-4-carboxamide | 4H), 2.45 (d, J = 1.8 Hz, 3H); LCMS (M + 1): 464.40 |
| 432 | N-((2-bromo-5- | ¹H-NMR (400 MHz, CHLOROFORM-D) δ 10.03 (s, |
| | (trifluoromethyl)phenyl)sulfonyl)-1- | 1H), 8.65 (d, J = 1.8 Hz, 1H), 7.90 (d, J = 8.3 Hz, 1H), |
| | (4-chloro-2-fluorophenyl)-5-methyl- | 7.72 (dd, J = 8.3, 2.1 Hz, 1H), 7.46-7.38 (m, 3H), 2.45 (d, |
| | 1H-1,2,3-triazole-4-carboxamide | J = 1.5 Hz, 3H); LCMS (M − 1): 540.65 |
| 433 | 1-(4-chloro-2-fluorophenyl)-N-((2,5- | ¹H-NMR (400 MHz, CHLOROFORM-D) δ 9.72 (s, 1H), |
| | dimethylphenyl)sulfonyl)-5-methyl- | 8.08 (d, J = 0.9 Hz, 1H), 7.45-7.36 (m, 3H), 7.34-7.32 (m, |
| | 1H-1,2,3-triazole-4-carboxamide | 1H), 7.21 (d, J = 7.6 Hz, 1H), 2.69 (s, 3H), 2.44 (d, J = |
| | | 1.8 Hz, 3H), 2.42 (s, 3H); LCMS (M + 1): 422.90 |
| 434 | 1-(4-chloro-2-fluorophenyl)-N-((2- | ¹H-NMR (400 MHz, CHLOROFORM-D) δ 9.94 (s, 1H), |
| | chlorophenyl)sulfonyl)-5-methyl- | 8.37 (dd, J = 7.9, 1.5 Hz, 1H), 7.61-7.49 (m, 3H), 7.46- |
| | 1H-1,2,3-triazole-4-carboxamide | 7.37 (m, 3H), 2.43 (d, J = 1.8 Hz, 3H); LCMS (M + 1): |
| | | 430.80 |
| 435 | 1-(4-chloro-2-fluorophenyl)-5- | ¹H-NMR (400 MHz, CHLOROFORM-D) δ 9.96 (s, 1H), |
| | methyl-N-((2,4,5- | 8.44 (s, 1H), 7.64 (s, 1H), 7.46-7.38 (m, 3H), 2.46 (d, J = |
| | trichlorophenyl)sulfonyl)-1H-1,2,3- | 1.5 Hz, 3H); LCMS (M + 1): 498.80 |
| | triazole-4-carboxamide | |
| 436 | 1-(4-chloro-2-fluorophenyl)-5- | ¹H-NMR (400 MHz, CHLOROFORM-D) δ 10.02 (s, |
| | methyl-N-((2,4,6- | 1H), 7.46-7.38 (m, 3H), 6.87-6.81 (m, 2H), 2.47 (d, J = |
| | trifluorophenyl)sulfonyl)-1H-1,2,3- | 1.5 Hz, 3H); LCMS (M + 1): 448.90 |
| | triazole-4-carboxamide | |
| 437 | 1-(4-chloro-2-fluorophenyl)-N-((3- | ¹H-NMR (400 MHz, CHLOROFORM-D) δ 9.76 (s, 1H), |
| | chloro-4-fluorophenyl)sulfonyl)-5- | 8.26 (dd, J = 6.6, 2.3 Hz, 1H), 8.10 (qd, J = 4.3, 2.3 Hz, |
| | methyl-1H-1,2,3-triazole-4- | 1H), 7.44-7.31 (m, 4H), 2.48 (d, J = 1.8 Hz, 3H); LCMS |
| | carboxamide | (M + 1): 446.90 |
| 438 | 1-(4-chloro-2-fluorophenyl)-N-((2,4- | ¹H-NMR (400 MHz, CHLOROFORM-D) δ 9.95 (s, 1H), |
| | dichlorophenyl)sulfonyl)-5-methyl- | 8.33-8.27 (m, 1H), 7.55 (d, J = 1.8 Hz, 1H), 7.48 (dd, J = |
| | 1H-1,2,3-triazole-4-carboxamide | 8.6, 2.1 Hz, 1H), 7.46-7.38 (m, 3H), 2.44 (d, J = 1.8 Hz, |
| | | 3H); LCMS (M − 1): 462.90 |
| 439 | 1-(4-chloro-2-fluorophenyl)-N-((4- | ¹H-NMR (400 MHz, CHLOROFORM-D) δ 9.68 (s, 1H), |
| | fluorophenyl)sulfonyl)-5-methyl-1H- | 8.21 (tt, J = 7.3, 2.4 Hz, 2H), 7.44-7.37 (m, 3H), 7.25- |
| | 1,2,3-triazole-4-carboxamide | 7.21 (m, 2H), 2.47 (d, J = 1.8 Hz, 3H); LCMS (M + 1): |
| | | 412.95 |
| 440 | N-((4-chloro-2,5- | ¹H-NMR (400 MHz, CHLOROFORM-D) δ 9.75 (s, 1H), |
| | dimethylphenyl)sulfonyl)-1-(4- | 8.13 (s, 1H), 7.45-7.37 (m, 3H), 7.31 (s, 1H), 2.66 (s, 3H), |
| | chloro-2-fluorophenyl)-5-methyl- | 2.45-2.44 (m, 6H); LCMS (M + 1): 456.90 |
| | 1H-1,2,3-triazole-4-carboxamide | |
| 441 | N-((2,5- | ¹H-NMR (400 MHz, CHLOROFORM-D) δ 9.90 (s, 1H), |
| | bis(trifluoromethyl)phenyl)sulfonyl)- | 8.87 (s, 1H), 8.07-8.02 (m, 2H), 7.45-7.37 (m, 3H), 2.45 |
| | 1-(4-chloro-2-fluorophenyl)-5- | (d, J = 1.8 Hz, 3H); LCMS (M + 1): 530.90 |
| | methyl-1H-1,2,3-triazole-4- | |
| | carboxamide | |
| 442 | N-((3,5- | ¹H-NMR (400 MHz, CHLOROFORM-D) δ 9.96 (s, 1H), |
| | bis(trifluoromethyl)phenyl)sulfonyl)- | 8.66 (s, 2H), 8.15 (s, 1H), 7.44-7.37 (m, 3H), 2.48 (d, J = |
| | 1-(4-chloro-2-fluorophenyl)-5- | 1.5 Hz, 3H); LCMS (M − 1): 528.95 |
| | methyl-1H-1,2,3-triazole-4- | |
| | carboxamide | |
| 443 | 1-(4-chloro-2-fluorophenyl)-5- | ¹H-NMR (400 MHz, CHLOROFORM-D) δ 9.81 (s, 1H), |
| | methyl-N-((2- | 8.63-8.61 (m, 1H), 7.92-7.89 (m, 1H), 7.84-7.77 (m, 2H), |
| | (trifluoromethyl)phenyl)sulfonyl)- | 7.45-7.36 (m, 3H), 2.44 (d, J = 1.5 Hz, 3H); LCMS |
| | 1H-1,2,3-triazole-4-carboxamide | (M + 1): 462.95 |
| 444 | 1-(4-chloro-2-fluorophenyl)-N-((4- | ¹H-NMR (400 MHz, CHLOROFORM-D) δ 9.71 (s, 1H), |
| | chlorophenyl)sulfonyl)-5-methyl- | 8.12 (dt, J = 9.1, 2.3 Hz, 2H), 7.54 (dt, J = 9.2, 2.2 Hz, |
| | 1H-1,2,3-triazole-4-carboxamide | 2H), 7.44-7.37 (m, 3H), 2.47 (d, J = 1.8 Hz, 3H); LCMS |
| | | (M + 1): 430.90 |
| 445 | 1-(4-chloro-2-fluorophenyl)-N-((2- | ¹H-NMR (400 MHz, CHLOROFORM-D) δ 9.90 (s, 1H), |
| | fluorophenyl)sulfonyl)-5-methyl-1H- | 8.19 (td, J = 7.6, 1.5 Hz, 1H), 7.69-7.63 (m, 1H), 7.45- |
| | 1,2,3-triazole-4-carboxamide | 7.35 (m, 4H), 7.22 (dd, J = 9.9, 1.1 Hz, 1H), 2.44 (d, J = |
| | | 1.8 Hz, 3H); LCMS (M + 1): 412.85 |
| 446 | 1-(4-chloro-2-fluorophenyl)-N-((2- | ¹H-NMR (400 MHz, CHLOROFORM-D) δ 10.00 (s, |
| | chloro-5- | 1H), 8.63 (d, J = 1.8 Hz, 1H), 7.82 (dd, J = 8.3, 2.1 Hz, |
| | (trifluoromethyl)phenyl)sulfonyl)-5- | 1H), 7.68 (d, J = 8.3 Hz, 1H), 7.46-7.38 (m, 3H), 2.45 (d, |
| | methyl-1H-1,2,3-triazole-4- | J = 1.8 Hz, 3H); LCMS (M + 1): 496.90 |
| | carboxamide | |
| 447 | N-((3,5- | ¹H-NMR (400 MHz, DMSO-d₆) δ 8.48 (q, J = 0.7 Hz, |
| | bis(trifluoromethyl)phenyl)sulfonyl)- | 1H), 8.40 (s, 2H), 8.34 (s, 1H), 8.14 (dd, J = 2.9, 1.1 Hz, |
| | 1-(2-chloro-4-cyanophenyl)-5- | 2H); LCMS (M − 2): 589.55 |
| | (trifluoromethyl)-1H-1,2,3-triazole- | |
| | 4-carboxamide | |
| 448 | 1-(4-chloro-2-fluorophenyl)-N-((2- | ¹H-NMR (400 MHz, CHLOROFORM-D) δ 9.95 (s, 1H), |
| | chloro-5-methoxyphenyl)sulfonyl)- | 7.85 (d, J = 3.1 Hz, 1H), 7.46-7.37 (m, 4H), 7.09 (dd, J = |
| | 5-methyl-1H-1,2,3-triazole-4- | 8.9, 3.1 Hz, 1H), 3.90 (s, 3H), 2.45 (d, J = 1.8 Hz, 3H); |
| | carboxamide | LCMS (M + 1): 458.85 |
| 449 | 1-(4-chloro-2-fluorophenyl)-5- | ¹H-NMR (400 MHz, CHLOROFORM-D) δ 9.88 (s, 1H), |
| | methyl-N-((2- | 8.33 (dd, J = 7.9, 1.8 Hz, 1H), 7.73-7.69 (m, 1H), 7.53- |
| | (trifluoromethoxy)phenyl)sulfonyl)- | 7.48 (m, 1H), 7.46-7.41 (m, 2H), 7.38 (dt, J = 9.8, 1.8 Hz, |
| | 1H-1,2,3-triazole-4-carboxamide | 2H), 2.44 (d, J = 1.8 Hz, 3H); LCMS (M + 1): 478.90 |
| 450 | 1-(4-chloro-2-fluorophenyl)-5- | ¹H-NMR (400 MHz, CHLOROFORM-D) δ 9.72 (s, 1H), |
| | methyl-N-((4- | 8.24 (dt, J = 9.6, 2.4 Hz, 2H), 7.44-7.37 (m, 5H), 2.47 (d, |
| | (trifluoromethoxy)phenyl)sulfonyl)- | J = 1.8 Hz, 3H); LCMS (M − 1): 476.80 |
| | 1H-1,2,3-triazole-4-carboxamide | |
| 451 | N-((5-chloro-2,4- | ¹H-NMR (400 MHz, CHLOROFORM-D) δ 9.97 (s, 1H), |
| | difluorophenyl)sulfonyl)-1-(4- | 8.27 (t, J = 7.5 Hz, 1H), 7.46-7.38 (m, 3H), 7.08 (t, J = |
| | chloro-2-fluorophenyl)-5-methyl- | 8.7 Hz, 1H), 2.48-2.46 (m, 3H); LCMS (M − 1): 464.55 |
| | 1H-1,2,3-triazole-4-carboxamide | |
| 452 | N-((2-chloro-5- | ¹H-NMR (400 MHz, DMSO-d₆) δ 13.30 (s, 1H), 8.33 (dd, |
| | methoxyphenyl)sulfonyl)-1-(4- | J = 10.1, 1.5 Hz, 1H), 8.04-7.96 (m, 2H), 7.62 (d, J = 3.4 |
| | cyano-2-fluorophenyl)-5-methyl-1H- | Hz, 1H), 7.58 (d, J = 8.9 Hz, 1H), 7.28 (dd, J = 8.9, 3.1 |
| | 1,2,3-triazole-4-carboxamide | Hz, 1H), 3.84 (s, 3H), 2.35 (s, 3H); LCMS (M+): 449.85 |
| 453 | 1-(4-cyano-2-fluorophenyl)-N-((2,5- | ¹H-NMR (400 MHz, DMSO-d₆) δ 8.33 (dd, J = 9.9, 1.7 |
| | difluorophenyl)sulfonyl)-5-methyl- | Hz, 1H), 8.02 (dd, J = 8.4, 1.7 Hz, 1H), 7.97 (t, J = 7.8 |
| | 1H-1,2,3-triazole-4-carboxamide | Hz, 1H), 7.78-7.74 (m, 1H), 7.67-7.62 (m, 1H), 7.53 (td, |
| | | J = 9.3, 4.0 Hz, 1H), 2.36 (s, 3H); LCMS (M+): 421.85 |
| 454 | 1-(4-cyano-2-fluorophenyl)-N-((4- | ¹H-NMR (400 MHz, DMSO-d₆) δ 12.84 (s, 1H), 8.32 (dd, |
| | fluorophenyl)sulfonyl)-5-methyl-1H- | J = 9.9, 1.7 Hz, 1H), 8.11-8.06 (m, 2H), 8.03-8.00 (m, |
| | 1,2,3-triazole-4-carboxamide | 1H), 7.96 (t, J = 7.8 Hz, 1H), 7.52-7.46 (m, 2H), 2.36 (s, |
| | | 3H); LCMS (M+): 403.90 |
| 455 | N-((2-bromo-5- | ¹H-NMR (400 MHz, DMSO-d₆) δ 8.37 (d, J = 1.8 Hz, |
| | (trifluoromethyl)phenyl)sulfonyl)-1- | 1H), 8.32 (dd, J = 9.9, 1.4 Hz, 1H), 8.09 (d, J = 8.3 Hz, |
| | (4-cyano-2-fluorophenyl)-5-methyl- | 1H), 8.03-7.91 (m, 3H), 2.36 (s, 3H); LCMS (M+): |
| | 1H-1,2,3-triazole-4-carboxamide | 533.80 |
| 456 | 1-(4-cyano-2-fluorophenyl)-5- | ¹H-NMR (400 MHz, DMSO-d₆) δ 12.91 (s, 1H), 8.32 (dd, |
| | methyl-N-((4- | J = 10.1, 1.5 Hz, 1H), 8.15 (dt, J = 9.6, 2.4 Hz, 2H), 8.02 |
| | (trifluoromethoxy)phenyl)sulfonyl)- | (dd, J = 8.3, 1.2 Hz, 1H), 7.95 (t, J = 7.8 Hz, 1H), 7.65 (d, |
| | 1H-1,2,3-triazole-4-carboxamide | J = 7.9 Hz, 2H), 2.36 (s, 3H); LCMS (M+): 469.90 |
| 457 | N-((4-chlorophenyl)sulfonyl)-1-(4- | ¹H-NMR (400 MHz, DMSO-d₆) δ 12.94 (s, 1H), 8.32 (dd, |
| | cyano-2-fluorophenyl)-5-methyl-1H- | J = 9.9, 1.7 Hz, 1H), 8.03-7.99 (m, 3H), 7.95 (t, J = 7.8 |
| | 1,2,3-triazole-4-carboxamide | Hz, 1H), 7.75-7.68 (m, 2H), 2.36 (s, 3H); LCMS (M+): |
| | | 419.85 |
| 458 | N-((2-bromo-5- | ¹H-NMR (400 MHz, DMSO-d₆) δ 8.49 (d, J = 1.8 Hz, |
| | (trifluoromethyl)phenyl)sulfonyl)-1- | 1H), 8.37 (d, J = 2.1 Hz, 1H), 8.15 (dd, J = 8.3, 1.8 Hz, |
| | (2-chloro-4-cyanophenyl)-5-methyl- | 1H), 8.09 (d, J = 8.3 Hz, 1H), 7.98-7.94 (m, 2H), 2.29 (s, |
| | 1H-1,2,3-triazole-4-carboxamide | 3H); LCMS (M + 1): 549.75 |
| 459 | 1-(2-chloro-4-cyanophenyl)-5- | ¹H-NMR (400 MHz, DMSO-d₆) δ 13.06 (s, 1H), 8.49 (d, |
| | methyl-N-(o-tolylsulfonyl)-1H- | J = 1.8 Hz, 1H), 8.15 (dd, J = 8.3, 1.5 Hz, 1H), 8.05 (dd, |
| | 1,2,3-triazole-4-carboxamide | J = 7.9, 1.2 Hz, 1H), 7.96 (d, J = 8.3 Hz, 1H), 7.60-7.56 |
| | | (m, 1H), 7.47-7.40 (m, 2H), 2.65 (s, 3H), 2.6 (s, 3H); |
| | | LCMS (M+): 415.85 |
| 460 | N-((4-chloro-2,5- | ¹H-NMR (400 MHz, DMSO-d₆) δ 13.15 (s, 1H), 8.49 (d, |
| | dimethylphenyl)sulfonyl)-1-(2- | J = 1.8 Hz, 1H), 8.16 (dd, J = 8.3, 1.8 Hz, 1H), 8.00 (s, |
| | chloro-4-cyanophenyl)-5-methyl- | 1H), 7.96 (d, J = 8.3 Hz, 1H), 7.52 (s, 1H), 2.59 (s, 3H), |
| | 1H-1,2,3-triazole-4-carboxamide | 2.39 (s, 3H), 2.27 (s, 3H); LCMS (M+): 464.00 |
| 461 | 1-(2-chloro-4-cyanophenyl)-5- | ¹H-NMR (400 MHz, DMSO-d₆) δ 8.49 (d, J = 1.5 Hz, |
| | methyl-N-((2,4,5- | 1H), 8.25 (s, 1H), 8.15 (dd, J = 8.3, 1.8 Hz, 1H), 8.12 (s, |
| | trichlorophenyl)sulfonyl)-1H-1,2,3- | 1H), 7.97 (d, J = 8.3 Hz, 1H), 2.29 (s, 3H); LCMS (M+): |
| | triazole-4-carboxamide | 505.75 |
| 462 | 5-methyl-N-(o-tolylsulfonyl)-1- | ¹H-NMR (400 MHz, CHLOROFORM-D) δ 9.77 (s, 1H), |
| | (2,4,6-trichlorophenyl)-1H-1,2,3- | 8.30 (dd, J = 8.1, 1.5 Hz, 1H), 7.58-7.56 (m, 2H), 7.54- |
| | triazole-4-carboxamide | 7.52 (m, 1H), 7.43 (td, J = 7.7, 0.7 Hz, 1H), 7.35 (d, J = |
| | | 7.6 Hz, 1H), 2.76 (s, 3H), 2.35 (s, 3H); LCMS (M + 1): |
| | | 460.45 |
| 463 | 5-methyl-1-(2,4,6-trichlorophenyl)- | ¹H-NMR (400 MHz, CHLOROFORM-D) δ 9.98 (s, 1H), |
| | N-((2,4,5-trichlorophenyl)sulfonyl)- | 8.45 (s, 1H), 7.66 (s, 1H), 7.59 (s, 2H), 2.37 (s, 3H); |
| | 1H-1,2,3-triazole-4-carboxamide | LCMS (M + 1): 548.40 |
| 464 | 5-methyl-1-(2,4,6-trichlorophenyl)- | ¹H-NMR (400 MHz, CHLOROFORM-D) δ 9.98 (s, 1H), |
| | N-((2,4,6-trifluorophenyl)sulfonyl)- | 8.45 (s, 1H), 7.66 (s, 1H), 7.58 (s, 2H), 2.37 (s, 3H); |
| | 1H-1,2,3-triazole-4-carboxamide | LCMS (M + 1): 500.45 |
| 465 | N-((3-chloro-4- | ¹H-NMR (400 MHz, CHLOROFORM-D) δ 9.75 (s, 1H), |
| | fluorophenyl)sulfonyl)-5-methyl-1- | 8.28 (dd, J = 6.7, 2.4 Hz, 1H), 8.12 (qd, J = 4.3, 2.4 Hz, |
| | (2,4,6-trichlorophenyl)-1H-1,2,3- | 1H), 7.59 (s, 2H), 7.34 (t, J = 8.6 Hz, 1H), 2.39 (s, 3H); |
| | triazole-4-carboxamide | LCMS (M + 1): 498.45 |
| 466 | N-((2,4-dichlorophenyl)sulfonyl)-5- | ¹H-NMR (400 MHz, CHLOROFORM-D) δ 9.95 (s, 1H), |
| | methyl-1-(2,4,6-trichlorophenyl)- | 8.31 (d, J = 8.6 Hz, 1H), 7.59 (d, J = 4.0 Hz, 2H), 7.57 (t, |
| | 1H-1,2,3-triazole-4-carboxamide | J = 2.6 Hz, 1H), 7.49 (dd, J = 8.6, 2.1 Hz, 1H), 2.36 (s, |
| | | 3H); LCMS (M + 1): 514.40 |
| 467 | N-((2,5-difluorophenyl)sulfonyl)-5- | ¹H-NMR (400 MHz, CHLOROFORM-D) δ 9.99 (s, 1H), |
| | methyl-1-(2,4,6-trichlorophenyl)- | 7.92-7.88 (m, 1H), 7.58 (t, J = 3.7 Hz, 2H), 7.38-7.32 (m, |
| | 1H-1,2,3-triazole-4-carboxamide | 1H), 7.25-7.20 (m, 1H), 2.37 (s, 3H); LCMS (M + 1): |
| | | 482.40 |
| 468 | N-((4-chloro-2,5- | ¹H-NMR (400 MHz, CHLOROFORM-D) δ 9.76 (s, 1H), |
| | dimethylphenyl)sulfonyl)-5-methyl- | 8.14 (s, 1H), 7.58 (s, 2H), 7.32 (s, 1H), 2.68 (s, 3H), 2.44 |
| | 1-(2,4,6-trichlorophenyl)-1H-1,2,3- | (s, 3H), 2.36 (s, 3H); LCMS (M + 1): 508.45 |
| | triazole-4-carboxamide | |
| 469 | 5-methyl-1-(2,4,6-trichlorophenyl)- | ¹H-NMR (400 MHz, CHLOROFORM-D) δ 9.81 (s, 1H), |
| | N-((2- | 8.63 (dd, J = 7.5, 1.7 Hz, 1H), 7.93-7.90 (m, 1H), 7.85- |
| | (trifluoromethyl)phenyl)sulfonyl)- | 7.77 (m, 2H), 7.58 (s, 2H), 2.35 (s, 3H); LCMS (M + 1): |
| | 1H-1,2,3-triazole-4-carboxamide | 514.45 |
| 470 | 1-(4-chloro-2-fluorophenyl)-N-((2,4- | ¹H-NMR (400 MHz, CHLOROFORM-D) δ 9.90 (s, 1H), |
| | difluorophenyl)sulfonyl)-5-methyl- | 8.24-8.19 (m, 1H), 7.45-7.37 (m, 3H), 7.11-7.06 (m, 1H), |
| | 1H-1,2,3-triazole-4-carboxamide | 7.00-6.95 (m, 1H), 2.45 (d, J = 1.5 Hz, 3H); LCMS |
| | | (M + 1): 430.85 |
| 471 | 1-(4-chloro-2-fluorophenyl)-N-((4- | ¹H-NMR (400 MHz, CHLOROFORM-D) δ 9.92 (s, 1H), |
| | chloro-2-fluorophenyl)sulfonyl)-5- | 8.15 (q, J = 2.9 Hz, 1H), 7.60 (qd, J = 4.3, 2.7 Hz, 1H), |
| | methyl-1H-1,2,3-triazole-4- | 7.46-7.38 (m, 3H), 7.19 (t, J = 9.0 Hz, 1H), 2.46 (d, J = |
| | carboxamide | 1.4 Hz, 3H); LCMS (M + 1): 446.85 |
| 472 | 1-(4-cyano-2-fluorophenyl)-5- | ¹H-NMR (400 MHz, DMSO-d₆) δ 8.32 (dd, J = 10.0, 1.5 |
| | methyl-N-(o-tolylsulfonyl)-1H- | Hz, 1H), 8.03 (ddd, J = 11.8, 8.1, 1.4 Hz, 2H), 7.99-7.96 |
| | 1,2,3-triazole-4-carboxamide | (m, 1H), 7.58 (td, J = 7.5, 1.3 Hz, 1H), 7.47-7.40 (m, 2H), |
| | | 2.63 (s, 3H), 2.33 (s, 3H); LCMS (M+) = 399.45 |
| 473 | N-((2-bromo-5- | ¹H-NMR (400 MHz, DMSO-d₆) δ 8.32 (d, J = 2.1 Hz, |
| | (trifluoromethyl)phenyl)sulfonyl)-1- | 1H), 8.00 (d, J = 8.3 Hz, 1H), 7.83 (dd, J = 8.3, 1.8 Hz, |
| | (2-chloro-4-methoxyphenyl)-5- | 1H), 7.77 (d, J = 8.9 Hz, 1H), 7.38 (d, J = 2.8 Hz, 1H), |
| | (trifluoromethyl)-1H-1,2,3-triazole- | 7.14 (dd, J = 8.9, 2.8 Hz, 1H), 3.88 (s, 3H); LCMS(M+): |
| | 4-carboxamide | 608.75 |
| 474 | 1-(2-chloro-4-methoxyphenyl)-N- | ¹H-NMR (400 MHz, DMSO-d₆) δ 8.12 (d, J = 8.9 Hz, |
| | ((2,4-dichlorophenyl)sulfonyl)-5- | 1H), 7.83 (d, J = 2.1 Hz, 1H), 7.79 (d, J = 8.9 Hz, 1H), |
| | (trifluoromethyl)-1H-1,2,3-triazole- | 7.66 (dd, J = 8.6, 2.1 Hz, 1H), 7.39 (d, J = 2.8 Hz, 1H), |
| | 4-carboxamide | 7.16 (dd, J = 8.9, 2.8 Hz, 1H), 3.88 (s, 3H); LCMS(M+): |
| | | 530.80 |
| 475 | 1-(4-chloro-2-fluorophenyl)-5- | ¹H-NMR (400 MHz, CHLOROFORM-D) δ 9.74 (s, 1H), |
| | methyl-N-(o-tolylsulfonyl)-1H- | 8.28 (dd, J = 7.9, 1.3 Hz, 1H), 7.55-7.51 (m, 1H), 7.46- |
| | 1,2,3-triazole-4-carboxamide | 7.36 (m, 4H), 7.33 (d, J = 7.6 Hz, 1H), 2.74 (s, 3H), 2.44 |
| | | (d, J = 1.7 Hz, 3H); LCMS (M + 1): 408.90 |
| 476 | N-((2-bromo-5- | ¹H-NMR (400 MHz, DMSO-d₆) δ 8.38 (d, J = 2.2 Hz, |
| | (trifluoromethyl)phenyl)sulfonyl)-1- | 1H), 8.12 (d, J = 8.3 Hz, 1H), 7.98 (dd, J = 8.3, 2.0 Hz, |
| | (2-chloro-4-methylphenyl)-5- | 1H), 7.64 (s, 1H), 7.59-7.56 (m, 1H), 7.42 (dd, J = 8.1, |
| | methyl-1H-1,2,3-triazole-4- | 1.0 Hz, 1H), 2.41 (d, J = 10.6 Hz, 3H), 2.23 (s, 3H); |
| | carboxamide | LCMS(M + 1): 538.8 |
| 477 | N-((3,5- | ¹H-NMR (400 MHz, DMSO-d₆) δ 8.55 (s, 3H), 7.64 (s, |
| | bis(trifluoromethyl)phenyl)sulfonyl)- | 1H), 7.55 (d, J = 8.1 Hz, 1H), 7.42 (d, J = 7.1 Hz, 1H), |
| | 1-(2-chloro-4-methylphenyl)-5- | 2.42 (s, 3H), 2.25 (s, 3H); LCMS(M + 1): 527.0 |
| | methyl-1H-1,2,3-triazole-4- | |
| | carboxamide | |
| 478 | 1-(2-chloro-4-methylphenyl)-N- | ¹H-NMR (400 MHz, DMSO-d₆) δ 7.72-7.83 (1H), 7.62- |
| | ((2,5-difluorophenyl)sulfonyl)-5- | 7.70 (2H), 7.50-7.60 (2H), 7.38-7.46 (1H), 2.38-2.45 |
| | methyl-1H-1,2,3-triazole-4- | (3H), 2.18-2.28 (3H); LCMS(M+): 426.9 |
| | carboxamide | |
| 479 | 1-(2-chloro-4-methylphenyl)-N- | ¹H-NMR (400 MHz, DMSO-d₆) δ 8.11 (d, J = 2.7 Hz, |
| | ((2,5-dichlorophenyl)sulfonyl)-5- | 1H), 7.80 (dd, J = 8.6, 2.7 Hz, 1H), 7.72 (d, J = 8.6 Hz, |
| | methyl-1H-1,2,3-triazole-4- | 1H), 7.65 (s, 1H), 7.58 (d, J = 8.1 Hz, 1H), 7.42 (d, J = |
| | carboxamide | 7.3 Hz, 1H), 2.42 (s, 3H), 2.24 (s, 3H); LCMS(M + 1): |
| | | 460.8 |
| 480 | 1-(2-chloro-4-methylphenyl)-5- | ¹H-NMR (400 MHz, DMSO-d₆) δ 8.14 (dd, J = 7.9, 1.6 |
| | methyl-N-((2- | Hz, 1H), 7.86-7.82 (m, 1H), 7.65-7.57 (m, 4H), 7.42 (d, |
| | (trifluoromethoxy)phenyl)sulfonyl)- | J = 7.1 Hz, 1H), 2.40 (d, J = 16.1 Hz, 3H), 2.22 (s, 3H); |
| | 1H-1,2,3-triazole-4-carboxamide | LCMS(M+): 474.9 |
| 481 | 1-(2-chloro-4-methylphenyl)-N-((2- | ¹H-NMR (400 MHz, DMSO-d₆) δ 8.18 (dd, J = 7.9, 1.3 |
| | chlorophenyl)sulfonyl)-5-methyl- | Hz, 1H), 7.73-7.66 (m, 2H), 7.65-7.61 (m, 2H), 7.60-7.57 |
| | 1H-1,2,3-triazole-4-carboxamide | (m, 1H), 7.42 (dd, J = 8.2, 1.1 Hz, 1H), 2.40 (d, J = 15.9 |
| | | Hz, 3H), 2.20 (d, J = 15.9 Hz, 3H); LCMS(M − 1): 424.9 |
| 482 | 1-(2-chloro-4-methylphenyl)-N-((2- | ¹H-NMR (400 MHz, DMSO-d₆) δ 8.00 (td, J = 7.7, 1.6 |
| | fluorophenyl)sulfonyl)-5-methyl-1H- | Hz, 1H), 7.80-7.74 (m, 1H), 7.65 (d, J = 1.0 Hz, 1H), 7.57 |
| | 1,2,3-triazole-4-carboxamide | (d, J = 8.1 Hz, 1H), 7.48-7.41 (m, 3H), 2.41 (d, J = 13.9 |
| | | Hz, 3H), 2.23 (s, 3H); LCMS(M+): 408.9 |
| 483 | 1-(2-chloro-4-methylphenyl)-N-((2- | ¹H-NMR (400 MHz, CHLOROFORM-D) δ 7.86 (d, J = |
| | chloro-5-methoxyphenyl)sulfonyl)- | 2.9 Hz, 1H), 7.43 (s, 1H), 7.41 (d, J = 8.8 Hz, 1H), 7.27 |
| | 5-methyl-1H-1,2,3-triazole-4- | (d, J = 0.7 Hz, 2H), 7.09 (dd, J = 8.8, 3.2 Hz, 1H), 3.90 |
| | carboxamide | (s, 3H), 2.46 (s, 3H), 2.38 (s, 3H); LCMS(M + 2): 458.9 |
| 484 | 1-(2-chloro-4-methylphenyl)-5- | ¹H-NMR (400 MHz, DMSO-d₆) δ 7.65 (s, 1H), 7.58 (d, J |
| | methyl-N-((2,4,6- | = 7.8 Hz, 1H), 7.48-7.42 (m, 3H), 2.43 (s, 3H), 2.26 (s, |
| | trifluorophenyl)sulfonyl)-1H-1,2,3- | 3H); LCMS(M+): 444.3 |
| | triazole-4-carboxamide | |
| 485 | 1-(2-chloro-4-methylphenyl)-N-((2- | ¹H-NMR (400 MHz, CHLOROFORM-D) δ 8.63 (d, J = |
| | chloro-5- | 2.0 Hz, 1H), 7.82 (dd, J = 8.3, 1.7 Hz, 1H), 7.69 (d, J = |
| | (trifluoromethyl)phenyl)sulfonyl)-5- | 8.3 Hz, 1H), 7.43 (s, 1H), 7.27 (s, 2H), 2.46 (s, 3H), 2.39 |
| | methyl-1H-1,2,3-triazole-4- | (d, J = 13.4 Hz, 3H); LCMS(M − 1): 492.9 |
| | carboxamide | |
| 486 | 1-(2-chloro-4-methylphenyl)-N- | ¹H-NMR (400 MHz, DMSO-d₆) δ 8.07 (td, J = 8.6, 6.4 |
| | ((2,4-difluorophenyl)sulfonyl)-5- | Hz, 1H), 7.65 (d, J = 0.7 Hz, 1H), 7.59-7.54 (m, 2H), 7.42 |
| | methyl-1H-1,2,3-triazole-4- | (dd, J = 8.1, 1.0 Hz, 1H), 7.36 (td, J = 8.5, 2.1 Hz, 1H), |
| | carboxamide | 2.41 (d, J = 11.5 Hz, 3H), 2.23 (s, 3H); LCMS(M+): 426.8 |
| 487 | 1-(2-chloro-4-methylphenyl)-5- | ¹H-NMR (400 MHz, DMSO-d₆) δ 8.07 (td, J = 8.6, 6.4 |
| | methyl-N-((2- | Hz, 1H), 7.65 (d, J = 0.7 Hz, 1H), 7.59-7.54 (m, 2H), 7.42 |
| | (trifluoromethyl)phenyl)sulfonyl)- | (dd, J = 8.1, 1.0 Hz, 1H), 7.36 (td, J = 8.5, 2.1 Hz, 1H), |
| | 1H-1,2,3-triazole-4-carboxamide | 2.41 (d, J = 11.5 Hz, 3H), 2.23 (s, 3H); LCMS(M+): 459.1 |
| 488 | 1-(2-chloro-4-methylphenyl)-N- | ¹H-NMR (400 MHz, DMSO-d₆) δ 7.85 (s, 1H), 7.64 (s, |
| | ((2,5-dimethylphenyl)sulfonyl)-5- | 1H), 7.57 (d, J = 8.1 Hz, 1H), 7.43-7.38 (m, 2H), 7.28 (d, |
| | methyl-1H-1,2,3-triazole-4- | J = 8.1 Hz, 1H), 2.59 (s, 3H), 2.42 (s, 3H), 2.36 (s, 3H), |
| | carboxamide | 2.22 (s, 3H); LCMS(M+): 418.4 |
| 489 | 1-(2-chloro-4-methylphenyl)-N- | ¹H-NMR (400 MHz, DMSO-d₆) δ 8.16 (d, J = 8.6 Hz, |
| | ((2,4-dichlorophenyl)sulfonyl)-5- | 1H), 7.90 (d, J = 2.0 Hz, 1H), 7.72 (dd, J = 8.6, 2.2 Hz, |
| | methyl-1H-1,2,3-triazole-4- | 1H), 7.65 (s, 1H), 7.58 (d, J = 8.1 Hz, 1H), 7.42 (d, J = |
| | carboxamide | 7.1 Hz, 1H), 2.42 (s, 3H), 2.23 (s, 3H); LCMS(M + 1): |
| | | 460.7 |
| 490 | N-((3-chloro-4- | ¹H-NMR (400 MHz, DMSO-d₆) δ 8.18 (dd, J = 6.8, 2.2 |
| | fluorophenyl)sulfonyl)-1-(2-chloro- | Hz, 1H), 8.04 (qd, J = 4.4, 2.3 Hz, 1H), 7.71 (t, J = 8.9 |
| | 4-methylphenyl)-5-methyl-1H-1,2,3- | Hz, 1H), 7.65 (d, J = 1.0 Hz, 1H), 7.56 (d, J = 8.1 Hz, |
| | triazole-4-carboxamide | 1H), 7.42 (dd, J = 8.1, 1.0 Hz, 1H), 2.42 (s, 3H), 2.26 (s, |
| | | 3H), LCMS(M − 1): 442.8 |
| 491 | 1-(2-chloro-4-methylphenyl)-5- | ¹H-NMR (400 MHz, DMSO-d₆) δ 8.17-8.13 (m, 2H), |
| | methyl-N-((4- | 7.66 (d, J = 8.3 Hz, 3H), 7.56 (d, J = 7.9 Hz, 1H), 7.42 |
| | (trifluoromethoxy)phenyl)sulfonyl)- | (dd, J = 8.1, 1.1 Hz, 1H), 2.42 (s, 3H), 2.25 (s, 3H); |
| | 1H-1,2,3-triazole-4-carboxamide | LCMS(M+): 474.4 |
| 492 | 1-(2-chloro-4-methylphenyl)-5- | ¹H-NMR (400 MHz, DMSO-d₆) δ 8.05 (dd, J = 8.1, 1.2 |
| | methyl-N-(o-tolylsulfonyl)-1H- | Hz, 1H), 7.64 (d, J = 1.0 Hz, 1H), 7.60-7.56 (m, 2H), |
| | 1,2,3-triazole-4-carboxamide | 7.47-7.40 (m, 3H), 2.65 (s, 3H), 2.42 (s, 3H), 2.22 (s, 3H); |
| | | LCMS(M+): 404.9 |
| 493 | N-((4-chloro-2,5- | ¹H-NMR (400 MHz, DMSO-d₆) δ 7.99 (s, 1H), 7.65 (s, |
| | dimethylphenyl)sulfonyl)-1-(2- | 1H), 7.57 (d, J = 8.1 Hz, 1H), 7.52 (s, 1H), 7.42 (d, J = |
| | chloro-4-methylphenyl)-5-methyl- | 6.8 Hz, 1H), 2.59 (s, 3H), 2.40 (d, J = 14.9 Hz, 6H), 2.22 |
| | 1H-1,2,3-triazole-4-carboxamide | (s, 3H); LCMS(M − 1): 452.4 |
| 494 | 1-(2-chloro-4-methylphenyl)-5- | ¹H-NMR (400 MHz, DMSO-d₆) δ 7.65 (s, 1H), 7.59-7.56 |
| | methyl-N-((2,4,5- | (m, 1H), 7.45 (dd, J = 19.1, 9.0 Hz, 3H), 2.43 (s, 3H), |
| | trichlorophenyl)sulfonyl)-1H-1,2,3- | 2.32-2.26 (m, 3H); LCMS(M+): 494.2 |
| | triazole-4-carboxamide | |
| 495 | N-((5-chloro-2- | ¹H-NMR (400 MHz, DMSO-d₆) δ 7.93 (q, J = 2.9 Hz, |
| | fluorophenyl)sulfonyl)-1-(2-chloro- | 1H), 7.85-7.81 (m, 1H), 7.64 (s, 1H), 7.57 (d, J = 8.1 Hz, |
| | 4-methylphenyl)-5-methyl-1H-1,2,3- | 1H), 7.52 (t, J = 9.3 Hz, 1H), 7.42 (d, J = 8.1 Hz, 1H), |
| | triazole-4-carboxamide | 2.43 (s, 3H), 2.25 (s, 3H); LCMS(M − 1): 442.8 |
| 496 | N-((2,5- | ¹H-NMR (400 MHz, DMSO-d₆) δ 8.62 (s, 1H), 8.30 (d, |
| | bis(trifluoromethyl)phenyl)sulfonyl)- | J = 8.3 Hz, 1H), 8.23 (d, J = 8.6 Hz, 1H), 7.64 (s, 1H), 7.56 |
| | 1-(2-chloro-4-methylphenyl)-5- | (d, J = 7.8 Hz, 1H), 7.43-7.41 (m, 1H), 2.42 (s, 3H), 2.23 |
| | methyl-1H-1,2,3-triazole-4- | (d, J = 8.3 Hz, 3H); LCMS(M + 1): 527.1 |
| | carboxamide | |
| 497 | N-((5-chloro-2- | ¹H-NMR (400 MHz, DMSO-d₆) δ 8.49 (d, J = 1.8 Hz, |
| | fluorophenyl)sulfonyl)-1-(2-chloro- | 1H), 8.16 (td, J = 4.1, 1.6 Hz, 1H), 7.98-7.94 (m, 2H), |
| | 4-cyanophenyl)-5-methyl-1H-1,2,3- | 7.86 (qd, J = 4.3, 2.8 Hz, 1H), 7.55 (t, J = 9.3 Hz, 1H), |
| | triazole-4-carboxamide | 2.30 (d, J = 8.3 Hz, 3H); LCMS (M − 1): 453.30 |
| 498 | N-((5-chloro-2,4- | ¹H-NMR (400 MHz, DMSO-d₆) δ 8.49 (t, J = 2.1 Hz, 1H), |
| | difluorophenyl)sulfonyl)-1-(2- | 8.17-8.12 (m, 2H), 7.98-7.95 (m, 1H), 7.85 (t, J = 9.6 Hz, |
| | chloro-4-cyanophenyl)-5-methyl- | 1H), 2.29 (s, 3H); LCMS (M − 1): 471.35 |
| | 1H-1,2,3-triazole-4-carboxamide | |
| 499 | N-((4-chloro-3- | ¹H-NMR (400 MHz, DMSO-d₆) δ 8.49 (d, J = 1.8 Hz, |
| | fluorophenyl)sulfonyl)-1-(2-chloro- | 1H), 8.18-8.14 (m, 2H), 8.03 (qd, J = 4.4, 2.4 Hz, 1H), |
| | 4-cyanophenyl)-5-methyl-1H-1,2,3- | 7.95 (d, J = 7.9 Hz, 1H), 7.71 (t, J = 8.9 Hz, 1H), 2.31 (s, |
| | triazole-4-carboxamide | 3H); LCMS (M − 1): 453.30 |
| 500 | N-((4-chlorophenyl)sulfonyl)-1-(2,5- | ¹H-NMR (400 MHz, DMSO-d₆) δ 12.75 (s, 1H), 8.02 (dt, |
| | dichlorophenyl)-5-methyl-1H-1,2,3- | J = 9.3, 2.3 Hz, 2H), 7.98-7.96 (m, 1H), 7.86 (q, J = 3.0 |
| | triazole-4-carboxamide | Hz, 1H), 7.84-7.80 (m, 1H), 7.74 (dt, J = 9.2, 2.3 Hz, 2H), |
| | | 2.26 (s, 3H); LCMS (M + 1): 446.30 |
| 501 | 1-(2,5-dichlorophenyl)-N-((4- | ¹H-NMR (400 MHz, DMSO-d₆) δ 12.48 (s, 1H), 8.12- |
| | fluorophenyl)sulfonyl)-5-methyl-1H- | 8.06 (m, 2H), 7.97 (d, J = 2.1 Hz, 1H), 7.88-7.80 (m, 2H), |
| | 1,2,3-triazole-4-carboxamide | 7.52-7.47 (m, 2H), 2.28 (s, 3H); LCMS (M + 1): 430.30 |
| 502 | N-((2-bromo-5- | ¹H-NMR (400 MHz, DMSO-d₆) δ 8.34 (d, J = 2.1 Hz, |
| | (trifluoromethyl)phenyl)sulfonyl)-1- | 1H), 8.05 (d, J = 8.3 Hz, 1H), 7.88 (dd, J = 8.6, 2.1 Hz, |
| | (2-chloro-4-methylphenyl)-5- | 1H), 7.73 (d, J = 7.9 Hz, 1H), 7.65 (d, J = 0.9 Hz, 1H), |
| | (trifluoromethyl)-1H-1,2,3-triazole- | 7.44-7.41 (m, 1H), 2.43 (s, 3H); LCMS(M − 1): 590.1 |
| | 4-carboxamide | |
| 503 | 1-(2-chloro-4-methylphenyl)-N- | ¹H-NMR (400 MHz, DMSO-d₆) δ 8.07 (d, J = 2.4 Hz, |
| | ((2,5-dichlorophenyl)sulfonyl)-5- | 1H), 7.75 (dd, J = 8.4, 2.3 Hz, 2H), 7.69-7.63 (m, 2H), |
| | (trifluoromethyl)-1H-1,2,3-triazole- | 7.43 (dd, J = 7.9, 0.9 Hz, 1H), 2.43 (s, 3H), LCMS(M − 1): |
| | 4-carboxamide | 512.1 |
| 504 | 1-(2-chloro-4-methylphenyl)-N-((2- | ¹H-NMR (400 MHz, DMSO-d₆) δ 8.13 (dd, J = 7.9, 1.5 |
| | (trifluoromethoxy)phenyl)sulfonyl)- | Hz, 1H), 7.81 (td, J = 7.9, 1.6 Hz, 1H), 7.75 (d, J = 7.9 |
| | 5-(trifluoromethyl)-1H-1,2,3- | Hz, 1H), 7.66 (d, J = 0.9 Hz, 1H), 7.62-7.60 (m, 1H), 7.57 |
| | triazole-4-carboxamide | (d, J = 9.8 Hz, 1H), 7.43 (dt, J = 8.0, 0.9 Hz, 1H), 2.43 (s, |
| | | 3H), LCMS(M+): 528.4 |
| 505 | 1-(2-chloro-4-methylphenyl)-N-((2- | ¹H-NMR (400 MHz, DMSO-d₆) δ 8.16-8.14 (m, 1H), |
| | chlorophenyl)sulfonyl)-5- | 7.75 (d, J = 7.9 Hz, 1H), 7.69-7.67 (m, 1H), 7.65 (d, J = |
| | (trifluoromethyl)-1H-1,2,3-triazole- | 5.8 Hz, 1H), 7.60-7.56 (m, 1H), 7.43 (dd, J = 8.1, 1.1 Hz, |
| | 4-carboxamide | 1H), 2.43 (s, 3H); LCMS(M − 2): 476.3 |
| 506 | 1-(2-chloro-4-methylphenyl)-N-((2- | ¹H-NMR (400 MHz, DMSO-d₆) δ 7.99 (td, J = 7.6, 1.7 |
| | fluorophenyl)sulfonyl)-5- | Hz, 1H), 7.77-7.74 (m, 2H), 7.66 (d, J = 0.9 Hz, 1H), |
| | (trifluoromethyl)-1H-1,2,3-triazole- | 7.47-7.42 (m, 3H), 2.43 (s, 3H); LCMS(M+): 462.4 |
| | 4-carboxamide | |
| 507 | 1-(2-chloro-4-methylphenyl)-N-((2- | ¹H-NMR (400 MHz, DMSO-d₆) δ 7.76 (d, J = 7.9 Hz, |
| | chloro-5-methoxyphenyl)sulfonyl)- | 1H), 7.66 (d, J = 0.9 Hz, 1H), 7.61 (d, J = 3.1 Hz, 1H), |
| | 5-(trifluoromethyl)-1H-1,2,3- | 7.56 (d, J = 8.9 Hz, 1H), 7.45-7.42 (m, 1H), 7.25 (dd, J = |
| | triazole-4-carboxamide | 8.9, 3.1 Hz, 1H), 3.83 (d, J = 14.4 Hz, 3H), 2.43 (s, 3H); |
| | | LCMS(M − 1): 508.3 |
| 508 | 1-(2-chloro-4-methylphenyl)-5- | ¹H-NMR (400 MHz, DMSO-d₆) δ 7.73 (d, J = 7.9 Hz, |
| | (trifluoromethyl)-N-((2,4,6- | 1H), 7.65 (d, J = 0.9 Hz, 1H), 7.44-7.41 (m, 1H), 7.33 (t, |
| | trifluorophenyl)sulfonyl)-1H-1,2,3- | J = 9.2 Hz, 2H), 2.43 (s, 3H); LCMS(M − 2): 496.3 |
| | triazole-4-carboxamide | |
| 509 | 1-(2-chloro-4-methylphenyl)-N-((2- | ¹H-NMR (400 MHz, DMSO-d₆) δ 8.29 (d, J = 2.1 Hz, |
| | chloro-5- | 1H), 7.95 (dd, J = 8.4, 2.0 Hz, 1H), 7.82 (d, J = 8.3 Hz, |
| | (trifluoromethyl)phenyl)sulfonyl)-5- | 1H), 7.70 (d, J = 7.9 Hz, 1H), 7.62 (d, J = 0.9 Hz, 1H), |
| | (trifluoromethyl)-1H-1,2,3-triazole- | 7.40-7.38 (m, 1H), 2.40 (s, 3H); LCMS(M − 2): 545.3 |
| | 4-carboxamide | |
| 510 | 1-(2-chloro-4-methylphenyl)-N- | ¹H-NMR (400 MHz, DMSO-d₆) δ 8.03 (td, J = 8.6, 6.4 |
| | ((2,4-difluorophenyl)sulfonyl)-5- | Hz, 1H), 7.73 (d, J = 7.9 Hz, 1H), 7.66 (d, J = 0.9 Hz, |
| | (trifluoromethyl)-1H-1,2,3-triazole- | 1H), 7.52-7.47 (m, 1H), 7.44-7.42 (m, 1H), 7.30 (td, J = |
| | 4-carboxamide | 8.5, 2.0 Hz, 1H), 2.43 (s, 3H); LCMS(M − 2): 478.3 |
| 511 | 1-(2-chloro-4-methylphenyl)-5- | ¹H-NMR (400 MHz, DMSO-d₆) δ 8.35 (d, J = 7.6 Hz, |
| | (trifluoromethyl)-N-((2- | 1H), 7.95 (d, J = 7.6 Hz, 1H), 7.92-7.88 (m, 1H), 7.85 (d, |
| | (trifluoromethyl)phenyl)sulfonyl)- | J = 7.3 Hz, 1H), 7.73 (d, J = 8.3 Hz, 1H), 7.65 (s, 1H), |
| | 1H-1,2,3-triazole-4-carboxamide | 7.44-7.42 (m, 1H), 2.43 (s, 3H); LCMS(M − 2): 510.3 |
| 512 | 1-(2-chloro-4-methylphenyl)-N- | ¹H-NMR (400 MHz, DMSO-d₆) δ 10.00 (s, 1H), 9.91 (d, |
| | ((2,5-dimethylphenyl)sulfonyl)-5- | J = 8.3 Hz, 1H), 9.83 (d, J = 0.9 Hz, 1H), 9.61-9.55 (m, |
| | (trifluoromethyl)-1H-1,2,3-triazole- | 2H), 9.47 (d, J = 7.6 Hz, 1H), 4.76 (s, 3H), 4.59 (s, 3H), |
| | 4-carboxamide | 4.53 (s, 3H); LCMS(M − 1): 496.3 |
| 513 | N-((3-chloro-2- | ¹H-NMR (400 MHz, DMSO-d₆) δ 8.49 (d, J = 1.5 Hz, |
| | methylphenyl)sulfonyl)-1-(2-chloro- | 1H), 8.16 (dd, J = 8.1, 1.7 Hz, 1H), 8.07 (dd, J = 7.9, 1.1 |
| | 4-cyanophenyl)-5-methyl-1H-1,2,3- | Hz, 1H), 7.96 (d, J = 8.6 Hz, 1H), 7.79 (d, J = 7.3 Hz, |
| | triazole-4-carboxamide | 1H), 7.49 (t, J = 7.8 Hz, 1H), 2.66 (s, 3H), 2.26 (s, 3H); |
| | | LCMS (M − 1): 449.35 |
| 514 | 1-(2-chloro-4-cyanophenyl)-N-((2- | ¹H-NMR (400 MHz, DMSO-d₆) δ 12.46 (s, 1H), 8.49 (d, |
| | methoxy-5-methylphenyl)sulfonyl)- | J = 1.5 Hz, 1H), 8.16 (dd, J = 8.2, 1.8 Hz, 1H), 7.97 (d, |
| | 5-methyl-1H-1,2,3-triazole-4- | J = 8.3 Hz, 1H), 7.71 (d, J = 1.7 Hz, 1H), 7.47 (dd, J = 8.6, |
| | carboxamide | 1.7 Hz, 1H), 7.13 (d, J = 8.6 Hz, 1H), 3.83 (s, 3H), 2.32 |
| | | (s, 3H), 2.26 (s, 3H); LCMS (M+): 445.40 |
| 515 | N-((5-chloro-2- | ¹H-NMR (400 MHz, DMSO-d₆) δ 8.50 (d, J = 1.7 Hz, |
| | methoxyphenyl)sulfonyl)-1-(2- | 1H), 8.16 (dd, J = 8.2, 1.8 Hz, 1H), 7.97 (d, J = 8.3 Hz, |
| | chloro-4-cyanophenyl)-5-methyl- | 1H), 7.84 (d, J = 2.7 Hz, 1H), 7.75 (dd, J = 9.0, 2.7 Hz, |
| | 1H-1,2,3-triazole-4-carboxamide | 1H), 7.30 (d, J = 9.0 Hz, 1H), 3.88 (s, 3H), 2.27 (s, 3H); |
| | | LCMS (M − 1): 465.50 |
| 516 | 1-(2-chloro-4-cyanophenyl)-N-((5- | ¹H-NMR (400 MHz, DMSO-d₆) δ 12.66-13.84 (1H), 8.49 |
| | fluoro-2-methylphenyl)sulfonyl)-5- | (d, J = 1.5 Hz, 1H), 8.17-8.14 (m, 1H), 7.96 (d, J = 8.1 |
| | methyl-1H-1,2,3-triazole-4- | Hz, 1H), 7.79 (dd, J = 8.6, 2.0 Hz, 1H), 7.52-7.45 (m, |
| | carboxamide | 2H), 2.61 (s, 3H), 2.27 (s, 3H); LCMS (M+): 433.35 |
| 517 | 1-(2-chloro-5-methoxyphenyl)-N- | ¹H-NMR (400 MHz, DMSO-d₆) δ 8.18-8.16 (m, 1H), |
| | ((2-chlorophenyl)sulfonyl)-5- | 7.72-7.66 (m, 3H), 7.63-7.58 (m, 2H), 7.31 (dd, J = 8.9, |
| | (trifluoromethyl)-1H-1,2,3-triazole- | 3.1 Hz, 1H), 3.80 (s, 3H); LCMS(M + 1): 494.35 |
| | 4-carboxamide | |
| 518 | 1-(2-chloro-5-methoxyphenyl)-N- | ¹H-NMR (400 MHz, DMSO-d₆) δ 8.14 (dd, J = 7.9, 1.8 |
| | ((2- | Hz, 1H), 7.82 (td, J = 7.9, 1.5 Hz, 1H), 7.70 (d, J = 9.2 |
| | (trifluoromethoxy)phenyl)sulfonyl)- | Hz, 1H), 7.64-7.57 (m, 3H), 7.31 (dd, J = 8.9, 3.1 Hz, |
| | 5-(trifluoromethyl)-1H-1,2,3- | 1H), 3.80 (s, 3H); LCMS(M+): 544.30 |
| | triazole-4-carboxamide | |
| 519 | 1-(2-chloro-5-methoxyphenyl)-N- | ¹H-NMR (400 MHz, DMSO-d₆) δ 7.71-7.67 (m, 2H), |
| | ((2,5-difluorophenyl)sulfonyl)-5- | 7.58-7.53 (m, 2H), 7.46 (td, J = 9.3, 4.2 Hz, 1H), 7.30 (dd, |
| | (trifluoromethyl)-1H-1,2,3-triazole- | J = 9.2, 3.1 Hz, 1H), 3.80 (s, 3H); LCMS(M + 2): 498.75 |
| | 4-carboxamide | |
| 520 | 1-(2-chloro-5-methoxyphenyl)-N- | ¹H-NMR (400 MHz, DMSO-d₆) δ 8.05 (td, J = 8.7, 6.3 |
| | ((2,4-difluorophenyl)sulfonyl)-5- | Hz, 1H), 7.70 (d, J = 9.2 Hz, 1H), 7.59 (d, J = 3.1 Hz, |
| | (trifluoromethyl)-1H-1,2,3-triazole- | 1H), 7.56-7.51 (m, 1H), 7.35-7.30 (m, 2H), 3.81 (d, J = |
| | 4-carboxamide | 10.7 Hz, 3H); LCMS(M − 2): 494.40 |
| 521 | 1-(2-chloro-5-methoxyphenyl)-5- | ¹H-NMR (400 MHz, DMSO-d₆) δ 7.69 (d, J = 8.9 Hz, |
| | (trifluoromethyl)-N-((2,4,6- | 1H), 7.58 (d, J = 3.1 Hz, 1H), 7.37-7.28 (m, 3H), 3.80 (s, |
| | trifluorophenyl)sulfonyl)-1H-1,2,3- | 3H); LCMS(M − 1): 512.35 |
| | triazole-4-carboxamide | |
| 522 | N-((2,5- | ¹H-NMR (400 MHz, DMSO-d₆) δ 8.54 (s, 1H), 8.13-8.08 |
| | bis(trifluoromethyl)phenyl)sulfonyl)- | (m, 2H), 7.67 (d, J = 8.9 Hz, 1H), 7.55 (d, J = 3.1 Hz, 1H), |
| | 1-(2-chloro-5-methoxyphenyl)-5- | 7.28 (dd, J = 8.9, 3.1 Hz, 1H), 3.80 (s, 3H); LCMS(M − 2): |
| | (trifluoromethyl)-1H-1,2,3-triazole- | 594.25 |
| | 4-carboxamide | |
| 523 | N-((2,5- | ¹H-NMR (400 MHz, DMSO-d₆) δ 8.55 (s, 1H), 8.15 (dd, |
| | bis(trifluoromethyl)phenyl)sulfonyl)- | J = 14.5, 8.7 Hz, 2H), 7.72 (t, J = 4.1 Hz, 1H), 7.64 (d, |
| | 1-(2-chloro-4-methylphenyl)-5- | J = 0.9 Hz, 1H), 7.41 (dt, J = 9.0, 0.9 Hz, 1H), 2.42 (s, 3H), |
| | (trifluoromethyl)-1H-1,2,3-triazole- | LCMS(M − 2): 578.3 |
| | 4-carboxamide | |
| 524 | 1-(2-chloro-4-methylphenyl)-N- | ¹H-NMR (400 MHz, DMSO-d₆) δ 8.14 (d, J = 8.6 Hz, |
| | ((2,4-dichlorophenyl)sulfonyl)-5- | 1H), 7.87 (d, J = 1.8 Hz, 1H), 7.75 (d, J = 8.3 Hz, 1H), |
| | (trifluoromethyl)-1H-1,2,3-triazole- | 7.70-7.66 (m, 2H), 7.43 (dd, J = 7.9, 0.9 Hz, 1H), 2.43 (s, |
| | 4-carboxamide | 3H), LCMS(M − 1): 512.2 |
| 525 | N-((3-chloro-4- | ¹H-NMR (400 MHz, DMSO-d₆) δ 8.13 (dd, J = 6.7, 2.4 |
| | fluorophenyl)sulfonyl)-1-(2-chloro- | Hz, 1H), 8.01 (qd, J = 4.4, 2.4 Hz, 1H), 7.75-7.68 (m, |
| | 4-methylphenyl)-5-(trifluoromethyl)- | 2H), 7.66 (d, J = 0.9 Hz, 1H), 7.45-7.42 (m, 1H), 2.43 (s, |
| | 1H-1,2,3-triazole-4-carboxamide | 3H), LCMS(M + 1): 498.1 |
| 526 | 1-(2-chloro-4-methylphenyl)-N-((4- | ¹H-NMR (400 MHz, DMSO-d₆) δ 8.10 (dd, J = 6.9, 2.0 |
| | (trifluoromethoxy)phenyl)sulfonyl)- | Hz, 2H), 7.73 (d, J = 8.3 Hz, 1H), 7.65-7.61 (m, 3H), 7.43 |
| | 5-(trifluoromethyl)-1H-1,2,3- | (dd, J = 8.1, 1.1 Hz, 1H), 2.43 (s, 3H), LCMS(M − 2): 526.3 |
| | triazole-4-carboxamide | |
| 527 | N-((4-chloro-2,5- | ¹H-NMR (400 MHz, DMSO-d₆) δ 7.99 (s, 1H), 7.75 (d, |
| | dimethylphenyl)sulfonyl)-1-(2- | J = 8.3 Hz, 1H), 7.67 (d, J = 0.9 Hz, 1H), 7.55 (s, 1H), 7.45- |
| | chloro-4-methylphenyl)-5- | 7.43 (m, 1H), 2.60 (s, 3H), 2.43 (s, 3H), 2.39 (s, 3H), |
| | (trifluoromethyl)-1H-1,2,3-triazole- | LCMS(M − 2): 478.3 |
| | 4-carboxamide | |
| 528 | N-((5-chloro-2,4- | ¹H-NMR (400 MHz, DMSO-d₆) δ 8.05 (dd, J = 8.1, 7.2 |
| | difluorophenyl)sulfonyl)-1-(2- | Hz, 1H), 7.78-7.72 (m, 2H), 7.65 (d, J = 0.9 Hz, 1H), |
| | chloro-4-methylphenyl)-5- | 7.44-7.41 (m, 1H), 2.43 (s, 3H), LCMS(M − 3): 512.2 |
| | (trifluoromethyl)-1H-1,2,3-triazole- | |
| | 4-carboxamide | |
| 529 | N-((5-chloro-2- | ¹H-NMR (400 MHz, DMSO-d₆) δ 7.88 (q, J = 3.0 Hz, |
| | fluorophenyl)sulfonyl)-1-(2-chloro- | 1H), 7.78-7.72 (m, 2H), 7.65 (d, J = 0.9 Hz, 1H), 7.48- |
| | 4-methylphenyl)-5-(trifluoromethyl)- | 7.41 (m, 2H), 2.43 (s, 3H), LCMS(M − 1): 496.3 |
| | 1H-1,2,3-triazole-4-carboxamide | |
| 530 | 1-(2-chloro-4-methylphenyl)-N- | ¹H-NMR (400 MHz, DMSO-d₆) δ 7.75-7.71 (m, 2H), |
| | ((2,5-difluorophenyl)sulfonyl)-5- | 7.66 (d, J = 0.8 Hz, 1H), 7.64-7.58 (m, 1H), 7.50 (td, J = |
| | (trifluoromethyl)-1H-1,2,3-triazole- | 9.2, 4.2 Hz, 1H), 7.44-7.42 (m, 1H), 2.43 (s, 3H), |
| | 4-carboxamide | LCMS(M − 2): 478.3 |
| 531 | 1-(2-chloro-4-methylphenyl)-N-((4- | ¹H-NMR (400 MHz, DMSO-d₆) δ 8.01 (dt, J = 9.1, 2.3 |
| | chlorophenyl)sulfonyl)-5- | Hz, 2H), 7.75-7.72 (m, 3H), 7.66 (d, J = 0.9 Hz, 1H), |
| | (trifluoromethyl)-1H-1,2,3-triazole- | 7.45-7.42 (m, 1H), 2.43 (s, 3H), LCMS(M − 3): 476.3 |
| | 4-carboxamide | |
| 532 | 1-(2-chloro-4-methylphenyl)-N-((4- | ¹H-NMR (400 MHz, DMSO-d₆) δ 8.09-8.05 (m, 2H), |
| | fluorophenyl)sulfonyl)-5- | 7.74 (d, J = 8.3 Hz, 1H), 7.66 (d, J = 0.9 Hz, 1H), 7.53- |
| | (trifluoromethyl)-1H-1,2,3-triazole- | 7.48 (m, 2H), 7.44 (dd, J = 8.3, 0.9 Hz, 1H), 2.43 (s, 3H), |
| | 4-carboxamide | LCMS(M+): 462.5 |
| 533 | N-((2-chloro-5- | ¹H-NMR (400 MHz, CHLOROFORM-D) δ 10.06 (s, |
| | methoxyphenyl)sulfonyl)-1-(2,4- | 1H), 7.86 (d, J = 3.2 Hz, 1H), 7.48 (dd, J = 2.2, 0.5 Hz, |
| | dichloro-6-methylphenyl)-5- | 1H), 7.43 (d, J = 8.8 Hz, 1H), 7.35 (dd, J = 2.2, 0.5 Hz, |
| | (trifluoromethyl)-1H-1,2,3-triazole- | 1H), 7.11 (dd, J = 8.9, 3.1 Hz, 1H), 3.91 (s, 3H), 2.03 (s, |
| | 4-carboxamide | 3H); LCMS (M + 1): 544.2 |
| 534 | N-((2-chloro-5- | ¹H-NMR (400 MHz, CHLOROFORM-D) δ 10.10 (s, |
| | (trifluoromethyl)phenyl)sulfonyl)-1- | 1H), 8.64 (d, J = 1.8 Hz, 1H), 7.86 (dd, J = 8.3, 1.8 Hz, |
| | (2,4-dichloro-6-methylphenyl)-5- | 1H), 7.72 (d, J = 8.3 Hz, 1H), 7.52-7.48 (m, 1H), 7.35 (t, |
| | (trifluoromethyl)-1H-1,2,3-triazole- | J = 1.1 Hz, 1H), 2.03 (s, 3H); LCMS (M − 1): 580.80 |
| | 4-carboxamide | |
| 535 | N-((2-chloro-5- | ¹H-NMR (400 MHz, DMSO-d₆) δ 8.37-8.31 (m, 2H), |
| | (trifluoromethyl)phenyl)sulfonyl)-1- | 8.15-8.11 (m, 1H), 8.05 (dd, J = 8.2, 0.9 Hz, 1H), 7.98 |
| | (4-cyano-2-fluorophenyl)-5- | (dd, J = 8.4, 2.1 Hz, 1H), 7.84 (d, J = 8.3 Hz, 1H); LCMS |
| | (trifluoromethyl)-1H-1,2,3-triazole- | (M − 2): 539.30 |
| | 4-carboxamide | |
| 536 | 1-(4-cyano-2-fluorophenyl)-N-((2,5- | ¹H-NMR (400 MHz, DMSO-d₆) δ 8.37 (dd, J = 9.8, 1.5 |
| | dimethylphenyl)sulfonyl)-5- | Hz, 1H), 8.14-8.05 (m, 2H), 7.85 (d, J = 0.9 Hz, 1H), 7.42 |
| | (trifluoromethyl)-1H-1,2,3-triazole- | (dd, J = 7.8, 1.1 Hz, 1H), 7.32 (d, J = 7.9 Hz, 1H), 2.58 s, |
| | 4-carboxamide | 3H), 2.37 (s, 3H); LCMS (M − 2): 465.40 |
| 537 | 1-(4-cyano-2-fluorophenyl)-N-((2,5- | ¹H-NMR (400 MHz, DMSO-d₆) δ 8.36 (dd, J = 9.9, 1.6 |
| | difluorophenyl)sulfonyl)-5- | Hz, 1H), 8.14-8.11 (m, 1H), 8.06-8.04 (m, 1H), 7.71-7.67 |
| | (trifluoromethyl)-1H-1,2,3-triazole- | (m, 1H), 7.59-7.53 (m, 1H), 7.46 (td, J = 9.2, 4.1 Hz, 1H); |
| | 4-carboxamide | LCMS (M − 2): 473.30 |
| 538 | N-((2-chloro-5- | ¹H-NMR (400 MHz, DMSO-d₆) δ 8.37 (dd, J = 9.8, 1.5 |
| | methoxyphenyl)sulfonyl)-1-(4- | Hz, 1H), 8.14 (t, J = 7.7 Hz, 1H), 8.06 (dd, J = 8.3, 1.0 |
| | cyano-2-fluorophenyl)-5- | Hz, 1H), 7.60 (d, J = 2.9 Hz, 1H), 7.55 (d, J = 8.8 Hz, |
| | (trifluoromethyl)-1H-1,2,3-triazole- | 1H), 7.25 (dd, J = 8.8, 3.2 Hz, 1H), 3.84 (s, 3H); LCMS |
| | 4-carboxamide | (M − 2): 501.35 |
| 539 | N-((2-bromo-5- | ¹H-NMR (400 MHz, CHLOROFORM-D) δ 10.12 (s, |
| | (trifluoromethyl)phenyl)sulfonyl)-1- | 1H), 8.66 (d, J = 2.0 Hz, 1H), 7.93 (d, J = 8.3 Hz, 1H), |
| | (2,4-dichloro-6-methylphenyl)-5- | 7.76-7.74 (m, 1H), 7.48 (dd, J = 2.2, 0.5 Hz, 1H), 7.35 (q, |
| | (trifluoromethyl)-1H-1,2,3-triazole- | J = 1.0 Hz, 1H), 2.04 (s, 3H); LCMS (M − 1): 624.10 |
| | 4-carboxamide | |
| 540 | 1-(2,4-dichloro-6-methylphenyl)-N- | ¹H-NMR (400 MHz, CHLOROFORM-D) δ 10.02 (s, |
| | ((2,5-dimethylphenyl)sulfonyl)-5- | 1H), 8.39 (dd, J = 7.9, 1.5 Hz, 1H), 7.64-7.51 (m, 3H), |
| | (trifluoromethyl)-1H-1,2,3-triazole- | 7.47 (d, J = 2.1 Hz, 1H), 7.35 (d, J = 1.8 Hz, 1H), 2.03 (s, |
| | 4-carboxamide | 3H); LCMS (M − 1): 505.05 |
| 541 | N-((5-chloro-2,4- | ¹H-NMR (400 MHz, CHLOROFORM-D) δ 10.06 (s, |
| | difluorophenyl)sulfonyl)-1-(2,4- | 1H), 8.29 (t, J = 7.5 Hz, 1H), 7.48 (dd, J = 2.2, 0.5 Hz, |
| | dichloro-6-methylphenyl)-5- | 1H), 7.36-7.35 (m, 1H), 7.11 (dd, J = 9.3, 8.3 Hz, 1H), |
| | (trifluoromethyl)-1H-1,2,3-triazole- | 2.03 (s, 3H); LCMS (M − 1): 548.15 |
| | 4-carboxamide | |
| 542 | N-((2-bromo-5- | ¹H-NMR (400 MHz, DMSO-d₆) δ 9.10 (d, J = 13.8 Hz, |
| | (trifluoromethyl)phenyl)sulfonyl)-1- | 1H), 8.38 (s, 1H), 8.10 (d, J = 8.3 Hz, 1H), 7.97 (d, J = |
| | (2-chloro-4-methylphenyl)-1H-1,2,3- | 7.0 Hz, 1H), 7.61 (d, J = 8.3 Hz, 2H), 7.40 (d, J = 7.6 Hz, |
| | triazole-4-carboxamide | 1H), 2.41 (s, 3H); LCMS(M + 1): 524.2 |
| 543 | 1-(2-chloro-4-methylphenyl)-N- | ¹H-NMR (400 MHz, DMSO-d₆) δ 9.16 (d, J = 6.7 Hz, |
| | ((2,5-difluorophenyl)sulfonyl)-1H- | 1H), 7.80-7.76 (m, 1H), 7.70-7.64 (m, 1H), 7.63-7.60 (m, |
| | 1,2,3-triazole-4-carboxamide | 2H), 7.59-7.52 (m, 1H), 7.41-7.39 (m, 1H), 2.41 (s, 3H); |
| | | LCMS(M + 1): 413.0 |
| 544 | N-((2-chlorophenyl)sulfonyl)-1-(2,4- | ¹H-NMR (400 MHz, CHLOROFORM-D) δ 10.02 (s, |
| | dichloro-6-methylphenyl)-5- | 1H), 8.39 (dd, J = 7.9, 1.5 Hz, 1H), 7.64-7.51 (m, 3H), |
| | (trifluoromethyl)-1H-1,2,3-triazole- | 7.47 (d, J = 2.1 Hz, 1H), 7.35 (d, J = 1.8 Hz, 1H), 2.03 (s, |
| | 4-carboxamide | 3H); LCMS (M − 1): 512.90 |
| 545 | 1-(2,4-dichloro-6-methylphenyl)-N- | ¹H-NMR (400 MHz, CHLOROFORM-D) δ 10.02 (s, |
| | ((2- | 1H), 8.39 (dd, J = 7.9, 1.5 Hz, 1H), 7.64-7.51 (m, 3H), |
| | (trifluoromethoxy)phenyl)sulfonyl)- | 7.47 (d, J = 2.1 Hz, 1H), 7.35 (d, J = 1.8 Hz, 1H), 2.03 (s, |
| | 5-(trifluoromethyl)-1H-1,2,3- | 3H); LCMS (M − 1): 560.90 |
| | triazole-4-carboxamide | |
| 546 | 1-(2,4-dichloro-6-methylphenyl)-N- | ¹H-NMR (400 MHz, CHLOROFORM-D) δ 9.81 (s, 1H), |
| | ((4- | 8.29-8.26 (m, 2H), 7.47 (d, J = 1.8 Hz, 1H), 7.43-7.40 (m, |
| | (trifluoromethoxy)phenyl)sulfonyl)- | 2H), 7.35 (d, J = 1.5 Hz, 1H), 2.02 (s, 3H); LCMS (M − 1): |
| | 5-(trifluoromethyl)-1H-1,2,3- | 560.90 |
| | triazole-4-carboxamide | |
| 547 | 1-(2,4-dichloro-6-methylphenyl)-N- | ¹H-NMR (400 MHz, CHLOROFORM-D) δ 9.92 (s, 1H), |
| | ((2,4-difluorophenyl)sulfonyl)-5- | 8.24 (td, J = 8.5, 6.1 Hz, 1H), 7.48 (d, J = 1.8 Hz, 1H), |
| | (trifluoromethyl)-1H-1,2,3-triazole- | 7.46-7.47 (0H), 7.35 (q, J = 1.0 Hz, 1H), 7.13-7.08 (m, |
| | 4-carboxamide | 1H), 7.03-6.98 (m, 1H), 2.03 (s, 3H); LCMS (M − 1): |
| | | 513.00 |
| 548 | 1-(2,4-dichloro-6-methylphenyl)-N- | ¹H-NMR (400 MHz, CHLOROFORM-D) δ 10.01 (s, |
| | ((2-fluorophenyl)sulfonyl)-5- | 1H), 8.23-8.19 (m, 1H), 7.72-7.66 (m, 1H), 7.47 (dd, J = |
| | (trifluoromethyl)-1H-1,2,3-triazole- | 2.2, 0.5 Hz, 1H), 7.39 (td, J = 7.7, 1.0 Hz, 1H), 7.35 (q, |
| | 4-carboxamide | J = 1.0 Hz, 1H), 7.29 (m, 1H), 2.03 (s, 3H); LCMS (M + 1): |
| | | 496.35 |
| 549 | 1-(2,4-dichloro-6-methylphenyl)-N- | ¹H-NMR (400 MHz, CHLOROFORM-D) δ 10.08 (s, |
| | ((2,4,5-trichlorophenyl)sulfonyl)-5- | 1H), 8.45 (s, 1H), 7.67 (s, 1H), 7.48 (q, J = 1.0 Hz, 1H), |
| | (trifluoromethyl)-1H-1,2,3-triazole- | 7.35 (q, J = 1.0 Hz, 1H), 2.04 (s, 3H); LCMS (M − 1): |
| | 4-carboxamide | 580.15 |
| 550 | N-((5-chloro-2- | ¹H-NMR (400 MHz, CHLOROFORM-D) δ 10.04 (s, |
| | fluorophenyl)sulfonyl)-1-(2,4- | 1H), 8.17 (q, J = 2.9 Hz, 1H), 7.63 (qd, J = 4.3, 2.6 Hz, |
| | dichloro-6-methylphenyl)-5- | 1H), 7.48-7.48 (m, 1H), 7.36-7.35 (m, 1H), 7.24-7.20 (m, |
| | (trifluoromethyl)-1H-1,2,3-triazole- | 1H), 2.03 (s, 3H); LCMS (M − 1): 530.80 |
| | 4-carboxamide | |
| 551 | 1-(2,4-dichloro-6-methylphenyl)-5- | ¹H-NMR (400 MHz, CHLOROFORM-D) δ 10.16 (s, |
| | (trifluoromethyl)-N-((2,4,6- | 1H), 7.48 (d, J = 2.1 Hz, 1H), 7.36 (t, J = 1.1 Hz, 1H), |
| | trifluorophenyl)sulfonyl)-1H-1,2,3- | 6.90-6.83 (m, 2H), 2.04 (s, 3H); LCMS (M + 1): 534.85 |
| | triazole-4-carboxamide | |
| 552 | N-((3-chloro-4- | ¹H-NMR (400 MHz, CHLOROFORM-D) δ 9.84 (s, 1H), |
| | fluorophenyl)sulfonyl)-1-(2,4- | 8.27 (dd, J = 6.6, 2.3 Hz, 1H), 8.15 (qd, J = 4.4, 2.4 Hz, |
| | dichloro-6-methylphenyl)-5- | 1H), 7.48 (d, J = 2.1 Hz, 1H), 7.38-7.34 (m, 2H), 2.02 (s, |
| | (trifluoromethyl)-1H-1,2,3-triazole- | 3H); LCMS (M + 1): 532.80 |
| | 4-carboxamide | |
| 553 | 1-(2,4-dichloro-6-methylphenyl)-N- | ¹H-NMR (400 MHz, CHLOROFORM-D) δ 10.03 (s, |
| | ((2,4-dichlorophenyl)sulfonyl)-5- | 1H), 8.32 (d, J = 8.6 Hz, 1H), 7.58 (d, J = 1.8 Hz, 1H), |
| | (trifluoromethyl)-1H-1,2,3-triazole- | 7.50 (dd, J = 8.7, 2.0 Hz, 1H), 7.48 (d, J = 1.8 Hz, 1H), |
| | 4-carboxamide | 7.35 (d, J = 1.5 Hz, 1H), 2.03 (s, 3H); LCMS (M − 1): |
| | | 546.80 |
| 554 | 1-(2,4-dichloro-6-methylphenyl)-N- | ¹H-NMR (400 MHz, CHLOROFORM-D) δ 9.77 (s, 1H), |
| | ((4-fluorophenyl)sulfonyl)-5- | 8.27-8.22 (m, 2H), 7.47 (d, J = 1.8 Hz, 1H), 7.34 (q, J = |
| | (trifluoromethyl)-1H-1,2,3-triazole- | 0.9 Hz, 1H), 7.29-7.27 (m, 2H), 2.01 (s, 3H); LCMS |
| | 4-carboxamide | (M + 1): 496.90 |
| 555 | N-((4-chloro-2,5- | ¹H-NMR (400 MHz, CHLOROFORM-D) δ 9.82 (s, 1H), |
| | dimethylphenyl)sulfonyl)-1-(2,4- | 8.15 (s, 1H), 7.48 (q, J = 0.9 Hz, 1H), 7.35-7.34 (m, 2H), |
| | dichloro-6-methylphenyl)-5- | 2.69 (s, 3H), 2.44 (s, 3H), 2.02 (s, 3H); LCMS (M − 1): |
| | (trifluoromethyl)-1H-1,2,3-triazole- | 540.95 |
| | 4-carboxamide | |
| 556 | N-((2,5- | ¹H-NMR (400 MHz, CHLOROFORM-D) δ 9.55-10.24 |
| | bis(trifluoromethyl)phenyl)sulfonyl)- | (1H), 8.88 (s, 1H), 8.08 (s, 2H), 7.48-7.47 (m, 1H), 7.35 |
| | 1-(2,4-dichloro-6-methylphenyl)-5- | (q, J = 1.1 Hz, 1H), 2.02 (s, 3H); LCMS (M − 1): 612.95 |
| | (trifluoromethyl)-1H-1,2,3-triazole- | |
| | 4-carboxamide | |
| 557 | 1-(2,4-dichloro-6-methylphenyl)-5- | ¹H-NMR (400 MHz, CHLOROFORM-D) δ 9.91 (s, 1H), |
| | (trifluoromethyl)-N-((2- | 8.65 (dd, J = 7.3, 1.8 Hz, 1H), 7.94-7.90 (m, 1H), 7.87- |
| | (trifluoromethyl)phenyl)sulfonyl)- | 7.80 (m, 2H), 7.47-7.47 (m, 1H), 7.34-7.34 (m, 1H), 2.02 |
| | 1H-1,2,3-triazole-4-carboxamide | (s, 3H); LCMS (M − 1): 544.95 |
| 558 | N-((4-chlorophenyl)sulfonyl)-1-(2,4- | ¹H-NMR (400 MHz, CHLOROFORM-D) δ 9.77 (s, 1H), |
| | dichloro-6-methylphenyl)-5- | 8.17-8.14 (m, 2H), 7.57 (dt, J = 9.1, 2.3 Hz, 2H), 7.48- |
| | (trifluoromethyl)-1H-1,2,3-triazole- | 7.47 (m, 1H), 7.34 (q, J = 0.9 Hz, 1H), 2.01 (s, 3H); |
| | 4-carboxamide | LCMS (M − 1): 512.85 |
| 559 | 1-(2,4-dichloro-6-methylphenyl)-N- | ¹H-NMR (400 MHz, CHLOROFORM-D) δ 10.05 (s, |
| | ((2,5-difluorophenyl)sulfonyl)-5- | 1H), 7.91 (td, J = 6.2, 3.3 Hz, 1H), 7.48 (dd, J = 2.2, 0.5 |
| | (trifluoromethyl)-1H-1,2,3-triazole- | Hz, 1H), 7.41-7.35 (m, 2H), 7.25-7.22 (m, 1H), 2.03 (s, |
| | 4-carboxamide | 3H); LCMS (M − 1): 512.30 |
| 560 | N-((3,5- | ¹H-NMR (400 MHz, CHLOROFORM-D) δ 10.16 (s, |
| | bis(trifluoromethyl)phenyl)sulfonyl)- | 1H), 8.67 (s, 2H), 8.18 (s, 1H), 7.47 (d, J = 1.8 Hz, 1H), |
| | 1-(2,4-dichloro-6-methylphenyl)-5- | 7.35 (q, J = 0.9 Hz, 1H), 2.63 (s, 6H), 2.02 (s, 3H); LCMS |
| | (trifluoromethyl)-1H-1,2,3-triazole- | (M − 1): 612.85 |
| | 4-carboxamide | |
| 561 | 1-(4-cyano-2-fluorophenyl)-N-((2,5- | ¹H-NMR (400 MHz, DMSO-d₆) δ 8.34 (dd, J = 9.8, 1.8 |
| | dichlorophenyl)sulfonyl)-5- | Hz, 1H), 8.13 (t, J = 7.8 Hz, 1H), 8.04 (dt, J = 8.3, 0.9 Hz, |
| | (trifluoromethyl)-1H-1,2,3-triazole- | 1H), 8.02 (d, J = 2.4 Hz, 1H), 7.64 (dd, J = 8.6, 2.8 Hz, |
| | 4-carboxamide | 1H), 7.58 (d, J = 8.6 Hz, 1H); LCMS (M − 1): 507.75 |
| 562 | 1-(4-cyano-2-fluorophenyl)-5- | ¹H-NMR (400 MHz, DMSO-d₆) δ 8.36 (dd, J = 9.6, 1.4 |
| | (trifluoromethyl)-N-((2- | Hz, 2H), 8.13 (t, J = 7.8 Hz, 1H), 8.05 (dt, J = 8.4, 0.8 Hz, |
| | (trifluoromethyl)phenyl)sulfonyl)- | 1H), 7.97-7.95 (m, 1H), 7.93-7.89 (m, 1H), 7.86 (t, J = |
| | 1H-1,2,3-triazole-4-carboxamide | 7.5 Hz, 1H); LCMS (M − 2): 505.90 |
| 563 | N-((2-chlorophenyl)sulfonyl)-1-(4- | ¹H-NMR (400 MHz, DMSO-d₆) δ 8.37 (dd, J = 9.8, 1.5 |
| | cyano-2-fluorophenyl)-5- | Hz, 1H), 8.18-8.12 (m, 2H), 8.06 (dd, J = 8.3, 0.9 Hz, |
| | (trifluoromethyl)-1H-1,2,3-triazole- | 1H), 7.73-7.65 (m, 2H), 7.63-7.57 (m, 1H); LCMS (M − |
| | 4-carboxamide | 2): 471.90 |
| 564 | 1-(4-cyano-2-fluorophenyl)-N-((2- | ¹H-NMR (400 MHz, DMSO-d₆) δ 8.36 (dd, J = 9.8, 1.5 |
| | fluorophenyl)sulfonyl)-5- | Hz, 1H), 8.12 (t, J = 7.8 Hz, 1H), 8.06 (dd, J = 8.3, 1.2 |
| | (trifluoromethyl)-1H-1,2,3-triazole- | Hz, 1H), 7.98 (td, J = 7.6, 1.6 Hz, 1H), 7.77-7.71 (m, 1H), |
| | 4-carboxamide | 7.45-7.41 (m, 2H); LCMS (M+): 457.85 |
| 565 | 1-(2-chloro-4-methylphenyl)-N-((2- | ¹H-NMR (400 MHz, CHLOROFORM-D) δ 9.88 (s, 1H), |
| | (trifluoromethoxy)phenyl)sulfonyl)- | 8.45 (s, 1H), 8.34 (dd, J = 8.1, 1.7 Hz, 1H), 7.73-7.69 (m, |
| | 1H-1,2,3-triazole-4-carboxamide | 1H), 7.53-7.47 (m, 2H), 7.44-7.40 (m, 2H), 7.28 (d, J = |
| | | 1.2 Hz, 1H), 2.44 (s, 3H); LCMS(M+): 460.5 |
| 566 | 1-(2-chloro-4-methylphenyl)-N-((2- | ¹H-NMR (400 MHz, DMSO-d₆) δ 9.18 (d, J = 8.3 Hz, |
| | chlorophenyl)sulfonyl)-1H-1,2,3- | 1H), 8.19 (dd, J = 7.9, 1.5 Hz, 1H), 7.74-7.70 (m, 1H), |
| | triazole-4-carboxamide | 7.68 (dd, J = 7.9, 1.5 Hz, 1H), 7.65-7.59 (m, 2H), 7.40 |
| | | (dt, J = 8.0, 0.9 Hz, 1H), 2.42 (s, 3H); LCMS(M − 1): 410.5 |
| 567 | 1-(2-chloro-4-methylphenyl)-N- | ¹H-NMR (400 MHz, DMSO-d₆) δ 9.14 (s, 1H), 8.11 (d, |
| | ((2,5-dichlorophenyl)sulfonyl)-1H- | J = 2.4 Hz, 1H), 7.81-7.78 (m, 2H), 7.72 (s, 1H), 7.69 (q, |
| | 1,2,3-triazole-4-carboxamide | J = 1.3 Hz, 1H), 7.62 (d, J = 8.3 Hz, 2H), 7.40 (dt, J = 9.1, |
| | | 0.9 Hz, 1H), 2.41 (s, 3H); LCMS(M + 1) 446.4 |
| 568 | 1-(2-chloro-4-methylphenyl)-N-((2- | ¹H-NMR (400 MHz, DMSO-d₆) δ 9.17 (s, 1H), 8.02 (td, |
| | fluorophenyl)sulfonyl)-1H-1,2,3- | J = 7.6, 1.6 Hz, 1H), 7.81-7.76 (m, 1H), 7.63-7.59 (m, |
| | triazole-4-carboxamide | 2H), 7.49-7.44 (m, 2H), 7.41-7.39 (m, 1H), 2.41 (s, 3H); |
| | | LCMS(M+): 394.4 |
| 569 | 1-(2-chloro-4-methylphenyl)-N-((2- | ¹H-NMR (400 MHz, DMSO-d₆) δ 9.17 (d, J = 2.4 Hz, |
| | chloro-5-methoxyphenyl)sulfonyl)- | 1H), 7.62 (dd, J = 7.6, 3.1 Hz, 2H), 7.60-7.56 (m, 1H), |
| | 1H-1,2,3-triazole-4-carboxamide | 7.40 (dt, J = 9.0, 0.9 Hz, 1H), 7.29 (d, J = 8.9 Hz, 1H), |
| | | 3.85 (s, 3H), 2.40 (d, J = 13.1 Hz, 3H); LCMS(M − 1): |
| | | 440.4 |
| 570 | 1-(2-chloro-4-methylphenyl)-N- | ¹H-NMR (400 MHz, DMSO-d₆) δ 9.13 (s, 1H), 7.62-7.60 |
| | ((2,4,6-trifluorophenyl)sulfonyl)-1H- | (m, 2H), 7.46 (t, J = 9.5 Hz, 2H), 7.41-7.39 (m, 2H), 2.42 |
| | 1,2,3-triazole-4-carboxamide | (s, 3H); LCMS(M+): 430.5 |
| 571 | 1-(2-chloro-4-methylphenyl)-N-((2- | ¹H-NMR (400 MHz, DMSO-d₆) δ 9.11 (s, 1H), 8.37 (d, |
| | chloro-5- | J = 2.1 Hz, 1H), 8.09 (dd, J = 8.6, 1.8 Hz, 1H), 7.93 (d, J = |
| | (trifluoromethyl)phenyl)sulfonyl)- | 8.3 Hz, 1H), 7.61 (d, J = 8.3 Hz, 2H), 7.39 (dd, J = 7.9, |
| | 1H-1,2,3-triazole-4-carboxamide | 0.9 Hz, 1H), 2.41 (s, 3H); LCMS(M − 1): 478.5 |
| 572 | 1-(2-chloro-4-methylphenyl)-N- | ¹H-NMR (400 MHz, DMSO-d₆) δ 9.16 (d, J = 3.4 Hz, |
| | ((2,4-difluorophenyl)sulfonyl)-1H- | 1H), 8.08 (td, J = 8.6, 6.2 Hz, 1H), 7.63-7.59 (m, 3H), |
| | 1,2,3-triazole-4-carboxamide | 7.57-7.54 (m, 1H), 7.41-7.39 (m, 2H), 7.35 (dd, J = 8.7, |
| | | 2.0 Hz, 1H), 2.41 (s, 3H); LCMS(M+): 412.5 |
| 573 | 1-(2-chloro-4-methylphenyl)-N-((2- | ¹H-NMR (400 MHz, DMSO-d₆) δ 9.16 (s, 1H), 8.39 (d, |
| | (trifluoromethyl)phenyl)sulfonyl)- | J = 7.6 Hz, 1H), 8.02-7.98 (m, 1H), 7.97-7.94 (m, 1H), 7.89 |
| | 1H-1,2,3-triazole-4-carboxamide | (q, J = 7.4 Hz, 1H), 7.63-7.60 (m, 2H), 7.41-7.39 (m, 1H), |
| | | 2.41 (s, 3H); LCMS(M+): 444.4 |
| 574 | 1-(2-chloro-4-methylphenyl)-N- | ¹H-NMR (400 MHz, DMSO-d₆) δ 9.17 (s, 1H), 7.79 (dd, |
| | ((2,5-dimethylphenyl)sulfonyl)-1H- | J = 8.7, 2.3 Hz, 1H), 7.62-7.59 (m, 2H), 7.52-7.48 (m, |
| | 1,2,3-triazole-4-carboxamide | 2H), 7.40 (dd, J = 8.1, 1.1 Hz, 1H), 2.58 (d, J = 9.8 Hz, |
| | | 3H), 2.49 (q, J = 1.8 Hz, 3H), 2.41 (s, 3H); LCMS(M + 3): |
| | | 407.9 |
| 575 | N-((5-chloro-2,4- | ¹H-NMR (400 MHz, DMSO-d₆) δ 7.98 (t, J = 7.7 Hz, 1H), |
| | difluorophenyl)sulfonyl)-1-(2- | 7.75 (d, J = 8.8 Hz, 1H), 7.66 (t, J = 9.5 Hz, 1H), 7.37 (d, |
| | chloro-4-methoxyphenyl)-5- | J = 2.7 Hz, 1H), 7.14 (dd, J = 9.0, 2.7 Hz, 1H), 3.88 (s, |
| | (trifluoromethyl)-1H-1,2,3-triazole- | 3H); LCMS(M+): 530.95 |
| | 4-carboxamide | |
| 576 | 1-(2-chloro-4-methoxyphenyl)-N- | ¹H-NMR (400 MHz, DMSO-d₆) δ 8.16 (d, J = 4.9 Hz, |
| | ((2,4,5-trichlorophenyl)sulfonyl)-5- | 1H), 7.99 (s, 1H), 7.77 (d, J = 8.8 Hz, 1H), 7.38 (d, J = |
| | (trifluoromethyl)-1H-1,2,3-triazole- | 2.7 Hz, 1H), 7.15 (dd, J = 8.9, 2.8 Hz, 1H), 3.88 (d, J = |
| | 4-carboxamide | 4.6 Hz, 3H); LCMS(M − 2): 562.10 |
| 577 | 1-(2-chloro-4-methoxyphenyl)-N- | ¹H-NMR (400 MHz, DMSO-d₆) δ 7.89 (d, J = 8.3 Hz, |
| | tosyl-5-(trifluoromethyl)-1H-1,2,3- | 2H), 7.79 (d, J = 9.0 Hz, 1H), 7.45 (d, J = 8.1 Hz, 2H), |
| | triazole-4-carboxamide | 7.39 (d, J = 2.7 Hz, 1H), 7.17 (dd, J = 9.0, 2.7 Hz, 1H), |
| | | 3.88 (d, J = 4.9 Hz, 3H), 2.40 (s, 3H); LCMS(M − 2): |
| | | 472.35 |
| 578 | 1-(2-chloro-4-methoxyphenyl)-N- | ¹H-NMR (400 MHz, DMSO-d₆) δ 8.06 (dd, J = 8.9, 5.3 |
| | ((4-fluorophenyl)sulfonyl)-5- | Hz, 2H), 7.78 (d, J = 8.8 Hz, 1H), 7.49 (t, J = 8.9 Hz, 2H), |
| | (trifluoromethyl)-1H-1,2,3-triazole- | 7.39 (d, J = 2.7 Hz, 1H), 7.16 (dd, J = 8.9, 2.8 Hz, 1H), |
| | 4-carboxamide | 3.88 (s, 3H); LCMS(M − 2): 476.25 |
| 579 | 1-(2-chloro-5-methoxyphenyl)-N- | ¹H-NMR (400 MHz, DMSO-d₆) δ 7.80-7.77 (m, 1H), |
| | ((5-fluoro-2-methylphenyl)sulfonyl)- | 7.69 (d, J = 8.8 Hz, 1H), 7.49-7.47 (m, 2H), 7.36 (d, J = |
| | 5-methyl-1H-1,2,3-triazole-4- | 2.9 Hz, 1H), 7.27 (dd, J = 8.9, 3.1 Hz, 1H), 3.80 (s, 3H), |
| | carboxamide | 2.61 (s, 3H), 2.25 (s, 3H); LCMS(M+): 438.35 |
| 580 | 1-(2-chloro-5-methoxyphenyl)-N- | ¹H-NMR (400 MHz, DMSO-d₆) δ 8.16 (d, J = 8.8 Hz, |
| | ((2,4-dichlorophenyl)sulfonyl)-5- | 1H), 7.89 (d, J = 2.0 Hz, 1H), 7.72-7.68 (m, 2H), 7.37 (d, |
| | methyl-1H-1,2,3-triazole-4- | J = 2.9 Hz, 1H), 7.27 (dd, J = 9.0, 2.9 Hz, 1H), 3.80 (s, |
| | carboxamide | 3H), 2.25 (s, 3H); LCMS(M + 1): 476.20 |
| 581 | N-((5-chloro-2- | ¹H-NMR (400 MHz, DMSO-d₆) δ 7.94 (q, J = 2.9 Hz, |
| | fluorophenyl)sulfonyl)-1-(2-chloro- | 1H), 7.87-7.83 (m, 1H), 7.69 (d, J = 8.8 Hz, 1H), 7.54 (t, |
| | 5-methoxyphenyl)-5-methyl-1H- | J = 9.3 Hz, 1H), 7.36 (d, J = 3.2 Hz, 1H), 7.27 (dd, J = |
| | 1,2,3-triazole-4-carboxamide | 9.0, 3.2 Hz, 1H), 3.80 (s, 3H), 2.27 (s, 3H); LCMS(M+): |
| | | 458.30 |
| 582 | N-((5-chloro-2,4- | ¹H-NMR (400 MHz, DMSO-d₆) δ 8.13 (t, J = 7.6 Hz, 1H), |
| | difluorophenyl)sulfonyl)-1-(2- | 7.85 (t, J = 9.5 Hz, 1H), 7.69 (d, J = 9.0 Hz, 1H), 7.36 (d, |
| | chloro-5-methoxyphenyl)-5-methyl- | J = 2.9 Hz, 1H), 7.27 (dd, J = 9.0, 2.9 Hz, 1H), 3.80 (s, |
| | 1H-1,2,3-triazole-4-carboxamide | 3H), 2.27 (s, 3H); LCMS(M+): 476.30 |
| 583 | 1-(2-chloro-5-methoxyphenyl)-5- | ¹H-NMR (400 MHz, DMSO-d₆) δ 8.24 (s, 1H), 8.11 (s, |
| | methyl-N-((2,4,5- | 1H), 7.69 (d, J = 9.0 Hz, 1H), 7.36 (d, J = 2.9 Hz, 1H), |
| | trichlorophenyl)sulfonyl)-1H-1,2,3- | 7.27 (dd, J = 9.0, 3.2 Hz, 1H), 3.80 (s, 3H), 2.27 (s, 3H); |
| | triazole-4-carboxamide | LCMS(M+): 510.20 |
| 584 | 1-(4-cyano-2-fluorophenyl)-N-(o- | ¹H-NMR (400 MHz, DMSO-d₆) δ 8.36 (dd, J = 9.9, 1.4 |
| | tolylsulfonyl)-5-(trifluoromethyl)- | Hz, 1H), 8.14-8.10 (m, 1H), 8.07-8.02 (m, 2H), 7.60-7.56 |
| | 1H-1,2,3-triazole-4-carboxamide | (m, 1H), 7.43 (q, J = 7.7 Hz, 2H), 2.65 (s, 3H); LCMS |
| | | (M − 2): 451.30 |
| 585 | 1-(4-cyano-2-fluorophenyl)-N-((2,4- | ¹H-NMR (400 MHz, DMSO-d₆) δ 8.36 (dd, J = 9.8, 1.5 |
| | difluorophenyl)sulfonyl)-5- | Hz, 1H), 8.12 (t, J = 7.8 Hz, 1H), 8.06-8.00 (m, 2H), 7.52- |
| | (trifluoromethyl)-1H-1,2,3-triazole- | 7.47 (m, 1H), 7.30 (td, J = 8.6, 2.0 Hz, 1H); LCMS (M − |
| | 4-carboxamide | 2): 473.30 |
| 586 | N-((5-chloro-2- | ¹H-NMR (400 MHz, CHLOROFORM-D) δ 9.84 (s, 1H), |
| | methoxyphenyl)sulfonyl)-1-(2- | 8.15 (d, J = 2.7 Hz, 1H), 7.55 (dd, J = 8.8, 2.7 Hz, 1H), |
| | chloro-4-(trifluoromethoxy)phenyl)- | 7.52 (d, J = 2.0 Hz, 1H), 7.47 (d, J = 8.8 Hz, 1H), 7.38- |
| | 5-methyl-1H-1,2,3-triazole-4- | 7.35 (m, 1H), 6.98 (d, J = 9.0 Hz, 1H), 3.98 (s, 3H), 2.42 |
| | carboxamide | (s, 3H); LCMS(M+): 524.95 |
| 587 | 1-(2-chloro-4- | ¹H-NMR (400 MHz, CHLOROFORM-D) δ 9.83 (s, 1H), |
| | (trifluoromethoxy)phenyl)-N-((2- | 7.96 (d, J = 1.7 Hz, 1H), 7.51 (d, J = 2.0 Hz, 1H), 7.47 (d, |
| | methoxy-5-methylphenyl)sulfonyl)- | J = 8.6 Hz, 1H), 7.40-7.35 (m, 2H), 6.93 (d, J = 8.6 Hz, |
| | 5-methyl-1H-1,2,3-triazole-4- | 1H), 3.95 (s, 3H), 2.41 (s, 3H), 2.38 (s, 3H); |
| | carboxamide | LCMS(M + 1): 505.15 |
| 588 | 1-(2-chloro-4- | ¹H-NMR (400 MHz, CHLOROFORM-D) δ 9.78 (s, 1H), |
| | (trifluoromethoxy)phenyl)-N-((5- | 8.01 (dd, J = 8.3, 2.7 Hz, 1H), 7.52 (d, J = 2.7 Hz, 1H), |
| | fluoro-2-methylphenyl)sulfonyl)-5- | 7.47 (d, J = 8.8 Hz, 1H), 7.38-7.36 (m, 1H), 7.31 (dd, J = |
| | methyl-1H-1,2,3-triazole-4- | 8.7, 5.3 Hz, 1H), 7.24-7.22 (m, 1H), 2.71 (s, 3H), 2.42 (s, |
| | carboxamide | 3H); LCMS(M + 1): 493.10 |
| 589 | N-((5-chloro-2- | ¹H-NMR (400 MHz, CHLOROFORM-D) δ 9.89 (s, 1H), |
| | methoxyphenyl)sulfonyl)-1-(2- | 8.14 (d, J = 2.7 Hz, 1H), 7.57 (dd, J = 8.8, 2.7 Hz, 1H), |
| | chloro-4-(trifluoromethoxy)phenyl)- | 7.52-7.50 (m, 2H), 7.37 (dd, J = 8.8, 1.5 Hz, 1H), 7.00 (t, |
| | 5-(trifluoromethyl)-1H-1,2,3- | J = 4.4 Hz, 1H), 4.00 (s, 3H); LCMS(M+): 578.90 |
| | triazole-4-carboxamide | |
| 590 | 1-(2-chloro-4- | ¹H-NMR (400 MHz, CHLOROFORM-D) δ 9.88 (s, 1H), |
| | (trifluoromethoxy)phenyl)-N-((2- | 7.96 (d, J = 1.7 Hz, 1H), 7.51 (dd, J = 5.3, 3.3 Hz, 2H), |
| | methoxy-5-methylphenyl)sulfonyl)- | 7.42-7.35 (m, 2H), 6.94 (d, J = 8.3 Hz, 1H), 3.97 (s, 3H), |
| | 5-(trifluoromethyl)-1H-1,2,3- | 2.38 (s, 3H); LCMS(M+): 559.05 |
| | triazole-4-carboxamide | |
| 591 | 1-(2-chloro-4-methylphenyl)-N- | ¹H-NMR (400 MHz, CHLOROFORM-D) δ 9.96 (s, 1H), |
| | ((2,4-dichlorophenyl)sulfonyl)-1H- | 8.45 (s, 1H), 8.31 (d, J = 8.9 Hz, 1H), 8.05 (d, J = 8.6 Hz, |
| | 1,2,3-triazole-4-carboxamide | 0H), 7.55 (dd, J = 7.0, 1.8 Hz, 1H), 7.50-7.46 (m, 1H), |
| | | 7.42-7.40 (m, 1H), 7.28 (q, J = 0.8 Hz, 1H); LCMS(M − |
| | | 1): 444.8 |
| 592 | 1-(2-chloro-4-methylphenyl)-N-((2- | ¹H-NMR (400 MHz, CHLOROFORM-D) δ 9.81 (s, 1H), |
| | methoxy-5-methylphenyl)sulfonyl)- | 8.44 (s, 1H), 7.96 (d, J = 1.8 Hz, 1H), 7.73 (d, J = 2.1 Hz, |
| | 1H-1,2,3-triazole-4-carboxamide | 1H), 7.47 (d, J = 8.3 Hz, 1H), 7.34-7.32 (m, 1H), 6.93 |
| | | (dd, J = 11.9, 8.3 Hz, 2H), 3.93 (s, 3H), 2.37 (s, 3H), 2.34 |
| | | (s, 3H); LCMS(M+): 420.9 |
| 593 | 1-(4-cyano-2-fluorophenyl)-5- | ¹H-NMR (400 MHz, DMSO-d₆) δ 8.34 (dd, J = 9.8, 1.7 |
| | (trifluoromethyl)-N-((2,4,6- | Hz, 1H), 8.13 (t, J = 7.7 Hz, 1H), 8.04 (dd, J = 8.2, 0.9 |
| | trifluorophenyl)sulfonyl)-1H-1,2,3- | Hz, 1H), 7.28 (t, J = 9.0 Hz, 2H); LCMS (M − 1): 491.90 |
| | triazole-4-carboxamide | |
| 594 | 1-(4-cyano-2-fluorophenyl)-N-((4- | ¹H-NMR (400 MHz, DMSO-d₆) δ 8.36 (dd, J = 9.9, 1.6 |
| | fluorophenyl)sulfonyl)-5- | Hz, 1H), 8.13-8.05 (m, 4H), 7.51-7.47 (m, 2H); LCMS |
| | (trifluoromethyl)-1H-1,2,3-triazole- | (M − 1): 455.90 |
| | 4-carboxamide | |
| 595 | N-((5-chloro-2- | ¹H-NMR (400 MHz, DMSO-d₆) δ 8.35 (dd, J = 9.9, 1.6 |
| | fluorophenyl)sulfonyl)-1-(4-cyano- | Hz, 1H), 8.14-8.10 (m, 1H), 8.06-8.03 (m, 1H), 7.85 (q, |
| | 2-fluorophenyl)-5-(trifluoromethyl)- | J = 2.9 Hz, 1H), 7.71 (ddd, J = 8.8, 4.0, 2.8 Hz, 1H), 7.41 |
| | 1H-1,2,3-triazole-4-carboxamide | (t, J = 9.3 Hz, 1H); LCMS (M+): 491.90 |
| 596 | N-((3-chloro-2- | ¹H-NMR (400 MHz, CHLOROFORM-D) δ 9.88 (s, 1H), |
| | methylphenyl)sulfonyl)-1-(2-chloro- | 8.27 (dd, J = 8.1, 1.0 Hz, 1H), 7.68 (dd, J = 7.7, 1.1 Hz, |
| | 4-(trifluoromethoxy)phenyl)-5- | 1H), 7.52-7.49 (m, 2H), 7.41-7.35 (m, 2H), 2.80 (s, 3H); |
| | (trifluoromethyl)-1H-1,2,3-triazole- | LCMS(M − 2): 560.90 |
| | 4-carboxamide | |
| 597 | 1-(2-chloro-4- | ¹H-NMR (400 MHz, CHLOROFORM-D) δ 9.86 (s, 1H), |
| | (trifluoromethoxy)phenyl)-N-((5- | 8.02 (dd, J = 8.3, 2.7 Hz, 1H), 7.50 (d, J = 8.8 Hz, 2H), |
| | fluoro-2-methylphenyl)sulfonyl)-5- | 7.37 (dq, J = 8.9, 1.2 Hz, 1H), 7.33 (dd, J = 8.4, 5.3 Hz, |
| | (trifluoromethyl)-1H-1,2,3-triazole- | 1H), 7.28 (d, J = 2.7 Hz, 1H), 2.72 (s, 3H); LCMS(M − 1): |
| | 4-carboxamide | 544.90 |
| 598 | N-((5-chloro-2- | ¹H-NMR (400 MHz, CHLOROFORM-D) δ 9.89 (s, 1H), |
| | methoxyphenyl)sulfonyl)-1-(2,4- | 8.14 (d, J = 2.4 Hz, 1H), 7.66 (d, J = 2.2 Hz, 1H), 7.57 |
| | dichlorophenyl)-5-(trifluoromethyl)- | (dd, J = 8.8, 2.7 Hz, 1H), 7.52-7.49 (m, 1H), 7.39 (d, J = |
| | 1H-1,2,3-triazole-4-carboxamide | 8.3 Hz, 1H), 7.00-6.98 (m, 1H), 4.00 (s, 3H); |
| | | LCMS(M + 1): 530.75 |
| 599 | 1-(2,4-dichlorophenyl)-N-((2- | ¹H-NMR (400 MHz, CHLOROFORM-D) δ 9.87 (s, 1H), |
| | methoxy-5-methylphenyl)sulfonyl)- | 7.96 (d, J = 1.5 Hz, 1H), 7.65 (d, J = 2.2 Hz, 1H), 7.49 |
| | 5-(trifluoromethyl)-1H-1,2,3- | (dd, J = 8.6, 2.2 Hz, 1H), 7.42-7.38 (m, 2H), 6.94 (d, J = |
| | triazole-4-carboxamide | 8.6 Hz, 1H), 3.97 (s, 3H), 2.37 (s, 3H); LCMS(M+): |
| | | 508.85 |
| 600 | N-((3-chloro-2- | ¹H-NMR (400 MHz, CHLOROFORM-D) δ 9.88 (s, 1H), |
| | methylphenyl)sulfonyl)-1-(2,4- | 8.27 (dd, J = 8.1, 1.0 Hz, 1H), 7.69-7.66 (m, 2H), 7.50 |
| | dichlorophenyl)-5-(trifluoromethyl)- | (dd, J = 8.3, 2.2 Hz, 1H), 7.40-7.36 (m, 2H), 2.80 (s, 3H); |
| | 1H-1,2,3-triazole-4-carboxamide | LCMS(M+): 512.80 |
| 601 | 1-(2,4-dichlorophenyl)-N-((5-fluoro- | ¹H-NMR (400 MHz, CHLOROFORM-D) δ 9.85 (s, 1H), |
| | 2-methylphenyl)sulfonyl)-5- | 8.01 (dd, J = 8.3, 2.7 Hz, 1H), 7.66 (d, J = 2.2 Hz, 1H), |
| | (trifluoromethyl)-1H-1,2,3-triazole- | 7.50 (dd, J = 8.3, 2.2 Hz, 1H), 7.38 (d, J = 8.6 Hz, 1H), |
| | 4-carboxamide | 7.32 (dd, J = 8.4, 5.3 Hz, 1H), 7.28 (d, J = 2.7 Hz, 1H), |
| | | 2.71 (s, 3H); LCMS(M+): 496.90 |
| 602 | 1-(4-cyano-2-fluorophenyl)-N-((5- | ¹H-NMR (400 MHz, DMSO-d₆) δ 8.36 (dd, J = 9.8, 1.5 |
| | fluoro-2-methylphenyl)sulfonyl)-5- | Hz, 1H), 8.12 (t, J = 7.7 Hz, 1H), 8.06 (dd, J = 8.3, 1.2 |
| | (trifluoromethyl)-1H-1,2,3-triazole- | Hz, 1H), 7.76 (d, J = 8.3 Hz, 1H), 7.47-7.45 (m, 2H), 2.60 |
| | 4-carboxamide | (s, 3H); LCMS (M − 2): 469.55 |
| 603 | 1-(4-cyano-2-fluorophenyl)-N-((4- | ¹H-NMR (400 MHz, DMSO-d₆) δ 8.36 (dd, J = 9.8, 1.5 |
| | (trifluoromethoxy)phenyl)sulfonyl)- | Hz, 1H), 8.13-8.04 (m, 4H), 7.63 (d, J = 8.1 Hz, 2H); |
| | 5-(trifluoromethyl)-1H-1,2,3- | LCMS (M − 2): 521.50 |
| | triazole-4-carboxamide | |
| 604 | 1-(4-cyano-2-fluorophenyl)-N- | ¹H-NMR (400 MHz, DMSO-d₆) δ 8.34 (dd, J = 9.8, 1.5 |
| | ((2,4,5-trichlorophenyl)sulfonyl)-5- | Hz, 1H), 8.16-8.11 (m, 2H), 8.04 (d, J = 8.3 Hz, 1H), 7.96 |
| | (trifluoromethyl)-1H-1,2,3-triazole- | (s, 1H); LCMS (M − 2): 539.45 |
| | 4-carboxamide | |
| 605 | N-((4-chlorophenyl)sulfonyl)-1-(4- | ¹H-NMR (400 MHz, DMSO-d₆) δ 8.36 (dd, J = 9.8, 1.5 |
| | cyano-2-fluorophenyl)-5- | Hz, 1H), 8.13-8.04 (m, 2H), 8.00-7.97 (m, 2H), 7.74-7.71 |
| | (trifluoromethyl)-1H-1,2,3-triazole- | (m, 2H); LCMS (M − 2): 471.45 |
| | 4-carboxamide | |
| 606 | N-((3-chloro-2- | ¹H-NMR (400 MHz, DMSO-d₆) δ 9.16 (s, 1H), 8.07 (dd, |
| | methylphenyl)sulfonyl)-1-(2-chloro- | J = 7.9, 0.9 Hz, 1H), 7.80 (d, J = 7.0 Hz, 1H), 7.62-7.60 |
| | 4-methylphenyl)-1H-1,2,3-triazole- | (m, 2H), 7.50 (t, J = 7.9 Hz, 1H), 7.40 (dd, J = 7.9, 0.9 |
| | 4-carboxamide | Hz, 1H), 2.65 (d, J = 14.4 Hz, 3H), 2.40 (d, J = 13.8 Hz, |
| | | 3H); LCMS(M − 1): 424.9 |
| 607 | N-((5-chloro-2- | ¹H-NMR (400 MHz, DMSO-d₆) δ 9.18 (d, J = 9.2 Hz, |
| | methoxyphenyl)sulfonyl)-1-(2- | 1H), 7.87 (dd, J = 11.3, 2.8 Hz, 1H), 7.77-7.73 (m, 1H), |
| | chloro-4-methylphenyl)-1H-1,2,3- | 7.63-7.59 (m, 2H), 7.41 (dd, J = 8.1, 1.1 Hz, 1H), 7.30 (d, |
| | triazole-4-carboxamide | J = 8.9 Hz, 1H), 3.87 (t, J = 14.8 Hz, 3H), 2.40 (d, J = |
| | | 13.1 Hz, 3H); LCMS(M − 1): 440.8 |
| 608 | 1-(2-chloro-4-methylphenyl)-N-((4- | ¹H-NMR (400 MHz, DMSO-d₆) δ 9.15 (s, 1H), 8.14 (d, |
| | (trifluoromethoxy)phenyl)sulfonyl)- | J = 9.2 Hz, 2H), 7.65 (s, 1H), 7.63 (d, J = 4.6 Hz, 2H), 7.59 |
| | 1H-1,2,3-triazole-4-carboxamide | (d, J = 7.9 Hz, 1H), 7.40 (d, J = 8.3 Hz, 1H), 2.41 (s, 3H); |
| | | LCMS(M+): 460.8 |
| 609 | 1-(2-chloro-4-methylphenyl)-N-(o- | ¹H-NMR (400 MHz, DMSO-d₆) δ 9.15 (d, J = 4.9 Hz, |
| | tolylsulfonyl)-1H-1,2,3-triazole-4- | 1H), 8.05 (dd, J = 7.9, 1.2 Hz, 1H), 7.62-7.61 (m, 2H), |
| | carboxamide | 7.60 (dt, J = 5.8, 1.5 Hz, 1H), 7.58-7.57 (m, 1H), 7.45 (t, |
| | | J = 8.1 Hz, 1H), 7.42-7.38 (m, 2H), 2.64 (d, J = 7.6 Hz, |
| | | 3H), 2.39 (t, J = 7.0 Hz, 3H); LCMS(M+): 390.5 |
| 610 | N-((5-chloro-2,4- | ¹H-NMR (400 MHz, DMSO-d₆) δ 8.34 (dd, J = 9.8, 1.5 |
| | difluorophenyl)sulfonyl)-1-(4-cyano- | Hz, 1H), 8.12 (t, J = 7.6 Hz, 1H), 8.05-7.99 (m, 2H), 7.69 |
| | 2-fluorophenyl)-5-(trifluoromethyl)- | (t, J = 9.6 Hz, 1H); LCMS (M − 2): 507.75 |
| | 1H-1,2,3-triazole-4-carboxamide | |
| 611 | N-((3-chloro-4- | ¹H-NMR (400 MHz, DMSO-d₆) δ 8.35 (dd, J = 9.8, 1.5 |
| | fluorophenyl)sulfonyl)-1-(4-cyano- | Hz, 1H), 8.12-8.03 (m, 3H), 7.93 (qd, J = 4.4, 2.3 Hz, |
| | 2-fluorophenyl)-5-(trifluoromethyl)- | 1H), 7.62 (t, J = 8.9 Hz, 1H); LCMS (M − 2): 489.75 |
| | 1H-1,2,3-triazole-4-carboxamide | |
| 612 | 1-(2-chloro-4-fluorophenyl)-N-((4- | ¹H-NMR (400 MHz, DMSO-d₆) δ 8.87 (s, 1H), 7.98 (dd, |
| | fluorophenyl)sulfonyl)-1H-1,2,3- | J = 8.4, 5.7 Hz, 2H), 7.84-7.76 (m, 2H), 7.48 (d, J = 3.1 |
| | triazole-4-carboxamide | Hz, 1H), 7.36 (s, 2H); LCMS(M+): 398.8 |
| 613 | 1-(2-chloro-4-fluorophenyl)-N-(o- | ¹H-NMR (400 MHz, DMSO-d₆) δ 9.17 (s, 1H), 8.05 (dd, |
| | tolylsulfonyl)-1H-1,2,3-triazole-4- | J = 7.9, 1.2 Hz, 1H), 7.91-7.81 (m, 2H), 7.60-7.56 (m, |
| | carboxamide | 1H), 7.51 (td, J = 8.4, 2.5 Hz, 1H), 7.47-7.40 (m, 2H), |
| | | 2.66-2.63 (m, 3H); LCMS(M+): 394.8 |
| 614 | 1-(2-chloro-4-fluorophenyl)-N-((2- | ¹H-NMR (400 MHz, DMSO-d₆) δ 8.93 (s, 1H), 8.00 (d, |
| | methoxy-5-methylphenyl)sulfonyl)- | J = 7.9 Hz, 1H), 7.85-7.79 (m, 1H), 7.67 (d, J = 6.7 Hz, |
| | 1H-1,2,3-triazole-4-carboxamide | 1H), 7.49 (td, J = 8.4, 2.8 Hz, 1H), 7.40 (t, J = 7.8 Hz, |
| | | 1H), 7.07 (t, J = 51.2 Hz, 1H), 2.64 (s, 3H); LCMS(M+): |
| | | 424.4 |
| 615 | 1-(2-chloro-4-fluorophenyl)-N- | ¹H-NMR (400 MHz, DMSO-d₆) δ 9.10 (s, 1H), 8.25 (s, |
| | ((2,3,6-trichlorophenyl)sulfonyl)- | 1H), 8.09 (s, 1H), 7.87-7.82 (m, 2H), 7.51 (td, J = 8.5, 2.9 |
| | 1H-1,2,3-triazole-4-carboxamide | Hz, 1H); LCMS(M+): 484.7 |
| 616 | N-((5-chloro-2- | ¹H-NMR (400 MHz, DMSO-d₆) δ 9.13 (s, 1H), 7.95 (q, |
| | fluorophenyl)sulfonyl)-1-(2-chloro- | J = 3.0 Hz, 1H), 7.85 (qd, J = 4.3, 2.8 Hz, 1H), 7.63-7.60 |
| | 4-methylphenyl)-1H-1,2,3-triazole- | (m, 2H), 7.53 (t, J = 9.3 Hz, 1H), 7.40 (dt, J = 9.0, 0.9 Hz, |
| | 4-carboxamide | 1H), 2.41 (s, 3H); LCMS(M − 1): 428.8 |
| 617 | 1-(2-chloro-4-methylphenyl)-N-((5- | ¹H-NMR (400 MHz, DMSO-d₆) δ 9.15 (s, 1H), 7.79 (dd, |
| | fluoro-2-methylphenyl)sulfonyl)-1H- | J = 8.6, 2.1 Hz, 1H), 7.63-7.60 (m, 2H), 7.49-7.46 (m, |
| | 1,2,3-triazole-4-carboxamide | 2H), 7.40 (dd, J = 8.1, 1.1 Hz, 1H), 2.58 (d, J = 8.3 Hz, |
| | | 3H), 2.41 (s, 3H); LCMS(M+): 408.4 |
| 618 | 1-(2-chloro-4-methylphenyl)-N-((4- | ¹H-NMR (400 MHz, DMSO-d₆) δ 9.15 (s, 1H), 8.10-8.06 |
| | fluorophenyl)sulfonyl)-1H-1,2,3- | (m, 2H), 7.62-7.58 (m, 2H), 7.51-7.47 (m, 2H), 7.39 (dt, |
| | triazole-4-carboxamide | J = 9.1, 0.9 Hz, 1H), 2.41 (s, 3H); LCMS(M+): 394.8 |
| 619 | N-((2-chloro-5- | ¹H-NMR (400 MHz, CHLOROFORM-D) δ 9.93 (s, 1H), |
| | methoxyphenyl)sulfonyl)-1-(2,6- | 7.87 (d, J = 3.1 Hz, 1H), 7.44-7.41 (m, 3H), 7.10 (dd, J = |
| | dichloro-4- | 8.9, 3.1 Hz, 1H), 3.91 (s, 3H), 2.37 (d, J = 7.6 Hz, 3H); |
| | (trifluoromethoxy)phenyl)-5-methyl- | LCMS(M+): 560.80 |
| | 1H-1,2,3-triazole-4-carboxamide | |
| 620 | 1-(2,6-dichloro-4- | ¹H-NMR (400 MHz, CHLOROFORM-D) δ 9.95 (s, 1H), |
| | (trifluoromethoxy)phenyl)-N-((2,5- | 8.36 (d, J = 2.4 Hz, 1H), 7.55 (dd, J = 8.6, 2.4 Hz, 1H), |
| | dichlorophenyl)sulfonyl)-5-methyl- | 7.48 (d, J = 8.6 Hz, 1H), 7.45 (d, J = 0.6 Hz, 2H), 2.38 (d, |
| | 1H-1,2,3-triazole-4-carboxamide | J = 7.6 Hz, 3H); LCMS(M+): 564.40 |
| 621 | 1-(2,6-dichloro-4- | ¹H-NMR (400 MHz, CHLOROFORM-D) δ 9.72 (s, 1H), |
| | (trifluoromethoxy)phenyl)-N-((2,5- | 8.10 (d, J = 1.2 Hz, 1H), 7.44 (d, J = 0.6 Hz, 2H), 7.34 |
| | dimethylphenyl)sulfonyl)-5-methyl- | (dd, J = 7.8, 1.4 Hz, 1H), 7.22 (t, J = 4.0 Hz, 1H), 2.71 (s, |
| | 1H-1,2,3-triazole-4-carboxamide | 3H), 2.43 (s, 3H), 2.37 (t, J = 4.0 Hz, 3H); LCMS(M+): |
| | | 523.85 |
| 622 | 1-(2,6-dichloro-4- | ¹H-NMR (400 MHz, CHLOROFORM-D) δ 9.92 (s, 1H), |
| | (trifluoromethoxy)phenyl)-N-((2- | 8.20 (td, J = 7.6, 1.5 Hz, 1H), 7.70-7.64 (m, 1H), 7.44 (d, |
| | fluorophenyl)sulfonyl)-5-methyl-1H- | J = 0.6 Hz, 2H), 7.41-7.36 (m, 1H), 7.23 (t, J = 1.4 Hz, |
| | 1,2,3-triazole-4-carboxamide | 1H), 2.36 (t, J = 7.5 Hz, 3H); LCMS(M+): 512.75 |
| 623 | 1-(2,6-dichloro-4- | ¹H-NMR (400 MHz, CHLOROFORM-D) δ 9.82 (s, 1H), |
| | (trifluoromethoxy)phenyl)-5-methyl- | 8.65-8.63 (m, 1H), 7.93-7.91 (m, 1H), 7.85-7.78 (m, 2H), |
| | N-((2- | 7.44-7.41 (m, 2H), 2.37-2.32 (m, 3H); LCMS(M + 1): |
| | (trifluoromethyl)phenyl)sulfonyl)- | 564.75 |
| | 1H-1,2,3-triazole-4-carboxamide | |
| 624 | N-((2-chlorophenyl)sulfonyl)-1-(2,6- | ¹H-NMR (400 MHz, CHLOROFORM-D) δ 9.94 (s, 1H), |
| | dichloro-4- | 8.39 (dd, J = 7.8, 1.2 Hz, 1H), 7.62-7.50 (m, 3H), 7.44 (d, |
| | (trifluoromethoxy)phenyl)-5-methyl- | J = 1.0 Hz, 2H), 2.36 (d, J = 7.8 Hz, 3H); LCMS(M+): |
| | 1H-1,2,3-triazole-4-carboxamide | 528.60 |
| 625 | 1-(2,6-dichloro-4- | ¹H-NMR (400 MHz, CHLOROFORM-D) δ 9.79 (s, 1H), |
| | (trifluoromethoxy)phenyl)-N-((5- | 8.02 (dd, J = 8.6, 2.7 Hz, 1H), 7.44 (t, J = 0.9 Hz, 2H), |
| | fluoro-2-methylphenyl)sulfonyl)-5- | 7.32 (dd, J = 8.6, 5.4 Hz, 1H), 7.24-7.22 (m, 1H), 2.72 (s, |
| | methyl-1H-1,2,3-triazole-4- | 3H), 2.39-2.33 (m, 3H); LCMS(M+): 528.70 |
| | carboxamide | |
| 626 | 1-(2,6-dichloro-4- | ¹H-NMR (400 MHz, CHLOROFORM-D) δ 9.94 (s, 1H), |
| | (trifluoromethoxy)phenyl)-N-((2,5- | 7.93-7.89 (m, 1H), 7.45 (d, J = 1.0 Hz, 2H), 7.39-7.33 (m, |
| | difluorophenyl)sulfonyl)-5-methyl- | 1H), 7.22 (dd, J = 9.0, 3.9 Hz, 1H), 2.38 (d, J = 8.1 Hz, |
| | 1H-1,2,3-triazole-4-carboxamide | 3H); LCMS(M + 1): 532.70 |
| 627 | 1-(2,6-dichloro-4- | ¹H-NMR (400 MHz, CHLOROFORM-D) δ 9.92 (s, 1H), |
| | (trifluoromethoxy)phenyl)-N-((2,4- | 8.23 (td, J = 8.6, 6.1 Hz, 1H), 7.44 (t, J = 0.9 Hz, 2H), |
| | difluorophenyl)sulfonyl)-5-methyl- | 7.12-7.07 (m, 1H), 7.02-6.97 (m, 1H), 2.37 (d, J = 8.1 Hz, |
| | 1H-1,2,3-triazole-4-carboxamide | 3H); LCMS(M + 1): 530.80 |
| 628 | 1-(2,6-dichloro-4- | ¹H-NMR (400 MHz, CHLOROFORM-D) δ 10.02 (s, |
| | (trifluoromethoxy)phenyl)-5-methyl- | 1H), 7.45 (d, J = 0.7 Hz, 2H), 6.85 (t, J = 8.4 Hz, 2H), |
| | N-((2,4,6-trifluorophenyl)sulfonyl)- | 2.45-2.39 (m, 3H); LCMS(M + 1): 548.75 |
| | 1H-1,2,3-triazole-4-carboxamide | |
| 629 | 1-(2,6-dichloro-4- | ¹H-NMR (400 MHz, CHLOROFORM-D) δ 9.83 (s, 1H), |
| | (trifluoromethoxy)phenyl)-N-((2- | 7.97 (d, J = 1.7 Hz, 1H), 7.44 (d, J = 0.7 Hz, 2H), 7.41- |
| | methoxy-5-methylphenyl)sulfonyl)- | 7.38 (m, 1H), 6.93 (d, J = 8.6 Hz, 1H), 3.95 (d, J = 7.3 |
| | 5-methyl-1H-1,2,3-triazole-4- | Hz, 3H), 2.37 (d, J = 7.8 Hz, 6H); LCMS(M + 1): 538.45 |
| | carboxamide | |
| 630 | 1-(4-chloro-2-fluorophenyl)-N-((2- | ¹H-NMR (400 MHz, CHLOROFORM-D) δ 7.85 (d, J = |
| | chloro-5-methoxyphenyl)sulfonyl)- | 3.2 Hz, 1H), 7.47-7.38 (m, 4H), 7.11 (dd, J = 8.8, 2.9 Hz, |
| | 5-(trifluoromethyl)-1H-1,2,3- | 1H), 3.91 (s, 3H); LCMS (M − 1): 511.00 |
| | triazole-4-carboxamide | |
| 631 | 1-(4-chloro-2-fluorophenyl)-N-((2- | ¹H-NMR (400 MHz, CHLOROFORM-D) δ 8.62 (d, J = |
| | chloro-5- | 2.0 Hz, 1H), 7.85 (ddd, J = 8.3, 2.2, 0.5 Hz, 1H), 7.70 (d, |
| | (trifluoromethyl)phenyl)sulfonyl)-5- | J = 8.3 Hz, 1H), 7.47-7.38 (m, 3H); LCMS (M − 1): 548.95 |
| | (trifluoromethyl)-1H-1,2,3-triazole- | |
| | 4-carboxamide | |
| 632 | 1-(4-chloro-2-fluorophenyl)-N-((2,5- | ¹H-NMR (400 MHz, CHLOROFORM-D) δ 8.35 (d, J = |
| | dichlorophenyl)sulfonyl)-5- | 2.4 Hz, 1H), 7.56 (dd, J = 8.4, 2.6 Hz, 1H), 7.49-7.47 (m, |
| | (trifluoromethyl)-1H-1,2,3-triazole- | 1H), 7.44 (dd, J = 7.2, 0.9 Hz, 1H), 7.41-7.39 (m, 2H); |
| | 4-carboxamide | LCMS (M − 1): 516.75 |
| 633 | 1-(2-chloro-4-fluorophenyl)-N-((4- | ¹H-NMR (400 MHz, DMSO-d₆) δ 9.13 (s, 1H), 7.99 (dd, |
| | chlorophenyl)sulfonyl)-1H-1,2,3- | J = 6.7, 1.8 Hz, 2H), 7.87-7.80 (m, 2H), 7.71 (d, J = 8.8 |
| | triazole-4-carboxamide | Hz, 2H), 7.53-7.48 (m, 1H); LCMS(M − 1): 414.8 |
| 634 | 1-(2-chloro-4-fluorophenyl)-N-((5- | ¹H-NMR (400 MHz, DMSO-d₆) δ 9.19 (s, 1H), 7.88 (d, |
| | fluoro-2-methylphenyl)sulfonyl)-1H- | J = 2.7 Hz, 1H), 7.86-7.82 (m, 2H), 7.79 (dd, J = 8.7, 2.1 |
| | 1,2,3-triazole-4-carboxamide | Hz, 1H), 7.52-7.49 (m, 2H), 7.48-7.45 (m, 1H), 2.59 (s, |
| | | 3H); LCMS(M+): 412.9 |
| 635 | N-((2-chloro-5- | ¹H-NMR (400 MHz, DMSO-d₆) δ 8.36 (d, J = 2.2 Hz, |
| | (trifluoromethyl)phenyl)sulfonyl)-1- | 1H), 8.10 (dd, J = 8.3, 2.0 Hz, 1H), 7.94 (d, J = 8.3 Hz, |
| | (2,4-difluorophenyl)-5-methyl-1H- | 1H), 7.82 (td, J = 8.8, 5.9 Hz, 1H), 7.75-7.69 (m, 1H), |
| | 1,2,3-triazole-4-carboxamide | 7.43-7.38 (m, 1H), 2.31 (s, 3H); LCMS (M+): 481.10 |
| 636 | N-((2,5-dichlorophenyl)sulfonyl)-1- | ¹H-NMR (400 MHz, DMSO-d₆) δ 8.10 (d, J = 2.4 Hz, |
| | (2,4-difluorophenyl)-5-methyl-1H- | 1H), 7.85-7.78 (m, 2H), 7.75-7.69 (m, 2H), 7.43-7.38 (m, |
| | 1,2,3-triazole-4-carboxamide | 1H), 2.31 (s, 3H); LCMS (M − 1): 446.85 |
| 637 | 1-(2,4-difluorophenyl)-N-((2,5- | ¹H-NMR (400 MHz, DMSO-d₆) δ 12.93 (s, 1H), 7.85- |
| | dimethylphenyl)sulfonyl)-5-methyl- | 7.78 (m, 2H), 7.75-7.69 (m, 1H), 7.43-7.38 (m, 2H), 7.28 |
| | 1H-1,2,3-triazole-4-carboxamide | (d, J = 7.9 Hz, 1H), 2.58 (s, 3H), 2.36 (s, 3H), 2.28 (s, |
| | | 3H); LCMS (M − 1): 406.95 |
| 638 | N-((2-chloro-5- | ¹H-NMR (400 MHz, DMSO-d₆) δ 7.86-7.79 (m, 1H), |
| | methoxyphenyl)sulfonyl)-1-(2,4- | 7.75-7.69 (m, 1H), 7.62 (d, J = 3.4 Hz, 1H), 7.57 (d, J = |
| | difluorophenyl)-5-methyl-1H-1,2,3- | 8.9 Hz, 1H), 7.43-7.38 (m, 1H), 7.28 (dd, J = 8.9, 3.1 Hz, |
| | triazole-4-carboxamide | 1H), 3.83 (s, 3H), 2.31 (s, 3H); LCMS (M+): 442.90 |
| 639 | 1-(4-chloro-2-fluorophenyl)-N-((2,4- | ¹H-NMR (400 MHz, CHLOROFORM-D) δ 10.03 (s, |
| | dichlorophenyl)sulfonyl)-5- | 1H), 8.31 (d, J = 8.6 Hz, 1H), 7.56 (d, J = 2.0 Hz, 1H), |
| | (trifluoromethyl)-1H-1,2,3-triazole- | 7.52-7.38 (m, 4H); LCMS (M − 1): 516.80 |
| | 4-carboxamide | |
| 640 | 1-(4-chloro-2-fluorophenyl)-5- | ¹H-NMR (400 MHz, CHLOROFORM-D) δ 9.90 (s, 1H), |
| | (trifluoromethyl)-N-((2- | 8.65-8.62 (m, 1H), 7.92 (dd, J = 7.2, 2.1 Hz, 1H), 7.86- |
| | (trifluoromethyl)phenyl)sulfonyl)- | 7.79 (m, 2H), 7.46-7.37 (m, 3H); LCMS (M − 1): 515.05 |
| | 1H-1,2,3-triazole-4-carboxamide | |
| 641 | 1-(4-chloro-2-fluorophenyl)-N-((2,5- | ¹H-NMR (400 MHz, CHLOROFORM-D) δ 9.79 (s, 1H), |
| | dimethylphenyl)sulfonyl)-5- | 8.09 (d, J = 1.2 Hz, 1H), 7.46-7.33 (m, 4H), 7.22 (d, J = |
| | (trifluoromethyl)-1H-1,2,3-triazole- | 7.6 Hz, 1H), 2.69 (s, 3H), 2.43 (s, 3H); LCMS (M − 1): |
| | 4-carboxamide | 474.95 |
| 642 | 1-(4-chloro-2-fluorophenyl)-N-((5- | ¹H-NMR (400 MHz, CHLOROFORM-D) δ 9.86 (s, 1H), |
| | fluoro-2-methylphenyl)sulfonyl)-5- | 8.01 (dd, J = 8.3, 2.7 Hz, 1H), 7.46-7.38 (m, 3H), 7.34- |
| | (trifluoromethyl)-1H-1,2,3-triazole- | 7.29 (m, 1H), 7.24-7.22 (m, 1H), 2.70 (s, 3H); LCMS (M − |
| | 4-carboxamide | 1): 478.90 |
| 643 | 1-(4-chloro-2-fluorophenyl)-N-((3- | ¹H-NMR (400 MHz, CHLOROFORM-D) δ 9.89 (s, 1H), |
| | chloro-2-methylphenyl)sulfonyl)-5- | 8.26 (dd, J = 8.1, 1.2 Hz, 1H), 7.67 (dd, J = 8.1, 1.0 Hz, |
| | (trifluoromethyl)-1H-1,2,3-triazole- | 1H), 7.46-7.36 (m, 4H), 2.79 (s, 3H); LCMS (M − 1): |
| | 4-carboxamide | 494.90 |
| 644 | 1-(4-chloro-2-fluorophenyl)-N-((5- | ¹H-NMR (400 MHz, CHLOROFORM-D) δ 9.96 (s, 1H), |
| | chloro-2-fluorophenyl)sulfonyl)-5- | 8.16 (q, J = 2.9 Hz, 1H), 7.62 (qd, J = 4.3, 2.7 Hz, 1H), |
| | (trifluoromethyl)-1H-1,2,3-triazole- | 7.47-7.38 (m, 3H), 7.23-7.18 (m, 1H); LCMS (M − 1): |
| | 4-carboxamide | 498.85 |
| 645 | 1-(4-chloro-2-fluorophenyl)-N-((2- | ¹H-NMR (400 MHz, CHLOROFORM-D) δ 9.87 (s, 1H), |
| | methoxy-5-methylphenyl)sulfonyl)- | 7.95 (d, J = 1.7 Hz, 1H), 7.47-7.37 (m, 4H), 6.93 (d, J = |
| | 5-(trifluoromethyl)-1H-1,2,3- | 8.3 Hz, 1H), 3.96 (s, 3H), 2.37 (s, 3H); LCMS (M − 1): |
| | triazole-4-carboxamide | 490.90 |
| 646 | N-((4-chloro-2,5- | ¹H-NMR (400 MHz, CHLOROFORM-D) δ 9.82 (s, 1H), |
| | dimethylphenyl)sulfonyl)-1-(4- | 8.13 (s, 1H), 7.46-7.38 (m, 3H), 7.33 (s, 1H), 2.67 (s, 3H), |
| | chloro-2-fluorophenyl)-5- | 2.44 (s, 3H); LCMS (M − 1): 508.95 |
| | (trifluoromethyl)-1H-1,2,3-triazole- | |
| | 4-carboxamide | |
| 647 | 1-(4-chloro-2-fluorophenyl)-N- | ¹H-NMR (400 MHz, CHLOROFORM-D) δ 8.44 (s, 1H), |
| | ((2,4,5-trichlorophenyl)sulfonyl)-5- | 7.66 (s, 1H), 7.45-7.39 (m, 3H); LCMS (M − 1): 550.80 |
| | (trifluoromethyl)-1H-1,2,3-triazole- | |
| | 4-carboxamide | |
| 648 | 1-(4-chloro-2-fluorophenyl)-N-((3- | ¹H-NMR (400 MHz, CHLOROFORM-D) δ 8.25 (dd, J = |
| | chloro-4-fluorophenyl)sulfonyl)-5- | 6.6, 2.4 Hz, 1H), 8.13 (qd, J = 4.4, 2.4 Hz, 1H), 7.45-7.33 |
| | (trifluoromethyl)-1H-1,2,3-triazole- | (m, 4H); LCMS (M − 1): 498.85 |
| | 4-carboxamide | |
| 649 | N-((2,5-dimethylphenyl)sulfonyl)-5- | ¹H-NMR (400 MHz, DMSO-d₆) δ 8.30 (d, J = 3.1 Hz, |
| | methyl-1-(2,4,5-trichlorophenyl)- | 1H), 8.21 (s, 1H), 7.85 (s, 1H), 7.39 (d, J = 6.4 Hz, 1H), |
| | 1H-1,2,3-triazole-4-carboxamide | 7.29 (d, J = 7.9 Hz, 1H), 2.59 (s, 3H), 2.39-2.35 (m, 3H), |
| | | 2.27 (s, 3H); LCMS(M + 1): 474.9 |
| 650 | N-((2,5-dichlorophenyl)sulfonyl)-5- | ¹H-NMR (400 MHz, DMSO-d₆) δ 8.29 (s, 1H), 8.22 (s, |
| | methyl-1-(2,4,5-trichlorophenyl)- | 1H), 8.09 (d, J = 2.8 Hz, 1H), 7.79-7.77 (m, 1H), 7.70 (d, |
| | 1H-1,2,3-triazole-4-carboxamide | J = 8.6 Hz, 1H), 2.30 (s, 3H); LCMS(M + 1): 514.8 |
| 651 | N-((2-chloro-5- | ¹H-NMR (400 MHz, DMSO-d₆) δ 8.30 (d, J = 4.6 Hz, |
| | methoxyphenyl)sulfonyl)-5-methyl- | 1H), 8.23 (s, 1H), 7.62 (d, J = 3.1 Hz, 1H), 7.57 (d, J = |
| | 1-(2,4,5-trichlorophenyl)-1H-1,2,3- | 8.9 Hz, 1H), 7.27 (dd, J = 8.9, 3.1 Hz, 1H), 3.85 (s, 3H), |
| | triazole-4-carboxamide | 2.30 (d, J = 8.9 Hz, 3H); LCMS(M + 1): 510.8 |
| 652 | N-((2,5- | ¹H-NMR (400 MHz, DMSO-d₆) δ 8.61 (s, 1H), 8.28 (d, |
| | bis(trifluoromethyl)phenyl)sulfonyl)- | J = 8.3 Hz, 2H), 8.22 (d, J = 6.1 Hz, 2H), 2.31 (d, J = 4.9 |
| | 5-methyl-1-(2,4,5-trichlorophenyl)- | Hz, 3H); LCMS(M + 1): 582.9 |
| | 1H-1,2,3-triazole-4-carboxamide | |
| 653 | N-((2-methoxy-5- | ¹H-NMR (400 MHz, DMSO-d₆) δ 8.30 (s, 1H), 8.22 (s, |
| | methylphenyl)sulfonyl)-5-methyl-1- | 1H), 7.71 (d, J = 2.1 Hz, 1H), 7.47 (dd, J = 8.4, 1.7 Hz, |
| | (2,4,5-trichlorophenyl)-1H-1,2,3- | 1H), 7.13 (d, J = 8.6 Hz, 1H), 3.82 (s, 3H), 2.32 (s, 3H), |
| | triazole-4-carboxamide | 2.28 (s, 3H); LCMS(M + 1: 490.8 |
| 654 | 5-methyl-N-(o-tolylsulfonyl)-1- | ¹H-NMR (400 MHz, DMSO-d₆) δ 8.29 (s, 1H), 8.20 (d, |
| | (2,4,5-trichlorophenyl)-1H-1,2,3- | J = 2.8 Hz, 1H), 8.04 (d, J = 7.6 Hz, 1H), 7.57 (t, J = 7.0 |
| | triazole-4-carboxamide | Hz, 1H), 7.46-7.39 (m, 2H), 2.66-2.64 (m, 3H); 2.30-2.27 |
| | | (m, 3H), LCMS(M − 1): 458.9 |
| 655 | N-((3-chloro-4- | ¹H-NMR (400 MHz, DMSO-d₆) δ 8.29 (s, 1H), 8.19 (s, |
| | fluorophenyl)sulfonyl)-5-methyl-1- | 1H), 8.14 (dd, J = 6.7, 2.1 Hz, 1H), 7.99 (qd, J = 4.3, 2.2 |
| | (2,4,5-trichlorophenyl)-1H-1,2,3- | Hz, 1H), 7.67 (t, J = 8.9 Hz, 1H), 2.31 (s, 3H); |
| | triazole-4-carboxamide | LCMS(M + 1): 498.8 |
| 656 | N-((4-chlorophenyl)sulfonyl)-5- | ¹H-NMR (400 MHz, DMSO-d₆) δ 8.30 (d, J = 2.4 Hz, |
| | methyl-1-(2,4,5-trichlorophenyl)- | 1H), 8.20 (s, 1H), 8.02 (dt, J = 9.1, 2.3 Hz, 2H), 7.74 (dt, |
| | 1H-1,2,3-triazole-4-carboxamide | J = 9.1, 2.2 Hz, 2H), 2.30 (s, 3H); LCMS(M + 1): 480.7 |
| 657 | 5-methyl-1-(2,4,5-trichlorophenyl)- | ¹H-NMR (400 MHz, DMSO-d₆) δ 8.37 (d, J = 7.6 Hz, |
| | N-((2- | 1H), 8.30 (d, J = 4.3 Hz, 1H), 8.22 (d, J = 4.9 Hz, 1H), |
| | (trifluoromethyl)phenyl)sulfonyl)- | 8.01 (d, J = 7.6 Hz, 1H), 7.93 (td, J = 14.5, 6.8 Hz, 2H); |
| | 1H-1,2,3-triazole-4-carboxamide | 2.29 (s, 3H), LCMS(M − 1): 512.9 |
| 658 | 5-methyl-1-(2,4,5-trichlorophenyl)- | ¹H-NMR (400 MHz, DMSO-d₆) δ 8.28 (s, 1H), 8.18 (d, |
| | N-((4- | J = 3.7 Hz, 1H), 8.10 (d, J = 8.6 Hz, 2H), 7.59 (d, J = 8.3 |
| | (trifluoromethoxy)phenyl)sulfonyl)- | Hz, 2H), 2.31 (s, 3H); LCMS(M + 1): 530.8 |
| | 1H-1,2,3-triazole-4-carboxamide | |
| 659 | N-((3-chloro-2- | ¹H-NMR (400 MHz, DMSO-d₆) δ 8.30 (s, 1H), 8.20 (s, |
| | methylphenyl)sulfonyl)-5-methyl-1- | 1H), 8.08-8.06 (m, 1H), 7.79 (d, J = 7.6 Hz, 1H), 7.49 (t, |
| | (2,4,5-trichlorophenyl)-1H-1,2,3- | J = 7.9 Hz, 1H), 2.68-2.66 (m, 3H), 2.28 (s, 3H); |
| | triazole-4-carboxamide | LCMS(M + 1): 494.7 |
| 660 | N-((2,4-dichlorophenyl)sulfonyl)-5- | ¹H-NMR (400 MHz, DMSO-d₆) δ 8.30 (d, J = 4.6 Hz, |
| | methyl-1-(2,4,5-trichlorophenyl)- | 1H), 8.23 (d, J = 7.6 Hz, 1H), 8.16 (d, J = 8.6 Hz, 1H), |
| | 1H-1,2,3-triazole-4-carboxamide | 7.90 (d, J = 2.1 Hz, 1H), 7.72 (dd, J = 8.6, 2.1 Hz, 1H), |
| | | 2.28 (s, 3H); LCMS(M + 2): 516.5 |
| 661 | 1-(2,4-difluorophenyl)-N-((2- | ¹H-NMR (400 MHz, DMSO-d₆) δ 12.36 (s, 1H), 7.82 (td, |
| | methoxy-5-methylphenyl)sulfonyl)- | J = 8.7, 6.0 Hz, 1H), 7.75-7.70 (m, 2H), 7.47 (d, J = 7.3 |
| | 5-methyl-1H-1,2,3-triazole-4- | Hz, 1H), 7.43-7.38 (m, 1H), 7.13 (d, J = 8.6 Hz, 1H), 3.83 |
| | carboxamide | (s, 3H), 2.32 (s, 3H), 2.30 (s, 3H)); LCMS (M+): 422.95 |
| 662 | 1-(2,4-difluorophenyl)-N-((2,4- | ¹H-NMR (400 MHz, DMSO-d₆) δ 8.07 (td, J = 8.6, 6.4 |
| | difluorophenyl)sulfonyl)-5-methyl- | Hz, 1H), 7.82 (td, J = 8.7, 5.9 Hz, 1H), 7.75-7.69 (m, 1H), |
| | 1H-1,2,3-triazole-4-carboxamide | 7.58-7.53 (m, 1H), 7.43-7.33 (m, 2H), 2.31 (s, 3H); |
| | | LCMS (M+): 414.85 |
| 663 | N-((3-chloro-2- | ¹H-NMR (400 MHz, DMSO-d₆) δ 13.30 (s, 1H), 8.07 (d, |
| | methylphenyl)sulfonyl)-1-(2,4- | J = 7.0 Hz, 1H), 7.81 (td, J = 8.9, 6.1 Hz, 2H), 7.75-7.69 |
| | difluorophenyl)-5-methyl-1H-1,2,3- | (m, 1H), 7.49 (t, J = 8.1 Hz, 1H), 7.43-7.38 (m, 1H), 2.68 |
| | triazole-4-carboxamide | (s, 3H), 2.30 (s, 3H); LCMS (M+): 426.90 |
| 664 | 1-(2-chloro-4- | ¹H-NMR (400 MHz, CHLOROFORM-D) δ 9.95 (s, 1H), |
| | (difluoromethoxy)phenyl)-N-((2- | 7.86 (d, J = 2.9 Hz, 1H), 7.43-7.40 (m, 3H), 7.28 (d, J = |
| | chloro-5-methoxyphenyl)sulfonyl)- | 2.4 Hz, 1H), 7.09 (dd, J = 8.8, 2.9 Hz, 1H), 6.62 (t, J = |
| | 5-methyl-1H-1,2,3-triazole-4- | 71.9 Hz, 1H), 3.90 (s, 3H), 2.40 (s, 3H); LCMS(M + 1): |
| | carboxamide | 506.85 |
| 665 | 1-(2-chloro-4- | ¹H-NMR (400 MHz, CHLOROFORM-D) δ 10.00 (s, |
| | (difluoromethoxy)phenyl)-N-((2- | 1H), 8.63 (d, J = 2.2 Hz, 1H), 7.83 (dd, J = 8.4, 1.6 Hz, |
| | chloro-5- | 1H), 7.69 (d, J = 8.1 Hz, 1H), 7.43-7.41 (m, 2H), 7.28 (d, |
| | (trifluoromethyl)phenyl)sulfonyl)-5- | J = 2.4 Hz, 1H), 6.62 (t, J = 71.9 Hz, 1H), 2.40 (s, 3H); |
| | methyl-1H-1,2,3-triazole-4- | LCMS(M + 1): 544.90 |
| | carboxamide | |
| 666 | 1-(2-chloro-4- | ¹H-NMR (400 MHz, CHLOROFORM-D) δ 9.94 (s, 1H), |
| | (difluoromethoxy)phenyl)-N-((2,5- | 8.35 (d, J = 2.4 Hz, 1H), 7.54 (dd, J = 8.6, 2.4 Hz, 1H), |
| | dichlorophenyl)sulfonyl)-5-methyl- | 7.47 (d, J = 8.3 Hz, 1H), 7.43-7.41 (m, 2H), 7.28 (d, J = |
| | 1H-1,2,3-triazole-4-carboxamide | 2.4 Hz, 1H), 6.62 (t, J = 71.9 Hz, 1H), 2.40 (s, 3H); |
| | | LCMS(M + 1): 510.80 |
| 667 | 1-(2-chloro-4- | ¹H-NMR (400 MHz, CHLOROFORM-D) δ 9.73 (s, 1H), |
| | (difluoromethoxy)phenyl)-N-((2,5- | 8.09 (s, 1H), 7.42-7.40 (m, 2H), 7.33 (d, J = 7.6 Hz, 1H), |
| | dimethylphenyl)sulfonyl)-5-methyl- | 7.28 (s, 1H), 7.21 (d, J = 7.8 Hz, 1H), 6.62 (t, J = 71.9 Hz, |
| | 1H-1,2,3-triazole-4-carboxamide | 1H), 2.70 (d, J = 4.4 Hz, 3H), 2.43 (s, 3H), 2.40 (s, 3H); |
| | | LCMS(M + 1): 471.15 |
| 668 | 1-(2-chloro-4- | ¹H-NMR (400 MHz, CHLOROFORM-D) δ 9.91 (s, 1H), |
| | (difluoromethoxy)phenyl)-N-((2- | 8.21-8.17 (m, 1H), 7.69-7.64 (m, 1H), 7.42-7.35 (m, 3H), |
| | fluorophenyl)sulfonyl)-5-methyl-1H- | 7.27 (d, J = 2.7 Hz, 1H), 7.23 (d, J = 8.8 Hz, 1H), 6.62 (t, |
| | 1,2,3-triazole-4-carboxamide | J = 71.9 Hz, 1H), 2.40 (s, 3H); LCMS(M + 1): 460.95 |
| 669 | 1-(2-chloro-4- | ¹H-NMR (400 MHz, CHLOROFORM-D) δ 9.83 (s, 1H), |
| | (difluoromethoxy)phenyl)-5-methyl- | 8.64-8.62 (m, 1H), 7.91 (dd, J = 7.2, 1.8 Hz, 1H), 7.84- |
| | N-((2- | 7.77 (m, 2H), 7.42-7.40 (m, 2H), 7.27 (s, 1H), 7.24 (s, |
| | (trifluoromethyl)phenyl)sulfonyl)- | 1H), 6.62 (t, J = 71.9 Hz, 1H), 2.39 (s, 3H); LCMS(M + 1): |
| | 1H-1,2,3-triazole-4-carboxamide | 511.10 |
| 670 | 1-(2-chloro-4- | ¹H-NMR (400 MHz, CHLOROFORM-D) δ 9.73 (s, 1H), |
| | (trifluoromethyl)phenyl)-N-((2,5- | 8.09 (s, 1H), 7.91 (d, J = 2.0 Hz, 1H), 7.78 (dd, J = 8.2, |
| | dimethylphenyl)sulfonyl)-5-methyl- | 1.3 Hz, 1H), 7.57 (d, J = 8.1 Hz, 1H), 7.34 (d, J = 6.4 Hz, |
| | 1H-1,2,3-triazole-4-carboxamide | 1H), 7.22 (d, J = 7.8 Hz, 1H), 2.70 (s, 3H), 2.42 (d, J = |
| | | 4.9 Hz, 6H); LCMS(M + 1): 472.95 |
| 671 | 1-(2-chloro-4- | ¹H-NMR (400 MHz, CHLOROFORM-D) δ 9.96 (s, 1H), |
| | (difluoromethoxy)phenyl)-N-((2- | 8.38 (dt, J = 7.8, 0.9 Hz, 1H), 7.61-7.49 (m, 3H), 7.42 (dd, |
| | chlorophenyl)sulfonyl)-5-methyl- | J = 5.6, 3.2 Hz, 2H), 7.27 (d, J = 2.4 Hz, 1H), 6.62 (t, J = |
| | 1H-1,2,3-triazole-4-carboxamide | 72.0 Hz, 1H), 2.30-2.45 (3H); LCMS(M + 1): 476.90 |
| 672 | 1-(2-chloro-4- | ¹H-NMR (400 MHz, CHLOROFORM-D) δ 9.89 (s, 1H), |
| | (difluoromethoxy)phenyl)-N-((2,4- | 8.22 (td, J = 8.6, 6.1 Hz, 1H), 7.43-7.40 (m, 2H), 7.28 (d, |
| | difluorophenyl)sulfonyl)-5-methyl- | J = 2.4 Hz, 1H), 7.12-7.07 (m, 1H), 7.01-6.95 (m, 1H), |
| | 1H-1,2,3-triazole-4-carboxamide | 6.62 (t, J = 71.9 Hz, 1H), 2.40 (s, 3H); LCMS(M + 1): |
| | | 479.05 |
| 673 | 1-(2-chloro-4- | ¹H-NMR (400 MHz, CHLOROFORM-D) δ 9.75 (s, 0H), |
| | (difluoromethoxy)phenyl)-N-((3,5- | 7.74 (qd, J = 2.6, 1.0 Hz, 2H), 7.43-7.40 (m, 2H), 7.28 (d, |
| | difluorophenyl)sulfonyl)-5-methyl- | J = 2.4 Hz, 1H), 7.11 (tt, J = 8.4, 2.4 Hz, 1H), 6.62 (t, J = |
| | 1H-1,2,3-triazole-4-carboxamide | 71.9 Hz, 1H), 2.44 (s, 3H); LCMS(M + 1): 478.90 |
| 674 | 1-(2-chloro-4- | ¹H-NMR (400 MHz, CHLOROFORM-D) δ 7.92-7.88 |
| | (difluoromethoxy)phenyl)-N-((2,5- | (m, 1H), 7.43-7.41 (m, 2H), 7.38-7.32 (m, 1H), 7.28 (d, |
| | difluorophenyl)sulfonyl)-5-methyl- | J = 2.4 Hz, 1H), 7.22 (td, J = 9.0, 3.9 Hz, 1H), 6.62 (t, J = |
| | 1H-1,2,3-triazole-4-carboxamide | 71.9 Hz, 1H), 2.40 (d, J = 10.8 Hz, 3H); LCMS(M + 1): |
| | | 479.10 |
| 675 | 1-(2-chloro-4- | ¹H-NMR (400 MHz, CHLOROFORM-D) δ 9.94 (s, 1H), |
| | (difluoromethoxy)phenyl)-5-methyl- | 7.92-7.88 (m, 1H), 7.44-7.41 (m, 1H), 7.38-7.32 (m, 1H), |
| | N-((2,4,6-trifluorophenyl)sulfonyl)- | 7.28 (d, J = 1.2 Hz, 1H), 7.21 (q, J = 4.4 Hz, 1H), 6.86- |
| | 1H-1,2,3-triazole-4-carboxamide | 6.44 (m, 1H), 2.43-2.38 (m, 3H); LCMS(M + 1): 496.85 |
| 676 | 1-(2-chloro-4- | ¹H-NMR (400 MHz, CHLOROFORM-D) δ 9.95 (s, 1H), |
| | (difluoromethoxy)phenyl)-5-methyl- | 8.44 (s, 1H), 7.65 (s, 1H), 7.44-7.41 (m, 2H), 7.28 (d, J = |
| | N-((2,4,5-trichlorophenyl)sulfonyl)- | 2.4 Hz, 1H), 6.62 (t, J = 71.9 Hz, 1H), 2.41 (s, 3H); |
| | 1H-1,2,3-triazole-4-carboxamide | LCMS(M + 1): 546.75 |
| 677 | N-((2,5-difluorophenyl)sulfonyl)-5- | ¹H-NMR (400 MHz, DMSO-d₆) δ 8.30 (s, 1H), 8.21 (s, |
| | methyl-1-(2,4,5-trichlorophenyl)- | 1H), 7.78-7.74 (m, 1H), 7.67-7.62 (m, 1H), 7.53 (td, J = |
| | 1H-1,2,3-triazole-4-carboxamide | 9.3, 4.0 Hz, 1H), 2.30 (s, 3H); LCMS(M + 1): 482.7 |
| 678 | N-((5-chloro-2,4- | ¹H-NMR (400 MHz, DMSO-d₆) δ 8.30 (d, J = 4.6 Hz, |
| | difluorophenyl)sulfonyl)-5-methyl- | 1H), 8.21 (t, J = 4.1 Hz, 1H), 8.12 (t, J = 7.5 Hz, 1H), 7.84 |
| | 1-(2,4,5-trichlorophenyl)-1H-1,2,3- | (t, J = 9.6 Hz, 1H), 2.32-2.30 (m, 3H); LCMS(M + 1): |
| | triazole-4-carboxamide | 516.7 |
| 679 | 5-methyl-1-(2,4,5-trichlorophenyl)- | ¹H-NMR (400 MHz, DMSO-d₆) δ 8.29 (s, 1H), 8.25 (s, |
| | N-((2,4,5-trichlorophenyl)sulfonyl)- | 1H), 8.22 (d, J = 4.6 Hz, 1H), 8.12 (s, 1H), 2.32-2.30 (m, |
| | 1H-1,2,3-triazole-4-carboxamide | 3H); LCMS(M + 1): 550.5 |
| 680 | N-((2-fluorophenyl)sulfonyl)-5- | ¹H-NMR (400 MHz, DMSO-d₆) δ 8.30 (s, 1H), 8.21 (s, |
| | methyl-1-(2,4,5-trichlorophenyl)- | 1H), 8.00 (td, J = 7.8, 1.5 Hz, 1H), 7.77 (dd, J = 5.8, 1.8 |
| | 1H-1,2,3-triazole-4-carboxamide | Hz, 1H), 7.48-7.44 (m, 2H), 2.28 (s, 3H); LCMS(M + 1): |
| | | 464.6 |
| 681 | N-((5-fluoro-2- | ¹H-NMR (400 MHz, DMSO-d₆) δ 8.27 (d, J = 4.6 Hz, |
| | methylphenyl)sulfonyl)-5-methyl-1- | 1H), 8.18 (s, 1H), 7.76 (dd, J = 8.6, 1.8 Hz, 1H), 7.46- |
| | (2,4,5-trichlorophenyl)-1H-1,2,3- | 7.44 (m, 2H), 2.58 (s, 3H), 2.26 (s, 3H); LCMS(M + 1): |
| | triazole-4-carboxamide | 478.7 |
| 682 | N-((2-chlorophenyl)sulfonyl)-5- | ¹H-NMR (400 MHz, DMSO-d₆) δ 8.30 (s, 1H), 8.22 (s, |
| | methyl-1-(2,4,5-trichlorophenyl)- | 1H), 8.17 (dd, J = 7.8, 1.4 Hz, 1H), 7.73-7.66 (m, 2H), |
| | 1H-1,2,3-triazole-4-carboxamide | 7.63-7.59 (m, 1H), 2.28 (s, 3H); LCMS(M + 1): 480.7 |
| 683 | N-((2,4-difluorophenyl)sulfonyl)-5- | ¹H-NMR (400 MHz, DMSO-d₆) δ 8.30 (s, 1H), 8.21 (s, |
| | methyl-1-(2,4,5-trichlorophenyl)- | 1H), 8.07 (dd, J = 15.0, 8.6 Hz, 1H), 7.59-7.54 (m, 1H), |
| | 1H-1,2,3-triazole-4-carboxamide | 7.36 (t, J = 8.7 Hz, 1H), 2.29 (s, 3H); LCMS(M + 1): 482.7 |
| 684 | N-((3-chloro-2,5- | ¹H-NMR (400 MHz, DMSO-d₆) δ 8.30 (s, 1H), 8.20 (s, |
| | dimethylphenyl)sulfonyl)-5-methyl- | 1H), 7.99 (s, 1H), 7.52 (s, 1H), 2.59 (s, 3H), 2.39 (s, 3H), |
| | 1-(2,4,5-trichlorophenyl)-1H-1,2,3- | 2.28 (s, 3H); LCMS(M + 1): 508.8 |
| | triazole-4-carboxamide | |
| 685 | N-((5-chloro-2- | ¹H-NMR (400 MHz, DMSO-d₆) δ 8.29 (s, 1H), 8.21 (s, |
| | fluorophenyl)sulfonyl)-5-methyl-1- | 1H), 7.94 (q, J = 2.9 Hz, 1H), 7.84 (dt, J = 8.9, 3.4 Hz, |
| | (2,4,5-trichlorophenyl)-1H-1,2,3- | 1H), 7.53 (t, J = 9.3 Hz, 1H), 2.31 (d, J = 6.4 Hz, 3H); |
| | triazole-4-carboxamide | LCMS(M + 1): 498.7 |
| 686 | N-((4-fluorophenyl)sulfonyl)-5- | ¹H-NMR (400 MHz, DMSO-d₆) δ 8.29 (s, 1H), 8.21 (s, |
| | methyl-1-(2,4,5-trichlorophenyl)- | 1H), 7.94 (q, J = 2.9 Hz, 1H), 7.84 (dt, J = 8.9, 3.4 Hz, |
| | 1H-1,2,3-triazole-4-carboxamide | 1H), 7.53 (t, J = 9.3 Hz, 1H), 2.31 (d, J = 6.4 Hz, 3H); |
| | | LCMS(M + 1): 530.8 |
| 687 | 1-(2,4-difluorophenyl)-5-methyl-N- | ¹H-NMR (400 MHz, DMSO-d₆) δ 8.36 (d, J = 7.6 Hz, |
| | ((2- | 1H), 8.01-7.78 (m, 4H), 7.75-7.69 (m, 1H), 7.43-7.38 (m, |
| | (trifluoromethyl)phenyl)sulfonyl)- | 1H), 2.32 (s, 3H); LCMS (M + 1): 446.95 |
| | 1H-1,2,3-triazole-4-carboxamide | |
| 688 | 1-(2,4-difluorophenyl)-N-((2- | ¹H-NMR (400 MHz, DMSO-d₆) δ 8.00 (td, J = 7.6, 1.5 |
| | fluorophenyl)sulfonyl)-5-methyl-1H- | Hz, 1H), 7.85-7.69 (m, 3H), 7.48-7.38 (m, 3H), 2.31 (s, |
| | 1,2,3-triazole-4-carboxamide | 3H); LCMS (M+): 396.95 |
| 689 | N-((2-chlorophenyl)sulfonyl)-1-(2,4- | ¹H-NMR (400 MHz, DMSO-d₆) δ 8.16 (dd, J = 7.9, 1.2 |
| | difluorophenyl)-5-methyl-1H-1,2,3- | Hz, 1H), 7.82 (td, J = 8.8, 6.0 Hz, 1H), 7.76-7.65 (m, 3H), |
| | triazole-4-carboxamide | 7.62-7.58 (m, 1H), 7.43-7.37 (m, 1H), 2.31 (s, 3H); |
| | | LCMS (M+): 412.95 |
| 690 | N-((2,5- | ¹H-NMR (400 MHz, DMSO-d₆) δ 8.61 (s, 1H), 8.28 (d, |
| | bis(trifluoromethyl)phenyl)sulfonyl)- | J = 8.6 Hz, 1H), 8.21 (d, J = 8.3 Hz, 1H), 7.81 (td, J = 8.7, |
| | 1-(2,4-difluorophenyl)-5-methyl-1H- | 5.9 Hz, 1H), 7.74-7.68 (m, 1H), 7.42-7.37 (m, 1H), 2.32 |
| | 1,2,3-triazole-4-carboxamide | (s, 3H); LCMS (M+): 514.90 |
| 691 | N-((5-chloro-2- | ¹H-NMR (400 MHz, DMSO-d₆) δ 7.94 (q, J = 3.0 Hz, |
| | fluorophenyl)sulfonyl)-1-(2,4- | 1H), 7.86-7.79 (m, 2H), 7.75-7.69 (m, 1H), 7.53 (t, J = |
| | difluorophenyl)-5-methyl-1H-1,2,3- | 9.3 Hz, 1H), 7.43-7.38 (m, 1H), 2.32 (s, 3H); LCMS |
| | triazole-4-carboxamide | (M+): 431.00 |
| 692 | 1-(2,4-difluorophenyl)-N-((5-fluoro- | ¹H-NMR (400 MHz, DMSO-d₆) δ 13.16 (s, 1H), 7.84- |
| | 2-methylphenyl)sulfonyl)-5-methyl- | 7.77 (m, 2H), 7.74-7.69 (m, 1H), 7.48-7.46 (m, 2H), 7.42- |
| | 1H-1,2,3-triazole-4-carboxamide | 7.37 (m, 1H), 2.61 (s, 3H), 2.31 (s, 3H); LCMS (M+): |
| | | 410.95 |
| 693 | 1-(2,4-difluorophenyl)-5-methyl-N- | ¹H-NMR (400 MHz, DMSO-d₆) δ 8.14 (dd, J = 8.1, 1.7 |
| | ((2- | Hz, 1H), 7.82 (td, J = 8.7, 5.9 Hz, 2H), 7.75-7.69 (m, 1H), |
| | (trifluoromethoxy)phenyl)sulfonyl)- | 7.63 (d, J = 7.6 Hz, 1H), 7.60-7.57 (m, 1H), 7.40 (dd, J = |
| | 1H-1,2,3-triazole-4-carboxamide | 9.0, 6.6 Hz, 1H), 2.31 (s, 3H); LCMS (M + 1): 463.10 |
| 694 | N-((4-chloro-2,5- | ¹H-NMR (400 MHz, DMSO-d₆) δ 13.09 (s, 1H), 7.99 (s, |
| | dimethylphenyl)sulfonyl)-1-(2,4- | 1H), 7.81 (td, J = 8.8, 6.0 Hz, 1H), 7.75-7.69 (m, 1H), |
| | difluorophenyl)-5-methyl-1H-1,2,3- | 7.51-7.37 (m, 3H), 2.59 (s, 3H), 2.39 (s, 3H), 2.29 (s, 3H); |
| | triazole-4-carboxamide | LCMS (M + 1): 441.05 |
| 695 | N-((5-chloro-2,4- | ¹H-NMR (400 MHz, DMSO-d₆) δ 8.14 (t, J = 7.6 Hz, 1H), |
| | difluorophenyl)sulfonyl)-1-(2,4- | 7.88-7.79 (m, 2H), 7.75-7.69 (m, 1H), 7.43-7.38 (m, 1H), |
| | difluorophenyl)-5-methyl-1H-1,2,3- | 2.32 (s, 3H); LCMS (M+): 449.00 |
| | triazole-4-carboxamide | |
| 696 | 1-(2,4-difluorophenyl)-5-methyl-N- | ¹H-NMR (400 MHz, DMSO-d₆) δ 8.25 (s, 1H), 8.11 (s, |
| | ((2,4,5-trichlorophenyl)sulfonyl)- | 1H), 7.82 (td, J = 8.8, 5.9 Hz, 1H), 7.74-7.69 (m, 1H), |
| | 1H-1,2,3-triazole-4-carboxamide | 7.43-7.38 (m, 1H), 2.32 (s, 3H); LCMS (M+): 481.00 |
| 697 | 1-(2,4-difluorophenyl)-N-((4- | ¹H-NMR (400 MHz, DMSO-d₆) δ 12.76 (s, 1H), 8.09 (dd, |
| | fluorophenyl)sulfonyl)-5-methyl-1H- | J = 8.8, 5.1 Hz, 2H), 7.82 (td, J = 8.8, 5.9 Hz, 1H), 7.76- |
| | 1,2,3-triazole-4-carboxamide | 7.70 (m, 1H), 7.50 (t, J = 8.8 Hz, 2H), 7.44-7.39 (m, 1H), |
| | | 2.33 (s, 3H); LCMS (M+): 396.95 |
| 698 | N-((2,5-dimethylphenyl)sulfonyl)-1- | ¹H-NMR (400 MHz, DMSO-d₆) δ 8.30 (s, 1H), 8.20 (s, |
| | (2,4,5-trichlorophenyl)-5- | 1H), 8.10-8.07 (m, 2H), 7.52-7.47 (m, 2H), 2.30 (s, 3H); |
| | (trifluoromethyl)-1H-1,2,3-triazole- | LCMS(M + 1): 464.7 |
| | 4-carboxamide | |
| 699 | N-((2,5-dichlorophenyl)sulfonyl)-1- | ¹H-NMR (400 MHz, DMSO-d₆) δ 8.45 (s, 1H), 8.33 (d, |
| | (2,4,5-trichlorophenyl)-5- | J = 4.4 Hz, 1H), 8.03 (d, J = 2.4 Hz, 1H), 7.65 (dd, J = 8.6, |
| | (trifluoromethyl)-1H-1,2,3-triazole- | 2.7 Hz, 1H), 7.59 (d, J = 8.6 Hz, 1H); LCMS(M − 1): 567.0 |
| | 4-carboxamide | |
| 700 | N-((2-chloro-5- | ¹H-NMR (400 MHz, DMSO-d₆) δ 8.46 (s, 1H), 8.34 (s, |
| | methoxyphenyl)sulfonyl)-1-(2,4,5- | 1H), 7.60 (d, J = 3.2 Hz, 1H), 7.54 (d, J = 8.8 Hz, 1H), |
| | trichlorophenyl)-5-(trifluoromethyl)- | 7.23 (dd, J = 8.8, 3.2 Hz, 1H), 3.84 (s, 3H); LCMS(M − 1): |
| | 1H-1,2,3-triazole-4-carboxamide | 563.0 |
| 701 | N-((2,5- | ¹H-NMR (400 MHz, DMSO-d₆) δ 8.54 (s, 1H), 8.45 (s, |
| | bis(trifluoromethyl)phenyl)sulfonyl)- | 1H), 8.30 (s, 1H), 8.10-8.08 (m, 2H); LCMS(M − 1): 635.0 |
| | 1-(2,4,5-trichlorophenyl)-5- | |
| | (trifluoromethyl)-1H-1,2,3-triazole- | |
| | 4-carboxamide | |
| 702 | N-((2-methoxy-5- | ¹H-NMR (400 MHz, DMSO-d₆) δ 8.46 (s, 1H), 8.36 (s, |
| | methylphenyl)sulfonyl)-1-(2,4,5- | 1H), 7.71 (d, J = 1.7 Hz, 1H), 7.50 (dd, J = 8.6, 1.7 Hz, |
| | trichlorophenyl)-5-(trifluoromethyl)- | 1H), 7.16 (d, J = 8.6 Hz, 1H), 3.84 (s, 3H), 2.33 (s, 3H); |
| | 1H-1,2,3-triazole-4-carboxamide | LCMS(M + 2): 545.1 |
| 703 | N-(o-tolylsulfonyl)-1-(2,4,5- | ¹H-NMR (400 MHz, DMSO-d₆) δ 8.43 (d, J = 4.2 Hz, |
| | trichlorophenyl)-5-(trifluoromethyl)- | 1H), 8.35 (s, 1H), 8.04 (d, J = 7.8 Hz, 1H), 7.60-7.56 (m, |
| | 1H-1,2,3-triazole-4-carboxamide | 1H), 7.44 (dd, J = 15.4, 7.3 Hz, 2H), 2.65 (s, 3H); |
| | | LCMS(M − 1): 512.9 |
| 704 | N-((3-chloro-4- | ¹H-NMR (400 MHz, DMSO-d₆) δ 8.42 (s, 1H), 8.34 (s, |
| | fluorophenyl)sulfonyl)-1-(2,4,5- | 1H), 8.11 (dd, J = 6.8, 2.2 Hz, 1H), 7.98 (qd, J = 4.4, 2.3 |
| | trichlorophenyl)-5-(trifluoromethyl)- | Hz, 1H), 7.67 (t, J = 8.9 Hz, 1H); LCMS(M − 1): 551.0 |
| | 1H-1,2,3-triazole-4-carboxamide | |
| 705 | N-((4-chlorophenyl)sulfonyl)-1- | ¹H-NMR (400 MHz, DMSO-d₆) δ 8.42 (s, 1H), 8.33 (s, |
| | (2,4,5-trichlorophenyl)-5- | 1H), 7.99-7.96 (m, 2H), 7.70 (d, J = 8.6 Hz, 2H); |
| | (trifluoromethyl)-1H-1,2,3-triazole- | LCMS(M + 1): 535.0 |
| | 4-carboxamide | |
| 706 | 1-(2,4,5-trichlorophenyl)-5- | ¹H-NMR (400 MHz, DMSO-d₆) δ 8.45 (s, 1H), 8.33 (s, |
| | (trifluoromethyl)-N-((2- | 1H), 8.11 (d, J = 8.6 Hz, 1H), 7.81 (d, J = 2.1 Hz, 1H), |
| | (trifluoromethyl)phenyl)sulfonyl)- | 7.65 (dd, J = 8.6, 2.1 Hz, 1H); LCMS(M + 1): 583 |
| | 1H-1,2,3-triazole-4-carboxamide | |
| 707 | 1-(2,4,5-trichlorophenyl)-N-((4- | ¹H-NMR (400 MHz, DMSO-d₆) δ 8.41 (s, 1H), 8.34 (s, |
| | (trifluoromethoxy)phenyl)sulfonyl)- | 1H), 8.11 (dt, J = 9.6, 2.4 Hz, 2H), 7.62 (d, J = 7.9 Hz, |
| | 5-(trifluoromethyl)-1H-1,2,3- | 2H); LCMS(M − 1): 547.0 |
| | triazole-4-carboxamide | |
| 708 | N-((3-chloro-2- | ¹H-NMR (400 MHz, DMSO-d₆) δ 8.40 (s, 1H), 8.31 (s, |
| | methylphenyl)sulfonyl)-1-(2,4,5- | 1H), 8.01 (d, J = 8.3 Hz, 1H), 7.72 (d, J = 7.9 Hz, 1H), |
| | trichlorophenyl)-5-(trifluoromethyl)- | 7.43 (t, J = 7.9 Hz, 1H), 2.64 (d, J = 5.5 Hz, 3H); |
| | 1H-1,2,3-triazole-4-carboxamide | LCMS(M + 1): 547.0 |
| 709 | N-((2,4-dichlorophenyl)sulfonyl)-1- | ¹H-NMR (400 MHz, DMSO-d₆) δ 8.44 (s, 1H), 8.37-8.34 |
| | (2,4,5-trichlorophenyl)-5- | (m, 2H), 7.98-7.84 (m, 3H); LCMS(M + 1): 569.0 |
| | (trifluoromethyl)-1H-1,2,3-triazole- | |
| | 4-carboxamide | |
| 710 | N-((2-chloro-5- | ¹H-NMR (400 MHz, CHLOROFORM-D) δ 10.02 (s, |
| | methoxyphenyl)sulfonyl)-1-(2,4,6- | 1H), 7.86 (d, J = 2.9 Hz, 1H), 7.59 (s, 2H), 7.43 (d, J = |
| | trichlorophenyl)-5-(trifluoromethyl)- | 8.8 Hz, 1H), 7.11 (dd, J = 8.8, 3.2 Hz, 1H), 3.91 (s, 3H); |
| | 1H-1,2,3-triazole-4-carboxamide | LCMS (M − 1): 563.05 |
| 711 | N-((2-chloro-5- | ¹H-NMR (400 MHz, CHLOROFORM-D) δ 8.63 (s, 1H), |
| | (trifluoromethyl)phenyl)sulfonyl)-1- | 7.84 (dd, J = 8.3, 2.0 Hz, 1H), 7.71 (d, J = 8.3 Hz, 1H), |
| | (2,4,6-trichlorophenyl)-5- | 7.59 (t, J = 5.0 Hz, 2H); LCMS (M − 1): 601.05 |
| | (trifluoromethyl)-1H-1,2,3-triazole- | |
| | 4-carboxamide | |
| 712 | N-((2,5-dichlorophenyl)sulfonyl)-1- | ¹H-NMR (400 MHz, CHLOROFORM-D) δ 10.04 (s, |
| | (2,4,6-trichlorophenyl)-5- | 1H), 8.36 (d, J = 2.4 Hz, 1H), 7.59-7.56 (m, 3H), 7.50 (d, |
| | (trifluoromethyl)-1H-1,2,3-triazole- | J = 8.3 Hz, 1H); LCMS (M − 1): 567.00 |
| | 4-carboxamide | |
| 713 | N-((2,4-dichlorophenyl)sulfonyl)-1- | ¹H-NMR (400 MHz, CHLOROFORM-D) δ 10.00 (s, |
| | (2,4,6-trichlorophenyl)-5- | 1H), 8.32 (d, J = 8.8 Hz, 1H), 7.58 (t, J = 2.3 Hz, 3H), |
| | (trifluoromethyl)-1H-1,2,3-triazole- | 7.52-7.49 (m, 1H); LCMS (M − 1): 566.70 |
| | 4-carboxamide | |
| 714 | N-((2,5-dimethylphenyl)sulfonyl)-1- | ¹H-NMR (400 MHz, CHLOROFORM-D) δ 9.77 (s, 1H), |
| | (2,4,6-trichlorophenyl)-5- | 8.10 (s, 1H), 7.58 (s, 2H), 7.35 (d, J = 7.6 Hz, 1H), 7.23 |
| | (trifluoromethyl)-1H-1,2,3-triazole- | (d, J = 7.6 Hz, 1H), 2.71 (s, 3H), 2.43 (s, 3H); LCMS (M − |
| | 4-carboxamide | 1): 524.85 |
| 715 | N-((5-fluoro-2- | ¹H-NMR (400 MHz, CHLOROFORM-D) δ 9.83 (s, 1H), |
| | methylphenyl)sulfonyl)-1-(2,4,6- | 8.02 (dd, J = 8.3, 2.7 Hz, 1H), 7.59 (t, J = 3.9 Hz, 2H), |
| | trichlorophenyl)-5-(trifluoromethyl)- | 7.33 (dd, J = 8.6, 5.1 Hz, 1H), 7.28 (d, J = 2.7 Hz, 1H), |
| | 1H-1,2,3-triazole-4-carboxamide | 2.72 (s, 3H); LCMS (M − 1): 528.75 |
| 716 | N-((3-chloro-2- | ¹H-NMR (400 MHz, CHLOROFORM-D) δ 9.88 (s, 1H), |
| | methylphenyl)sulfonyl)-1-(2,4,6- | 8.28 (dd, J = 8.1, 1.2 Hz, 1H), 7.68 (dd, J = 8.1, 1.0 Hz, |
| | trichlorophenyl)-5-(trifluoromethyl)- | 1H), 7.58 (t, J = 3.9 Hz, 2H), 7.39 (t, J = 8.1 Hz, 1H), 2.81 |
| | 1H-1,2,3-triazole-4-carboxamide | (s, 3H); LCMS (M − 1): 546.65 |
| 717 | N-((5-chloro-2- | ¹H-NMR (400 MHz, CHLOROFORM-D) δ 9.99 (s, 1H), |
| | fluorophenyl)sulfonyl)-1-(2,4,6- | 8.17 (dd, J = 6.0, 2.6 Hz, 1H), 7.63 (qd, J = 4.4, 2.8 Hz, |
| | trichlorophenyl)-5-(trifluoromethyl)- | 1H), 7.58 (d, J = 9.0 Hz, 2H), 7.22 (t, J = 9.0 Hz, 1H); |
| | 1H-1,2,3-triazole-4-carboxamide | LCMS (M − 1): 550.70 |
| 718 | 1-(2,4,6-trichlorophenyl)-N-((2,4,5- | ¹H-NMR (400 MHz, CHLOROFORM-D) δ 10.06 (s, |
| | trichlorophenyl)sulfonyl)-5- | 1H), 8.45 (s, 1H), 7.67 (s, 1H), 7.59-7.58 (m, 2H); LCMS |
| | (trifluoromethyl)-1H-1,2,3-triazole- | (M − 1): 600.65 |
| | 4-carboxamide | |
| 719 | N-((4-chlorophenyl)sulfonyl)-1- | ¹H-NMR (400 MHz, CHLOROFORM-D) δ 9.78 (s, 1H), |
| | (2,4,6-trichlorophenyl)-5- | 8.17 (dt, J = 9.1, 2.2 Hz, 2H), 7.61-7.58 (m, 4H); LCMS |
| | (trifluoromethyl)-1H-1,2,3-triazole- | (M − 1): 532.75 |
| | 4-carboxamide | |
| 720 | N-((3-chloro-4- | ¹H-NMR (400 MHz, CHLOROFORM-D) δ 9.78 (d, J = |
| | fluorophenyl)sulfonyl)-1-(2,4,6- | 26.4 Hz, 1H), 8.27 (dd, J = 6.6, 2.4 Hz, 1H), 8.15 (qd, J = |
| | trichlorophenyl)-5-(trifluoromethyl)- | 4.4, 2.4 Hz, 1H), 7.58 (t, J = 3.9 Hz, 2H), 7.36 (t, J = 8.6 |
| | 1H-1,2,3-triazole-4-carboxamide | Hz, 1H); LCMS (M − 1): 550.80 |
| 721 | N-((2-methoxy-5- | ¹H-NMR (400 MHz, CHLOROFORM-D) δ 9.86 (s, 1H), |
| | methylphenyl)sulfonyl)-1-(2,4,6- | 7.96 (d, J = 2.0 Hz, 1H), 7.58 (s, 2H), 7.41 (dd, J = 8.4, |
| | trichlorophenyl)-5-(trifluoromethyl)- | 1.6 Hz, 1H), 6.95 (d, J = 8.3 Hz, 1H), 3.97 (s, 3H), 2.38 |
| | 1H-1,2,3-triazole-4-carboxamide | (s, 3H); LCMS (M − 1): 542.80 |
| 722 | 1-(2,4,6-trichlorophenyl)-N-((4- | ¹H-NMR (400 MHz, CHLOROFORM-D) δ 9.78 (s, 1H), |
| | (trifluoromethoxy)phenyl)sulfonyl)- | 8.29-8.26 (m, 2H), 7.58 (s, 2H), 7.43-7.40 (m, 2H); |
| | 5-(trifluoromethyl)-1H-1,2,3- | LCMS (M − 1): 580.85 |
| | triazole-4-carboxamide | |
| 723 | N-((2,5- | ¹H-NMR (400 MHz, CHLOROFORM-D) δ 9.89 (s, 1H), |
| | bis(trifluoromethyl)phenyl)sulfonyl)- | 8.88 (s, 1H), 8.10-8.05 (m, 2H), 7.58 (t, J = 3.9 Hz, 2H); |
| | 1-(2,4,6-trichlorophenyl)-5- | LCMS (M − 1): 634.85 |
| | (trifluoromethyl)-1H-1,2,3-triazole- | |
| | 4-carboxamide | |
| 724 | N-((4-chloro-2,5- | ¹H-NMR (400 MHz, CHLOROFORM-D) δ 9.79 (s, 1H), |
| | dimethylphenyl)sulfonyl)-1-(2,4,6- | 8.15 (s, 1H), 7.58 (s, 2H), 7.34 (s, 1H), 2.69 (s, 3H), 2.44 |
| | trichlorophenyl)-5-(trifluoromethyl)- | (s, 3H); LCMS (M − 1): 560.80 |
| | 1H-1,2,3-triazole-4-carboxamide | |
| 725 | N-((2-fluorophenyl)sulfonyl)-1- | ¹H-NMR (400 MHz, CHLOROFORM-D) δ 9.99 (s, 1H), |
| | (2,4,6-trichlorophenyl)-5- | 8.23-8.19 (m, 1H), 7.72-7.66 (m, 1H), 7.58 (t, J = 3.8 Hz, |
| | (trifluoromethyl)-1H-1,2,3-triazole- | 2H), 7.39 (td, J = 7.7, 1.0 Hz, 1H), 7.29 (d, J = 1.0 Hz, |
| | 4-carboxamide | 1H); LCMS (M − 1): 516.80 |
| 726 | N-((5-chloro-2,4- | ¹H-NMR (400 MHz, CHLOROFORM-D) δ 10.04 (s, |
| | difluorophenyl)sulfonyl)-1-(2,4,6- | 1H), 8.29 (t, J = 7.5 Hz, 1H), 7.59 (t, J = 3.9 Hz, 2H), 7.11 |
| | trichlorophenyl)-5-(trifluoromethyl)- | (dd, J = 9.0, 8.3 Hz, 1H); LCMS (M − 1): 550.80 |
| | 1H-1,2,3-triazole-4-carboxamide | |
| 727 | N-((2,5-difluorophenyl)sulfonyl)-1- | ¹H-NMR (400 MHz, CHLOROFORM-D) δ 10.06 (s, |
| | (2,4,6-trichlorophenyl)-5- | 1H), 7.93-7.89 (m, 1H), 7.59 (t, J = 3.9 Hz, 2H), 7.41- |
| | (trifluoromethyl)-1H-1,2,3-triazole- | 7.35 (m, 1H), 7.23 (t, J = 4.5 Hz, 1H); LCMS (M − 1): |
| | 4-carboxamide | 534.75 |
| 728 | N-((2,4-difluorophenyl)sulfonyl)-1- | ¹H-NMR (400 MHz, CHLOROFORM-D) δ 10.02 (s, |
| | (2,4,6-trichlorophenyl)-5- | 1H), 8.24 (td, J = 8.5, 5.8 Hz, 1H), 7.59 (s, 2H), 7.13-7.08 |
| | (trifluoromethyl)-1H-1,2,3-triazole- | (m, 1H), 7.03-6.98 (m, 1H); LCMS (M − 1): 534.75 |
| | 4-carboxamide | |
| 729 | N-((4-fluorophenyl)sulfonyl)-1- | ¹H-NMR (400 MHz, CHLOROFORM-D) δ 9.77 (s, 1H), |
| | (2,4,6-trichlorophenyl)-5- | 8.26-8.21 (m, 2H), 7.58 (t, J = 3.8 Hz, 2H), 7.30-7.27 (m, |
| | (trifluoromethyl)-1H-1,2,3-triazole- | 1H), 7.25 (d, J = 3.2 Hz, 1H); LCMS (M − 1): 516.75 |
| | 4-carboxamide | |
| 730 | N-((2-chlorophenyl)sulfonyl)-1- | ¹H-NMR (400 MHz, CHLOROFORM-D) δ 10.02 (s, |
| | (2,4,6-trichlorophenyl)-5- | 1H), 8.39 (dd, J = 7.9, 1.6 Hz, 1H), 7.63-7.55 (m, 4H), |
| | (trifluoromethyl)-1H-1,2,3-triazole- | 7.55-7.51 (m, 1H); LCMS (M − 1): 532.75 |
| | 4-carboxamide | |
| 731 | 1-(2,4-difluorophenyl)-N-((2,5- | ¹H-NMR (400 MHz, DMSO-d₆) δ 7.86-7.62 (m, 4H), |
| | difluorophenyl)sulfonyl)-5-methyl- | 7.54 (td, J = 9.3, 4.2 Hz, 1H), 7.43-7.38 (m, 1H), 2.32 s, |
| | 1H-1,2,3-triazole-4-carboxamide | 3H); LCMS (M+): 414.90 |
| 732 | 1-(2,4,6-trichlorophenyl)-5- | ¹H-NMR (400 MHz, CHLOROFORM-D) δ 9.91 (s, 1H), |
| | (trifluoromethyl)-N-((2- | 8.66-8.64 (m, 1H), 7.93 (dd, J = 7.2, 1.9 Hz, 1H), 7.86- |
| | (trifluoromethyl)phenyl)sulfonyl)- | 7.80 (m, 2H), 7.58 (s, 2H); LCMS (M − 1): 566.90 |
| | 1H-1,2,3-triazole-4-carboxamide | |
| 733 | 1-(2,4,6-trichlorophenyl)-N-((2- | ¹H-NMR (400 MHz, CHLOROFORM-D) δ 9.99 (s, 1H), |
| | (trifluoromethoxy)phenyl)sulfonyl)- | 8.34 (dd, J = 7.8, 1.7 Hz, 1H), 7.76-7.71 (m, 1H), 7.58 (t, |
| | 5-(trifluoromethyl)-1H-1,2,3- | J = 3.8 Hz, 2H), 7.54-7.50 (m, 1H), 7.46 (dt, J = 8.3, 1.3 |
| | triazole-4-carboxamide | Hz, 1H); LCMS (M − 1): 580.70 |
| 734 | 1-(2,4-difluorophenyl)-N-((2,5- | ¹H-NMR (400 MHz, DMSO-d₆) δ 7.98 (td, J = 8.9, 5.8 |
| | dimethylphenyl)sulfonyl)-5- | Hz, 1H), 7.85 (d, J = 1.2 Hz, 1H), 7.80-7.75 (m, 1H), |
| | (trifluoromethyl)-1H-1,2,3-triazole- | 7.47-7.40 (m, 2H), 7.32 (d, J = 7.9 Hz, 1H), 2.6 (s, 3H), |
| | 4-carboxamide | 2.37 (s, 3H); LCMS (M+): 460.95 |
| 735 | N-((2-chloro-5- | ¹H-NMR (400 MHz, DMSO-d₆) δ 8.00 (td, J = 8.7, 5.8 |
| | methoxyphenyl)sulfonyl)-1-(2,4- | Hz, 1H), 7.80-7.75 (m, 1H), 7.62-7.57 (m, 2H), 7.47-7.42 |
| | difluorophenyl)-5-(trifluoromethyl)- | (m, 1H), 7.28 (dd, J = 8.7, 3.2 Hz, 1H), 3.85 (s, 3H); |
| | 1H-1,2,3-triazole-4-carboxamide | LCMS (M+): 496.95 |
| 736 | 1-(2,4-difluorophenyl)-5- | ¹H-NMR (400 MHz, DMSO-d₆) δ 8.36 (d, J = 7.6 Hz, |
| | (trifluoromethyl)-N-((2- | 1H), 8.01-7.94 (m, 2H), 7.90 (q, J = 7.5 Hz, 2H), 7.79- |
| | (trifluoromethyl)phenyl)sulfonyl)- | 7.74 (m, 1H), 7.44 (d, J = 9.2 Hz, 1H); LCMS (M+): |
| | 1H-1,2,3-triazole-4-carboxamide | 500.90 |
| 737 | 1-(2-chloro-4- | ¹H-NMR (400 MHz, CHLOROFORM-D) δ 9.70 (s, 1H), |
| | (trifluoromethoxy)phenyl)-N-((2,5- | 8.12 (d, J = 2.0 Hz, 1H), 7.52-7.46 (m, 3H), 7.44 (d, J = |
| | diisopropylphenyl)sulfonyl)-5- | 8.1 Hz, 1H), 7.36 (ddd, J = 8.7, 2.6, 0.9 Hz, 1H), 3.96- |
| | methyl-1H-1,2,3-triazole-4- | 3.89 (m, 1H), 3.05-2.98 (m, 1H), 2.41 (s, 3H), 1.29 (dd, |
| | carboxamide | J = 8.6, 6.8 Hz, 12H); LCMS(M + 1): 545.05 |
| 738 | 1-(2-chloro-4- | ¹H-NMR (400 MHz, CHLOROFORM-D) δ 9.70 (s, 1H), |
| | (difluoromethoxy)phenyl)-N-((2,5- | 8.12 (d, J = 1.7 Hz, 1H), 7.52-7.41 (m, 4H), 6.62 (t, J = |
| | diisopropylphenyl)sulfonyl)-5- | 71.9 Hz, 1H), 3.96-3.89 (m, 1H), 3.05-2.98 (m, 1H), 2.39 |
| | methyl-1H-1,2,3-triazole-4- | (s, 3H), 1.29 (dd, J = 8.7, 6.7 Hz, 12H); LCMS(M + 1): |
| | carboxamide | 527.05 |
| 739 | 1-(2,4-dichlorophenyl)-N-((2,5- | ¹H-NMR (400 MHz, CHLOROFORM-D) δ 9.74 (s, 1H), |
| | diisopropylphenyl)sulfonyl)-5- | 8.11 (d, J = 2.0 Hz, 1H), 7.65 (d, J = 2.0 Hz, 1H), 7.50- |
| | (trifluoromethyl)-1H-1,2,3-triazole- | 7.48 (m, 2H), 7.44 (d, J = 8.1 Hz, 1H), 7.39 (d, J = 8.3 |
| | 4-carboxamide | Hz, 1H), 3.94-3.88 (m, 1H), 3.05-2.98 (m, 1H), 1.30-1.28 |
| | | (m, 12H); LCMS(M − 2): 547.00 |
| 740 | 1-(2-chloro-4- | ¹H-NMR (400 MHz, CHLOROFORM-D) δ 9.74 (s, 1H), |
| | (trifluoromethyl)phenyl)-N-((2,5- | 8.12 (d, J = 2.0 Hz, 1H), 7.91 (d, J = 2.0 Hz, 1H), 7.79 |
| | diisopropylphenyl)sulfonyl)-5- | (dd, J = 8.2, 1.3 Hz, 1H), 7.62 (d, J = 8.3 Hz, 1H), 7.50 |
| | (trifluoromethyl)-1H-1,2,3-triazole- | (dd, J = 8.2, 1.8 Hz, 1H), 7.45 (d, J = 8.3 Hz, 1H), 3.95- |
| | 4-carboxamide | 3.88 (m, 1H), 3.05-2.98 (m, 1H), 1.31-1.26 (m, 12H); |
| | | LCMS(M − 1): 580.95 |
| 741 | 1-(2-chloro-4- | ¹H-NMR (400 MHz, CHLOROFORM-D) δ 9.70 (s, 1H), |
| | (trifluoromethyl)phenyl)-N-((2,5- | 8.12 (d, J = 2.0 Hz, 1H), 7.91 (d, J = 1.7 Hz, 1H), 7.78 |
| | diisopropylphenyl)sulfonyl)-5- | (dd, J = 8.2, 1.3 Hz, 1H), 7.58 (d, J = 8.1 Hz, 1H), 7.49 |
| | methyl-1H-1,2,3-triazole-4- | (dd, J = 8.2, 1.8 Hz, 1H), 7.44 (d, J = 8.1 Hz, 1H), 3.96- |
| | carboxamide | 3.89 (m, 1H), 3.05-2.98 (m, 1H), 2.42 (s, 3H), 1.29 (dd, |
| | | J = 7.8, 6.8 Hz, 12H); LCMS(M + 1): 529.10 |
| 742 | 1-(2-chloro-4-methylphenyl)-N- | ¹H-NMR (400 MHz, CHLOROFORM-D) δ 9.71 (s, 1H), |
| | ((2,5-diisopropylphenyl)sulfonyl)-5- | 8.12 (d, J = 2.0 Hz, 1H), 7.47 (dd, J = 8.2, 1.8 Hz, 1H), |
| | methyl-1H-1,2,3-triazole-4- | 7.43 (d, J = 8.1 Hz, 2H), 7.27 (d, J = 1.0 Hz, 2H), 3.96- |
| | carboxamide | 3.89 (m, 1H), 3.05-2.98 (m, 1H), 2.46 (s, 3H), 2.37 (s, |
| | | 3H), 1.29 (dd, J = 8.7, 7.0 Hz, 12H); LCMS(M + 1): |
| | | 475.05 |
| 743 | 1-(2-chloro-4-methoxyphenyl)-N- | ¹H-NMR (400 MHz, CHLOROFORM-D) δ 9.71 (s, 1H), |
| | ((2,5-diisopropylphenyl)sulfonyl)-5- | 8.12 (d, J = 2.0 Hz, 1H), 7.47 (dd, J = 8.1, 2.0 Hz, 1H), |
| | methyl-1H-1,2,3-triazole-4- | 7.43 (d, J = 8.1 Hz, 1H), 7.29 (d, J = 8.8 Hz, 1H), 7.11 (d, |
| | carboxamide | J = 2.7 Hz, 1H), 6.97 (dd, J = 8.9, 2.8 Hz, 1H), 3.93 (q, |
| | | J = 6.8 Hz, 1H), 3.89 (s, 3H), 3.04-2.98 (m, 1H), 2.37 (s, |
| | | 3H), 1.29 (dd, J = 8.9, 7.0 Hz, 12H); LCMS(M + 1): |
| | | 491.05 |
| 744 | N-((2,5-diisopropylphenyl)sulfonyl)- | ¹H-NMR (400 MHz, CHLOROFORM-D) δ 9.70 (s, 1H), |
| | 5-methyl-1-(2,4,6-trichlorophenyl)- | 8.12 (d, J = 2.0 Hz, 1H), 7.58 (d, J = 3.9 Hz, 2H), 7.48 |
| | 1H-1,2,3-triazole-4-carboxamide | (dd, J = 8.2, 1.8 Hz, 1H), 7.44 (d, J = 8.1 Hz, 1H), 3.95- |
| | | 3.89 (m, 1H), 3.05-2.98 (m, 1H), 2.35 (s, 3H), 1.29 (dd, |
| | | J = 10.0, 6.8 Hz, 12H); LCMS(M + 1): 530.85 |
| 745 | 1-(2,4-dichloro-6-methylphenyl)-N- | ¹H-NMR (400 MHz, CHLOROFORM-D) δ 9.71 (s, 1H), |
| | ((2,5-diisopropylphenyl)sulfonyl)-5- | 8.12 (d, J = 2.0 Hz, 1H), 7.49-7.46 (m, 2H), 7.44 (d, J = |
| | methyl-1H-1,2,3-triazole-4- | 8.1 Hz, 1H), 7.34 (q, J = 1.0 Hz, 1H), 3.96-3.89 (m, 1H), |
| | carboxamide | 3.05-2.98 (m, 1H), 2.32 (s, 3H), 1.98 (s, 3H), 1.33-1.26 |
| | | (m, 12H); LCMS(M + 1): 509.00 |
| 746 | 1-(2,4-dichloro-6-methylphenyl)-N- | ¹H-NMR (400 MHz, CHLOROFORM-D) δ 9.75 (s, 1H), |
| | ((2,5-diisopropylphenyl)sulfonyl)-5- | 8.12 (d, J = 2.0 Hz, 1H), 7.51-7.44 (m, 3H), 7.34 (q, J = |
| | (trifluoromethyl)-1H-1,2,3-triazole- | 1.0 Hz, 1H), 3.95-3.88 (m, 1H), 3.05-2.98 (m, 1H), 2.01 |
| | 4-carboxamide | (s, 3H), 1.30-1.24 (m, 12H); LCMS(M − 2): 561.00 |
| 747 | N-((2,5-difluorophenyl)sulfonyl)-1- | ¹H-NMR (400 MHz, DMSO-d₆) δ 8.44 (s, 1H), 8.32 (s, |
| | (2,4,5-trichlorophenyl)-5- | 1H), 7.67-7.63 (m, 1H), 7.52-7.47 (m, 1H), 7.41 (td, J = |
| | (trifluoromethyl)-1H-1,2,3-triazole- | 9.2, 4.2 Hz, 1H); LCMS(M + 1): 536.7 |
| | 4-carboxamide | |
| 748 | N-((5-chloro-2,4- | ¹H-NMR (400 MHz, DMSO-d₆) δ 8.44 (s, 1H), 8.31 (s, |
| | difluorophenyl)sulfonyl)-1-(2,4,5- | 1H), 7.99-7.95 (m, 1H), 7.64 (t, J = 9.5 Hz, 1H); |
| | trichlorophenyl)-5-(trifluoromethyl)- | LCMS(M + 1): 570.7 |
| | 1H-1,2,3-triazole-4-carboxamide | |
| 749 | 1-(2,4,5-trichlorophenyl)-N-((2,4,5- | ¹H-NMR (400 MHz, DMSO-d₆) δ 8.45 (s, 1H), 8.32 (s, |
| | trichlorophenyl)sulfonyl)-5- | 1H), 8.17 (s, 1H), 7.99 (s, 1H); LCMS(M − 2): 600.7 |
| | (trifluoromethyl)-1H-1,2,3-triazole- | |
| | 4-carboxamide | |
| 750 | N-((2-fluorophenyl)sulfonyl)-1- | ¹H-NMR (400 MHz, DMSO-d₆) δ 8.44 (s, 1H), 8.34 (s, |
| | (2,4,5-trichlorophenyl)-5- | 1H), 7.98-7.94 (m, 1H), 7.71 (dd, J = 14.4, 7.8 Hz, 1H), |
| | (trifluoromethyl)-1H-1,2,3-triazole- | 7.42-7.38 (m, 2H); LCMS(M + 1): 516.7 |
| | 4-carboxamide | |
| 751 | N-((5-fluoro-2- | ¹H-NMR (400 MHz, DMSO-d₆) δ 8.43 (s, 1H), 8.35 (s, |
| | methylphenyl)sulfonyl)-1-(2,4,5- | 1H), 7.77 (d, J = 8.6 Hz, 1H), 7.49-7.47 (m, 2H), 2.62 (d, |
| | trichlorophenyl)-5-(trifluoromethyl)- | J = 9.5 Hz, 3H); LCMS(M + 1): 532.8 |
| | 1H-1,2,3-triazole-4-carboxamide | |
| 752 | N-((2-chlorophenyl)sulfonyl)-1- | ¹H-NMR (400 MHz, DMSO-d₆) δ 8.42 (s, 1H), 8.31 (s, |
| | (2,4,5-trichlorophenyl)-5- | 1H), 8.13-8.11 (m, 1H), 7.65-7.60 (m, 2H), 7.57-7.53 (m, |
| | (trifluoromethyl)-1H-1,2,3-triazole- | 1H); LCMS(M + 1): 534.7 |
| | 4-carboxamide | |
| 753 | N-((2,4-difluorophenyl)sulfonyl)-1- | ¹H-NMR (400 MHz, DMSO-d₆) δ 8.44 (s, 1H), 8.34 (s, |
| | (2,4,5-trichlorophenyl)-5- | 1H), 8.03 (td, J = 8.6, 6.4 Hz, 1H), 7.53-7.48 (m, 1H), |
| | (trifluoromethyl)-1H-1,2,3-triazole- | 7.31 (td, J = 8.5, 2.1 Hz, 1H); LCMS(M + 1): 536.7 |
| | 4-carboxamide | |
| 754 | N-((3-chloro-2,5- | ¹H-NMR (400 MHz, DMSO-d₆) δ 8.43 (s, 1H), 8.35 (s, |
| | dimethylphenyl)sulfonyl)-1-(2,4,5- | 1H), 7.98 (s, 1H), 7.53 (s, 1H), 2.59 (s, 3H), 2.39 (s, 3H); |
| | trichlorophenyl)-5-(trifluoromethyl)- | LCMS(M − 2): 560.8 |
| | 1H-1,2,3-triazole-4-carboxamide | |
| 755 | N-((5-chloro-2- | ¹H-NMR (400 MHz, DMSO-d₆) δ 8.44 (s, 1H), 8.33 (s, |
| | fluorophenyl)sulfonyl)-1-(2,4,5- | 1H), 7.86 (q, J = 2.9 Hz, 1H), 7.74-7.70 (m, 1H), 7.43 (t, |
| | trichlorophenyl)-5-(trifluoromethyl)- | J = 9.2 Hz, 1H); LCMS(M − 2): 550.8 |
| | 1H-1,2,3-triazole-4-carboxamide | |
| 756 | 1-(2,4,5-trichlorophenyl)-N-((2- | ¹H-NMR (400 MHz, DMSO-d₆) δ 8.46 (d, J = 4.6 Hz, |
| | (trifluoromethoxy)phenyl)sulfonyl)- | 1H), 8.34 (s, 1H), 8.12 (dd, J = 7.9, 1.6 Hz, 1H), 7.81- |
| | 5-(trifluoromethyl)-1H-1,2,3- | 7.77 (m, 1H), 7.61-7.54 (m, 2H); LCMS(M − 2): 580.8 |
| | triazole-4-carboxamide | |
| 757 | N-((4-fluorophenyl)sulfonyl)-1- | ¹H-NMR (400 MHz, DMSO-d₆) δ 8.42 (s, 1H), 8.35 (s, |
| | (2,4,5-trichlorophenyl)-5- | 1H), 8.08-8.04 (m, 2H), 7.48 (t, J = 8.8 Hz, 2H): |
| | (trifluoromethyl)-1H-1,2,3-triazole- | LCMS(M − 1): 516.7 |
| | 4-carboxamide | |
| 758 | 1-(2,4-dichlorophenyl)-N-((2,5- | ¹H-NMR (400 MHz, DMSO-d₆) δ 8.15 (d, J = 8.9 Hz, |
| | dimethylphenyl)(methyl)(oxo)-λ⁶- | 1H), 8.04 (d, J = 2.1 Hz, 1H), 7.86 (s, 1H), 7.75-7.72 (m, |
| | sulfaneylidene)-5-(trifluoromethyl)- | 1H), 7.49 (d, J = 7.9 Hz, 1H), 7.40 (d, J = 7.9 Hz, 1H), |
| | 1H-1,2,3-triazole-4-carboxamide | 3.63 (s, 3H), 2.61 (s, 3H), 2.39 (s, 3H); LCMS(M + 1): |
| | | 541.0 |
| 759 | 1-(2-chloro-4- | ¹H-NMR (400 MHz, DMSO-d₆) δ 8.05 (d, J = 2.1 Hz, |
| | (trifluoromethoxy)phenyl)-N-((2,5- | 1H), 7.85 (s, 1H), 7.78 (d, J = 8.3 Hz, 1H), 7.73 (dd, J = |
| | dimethylphenyl)(methyl)(oxo)-λ⁶- | 8.6, 2.1 Hz, 1H), 7.46 (d, J = 6.7 Hz, 1H), 7.37 (d, J = 7.9 |
| | sulfaneylidene)-5-(trifluoromethyl)- | Hz, 1H), 3.58 (s, 3H), 2.60 (d, J = 10.4 Hz, 3H), 2.39 (s, |
| | 1H-1,2,3-triazole-4-carboxamide | 3H), 2.33 (t, J = 3.1 Hz, 3H): LCMS(M = 1): 438.9 |
| 760 | 1-(2,4-dichlorophenyl)-N-((2,5- | ¹H-NMR (400 MHz, DMSO-d₆) δ 8.00 (d, J = 2.1 Hz, |
| | dimethylphenyl)(methyl)(oxo)-λ⁶- | 1H), 7.91 (d, J = 8.9 Hz, 1H), 7.85 (s, 1H), 7.69 (dq, J = |
| | sulfaneylidene)-5-methyl-1H-1,2,3- | 8.9, 1.2 Hz, 1H), 7.46 (d, J = 7.9 Hz, 1H), 7.37 (d, J = 7.9 |
| | triazole-4-carboxamide | Hz, 1H), 3.58 (s, 3H), 2.59 (s, 3H), 2.39 (s, 3H), 2.35 (s, |
| | | 3H); LCMS(M + 1): 487.2 |
| 761 | 1-(2-chloro-4- | ¹H-NMR (400 MHz, DMSO-d₆) δ 7.83 (t, J = 6.0 Hz, 2H), |
| | (trifluoromethoxy)phenyl)-N-((2,5- | 7.71 (s, 1H), 7.46 (d, J = 7.6 Hz, 1H), 7.40-7.35 (m, 2H), |
| | dimethylphenyl)(methyl)(oxo)-λ⁶- | 3.57 (s, 3H), 2.58 (s, 3H), 2.40 (d, J = 7.9 Hz, 3H), 2.37- |
| | sulfaneylidene)-5-methyl-1H-1,2,3- | 2.39 (3H); LCMS(M + 1): 405.3 |
| | triazole-4-carboxamide | |
| 762 | 1-(2,4-difluorophenyl)-N-((2,5- | ¹H-NMR (400 MHz, DMSO-d₆) δ 8.48 (d, J = 1.5 Hz, |
| | dimethylphenyl)(methyl)(oxo)-λ⁶- | 1H), 8.15 (dd, J = 8.1, 1.7 Hz, 1H), 7.98 (d, J = 8.3 Hz, |
| | sulfaneylidene)-5-methyl-1H-1,2,3- | 1H), 7.85 (s, 1H), 7.46 (d, J = 7.9 Hz, 1H), 7.37 (d, J = |
| | triazole-4-carboxamide | 7.9 Hz, 1H), 3.58 (s, 3H), 2.59 (s, 3H), 2.39 (s, 3H), 2.35 |
| | | (s, 3H); LCMS(M + 1): 428.1 |
| 763 | 1-(2-chloro-4-cyanophenyl)-N-((2,5- | ¹H-NMR (400 MHz, DMSO-d₆) δ 8.36 (dd, J = 9.9, 1.7 |
| | dimethylphenyl)(methyl)(oxo)-λ⁶- | Hz, 1H), 8.16 (t, J = 7.8 Hz, 1H), 8.07-8.05 (m, 1H), 7.85 |
| | sulfaneylidene)-5-methyl-1H-1,2,3- | (s, 1H), 7.49 (d, J = 7.6 Hz, 1H), 7.40 (d, J = 7.6 Hz, 1H), |
| | triazole-4-carboxamide | 3.63 (s, 3H), 2.61 (s, 3H), 2.39 (s, 3H): LCMS(M + 1): |
| | | 466.4 |
| 764 | 1-(4-cyano-2-fluorophenyl)-N-((2,5- | ¹H-NMR (400 MHz, DMSO-d₆) δ 8.18-8.13 (m, 2H), |
| | dimethylphenyl)(methyl)(oxo)-λ⁶- | 8.04 (d, J = 2.4 Hz, 1H), 7.92 (dd, J = 8.6, 2.4 Hz, 1H), |
| | sulfaneylidene)-5-(trifluoromethyl)- | 7.83 (d, J = 8.6 Hz, 1H), 7.73 (dt, J = 8.9, 1.2 Hz, 1H), |
| | 1H-1,2,3-triazole-4-carboxamide | 3.77 (t, J = 14.7 Hz, 3H); LCMS(M + 1): 582.8 |
| 765 | 1-(2-chloro-4- | ¹H-NMR (400 MHz, DMSO-d₆) δ 8.11 (d, J = 2.4 Hz, |
| | (trifluoromethoxy)phenyl)-N-((2,5- | 1H), 8.05 (d, J = 2.1 Hz, 1H), 7.89 (dd, J = 8.6, 2.8 Hz, |
| | dichlorophenyl)(methyl)(oxo)-λ⁶- | 1H), 7.80 (d, J = 5.5 Hz, 1H), 7.78 (d, J = 5.5 Hz, 1H), |
| | sulfaneylidene)-5-(trifluoromethyl)- | 7.73 (dd, J = 8.4, 2.3 Hz, 1H), 3.71 (s, 3H), 2.34 (s, 3H); |
| | 1H-1,2,3-triazole-4-carboxamide | LCMS(M + 1): 478.8 |
| 766 | 1-(2,4-dichlorophenyl)-N-((2,5- | ¹H-NMR (400 MHz, DMSO-d₆) δ 8.12 (d, J = 2.4 Hz, |
| | dichlorophenyl)(methyl)(oxo)-λ⁶- | 1H), 8.00 (d, J = 2.4 Hz, 1H), 7.92 (d, J = 8.9 Hz, 1H), |
| | sulfaneylidene)-5-methyl-1H-1,2,3- | 7.89 (dd, J = 8.6, 2.8 Hz, 1H), 7.80 (d, J = 8.6 Hz, 1H), |
| | triazole-4-carboxamide | 7.70 (s, 1H), 3.72 (s, 3H), 2.35 (s, 3H); LCMS(M + 1): |
| | | 528.9 |
| 767 | 1-(2-chloro-4- | ¹H-NMR (400 MHz, DMSO-d₆) δ 8.11 (d, J = 2.8 Hz, |
| | (trifluoromethoxy)phenyl)-N-((2,5- | 1H), 7.89 (dd, J = 8.6, 2.4 Hz, 1H), 7.86-7.79 (m, 2H), |
| | dichlorophenyl)(methyl)(oxo)-λ⁶- | 7.74-7.68 (m, 1H), 7.42-7.37 (m, 1H), 3.71 (s, 3H), 2.40 |
| | sulfaneylidene)-5-methyl-1H-1,2,3- | (d, J = 7.9 Hz, 3H); LCMS(M+): 445.0 |
| | triazole-4-carboxamide | |
| 768 | N-((2,5- | ¹H-NMR (400 MHz, DMSO-d₆) δ 8.48 (d, J = 1.5 Hz, |
| | dichlorophenyl)(methyl)(oxo)-λ⁶- | 1H), 8.16-8.11 (m, 2H), 7.98 (d, J = 8.3 Hz, 1H), 7.89 |
| | sulfaneylidene)-1-(2,4- | (dd, J = 8.6, 2.4 Hz, 1H), 7.80 (d, J = 8.6 Hz, 1H), 3.72 |
| | difluorophenyl)-5-methyl-1H-1,2,3- | (s, 3H), 2.35 (s, 3H); LCMS(M + 1): 469.9 |
| | triazole-4-carboxamide | |
| 769 | 1-(2-chloro-4-cyanophenyl)-N-((2,5- | ¹H-NMR (400 MHz, DMSO-d₆) δ 8.36 (dd, J = 9.9, 1.7 |
| | dichlorophenyl)(methyl)(oxo)-λ⁶- | Hz, 1H), 8.18-8.13 (m, 2H), 8.07-8.05 (m, 1H), 7.92 (dd, |
| | sulfaneylidene)-5-methyl-1H-1,2,3- | J = 8.6, 2.8 Hz, 1H), 7.83 (d, J = 8.6 Hz, 1H), 3.77 (s, |
| | triazole-4-carboxamide | 3H); LCMS(M + 1): 507.8 |
| 770 | 1-(4-cyano-2-fluorophenyl)-N-((2,5- | ¹H-NMR (400 MHz, DMSO-d₆) δ 8.36 (dd, J = 9.9, 1.7 |
| | dichlorophenyl)(methyl)(oxo)-λ⁶- | Hz, 1H), 8.18-8.13 (m, 2H), 8.07-8.05 (m, 1H), 7.92 (dd, |
| | sulfaneylidene)-5-(trifluoromethyl)- | J = 8.6, 2.8 Hz, 1H), 7.83 (d, J = 8.6 Hz, 1H), 3.77 (s, |
| | 1H-1,2,3-triazole-4-carboxamide | 3H); LCMS(M + 1): 506.25 |
| 771 | 1-(2,4-dichlorophenyl)-N-((2,5- | ¹H-NMR (400 MHz, DMSO-d₆) δ 8.13 (d, J = 2.4 Hz, |
| | dichlorophenyl)(methyl)(oxo)-λ⁶- | 1H), 8.08 (d, J = 2.1 Hz, 1H), 8.01 (d, J = 5.8 Hz, 1H), |
| | sulfaneylidene)-5-(trifluoromethyl)- | 7.92 (dd, J = 8.6, 2.4 Hz, 1H), 7.83 (d, J = 8.6 Hz, 1H), |
| | 1H-1,2,3-triazole-4-carboxamide | 7.77 (dd, J = 8.7, 2.3 Hz, 1H), 3.77 (s, 3H); LCMS(M + 1): |
| | | 532.9 |
| 772 | 1-(2,4-dichlorophenyl)-N- | ¹H-NMR (400 MHz, DMSO-d₆) δ 8.08 (d, J = 2.2 Hz, |
| | (methyl(oxo)(o-tolyl)-λ⁶- | 1H), 8.05 (d, J = 7.8 Hz, 1H), 8.01 (d, J = 8.6 Hz, 1H), |
| | sulfaneylidene)-5-(trifluoromethyl)- | 7.77 (dd, J = 8.6, 2.2 Hz, 1H), 7.69-7.66 (m, 1H), 7.54 |
| | 1H-1,2,3-triazole-4-carboxamide | (dd, J = 18.2, 7.7 Hz, 2H), 3.66 (s, 3H), 2.67 (d, J = 7.1 |
| | | Hz, 3H); LCMS(M − 1): 476.9 |
| 773 | 1-(2-chloro-4-cyanophenyl)-5- | ¹H-NMR (400 MHz, DMSO-d₆) δ 8.48 (d, J = 1.7 Hz, |
| | methyl-N-(methyl(oxo)(o-tolyl)-λ⁶- | 1H), 8.15 (dd, J = 8.1, 1.7 Hz, 1H), 8.04 (d, J = 7.8 Hz, |
| | sulfaneylidene)-1H-1,2,3-triazole-4- | 1H), 7.97 (d, J = 8.1 Hz, 1H), 7.65 (t, J = 7.5 Hz, 1H), |
| | carboxamide | 7.56-7.48 (m, 2H), 3.61 (s, 3H), 2.65 (s, 3H), 2.36 (s, 3H); |
| | | LCMS(M + 1): 413.9 |
| 774 | N-((2-chloro-5- | ¹H-NMR (400 MHz, DMSO-d₆) δ 8.30 (d, J = 1.8 Hz, |
| | (trifluoromethyl)phenyl)sulfonyl)-1- | 1H), 8.01-7.95 (m, 2H), 7.83 (d, J = 8.3 Hz, 1H), 7.78- |
| | (2,4-difluorophenyl)-5- | 7.72 (m, 1H), 7.44-7.40 (m, 1H); LCMS (M − 1): 532.90 |
| | (trifluoromethyl)-1H-1,2,3-triazole- | |
| | 4-carboxamide | |
| 775 | N-((2,5-dichlorophenyl)sulfonyl)-1- | ¹H-NMR (400 MHz, DMSO-d₆) δ 8.06 (d, J = 2.4 Hz, |
| | (2,4-difluorophenyl)-5- | 1H), 7.98 (td, J = 8.8, 5.9 Hz, 1H), 7.78-7.75 (m, 1H), |
| | (trifluoromethyl)-1H-1,2,3-triazole- | 7.73-7.70 (m, 1H), 7.65 (d, J = 8.6 Hz, 1H), 7.45-7.41 (m, |
| | 4-carboxamide | 1H); LCMS (M+): 500.85 |
| 776 | 1-(2,4-difluorophenyl)-N-(o- | ¹H-NMR (400 MHz, DMSO-d₆) δ 8.04 (d, J = 8.1 Hz, |
| | tolylsulfonyl)-5-(trifluoromethyl)- | 1H), 7.98 (td, J = 8.7, 5.7 Hz, 1H), 7.80-7.74 (m, 1H), |
| | 1H-1,2,3-triazole-4-carboxamide | 7.61-7.57 (m, 1H), 7.47-7.41 (m, 3H), 2.65 (s, 3H); |
| | | LCMS (M − 1): 444.95 |
| 777 | 1-(2,4-difluorophenyl)-N-((2- | ¹H-NMR (400 MHz, DMSO-d₆) δ 8.14 (dd, J = 7.9, 1.8 |
| | (trifluoromethoxy)phenyl)sulfonyl)- | Hz, 1H), 7.99 (td, J = 8.9, 5.8 Hz, 1H), 7.83 (td, J = 7.9, |
| | 5-(trifluoromethyl)-1H-1,2,3- | 1.3 Hz, 1H), 7.79-7.74 (m, 1H), 7.64-7.58 (m, 2H), 7.46- |
| | triazole-4-carboxamide | 7.41 (m, 1H); LCMS (M − 1): 514.95 |
| 778 | N-((2-chlorophenyl)sulfonyl)-1-(2,4- | ¹H-NMR (400 MHz, DMSO-d₆) δ 8.18 (dd, J = 7.9, 1.2 |
| | difluorophenyl)-5-(trifluoromethyl)- | Hz, 1H), 8.00 (td, J = 8.8, 5.9 Hz, 1H), 7.80-7.68 (m, 3H), |
| | 1H-1,2,3-triazole-4-carboxamide | 7.64-7.60 (m, 1H), 7.47-7.42 (m, 1H); LCMS (M + 1): |
| | | 466.85 |
| 779 | 1-(2,4-difluorophenyl)-N-((2- | ¹H-NMR (400 MHz, DMSO-d₆) δ 8.01-7.95 (m, 2H), |
| | fluorophenyl)sulfonyl)-5- | 7.80-7.74 (m, 2H), 7.48-7.42 (m, 3H); LCMS (M − 1): |
| | (trifluoromethyl)-1H-1,2,3-triazole- | 449.10 |
| | 4-carboxamide | |
| 780 | 1-(2-chloro-4- | ¹H-NMR (400 MHz, DMSO-d₆) δ 8.15 (d, J = 8.8 Hz, |
| | (trifluoromethoxy)phenyl)-N- | 1H), 8.06-8.04 (m, 2H), 7.73 (dd, J = 8.9, 1.3 Hz, 1H), |
| | (methyl(oxo)(o-tolyl)-26- | 7.68 (td, J = 7.5, 1.2 Hz, 1H), 7.54 (dd, J = 18.3, 7.8 Hz, |
| | sulfaneylidene)-5-(trifluoromethyl)- | 2H), 3.66 (s, 3H), 2.68 (s, 3H); LCMS(M + 1): 527.2 |
| | 1H-1,2,3-triazole-4-carboxamide | |
| 781 | 1-(2,4-dichlorophenyl)-5-methyl-N- | ¹H-NMR (400 MHz, DMSO-d₆) δ 8.05-8.03 (m, 2H), |
| | (methyl(oxo)(o-tolyl)-λ⁶- | 7.79-7.76 (m, 1H), 7.73 (dd, J = 8.4, 2.1 Hz, 1H), 7.65 |
| | sulfaneylidene)-1H-1,2,3-triazole-4- | (td, J = 7.5, 1.3 Hz, 1H), 7.55-7.48 (m, 2H), 3.60 (s, 3H), |
| | carboxamide | 2.65 (s, 3H), 2.36-2.32 (m, 3H); LCMS(M+): 423.0 |
| 782 | 1-(4-cyano-2-fluorophenyl)-N- | ¹H-NMR (400 MHz, DMSO-d₆) δ 8.36 (dd, J = 9.8, 1.5 |
| | (methyl(oxo)(o-tolyl)-λ⁶- | Hz, 1H), 8.17-8.13 (m, 1H), 8.07-8.03 (m, 2H), 7.68 (td, |
| | sulfaneylidene)-5-(trifluoromethyl)- | J = 7.5, 1.3 Hz, 1H), 7.54 (dd, J = 18.2, 7.7 Hz, 2H), 3.66 |
| | 1H-1,2,3-triazole-4-carboxamide | (s, 3H), 2.67 (s, 3H); LCMS(M + 1): 452.1 |
| 783 | 1-(2-chloro-4- | ¹H-NMR (400 MHz, DMSO-d₆) δ 8.05-8.00 (m, 2H), |
| | (trifluoromethoxy)phenyl)-5-methyl- | 7.91 (d, J = 8.6 Hz, 1H), 7.70-7.63 (m, 2H), 7.54 (d, J = |
| | N-(methyl(oxo)(o-tolyl)-λ⁶- | 7.8 Hz, 1H), 7.50 (t, J = 7.3 Hz, 1H), 3.61 (s, 3H), 2.65 |
| | sulfaneylidene)-1H-1,2,3-triazole-4- | (s, 3H), 2.35 (s, 3H); LCMS(M + 1): 473.1 |
| | carboxamide | |
| 784 | 1-(2,4-difluorophenyl)-5-methyl-N- | ¹H-NMR (400 MHz, DMSO-d₆) δ 8.03 (dd, J = 7.9, 1.2 |
| | (methyl(oxo)(o-tolyl)-λ⁶- | Hz, 1H), 7.82 (td, J = 8.8, 5.9 Hz, 1H), 7.73-7.63 (m, 2H), |
| | sulfaneylidene)-1H-1,2,3-triazole-4- | 7.55-7.48 (m, 1H), 7.42-7.37 (m, 1H), 3.60 (s, 3H), 2.66 |
| | carboxamide | (d, J = 5.5 Hz, 3H), 2.39 (s, 3H); LCMS(M + 1): 390.9 |
| 785 | 1-(2,4-dichlorophenyl)-N- | ¹H-NMR (400 MHz, DMSO-d₆) δ 8.33 (d, J = 7.6 Hz, |
| | (methyl(oxo)(2- | 1H), 8.14-7.99 (m, 4H), 7.78 (dd, J = 8.6, 2.1 Hz, 1H), |
| | (trifluoromethyl)phenyl)-λ⁶- | 3.69 (s, 3H); LCMS(M − 1): 530.9 |
| | sulfaneylidene)-5-(trifluoromethyl)- | |
| | 1H-1,2,3-triazole-4-carboxamide | |
| 786 | 1-(2-chloro-4-cyanophenyl)-5- | ¹H-NMR (400 MHz, DMSO-d₆) δ 8.49 (d, J = 1.8 Hz, |
| | methyl-N-(methyl(oxo)(2- | 1H), 8.32 (d, J = 7.6 Hz, 1H), 8.16 (dd, J = 8.3, 1.8 Hz, |
| | (trifluoromethyl)phenyl)-λ⁶- | 1H), 8.11 (dd, J = 7.6, 1.5 Hz, 1H), 8.05-7.96 (m, 2H), |
| | sulfaneylidene)-1H-1,2,3-triazole-4- | 3.62 (d, J = 14.1 Hz, 3H), 2.36 (s, 3H); |
| | carboxamide | LCMS(M + 1): 468.0 |
| 787 | 1-(2-chloro-4- | ¹H-NMR (400 MHz, DMSO-d₆) δ 8.35-8.33 (m, 1H), |
| | (trifluoromethoxy)phenyl)-N- | 8.18-8.12 (m, 2H), 8.10-7.99 (m, 2H), 7.74 (dq, J = 8.9, |
| | (methyl(oxo)(2- | 1.2 Hz, 1H), 3.68 (d, J = 14.4 Hz, 3H); LCMS(M + 1): |
| | (trifluoromethyl)phenyl)-λ⁶- | 580.9 |
| | sulfaneylidene)-5-(trifluoromethyl)- | |
| | 1H-1,2,3-triazole-4-carboxamide | |
| 788 | 1-(2,4-dichlorophenyl)-5-methyl-N- | ¹H-NMR (400 MHz, DMSO-d₆) δ 8.33-8.31 (m, 1H), |
| | (methyl(oxo)(2- | 8.11 (dd, J = 7.6, 1.5 Hz, 1H), 8.07-7.96 (m, 3H), 7.80- |
| | (trifluoromethyl)phenyl)-λ⁶- | 7.76 (m, 1H), 7.73 (dd, J = 8.4, 2.3 Hz, 1H), 3.62 (d, J = |
| | sulfaneylidene)-1H-1,2,3-triazole-4- | 14.1 Hz, 3H), 2.36-2.33 (m, 3H); LCMS(M − 1): 476.9 |
| | carboxamide | |
| 789 | 1-(4-cyano-2-fluorophenyl)-N- | ¹H-NMR (400 MHz, DMSO-d₆) δ 8.38-8.33 (m, 2H), |
| | (methyl(oxo)(2- | 8.19-8.12 (m, 2H), 8.08-8.01 (m, 3H), 3.70 (s, 3H); |
| | (trifluoromethyl)phenyl)-λ⁶- | LCMS(M + 1): 506.1 |
| | sulfaneylidene)-5-(trifluoromethyl)- | |
| | 1H-1,2,3-triazole-4-carboxamide | |
| 790 | 1-(2-chloro-4- | ¹H-NMR (400 MHz, DMSO-d₆) δ 8.32 (d, J = 7.6 Hz, |
| | (trifluoromethoxy)phenyl)-5-methyl- | 1H), 8.11 (dd, J = 7.5, 1.4 Hz, 1H), 8.05-7.96 (m, 3H), |
| | N-(methyl(oxo)(2- | 7.95-7.91 (m, 1H), 7.69 (dq, J = 8.8, 1.2 Hz, 1H), 3.64 (t, |
| | (trifluoromethyl)phenyl)-λ⁶- | J = 15.1 Hz, 3H), 2.40-2.33 (m, 3H); LCMS(M + 1): 526.9 |
| | sulfaneylidene)-1H-1,2,3-triazole-4- | |
| | carboxamide | |
| 791 | 1-(2,4-difluorophenyl)-5-methyl-N- | ¹H-NMR (400 MHz, DMSO-d₆) δ 8.31 (d, J = 7.3 Hz, |
| | (methyl(oxo)(2- | 1H), 8.12-8.10 (m, 1H), 8.04-7.96 (m, 2H), 7.83 (td, J = |
| | (trifluoromethyl)phenyl)-λ⁶- | 8.8, 5.9 Hz, 1H), 7.75-7.69 (m, 1H), 7.43-7.38 (m, 1H), |
| | sulfaneylidene)-1H-1,2,3-triazole-4- | 3.63 (t, J = 14.5 Hz, 3H), 2.38 (d, J = 14.4 Hz, 3H); |
| | carboxamide | LCMS(M + 1): 445.0 |
| 792 | N-((2-chlorophenyl)sulfonyl)-1-(2,6- | ¹H-NMR (400 MHz, CHLOROFORM-D) δ 9.87 (s, 1H), |
| | dichloro-4-(trifluoromethyl)phenyl)- | 8.39 (dd, J = 7.8, 1.2 Hz, 1H), 7.83 (d, J = 0.5 Hz, 2H), |
| | 5-methyl-1H-1,2,3-triazole-4- | 7.62-7.50 (m, 3H), 2.38 (s, 3H); LCMS(M+): 512.80 |
| | carboxamide | |
| 793 | 1-(2,6-dichloro-4- | ¹H-NMR (400 MHz, CHLOROFORM-D) δ 9.87 (s, 1H), |
| | (trifluoromethyl)phenyl)-N-((2,5- | 8.37 (d, J = 2.4 Hz, 1H), 7.84 (d, J = 0.5 Hz, 2H), 7.56 |
| | dichlorophenyl)sulfonyl)-5-methyl- | (dd, J = 8.6, 2.4 Hz, 1H), 7.49 (d, J = 8.6 Hz, 1H), 2.39 |
| | 1H-1,2,3-triazole-4-carboxamide | (s, 3H); LCMS(M + 1): 546.80 |
| 794 | 1-(2,6-dichloro-4- | ¹H-NMR (400 MHz, CHLOROFORM-D) δ 9.86 (s, 1H), |
| | (trifluoromethyl)phenyl)-N-((2,4- | 8.32 (d, J = 8.6 Hz, 1H), 7.83 (d, J = 0.7 Hz, 2H), 7.57 (d, |
| | dichlorophenyl)sulfonyl)-5-methyl- | J = 2.0 Hz, 1H), 7.52-7.48 (m, 1H), 2.38 (s, 3H); |
| | 1H-1,2,3-triazole-4-carboxamide | LCMS(M − 1): 546.75 |
| 795 | 1-(2,6-dichloro-4- | ¹H-NMR (400 MHz, CHLOROFORM-D) δ 9.81 (s, 1H), |
| | (trifluoromethyl)phenyl)-N-((2- | 8.23-8.19 (m, 1H), 7.83 (d, J = 0.5 Hz, 2H), 7.71-7.65 (m, |
| | fluorophenyl)sulfonyl)-5-methyl-1H- | 1H), 7.41-7.37 (m, 1H), 7.24 (s, 1H), 2.38 (s, 3H); |
| | 1,2,3-triazole-4-carboxamide | LCMS(M − 2): 494.80 |
| 796 | 1-(2,6-dichloro-4- | ¹H-NMR (400 MHz, CHLOROFORM-D) δ 9.81 (s, 1H), |
| | (trifluoromethyl)phenyl)-N-((2,4- | 8.23 (td, J = 8.4, 6.3 Hz, 1H), 7.83 (d, J = 0.5 Hz, 2H), |
| | difluorophenyl)sulfonyl)-5-methyl- | 7.12-7.08 (m, 1H), 7.02-6.97 (m, 1H), 2.39 (s, 3H); |
| | 1H-1,2,3-triazole-4-carboxamide | LCMS(M − 2): 512.85 |
| 797 | 1-(2,6-dichloro-4- | ¹H-NMR (400 MHz, CHLOROFORM-D) δ 9.75 (s, 1H), |
| | (trifluoromethyl)phenyl)-5-methyl- | 8.65-8.63 (m, 1H), 7.92 (dd, J = 7.2, 1.8 Hz, 1H), 7.86- |
| | N-((2- | 7.78 (m, 4H), 2.38 (s, 3H); LCMS(M+): 546.80 |
| | (trifluoromethyl)phenyl)sulfonyl)- | |
| | 1H-1,2,3-triazole-4-carboxamide | |
| 798 | N-((2-chloro-5- | ¹H-NMR (400 MHz, CHLOROFORM-D) δ 9.90 (s, 1H), |
| | (trifluoromethyl)phenyl)sulfonyl)-1- | 8.64 (d, J = 2.0 Hz, 1H), 7.85-7.83 (m, 3H), 7.70 (d, J = |
| | (2,6-dichloro-4- | 8.3 Hz, 1H), 2.39 (s, 3H); LCMS(M + 1): 580.80 |
| | (trifluoromethyl)phenyl)-5-methyl- | |
| | 1H-1,2,3-triazole-4-carboxamide | |
| 799 | N-((2-chloro-5- | ¹H-NMR (400 MHz, CHLOROFORM-D) δ 9.87 (s, 1H), |
| | methoxyphenyl)sulfonyl)-1-(2,6- | 7.87 (d, J = 3.2 Hz, 1H), 7.83 (d, J = 0.5 Hz, 2H), 7.43 (d, |
| | dichloro-4-(trifluoromethyl)phenyl)- | J = 8.8 Hz, 1H), 7.10 (dd, J = 8.8, 2.9 Hz, 1H), 3.91 (s, |
| | 5-methyl-1H-1,2,3-triazole-4- | 3H), 2.39 (s, 3H); LCMS(M+): 542.80 |
| | carboxamide | |
| 800 | 1-(2,6-dichloro-4- | ¹H-NMR (400 MHz, CHLOROFORM-D) δ 9.65 (s, 1H), |
| | (trifluoromethyl)phenyl)-N-((2,5- | 8.10 (s, 1H), 7.83 (d, J = 0.5 Hz, 2H), 7.36-7.33 (m, 1H), |
| | dimethylphenyl)sulfonyl)-5-methyl- | 7.23 (d, J = 7.8 Hz, 1H), 2.71 (s, 3H), 2.44 (s, 3H), 2.38 |
| | 1H-1,2,3-triazole-4-carboxamide | (s, 3H); LCMS(M + 1): 506.95 |
| 801 | 1-(2-chloro-4-cyanophenyl)-N-((2- | ¹H-NMR (400 MHz, DMSO-d₆) δ 8.48 (d, J = 1.8 Hz, |
| | chloro-5- | 1H), 8.15 (dd, J = 8.3, 1.8 Hz, 1H), 8.00-7.97 (m, 2H), |
| | methylphenyl)(methyl)(oxo)-λ⁶- | 7.63 (d, J = 7.9 Hz, 1H), 7.60-7.57 (m, 1H), 3.66 (s, 3H), |
| | sulfaneylidene)-5-methyl-1H-1,2,3- | 2.45 (s, 3H), 2.35 (s, 3H); LCMS(M − 1): 447.9 |
| | triazole-4-carboxamide | |
| 802 | 1-(2-chloro-4- | ¹H-NMR (400 MHz, DMSO-d₆) δ 8.18-8.14 (m, 1H), |
| | (trifluoromethoxy)phenyl)-N-((2- | 8.04 (d, J = 2.1 Hz, 1H), 8.01 (d, J = 1.5 Hz, 1H), 7.73 |
| | chloro-5- | (dq, J = 8.9, 1.1 Hz, 1H), 7.66 (d, J = 8.3 Hz, 1H), 7.61 |
| | methylphenyl)(methyl)(oxo)-λ⁶- | (dd, J = 8.3, 1.5 Hz, 1H), 3.70 (s, 3H), 2.45 (s, 3H); |
| | sulfaneylidene)-5-(trifluoromethyl)- | LCMS(M − 1): 560.9 |
| | 1H-1,2,3-triazole-4-carboxamide | |
| 803 | N-((2-chloro-5- | ¹H-NMR (400 MHz, DMSO-d₆) δ 8.04 (d, J = 2.1 Hz, |
| | methylphenyl)(methyl)(oxo)-λ⁶- | 1H), 8.00 (d, J = 1.5 Hz, 1H), 7.78 (d, J = 8.3 Hz, 1H), |
| | sulfaneylidene)-1-(2,4- | 7.73 (dd, J = 8.6, 2.1 Hz, 1H), 7.63 (d, J = 8.3 Hz, 1H), |
| | dichlorophenyl)-5-methyl-1H-1,2,3- | 7.59 (dd, J = 8.3, 1.5 Hz, 1H), 3.65 (s, 3H), 2.45 (s, 3H), |
| | triazole-4-carboxamide | 2.33 (d, J = 5.5 Hz, 3H); LCMS(M − 1): 456.9 |
| 804 | 1-(2-chloro-4- | ¹H-NMR (400 MHz, DMSO-d₆) δ 8.00 (s, 2H), 7.92 (d, |
| | (trifluoromethoxy)phenyl)-N-((2- | J = 8.9 Hz, 1H), 7.68 (dd, J = 8.6, 1.5 Hz, 1H), 7.63 (d, J = |
| | chloro-5- | 7.9 Hz, 1H), 7.59 (dd, J = 8.4, 1.7 Hz, 1H), 3.66 (s, 3H), |
| | methylphenyl)(methyl)(oxo)-λ⁶- | 2.45 (s, 3H), 2.35 (s, 3H); LCMS(M − 1): 506.9 |
| | sulfaneylidene)-5-methyl-1H-1,2,3- | |
| | triazole-4-carboxamide | |
| 805 | N-((2-chloro-5- | ¹H-NMR (400 MHz, DMSO-d₆) δ 8.00 (d, J = 1.5 Hz, |
| | methylphenyl)(methyl)(oxo)-λ⁶- | 1H), 7.82 (td, J = 8.8, 6.0 Hz, 1H), 7.74-7.68 (m, 1H), |
| | sulfaneylidene)-1-(2,4- | 7.63-7.57 (m, 2H), 7.42-7.37 (m, 1H), 3.65 (s, 3H), 2.45 |
| | difluorophenyl)-5-methyl-1H-1,2,3- | (s, 3H), 2.39 (s, 3H); LCMS(M + 1): 424.9 |
| | triazole-4-carboxamide | |
| 806 | 1-(2,4-dichlorophenyl)-N-((2,5- | ¹H-NMR (400 MHz, CHLOROFORM-D) δ 9.70 (s, 1H), |
| | diisopropylphenyl)sulfonyl)-5- | 8.12 (d, J = 2.0 Hz, 1H), 7.64 (d, J = 2.0 Hz, 1H), 7.50- |
| | methyl-1H-1,2,3-triazole-4- | 7.47 (m, 2H), 7.44 (d, J = 8.1 Hz, 1H), 7.35 (d, J = 8.6 |
| | carboxamide | Hz, 1H), 3.96-3.89 (m, 1H), 3.05-2.98 (m, 1H), 2.39 (s, |
| | | 3H), 1.29 (dd, J = 9.3, 6.8 Hz, 12H); LCMS(M − 2): 495.00 |

* Compound names generated using Chemdraw Professional 19.1

As described herein the compounds of formula (I) show high nematicidal activity which is exerted with respect to nematodes which attack important agricultural crops. The compounds of the present invention were assessed for their activity against one or more of the following nematodes.

### Biological Examples

### Biological Test Examples for Plant Parasitic Nematodes

### Example 1

Meloidogyne incognita Test:

The trial pots were filled with soil containing a mixture of sand:soil:cocopeat in a ratio of 1:1:1. To the soil of each pot 5 mL of a nematode suspension (Meloidogyne incognita, mix of 5000 eggs & J2s) was added. One hour later the respective test compound at the stated concentration was applied to each pot. 10 days old cucumber seedlings were transplanted into the pots one day after the application. The cucumber plants were allowed to grow at a temperature of 27° C. under greenhouse conditions. The evaluation of the gall rating was recorded 30 days after the application. The plants were carefully uprooted and the roots were thoroughly washed. The gall rating was done on a 0-10 scale as described by Zeck (1971) and shown below:
0=no galls
1=very few small galls
2=numerous small galls
3=numerous small galls of which some are grown together
4=numerous small and some big galls
5=25% of roots severely galled
6=50% of roots severely galled
7=75% of roots severely galled
8=no healthy roots but plant is still green
9=roots rotting and plant dying
10=plant and roots dead

Compounds 2, 7, 8, 17, 25, 33, 35, 52, 56, 57, 61, 76, 96, 97, 108, 132, 133, 134, 135, 140, 143, 168, 171, 209, 210, 217, 218, 227, 228, 229, 230, 231, 240, 241, 242, 245, 246, 250, 252, 253, 254, 259, 260, 261, 269, 271, 272, 278, 279, 280, 281, 311, 315, 316, 328, 335, 336, 337, 338, 339, 364, 370, 371, 378, 389, 390, 394, 395, 396, 410, 413, 415, 430, 432, 433, 441, 443, 445, 446,458, 459, 460, 497, 499, 513, 516, 536, 538, 563, 586, 587, 588, 590, 596, 597, 598, 599, 610, 630, 631, 640, 657, 664, 665, 666, 667, 668, 669, 670, 671, 672, 673, 674, 675, 676, 683, 687, 689, 690, 691, 709, 736, 774 and 776 at 1000 ppm recorded less than 4 of the gall rating scale whereas there was extensive galling (up to 8) in the untreated check.

1. A compound of formula (I) 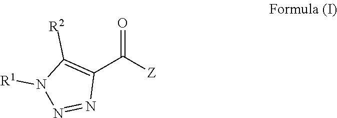
wherein,
R¹ is 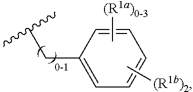
wherein [symbol] represents the point of attachment to the triazole ring;
or
R¹ is selected from the group consisting of C₁-C₆-haloalkyl, C₂-C₆-alkenyl, C₂-C₆-alkynyl, C₃-C₈-cycloalkyl, C₃-C₈-halocycloalkyl and C₃-C₈-cycloalkyl-C₁-C₆-alkyl; wherein said C₁-C₆-haloalkyl, C₂-C₆-alkenyl, C₂-C₆-alkynyl, C₃-C₈-cycloalkyl, C₃-C₈-halocycloalkyl and C₃-C₈-cycloalkyl-C₁-C₆-alkyl groups of R¹ may optionally be substituted by one or more groups selected from the group consisting of halogen, CN, R^{6a}, OR^{6a}, S(O)ₙR^{6a}, N(R^{6a})₂ and COOR^{6a};
R^{1a} is selected from the group consisting of hydrogen, halogen, CN, OR^{6a}, C₁-C₆-alkyl and C₁-C₆-haloalkyl; wherein each group of R^{1a} may optionally be substituted by one or more groups selected from the group consisting of halogen, CN, R^{6a}, OR^{6a}, S(O)ₙR^{6a}, N(R^{6a})₂, COOR^{6a} and CONR^{6a};
R^{1b} is selected from the group consisting of halogen, CN, C₁-C₆-alkyl, C₁-C₆-haloalkyl, C₂-C₆-alkenyl, C₂-C₆-alkynyl, C₁-C₆-alkoxy, C₁-C₆-haloalkoxy, C₃-C₈-cycloalkyl, C₃-C₈-halocycloalkyl, S(O)ₙR^{6a} and C₃-C₈-cycloalkyl-C₁-C₆-alkyl; wherein each group of R^{1b} may optionally be substituted by one or more groups selected from the group consisting of halogen, R^{6a}, OR^{6a}, S(O)ₙR^{6a}, N(R^{6a})₂, COOR^{6a} and CONR^{6a};
R² is selected from the group consisting of hydrogen, halogen, CN, C₁-C₆-alkyl, C₁-C₆-haloalkyl, C₁-C₆-alkoxy, C₃-C₆-cycloalkyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
Z is Z-1 or Z-2; 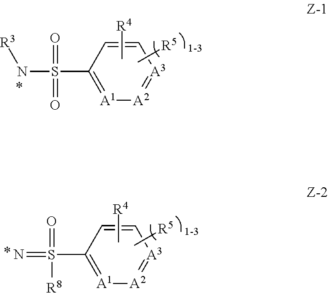
* represents the point of attachment to the carbonyl group attached to the triazole ring;
R³ is selected from the group consisting of hydrogen, C₁-C₆-alkyl, C₁-C₆-haloalkyl, C₁-C₆-alkoxy, C₃-C₆-cycloalkyl and C₃-C₈-cycloalkyl-C₁-C₆-alkyl; wherein each group of R³ may optionally be substituted with halogen;
A¹, A² and A³ represent C or N; wherein not more than two of A¹, A² and A³ represent N;
R⁴ is selected from the group consisting of hydrogen, halogen, CN, OR⁶, C₁-C₆-alkyl, C₁-C₆-haloalkyl, C₂-C₆-alkenyl and C₂-C₆-alkynyl;
R⁵ is selected from the group consisting of hydrogen, halogen, CN, nitro, (C═O)—R⁶, O(C═O)—R⁶, OR⁶, N(R⁶)₂, CR⁶═NR⁶, COOR⁶, CON(R⁶)₂, (C═S)—N(R⁶)₂, S(O)ₙR⁶, OS(O)ₙR⁶, S(O)ₙN(R⁶)₂, —S(═O)₀₋₁R⁷(═N—R⁶), —N═S(═O)₀₋₁(R⁷)₂, C₁-C₆-alkyl, C₁-C₆-haloalkyl, C₂-C₆-alkenyl, C₂-C₆-alkynyl, C₃-C₈-cycloalkyl, C₃-C₈-halocycloalkyl, C₃-C₈-cycloalkyl-C₁-C₆-alkyl, phenyl, C₃-C₆-carbocyclyl and a heterocyclic 3 to 6-membered ring; wherein each group of R⁵ may optionally be substituted by one or more groups selected from the group consisting of halogen, R^{6a}, OR^{6a}, S(O)ₙR^{6a}, N(R^{6a})₂, COOR^{6a} and CONR^{6a};
R⁶ is selected from the group consisting of hydrogen, hydroxy, C₁-C₆-alkyl, C₁-C₆-alkoxy, C₁-C₆-haloalkyl, C₂-C₆-alkenyl, C₂-C₆-alkynyl, C₃-C₈-cycloalkyl and C₃-C₈-cycloalkyl-C₁-C₆-alkyl;
R^{6a} is selected from the group consisting of hydrogen, C₁-C₆-alkyl, C₁-C₆-haloalkyl and C₃-C₈-cycloalkyl;
R⁷ and R⁸ are independently selected from the group consisting of hydrogen, C₁-C₆-alkyl, C₁-C₆-haloalkyl, C₂-C₆-alkenyl and C₃-C₈-cycloalkyl and C₃-C₈-cycloalkyl-C₁-C₆-alkyl;
n is an integer selected from 0 to 2;
or salts, stereoisomers, polymorphs or N-oxides thereof; with the proviso that the following compounds are excluded from the definition of compounds of formula (I): 1H-1,2,3-Triazole-4-carboxamide, 1-(2-cyclopropylethyl)-N-[(2,3-difluorophenyl)sulfonyl], 1H-1,2,3-Triazole-4-carboxamide, N-[(4-bromo-2-chlorophenyl)sulfonyl]-1-(2-cyclopropylethyl), 1H-1,2,3-Triazole-4-carboxamide, 1-(2-cyclopropylethyl)-N-[[3-[(dimethylamino)carbonyl]phenyl]sulfonyl], 1H-1,2,3-Triazole-4-carboxamide, 1-cyclohexyl-N-[[3-(4-methyl-2-thiazolyl)phenyl]sulfonyl] and 1H-1,2,3-Triazole-4-carboxamide, 1-cyclopropyl-5-(difluoromethyl)-N-[[4-methyl-3-(trifluoromethyl) phenyl]sulfonyl].

2. The compound of formula (I) according to claim 1, wherein R¹ is 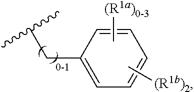
wherein [symbol] represents the point of attachment to the triazole ring.

3. The compound of formula (I) according to claim 1, wherein Z is Z-1.

4. The compound of formula (I) according to claim 1, wherein Z is Z-2.

5. The compound of formula (I) according to claim 1, wherein R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, methyl, ethyl, propyl, isopropyl, methoxy, ethoxy, difluoromethyl, and trifluoromethyl.

6. The compound of formula (I) according to claim 1, wherein said compound of formula (I) is represented by formula (Ia-1), 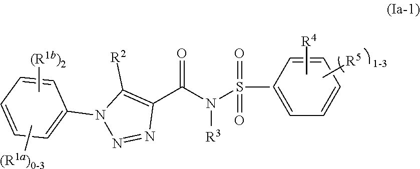
wherein,
R^{1a} is selected from the group consisting of hydrogen, halogen, CN, OR^{6a}, C₁-C₄-alkyl and C₁-C₄-haloalkyl; wherein each group of R^{1a} may optionally be substituted by one or more groups selected from the group consisting of halogen, CN and R^{6a};
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
R³ is selected from the group consisting of hydrogen, C₁-C₄-alkyl, C₁-C₄-haloalkyl and C₃-C₈-cycloalkyl-C₁-C₄-alkyl;
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, (C═O)—R⁶, OR⁶, N(R⁶)₂, S(O)ₙR⁶, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl, C₃-C₆-carbocyclyl and a heterocyclic 3 to 6-membered ring; wherein each group of R⁵ may optionally be substituted by one or more groups selected from the group consisting of halogen, R^{6a};
R^{6a} is selected from the group consisting of hydrogen, C₁-C₄-alkyl, C₁-C₄-haloalkyl and C₃-C₆-cycloalkyl;
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl and C₃-C₆-cycloalkyl and C₃-C₆-cycloalkyl-C₁-C₄-alkyl;
n is an integer selected from 0 to 2;
or salts, stereoisomers, polymorphs or N-oxides thereof.

7. The compound of formula (I) according to claim 1, wherein said compound of formula (I) is represented by formula (Ia-2), 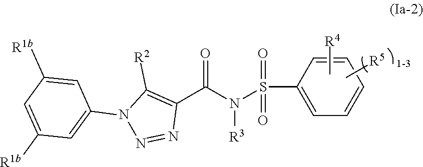
wherein,
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
R³ is selected from the group consisting of hydrogen, C₁-C₄-alkyl, C₁-C₄-haloalkyl and C₃-C₈-cycloalkyl-C₁-C₄-alkyl;
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, (C═O)—R⁶, OR⁶, N(R⁶)₂, S(O)ₙR⁶, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl, C₃-C₆-carbocyclyl and heterocyclic 3 to 6-membered ring; wherein each group of R⁵ may optionally be substituted by one or more groups selected from the group consisting of halogen, R^{6a};
R^{6a} is selected from the group consisting of hydrogen, C₁-C₄-alkyl, C₁-C₄-haloalkyl and C₃-C₆-cycloalkyl;
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl and C₃-C₆-cycloalkyl and C₃-C₆-cycloalkyl-C₁-C₄-alkyl;
n is an integer selected from 0 to 2;
or salts, stereoisomers, polymorphs or N-oxides thereof.

8. The compound of formula (I) according to claim 1, wherein said compound of formula (I) is represented by formula (Ia-3), 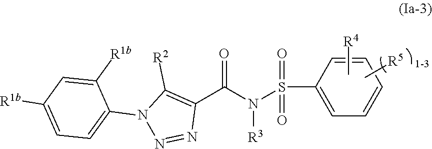
wherein,
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R² is selected from the group consisting of hydrogen, halogen, CN, C₁-C₄-alkyl, C₁-C₄-haloalkyl, C₁-C₄-alkoxy, cyclopropyl and phenyl; wherein said phenyl ring may optionally be substituted by one or more groups selected from the group consisting of halogen, CN, C₁-C₄-alkyl, C₁-C₄-alkoxy and C₁-C₄-haloalkyl;
R³ is selected from the group consisting of hydrogen, C₁-C₄-alkyl, C₁-C₄-haloalkyl and C₃-C₈-cycloalkyl-C₁-C₄-alkyl;
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, (C═O)—R⁶, OR⁶, N(R⁶)₂, S(O)ₙR⁶, C₁-C₄-alkyl, C₁-C₄-haloalkyl, C₃-C₆-cycloalkyl, C₃-C₆-halocycloalkyl, C₃-C₆-cycloalkyl-C₁-C₄-alkyl, phenyl, C₃-C₆-carbocyclyl and heterocyclic 3 to 6-membered ring; wherein each group of R⁵ may optionally be substituted by one or more groups selected from the group consisting of halogen, R^{6a};
R^{6a} is selected from the group consisting of hydrogen, C₁-C₄-alkyl, C₁-C₄-haloalkyl and C₃-C₆-cycloalkyl;
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl and C₃-C₆-cycloalkyl and C₃-C₆-cycloalkyl-C₁-C₄-alkyl;
n is an integer selected from 0 to 2;
or salts, stereoisomers, polymorphs or N-oxides thereof.

9. The compound of formula (I) according to claim 1, wherein,
R^{1a} is selected from the group consisting of hydrogen, halogen, CN, OR^{6a}, C₁-C₄-alkyl and C₁-C₄-haloalkyl; wherein each group of R^{1a} may optionally be substituted by one or more groups selected from the group consisting of halogen, CN and R^{6a};
R^{1b} is selected from the group consisting of halogen, CN, C₁-C₄-alkyl, C₁-C₄-haloalkyl, C₂-C₄-alkenyl, C₂-C₄-alkynyl, C₁-C₄-alkoxy, C₁-C₄-haloalkoxy, C₃-C₆-cycloalkyl, C₃-C₆-halocycloalkyl and C₃-C₆-cycloalkyl-C₁-C₄-alkyl; wherein each group of R^{1b} may optionally be substituted by one or more groups selected from the group consisting of halogen, R^{6a}, OR^{6a} and S(O)ₙR^{6a};
R⁴ is selected from the group consisting of hydrogen, halogen, CN, OR⁶, C₁-C₄-alkyl and C₁-C₄-haloalkyl;
R⁵ is selected from the group consisting of hydrogen, halogen, CN, nitro, OR⁶, C₁-C₄-alkyl, C₁-C₄-haloalkyl, C₃-C₆-cycloalkyl, C₃-C₆-cycloalkyl-C₁-C₄-alkyl and C₃-C₆-carbocyclyl; wherein each group of R⁵ may optionally be substituted by one or more groups selected from the group consisting of halogen and R^{6a};
R⁶ is selected from the group consisting of hydrogen, hydroxy, C₁-C₄-alkyl, C₁-C₄-alkoxy, C₁-C₄-haloalkyl, C₂-C₄-alkenyl, C₂-C₄-alkynyl, C₃-C₆-cycloalkyl and C₃-C₆-cycloalkyl-C₁-C₄-alkyl;
R^{6a} is selected from the group consisting of hydrogen, C₁-C₄-alkyl, C₁-C₄-haloalkyl and C₃-C₆-cycloalkyl.

10. A process for preparing a compound of formula (I) according to claim 1, wherein said method comprises the steps selected from a) to d):
reacting a compound of formula (11) with a compound of formula (9), in the presence of a suitable base and a suitable solvent, to obtain a compound of formula (13), which on further reaction with a hydrolyzing reagent in the presence of a suitable solvent provides a compound of formula (4y): 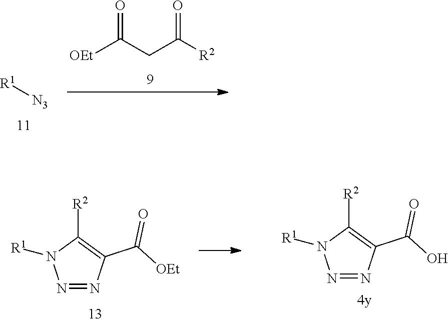
reacting a compound of formula (4y) with a compound of formula (2) in the presence of a suitable reagent, a suitable solvent and a suitable base to obtain a compound of formula (Ib-b): 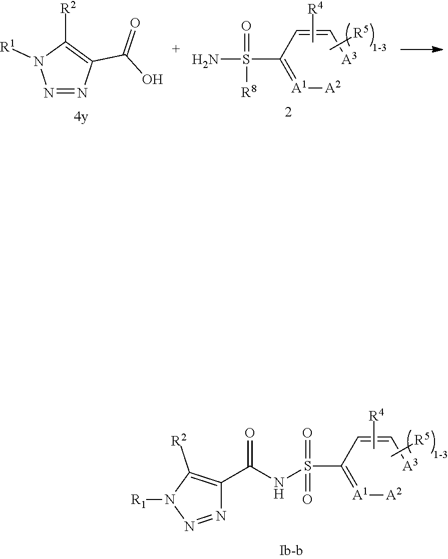
a compound of formula (Ib) is obtained by reacting a compound of formula (Ib-b) with a compound of formula (14), in the presence of a suitable solvent and a suitable base: 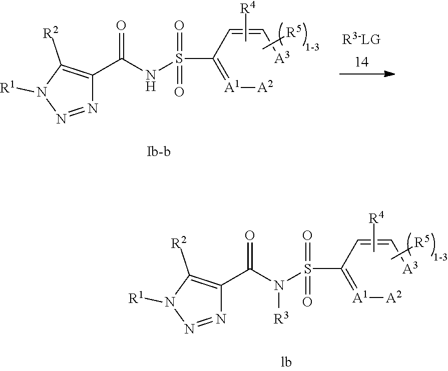
a compound of formula (Id) is obtained by reacting a compound of formula (4y) with a compound of formula (15) in the presence of a suitable coupling reagent, a suitable solvent and a suitable base:

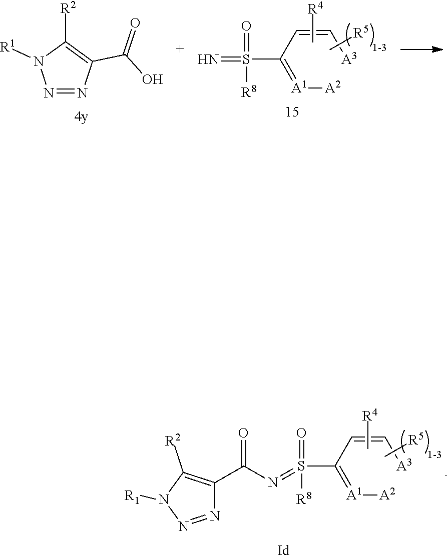

11. A composition comprising a compound of formula (I) according to claim 1 and one or more inert carriers.

12. The composition according to claim 11, wherein said composition may additionally comprises one or more active compatible compounds selected from fungicides, insecticides, nematicides, acaricides, biopesticides, herbicides, plant growth regulators, or fertilizers.

13. The composition according to claim 11, wherein the concentration of the compound of formula (I) ranges from 1 to 90% by weight with respect to the total weight of the composition, preferably from 5 to 50% by weight with respect to the total weight of the composition.

14. A combination comprising the compound of formula (I) according to claim 1 and one or more active compatible compounds selected from fungicides, insecticides, nematicides, acaricides, biopesticides, herbicides, plant growth regulators and fertilizers.

15. A method for controlling or preventing agricultural crops and/or horticultural crops against nematodes comprising applying the compound of formula (I) of claim 1 to the agricultural crops and/or horticultural crops.

16. The method according to claim 15, wherein said agricultural crops are selected from cereals, corn, rice, soybean and other leguminous plants, fruits and fruit trees, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers, other vegetables and ornamentals.

17. A seed comprising a compound of formula (I) according to claim 1, wherein the amount of the compound of the formula (I) is ranging from 0.1 g to 10 kg per 100 kg of seed.

18. A method of controlling or preventing an infestation of useful plants by phytopathogenic nematodes in agricultural crops and/or horticultural crops, wherein the composition according to claim 11 is applied to the plants, to parts thereof or a locus thereof.

19. A method of controlling or preventing an infestation of useful plants by phytopathogenic nematodes in agricultural crops and/or horticultural crops, wherein the combination according to claim 14 is applied to the seeds of the plants.

20. A method of controlling or preventing phytopathogenic nematodes in agricultural crops and or horticultural crops using the compound of formula (I) according to claim 1 comprising the step of applying an effective dosage of the compound, in amounts ranging from 1 g to 5 kg per hectare of agricultural crops and/or horticultural crops.
